SEQUENCE LISTING (151 sequences)

SEQ ID NO: 1 - protein, 535 aa, Oryza sativa
MEVAAMEISTSLLLTTVALSVIVCYALVFSRAGKARAPLPLPPGPRGWPVLGNLPQLGGKTHQTLHEMTKVYGPLIRLRFGSSDVVVAGSAPVAAQFLRTHDANFSSRPRNSGGEHMAYNGRDVVFGPYGPRWRAMRKICAVNLFSARALDDLRAFREREAVLMVRSLAEASAAPGSSSPAAVVLGKEVNVCTTNALSRAAVGRRVFAAGAGEGAREFKEIVLEVMEVGGVLNVGDFVPALRWLDPQGVVARMKKLHHRFDDMMNAIIAERRAGSLLKPTDSREEGKDLLGLLLAMVQEQEWLAAGEDDRITDTEIKALILNLFVAGTDTTSTIVEWTMAELIRHPDILKQAQEELDVVVGRDRLLLESDLSHLTFFHAIIKETFRLHPSTPLSLPRMASEECEIAGYRIPKGAELLVNVWGIARDPAIWPDPLEYKPSRFLPGGTHTDVDVKGNDFGLIPFGAGRRICAGLSWGLRMVTMTAATLVHAFDWQLPADQTPDKLNMDEAFTLLLQRAEPLVVHPVPRLLPSAYNIA

SEQ ID NO: 2 - DNA, 1608 bp, Oryza sativa
atggaggtcgccgccatggagatctctacctcattgctcctcaccaccgtggctctctccgtcatcgtgtgctacgccctggtcttctcccgcgccgggaaggcgcgtgcgccgctgccgctgccgcctggccccaggggatggccggtgctgggcaacctgccgcagctgggcgggaagacgcaccagacgctgcacgagatgaccaaggtgtacggcccgctgatccggctccggttcgggagctccgacgtggtggtcgccggctcggcgccggtggcggcgcagttcctccgcacccacgatgccaacttcagcagccggccacgcaactccggcggcgagcacatggcgtacaacggccgggacgtcgtgttcgggccgtacgggccgcggtggcgcgccatgcggaagatttgcgccgtcaacctcttctccgcgcgcgcgctcgacgacctgcgcgctttccgggagcgggaggccgtgctgatggttaggtcgctggcggaggcgagcgccgcccctgggtcgtcgtctccagcggcggtggtcctgggaaaggaggtgaatgtctgcacgacgaacgcgctgtcgcgcgccgcggtcgggcgccgcgtgttcgccgccggcgcgggcgagggcgcgagggagttcaaagagatcgtgctggaggtgatggaggtgggtggtgtgctgaacgtcggcgacttcgtgccggcgctccggtggctggacccgcagggcgtggtagcgaggatgaaaaagctgcaccaccggttcgacgacatgatgaacgcgatcatcgcggagaggagggccggatcactactcaaaccaaccgacagtcgtgaggaaggtaaggacttgcttggcttgctcctggctatggtgcaggagcaggagtggctcgccgccggcgaggacgacaggatcaccgacacggaaatcaaggcccttatcctgaatctattcgtggcgggcacagacacaacatcaaccatagttgagtggacaatggcagagctgattcgacacccagatatcctcaagcaggcccaagaggagctagatgttgttgtgggtcgtgataggctcctcttagagtcggatctatcacatctcaccttcttccatgctatcatcaaggagacattccgtcttcatccatcaaccccgctctcgctgccacgcatggcatctgaggagtgtgagatcgcaggctaccgtatccccaagggtgcagagttgctggtcaatgtgtgggggatcgcccgtgacccagccatatggcctgacccactagagtacaagccctctcggttcctccccggtgggacgcacactgatgtggatgtcaagggaaatgatttcggacttataccattcggtgcagggcgaaggatatgcgccggcctcagttggggcctgcggatggtcaccatgacagcggccacgctggtgcatgcattcgactggcagctaccagcggaccagacgccagacaagctcaatatggatgaggcgtttaccctcctgctgcaaagggcagagccattggtggttcacccggtaccaaggcttctcccatccgcttacaatattgcataa

SEQ ID NO: 3 - protein, 535 aa, Oryza sativa; identical to SEQ ID NO: 1

SEQ ID NO: 4 - DNA, 1608 bp, Oryza sativa; identical to SEQ ID NO: 2

SEQ ID NO: 5 - protein, 535 aa, Oryza sativa
MEVAAMEISTSLLLTTVALSVIVCYALVFSRAGKARAPLPLPPGPRGWPVLGNLPQLGGKTHQTLHEMTKVYGPLIRLRFGSSDVVVAGSAPVAAQFLRTHDANFSSRPRNSGGEHMAYNGRDVVFGPYGPRWRAMRKICAVNLFSARALDDLRAFREREAVLMVRSLAEASAAPGSSSPAAVVLGKEVNVCTTNALSRAAVGRRVFAAGAGEGAREFKEIVLEVMEVGGVLNVGDFVPALRWLDPQGVVARMKKLHRRFDDMMNAIIAERRAGSLLKPTDSREEGKDLLGLLLAMVQEQEWLAAGEDDRITDTEIKALILNLFVAGTDTTSTIVEWTMAELIRHPDILKHAQEELDVVVGRDRLLSESDLSHLTFFHAIIKETFRLHPSTPLSLPRMASEECEIAGYRIPKGAELLVNVWGIARDPAIWPDPLEYKPSRFLPGGTHTDVDVKGNDFGLIPFGAGRRICAGLSWGLRMVTMTAATLVHAFDWQLPADQTPDKLNMDEAFTLLLQRAEPLVVHPVPRLLPSAYNIA

SEQ ID NO: 6 - DNA, 1608 bp, Oryza sativa
atggaggtcgccgccatggagatctctacctcattgctcctcaccaccgtggctctctccgtcatcgtgtgctacgccctggtcttctcccgcgccgggaaggcgcgtgcgccgctgccgctgccgcctggccccaggggatggccggtgctgggcaacctgccgcagctgggcgggaagacgcaccagacgctgcacgagatgaccaaggtgtacggcccgctgatccggctccggttcgggagctccgacgtggtggtcgccggctcggcgccggtggcggcgcagttcctccgcacccacgatgccaacttcagcagccggccacgcaactccggcggcgagcacatggcgtacaacggccgggacgtcgtgttcgggccgtacgggccgcggtggcgcgccatgcggaagatttgcgccgtcaacctcttctccgcgcgcgcgctcgacgacctgcgcgctttccgggagcgggaggccgtgctgatggttaggtcgctggcggaggcgagcgccgcccctgggtcgtcgtctccagcggcggtggtcctgggaaaggaggtgaatgtctgcacgacgaacgcgctgtcgcgcgccgcggtcgggcgccgcgtgttcgccgccggcgcgggcgagggcgcgagggagttcaaagagatcgtgctggaggtgatggaggtgggtggtgtgctgaacgtcggcgacttcgtgccggcgctccggtggctggacccgcagggcgtggtagcgaggatgaaaaagctgcaccgccggttcgacgacatgatgaacgcgatcatcgcggagaggagggccggatcactactcaaaccaaccgacagtcgtgaggaaggtaaggacttgcttggcttgctcctggctatggtgcaggagcaggagtggctcgccgccggcgaggacgacaggatcaccgacacggaaatcaaggcccttatcctgaatctattcgtggcgggcacagacacaacatcaaccatagttgagtggacaatggcagagctgattcgacacccagatatcctcaagcacgcccaagaggagctagatgttgttgtgggtcgtgataggctcctctcagagtcggatctatcacatctcaccttcttccatgctatcatcaaggagacattccgtctacatccatcaacaccgctctcgctgccacgcatggcatctgaggagtgtgagatcgcaggctaccgtatccccaagggtgcagagttgctggtcaatgtgtgggggatcgcccgtgacccagccatatggcctgacccactagagtacaagccctctcggttcctccccggtgggacgcacactgatgtggatgtcaagggaaatgatttcggacttataccattcggtgcagggcgaaggatatgcgccggcctcagttggggcctgcggatggtcaccatgacagcggccacgctggtgcatgcattcgactggcagctaccagcggaccagacgccagacaagctcaatatggatgaggcgtttaccctcctgctgcaaagggcagagccattggtggttcacccggtaccaaggcttctcccatccgcttacaatattgcataa

SEQ ID NO: 7 - protein, 526 aa, Oryza sativa
MDVVPLPLLLGSLAVSAAVWYLVYFLRGGSGGDAARKRRPLPPGPRGWPVLGNLPQLGDKPHHTMCALARQYGPLFRLRFGCAEVVVAASAPVAAQFLRGHDANFSNRPPNSGAEHVAYNYQDLVFAPYGARWRALRKLCALHLFSAKALDDLRAVREGEVALMVRNLARQQAASVALGQEANVCATNTLARATIGHRVFAVDGGEGAREFKEMVVELMQLAGVFNVGDFVPALRWLDPQGVVAKMKRLHRRYDNMMNGFINERKAGAQPDGVAAGEHGNDLLSVLLARMQEEQKLDGDGEKITETDIKALLLNLFTAGTDTTSSTVEWALAELIRHPDVLKEAQHELDTVVGRGRLVSESDLPRLPYLTAVIKETFRLHPSTPLSLPREAAEECEVDGYRIPKGATLLVNVWAIARDPTQWPDPLQYQPSRFLPGRMHADVDVKGADFGLIPFGAGRRICAGLSWGLRMVTLMTATLVHGFDWTLANGATPDKLNMEEAYGLTLQRAVPLMVQPVPRLLPSAYGV

SEQ ID NO: 8 - DNA, 1863 bp, Oryza sativa
aaacccgcatttcccatcgtacaacgagcgagcggatcatacggtcatggacgttgtgcctctcccgctgctgctcggctccctggccgtgtccgccgccgtgtggtaccttgtgtacttcctccgcggcggcagcggcggcgacgcggcgaggaagcggcggcctttgccacccgggccacgcgggtggcccgtgctgggcaacctgccgcagctcggcgacaagccgcaccacaccatgtgcgccctggcgcggcagtacggcccgctgttccggctccggttcggctgcgccgaggtggtggtggccgcgtcggcgcccgtggctgcgcagttcctgcgcgggcacgatgccaacttcagcaaccgcccgcccaactcgggcgccgagcacgtcgcgtacaactaccaggacctcgtcttcgcgccctacggtgctcgctggcgcgccctgcggaagctgtgcgcgctccacctcttctcggccaaggcgctcgacgacctccgagcagtccgggagggcgaggtcgcgctcatggtgaggaacctcgctcggcagcaggcggcgtcagtggcgctggggcaggaagcgaacgtctgcgccacgaacacgctggcccgcgccaccatcggtcaccgggtgttcgccgtcgacggcggggaaggcgcaagggagttcaaggagatggttgtggagctgatgcagctcgccggcgttttcaacgtcggggacttcgtgccggcgctccggtggctcgacccgcagggcgtcgtggcaaagatgaagaggctgcaccgtcggtacgacaacatgatgaacggattcatcaacgaaaggaaggccggggcgcagcccgacggggtcgccgctggcgagcacggcaacgaccttctaagcgtgctgctggcgaggatgcaggaggagcagaagctggacggcgacggcgaaaagatcaccgaaactgacatcaaagctctgctcctgaacctattcactgcggggacggatacgacatcgagcacggtggagtgggcactggcggagctgatccggcacccggacgtcctcaaggaggcccagcatgagcttgacaccgtcgtcggtaggggtcgtctcgtgtccgagtctgaccttccacgcctcccctacctcaccgcggtgatcaaggagacgtttcggcttcacccgtcaacgccgctctcactgcctcgggaggctgcagaggagtgtgaggtggacggctaccgtatccccaagggcgctaccctcctagtcaacgtctgggctatagcccgtgacccgacccaatggcccgacccgctacagtaccagccttctcggtttctccccggcaggatgcatgcagacgtggatgtcaagggtgctgatttcggcctgataccattcggagcaggacggagaatatgcgctggccttagttggggcttgcggatggtcacactgatgactgccacgctagtgcacgggttcgactggaccttggctaacggcgcgactccggacaagctcaacatggaggaggcctatgggctcaccttgcagagggccgtgccgttgatggtccagcccgtgccaaggctgcttccatcggcttatggagtataaaaccggtctacttactagtaccactttaaattaaggtcagaaatcggtggagactacttgcagtgttggccgcattatatgacgtattattttgttttgtttgttggtggaaaaataaagtagtctatctcagtgttatctggcactaaaggaactctagaaatggtggcaaaatagagtactatcgtggaatcataaaaaaggattatttggtgtataatacagaaaaatttatg

SEQ ID NO: 9 - DNA, 1975 bp, Oryza sativa
taaacccgcatttcccatcgtacaacgagcgagcggatcatacggtcatggacgttgtgcctctcccgctgctgctcggctccctggccgtgtccgccgccgtgtggtaccttgtgtacttcctccgcggcggcagcggcggcgacgcggcgaggaagcggcggcctttgccacccgggccacgcgggtggcccgtgctgggcaacctgccgcagctcggcgacaagccgcaccacaccatgtgcgccctggcgcggcagtacggcccgctgttccggctccggttcggctgcgccgaggtggtggtggccgcgtcggcgcccgtggctgcgcagttcctgcgcgggcacgatgccaacttcagcaaccgcccgcccaactcgggcgccgagcacgtcgcgtacaactaccaggacctcgtcttcgcgccctacggtgctcgctggcgcgccctgcggaagctgtgcgcgctccacctcttctcggccaaggcgctcgacgacctccgagcagtccgggagggcgaggtcgcgctcatggtgaggaacctcgctcggcagcaggcggcgtcagtggcgctggggcaggaagcgaacgtctgcgccacgaacacgctggcccgcgccaccatcggtcaccgggtgttcgccgtcgacggcggggaaggcgcaagggagttcaaggagatggttgtggagctgatgcagctcgccggcgttttcaacgtcggggacttcgtgccggcgctccggtggctcgacccgcagggcgtcgtggcaaagatgaagaggctgcaccgtcggtacgacaacatgatgaacggattcatcaacgaaaggaaggccggggcgcagcccgacggggtcgccgctggcgagcacggcaacgaccttctaagcgtgctgctggcgaggatgcaggaggagcagaagctggacggcgacggcgaaaagatcaccgaaactgacatcaaagctctgctcctggtaagttcctgatgaccgtgccttttcagattatcgcaacaccacttccatgttgacatgatctttcttctttctttttgtggatcgtgatagaacctattcactgcggggacggatacgacatcgagcacggtggagtgggcactggcggagctgatccggcacccggacgtcctcaaggaggcccagcatgagcttgacaccgtcgtcggtaggggtcgtctcgtgtccgagtctgaccttccacgcctcccctacctcaccgcggtgatcaaggagacgtttcggcttcacccgtcaacgccgctctcactgcctcgggaggctgcagaggagtgtgaggtggacggctaccgtatccccaagggcgctaccctcctagtcaacgtctgggctatagcccgtgacccgacccaatggcccgacccgctacagtaccagccttctcggtttctccccggcaggatgcatgcagacgtggatgtcaagggtgctgatttcggcctgataccattcggagcaggacggagaatatgcgctggccttagttggggcttgcggatggtcacactgatgactgccacgctagtgcacgggttcgactggaccttggctaacggcgcgactccggacaagctcaacatggaggaggcctatgggctcaccttgcagagggccgtgccgttgatggtccagcccgtgccaaggctgcttccatcggcttatggagtataaaaccggtctacttactagtaccactttaaattaaggtcagaaatcggtggagactacttgcagtgttggccgcattatatgacgtattattttgttttgtttgttggtggaaaaataaagtagtctatctcagtgttatctggcactaaaggaactctagaaatggtggcaaaatagagtactatcgtggaatcataaaaaaggattatttggtgtataatacagaaaaatttatgaacacgctggtatatatg

SEQ ID NO: 10 - DNA, 10082 bp, Oryza sativa
ggtggtaggtaagggatctcaggatgggacctggcacccatatccaccaaccactgttgtccctgagataatagacgcgtgctttgcagagtgatccaaagctagctagtcctaccaacaatggaggtcgccgccatggagatctctacctcattgctcctcaccaccgtggctctctccgtcatcgtgtgctacgccctggtcttctcccgcgccgggaaggcgcgtgcgccgctgccgctgccgcctggccccaggggatggccggtgctgggcaacctgccgcagctgggcgggaagacgcaccagacgctgcacgagatgaccaaggtgtacggcccgctgatccggctccggttcgggagctccgacgtggtggtcgccggctcggcgccggtggcggcgcagttcctccgcacccacgatgccaacttcagcagccggccacgcaactccggcggcgagcacatggcgtacaacggccgggacgtcgtgttcgggccgtacgggccgcggtggcgcgccatgcggaagatttgcgccgtcaacctcttctccgcgcgcgcgctcgacgacctgcgcgctttccgggagcgggaggccgtgctgatggttaggtcgctggcggaggcgagcgccgcccctgggtcgtcgtctccagcggcggtggtcctgggaaaggaggtgaatgtctgcacgacgaacgcgctgtcgcgcgccgcggtcgggcgccgcgtgttcgccgccggcgcgggcgagggcgcgagggagttcaaagagatcgtgctggaggtgatggaggtgggtggtgtgctgaacgtcggcgacttcgtgccggcgctccggtggctggacccgcagggcgtggtagcgaggatgaaaaagctgcaccgccggttcgacgacatgatgaacgcgatcatcgcggagaggagggccggatcactactcaaaccaaccgacagtcgtgaggaaggtaaggacttgcttggcttgctcctggctatggtgcaggagcaggagtggctcgccgccggcgaggacgacaggatcaccgacacggaaatcaaggcccttatcctggttcgtgattcatgctctgatttagtagatagacactcactcgttcattgcatattaactaagtagctataattttttaagaaaaataataaaatatattagtatataatatattactttacaaacatataaattaaattaaatttgatttttataaataacatatagattataagatccacgttaaatgagtctacatccactcaattattagaatctgtcccctgaacttttttactgttgtctgtctacgtcatgtccaaaacactagtaatgtttgtttctcctttgttgagaatctgtttttcccacgtcgatgtgctttctgttcggaatttggtttgggaatttggggatatgcgtcgttttgcgcaccagaagaccagaacacgtacttgtcgtcatcaccactcatttgggtaacagattatcaagtagactggttgtcgggcttccaatagtaaaatctaattcgagagccctcctgttttagcgctatcattcgacgctggcagagccgtctgatcgctcgcgtcattatcgagtccatctgcgtaggcctcgcagtctactggtaattcttacgatcacagataaaatccgcaagcgcacgggtatacagatgtagcacttcccctacggagtattccaaagggtatcgaatccaaggaaacatgtgtggtcagttcttcctccggttcatccaagaacaccaagcaaaggatagggcgggatagcgaggattcactggtgagaaatagtgtctaggaaagtttaagtttaatcctaacgtaatacttcaggcactggtaacccgctattcccagatgttgctctactacgtacccggacagggaagacttaagtgatctcgagggctgtcaccacctctacacctacctcaaacgtactgtgggatacacagtaattactggataacaattacctaaacaccacgtctaagcaattaatatctactttagtatttataactcaccaaagcaatctctatatttcagttgattatagtgaacgataatcccgtatgctatttaggaactaaccaagagataattctcacaagataaatctaaattactcaggaagaatattatattgaaatcagagtaatgaacaaaataaaagaaatgagagaagattaccgacaactccagaattcttccgacttcttctactctactctcttcctattctagtatacaatatagtacaatagagcctcttataatttagctcaatcttggaagtgtgtgtaagagtgaaggagtgaaactccttatatagaggtaggtatgactgttacacgatgcgaattgtcggaaatgccccgcaaccgccatcaggagatgatcaggaccatccacgccaaaccccagcctgaacggctgagattttggttcggccgaaccaaggggttcggccgaacgtgggctaggcccacctggcctggccttcggcccatctcctccgctggtcctcctttatccatttctcggagttttgagctgagtctttgatattttgatgatcacaacccatccttgtatgaatacacgtttctcctcactttagtctgattttactcccaacttcggggttcaacacctgcatacaaatgaacaccaacactagtggaatatgtgagattaaacacctatcgctatattgaatgtgttattatctggactttatgcagaggttggcggtatagaatcagcatttaacagccgccaacatagtcgtaggcatggctttctggataaggatcgagatgaaaccactccaaccacacgtcacacgcaaatcctgctttagagagtcacagagagagaggttacagtttctcccccatgtccttttcctcttcaaacaatgttttctctcaaattacctatccgatcaacaatccgattacaccattgtgttcgttacaattaaatcatcacaacaagctctgacatgattatattacgatgaaaaaatatttatatttataaattacttttattatatatgtaagttacttttatcatacatataagttgcttttagatttgactaaattacttttatatttgacatataaaagtaaatttaataaagtctgaaagtagtttacatatattataaaattaacttaaaacaaaacggaagtaactttgtcacgacattagaagtaaattcgttgtagggataaaaatataactttatctaaaattaaatttaattagcacaaatcaacacacgtaagtttaacaatttttaaaagtaacttcaatgttaattggaagtaacttttgcatggttcaagttgaaaggagatactagatggagtacaaactagctaccactagctgtgtagtcacgtccaatgaaaaaatgcattaattaaagttactttctttttgttatagttacttctataatatatttaaattacttttaggctttattgaatttacttttatatgtctaagaagtaatttagtgaaatctaaaaataatttagatatattataaaagtaatttattatttttcatcaaaatataatcatgtgagatcttgttataaagatttaattgttacaaacacaacgatataatcggatcgtagatcagataactagtttaagagaaaattgtatttgaaatataggtggacaatgtctctcatatatggagtggtagctggtttagacaaatcagctaccacttgctgtgtagtcacgttcccaatcgtaaaatcctccttttccgtgggaataggataatgcaccacctaaattattttaatgctaccttactctaaacttcggatatccgcacggtcttcaaagcagtgacaaatctagaatttttattttggtgtgcccacacaatcctcaaaagccaaatcagagtcactcatatacacaaatacagtaaaagttctttttttaaaggaacacaacaagggagggccccattgctaaaagattattattaaaaaagaaagaaggttacaaagcaaattacaacaaaaggatccaatctctgactacatgttgatcactcactttcaacctaaacagcaaaaggaggaagtctcctttaagaagttgcctccaagaaaaaaacaaaggtaatctgttctcaaaaatgaaaccattcctttctttccaaatattccaagcccctaaaaggaacttttctataaagcattgcccattgtaaacatgtttagccttcatgatcatattgctcaaactgagagaactatcccacaagatgcctaaataagtgcaacaagtcaccgagaaagggcacttaaagaaaagatgcattaaagtgtcagtacatctctaccgacaaagaacacaaaacaaatcataatctggtggagcacagtgtttgtgatccagcataaattagtgttcaatctatccatgaaaacaagccagctaaatactttaaccttggagaaaaccttactcttccaaagccacacaaagtgctgagggggctgcaagtgcctaaaattcagagcataaaacttctgggaagtatatttcaaaccaccccaaatataggaccaaatatatggctatctcactctcctgagaaagatcaatagtgctaataaaatctgaaagagcctgaaactcagtataagcttggtgagacaaaggcagctgaaaattgtctgacatttgagctgacagatagccatgcacaaatgatattttgttggcagcaaatgaataaagtctaggcattgagaaactgagagggggattatcagaccaaatatcctcccaaaaagaacatgttaaaccattccccacattgcatctagcaatccctctatagaaatccatcagcttataaatatctctacaccaaaaagaccctttagagatgacaaatgaggggccacttgatcataataagaggaccagataagatcaacccatggcacattccttctattatagaatttgtccaaaaatttcaccaaaagagcttgattccgaatagaaaggttgataacaccaagccccccttttactttaggtttacaaactttgtcccaagctgctaaattttctgcttgaattcacatatgaacatctccaaagacaatgtttccttgcactatcaatattatcaattactgtcttaggcaacattatagtgcacatgtaattggttggcaaggaggagaaaactgaattaactagagtaagcctattaccatataacaagaaatctgagtttgcagacaatctcctttcaattccttcaactagaggagcaaaatcaaccactctaggcttagttgttcccaaaggcagaccaagataagtaaaggggagtgaaccaattttgcaaccagaaaccctagcaagcatctcatcttttccatcactcaagtttttagggattaaacatgatttttgatagttaacctttaatctagtaccttgagcaaaagattgaagcagaacttttagagtaaaaagctccttcccacaatccttaacatataaaagagtatcatctgcatattggaccactgggaagttattgtcctgactatagggcaatggcttggataacaaatacaaatggtgtgctatgtttattaaagattgcagaagatcagctcccacaacaaacaacggcggtgaaaggggtcttacaccccccctccccccccccccgcctacaatgaaaattctttctaggaactccattcagaagaactgaagagaagcagaagaaaagatcttctgaatccagtttaaccattttctaaaaaccccatagctttcatcaccaacaaaatagcttcatgttcaatagtatcaaaggctttctcaaagtccaactttaaaagaacgatttcccttcttgagtggtgacattgatgaagaaactcaaaattccaagcaaggcaatcctgaatagttctcccttttataaaactatattgatttggatgtattactgataggattaccatctgcaatctgccagccaacaacttcgtcaaaatcttcagagaaatccccataagagaaataggcctatagtgatttatagtctcaggacacaattttttaggcaccaaagtgatgaaagagtgattcaaaggattcaagtctagagagcctcaatgaaaatcagcgcataatttataataatcttgacatataatgggccagcatttcttaataaaaaggccattaaaaccatccgaatcaggagctctatcaatagggagattcttaataagcttatcaacctcattcttagaaaagggggcatccaggatttccaaaccgtcttgtgaagggattaaagaagggagatcaaaaggcatacattcaaaacaagaaattcccatcctcatcttaaaagcattccaagcagtatgggctttagcctcatgatcagagagggttgacccatccacatcaaccaaaatagctatagaattcttcctgtatgattcagttgccattgcatggaaaaaatttgtgttttcccctccaaatttagcccatctaatagtacaccttttcttccagtatgtttgcttatatttctaagtatgtaagcaacttagatctgacaaaaactctgaaattccattccaacacagtgagatctctatattcctcaatgatatcaaaaaaaaaaagatcacaacattacattttgtgatgagtttctgaagcttacatagattcctactccattttataagcccctttcttaaagctttgagtttagatgaaatatttctagcagcatccaaaaaactgccatgattagcccaaatagattggactaaatcaaaaaacctttcatgctcaacctataaattctcaaacctgaacacatttgatctaggaataacaactgcacaatacaggcggtatgatcagaagttgacctagctaaaggcttgaataaagtattaggaaaagaggaagaccaactagtagaagtgaaaaaccagtccaattgctcaagttgagggtgattttgcatgttactccaggtaaaggccatgcccttaatagggacttccactaaacccaaattactgatgacctcattaaaaagaaaaatatcattcatatccgctcctggcttatttctatttagaactgaacgatagaagttaaaatcaccaagcaacagccaaaaatcaccaacaggaatttggagactataaagccaagccaaaaaaattagacctttcaacaccagtacagggctcataaatagtaaccattgtccaagataaactagaatcattagaagtaaaagtgagctggacaacaaaactttcaattgtgatattcataacagagaagacataactattccagcaaaccaaaatacccccaaatgcacccctagaaggggaaaaaagcaaatttgtcaaaacgcttaggggtgaacttcctaatgaaggaatgatcaaaattacttctcttagtttcatgtaaacaaaaaaaaatgcacatccgctttcctctatcttattcctaatagccaaccacccatcatcagaatttatgcctcttacattctaacacagaacattccagtcctaaaagcaccattcattttaaacagtgatagtagtgtagatcatatcttacataacacaaacaaagaaatgaaacaatgttaatgaaacacttttttaagcttagaaatgcttgcacgcaccatcattaatgattcattgtaacttattgatttcacatttcacaaatatgcatcatccaaacaaagtaaaattgtaaaagctagaaaattagctgtgctacgagcctactaacttgcctgattaccccctatactggttaggataatggttataatagccgtaaccagcaaatcgagactaaagatctcgatctttagtaccggttgaaataacaagtactaaagatgcataccaagtcaaaaaactatatgtgggatgtgggactcaaacttacgatctctcaccccaatcctcacgtgcgttaccatcccacctagtacacacatctgacttagataaatatgctttctttttaatctaatcgtaaagacatctttagtaaaaatatcattagtccaagttagtattaccaacagagaataaagatcctccagcattatttttggttggtgataccaaccggtactaaagaagtatttttagtaccgattagtaacattaaccatgagtaaaactgtttctagggagttagacttttagaatcggtactaaagaatcttataccagttcttaatccaaccaagatattttttattttggataccacaataaaaagatcagttatatagtaaatgttggtgctacttgactggttggcggtgacaactaccgtcggccatagcgtgcctgcgtgtcgtgggctgcaccactacgctggactccatctcactgcccgttgtgcagcgctcgccgcttcgcatgtgccacaagacgagagatgagatacagagaaaaccctatacacgcacgtgcacgcccgatagattcgtgtgctggtggagtccagacaatgcagcggggccttgcactctatttcggcatttatttatttattgtgaccaggcaccaaccgatcatttaggcctattgctgcatactgctttttaaaattattttgcaaattttgggtatgctgccagtccataccgggaaggcaggaacccgtccgcccctctttgaagtactactaatacatgttacattatcatttttaataatatgtttataaaaatattgaaaacattgccacatgaatgtgtttatattataaatatattattatataccatttttcatatattaaattttagttatttttatatagacccatcctatgatgtcatatgtctttctgctgactatgcagaatctattcgtggcgggcacagacacaacatcaaccatagttgagtggacaatggcagagctgattcgacacccagatatcctcaagcacgcccaagaggagctagatgttgttgtgggtcgtgataggctcctctcagagtcggatctatcacatctcaccttcttccatgctatcatcaaggagacattccgtctacatccatcaacaccgctctcgctgccacgcatggcatctgaggagtgtgagatcgcaggctaccgtatccccaagggtgcagagttgctggtcaatgtgtgggggatcgcccgtgacccagccatatggcctgacccactagagtacaagccctctcggttcctccccggtgggacgcacactgatgtggatgtcaagggaaatgatttcggacttataccattcggtgcagggcgaaggatatgcgccggcctcagttggggcctgcggatggtcaccatgacagcggccacgctggtgcatgcattcgactggcagctaccagcggaccagacgccagacaagctcaatatggatgaggcgtttaccctcctgctgcaaagggcagagccattggtggttcacccggtaccaaggcttctcccatccgcttacaatattgcataaagatttacgagttgaatataattaacgaaaagttatttccgtgtgtgtggcatcaaataaatagagggtatgaacttttgtcatggtgttgcatcattgttgtatgttggtagattggtttttcacgagtatctatactccttataaaaaggagtagtggtgatgattctgctaccaccccactaccaactcttatctttttttaaggacatctaggattagtgggcccatatgtcattactctcaccaacttttattcttgtgaaaggttattaccgtgcgaataaatagtgggtttgaactgtcgttgtgttatatcattcgatgtatgttgattggttttgtttttcacaaggagtatacatatattaagggacagaataattgtcagtcgct

SEQ ID NO: 11 - DNA, 20 bp, Artificial Sequence
  Description of Artificial Sequence Synthetic oligonucleotide
tgcggcaggttgcccagcac

SEQ ID NO: 12 - DNA, 20 bp, Artificial Sequence
  Description of Artificial Sequence Synthetic oligonucleotide
ccgctgttccggctccggtt

SEQ ID NO: 13 - DNA, 20 bp, Artificial Sequence
  Description of Artificial Sequence Synthetic oligonucleotide
acttcgtgccggcgctccgg

SEQ ID NO: 14 - protein, 535 aa, Oryza sativa; identical to SEQ ID NO: 1

SEQ ID NO: 15 - protein, 526 aa, Oryza sativa; identical to SEQ ID NO: 7

SEQ ID NO: 16 - protein, 535 aa, Oryza sativa; identical to SEQ ID NO: 5

SEQ ID NO: 17 - protein, 526 aa, Oryza sativa; identical to SEQ ID NO: 7

SEQ ID NO: 18 - protein, 496 aa, Triticum aestivum
MDHSLLLLLASLAAVAVAAVWHLRSHGRRTKLPLPPGPRGWPVLGNLPQLGAMPHHTMAALARQHGPLFRLRFGSVEVVVTASAKVARSFLRAHDTNFSDRPPTSGAEHLAYNYQDLVFAPYGARWCALRKLCALHLFSARALDALRTIRQDEARLMVTHLLSSSSPAGVAVNLCAINVRATNALARAAIGGRMFGDGVGEGAREFKDMVVELMQLAGVLNIGDFVPALRWLDPQGVVAKMKRLHRRYDRMMDGFISERGQHAGEMEGNDLLSVMLATIRWQSPADAGEEDGIKFTEIDIKALLLNLFTAGTDTTSSTVEWALAELIRDPCILKQLQHELDGVETFRLHPATPLSLPRVAAEDCEVDGYHVSKGTTLIMNVWAIARDPASWGPDPLEFRPVRFLPGGLHESADVKGGDYELIPFGAGRRICAGLGWGLRMVTLMTATLVHAFDWSLVDGTMPEKLNMEEAYGQTLQRAVPLVVQPVPRLLSSAYTV

SEQ ID NO: 19 - protein, 521 aa, Triticum aestivum
MDHDLLLLLLASLVAVVAATVWHLRGHGSGARKPKLPLPPGPRGWPVLGNLPQLGDKPHHTMAALARHHGPLFRLRFGSAEVVVAASAKVAGSFLRAHDANFSDRPPNSGAEHVAYNYQDLVFAPYGARWRALRKLCAQHLFSARALDALRQVRQDEARLMVTRLLSSSDSPAGLAVGQEANVCATNALALAAVGRRVFGDGVGEGAREFKDMVVELMQLAGVFNIGDFVPALRWLDPQGVVGKMKRLHRRYDLMMDGFISERGDRADGDGNDLLSVMLGMMRQSPPAAGEEDGIKFNETDIKALLLNLFTAGTDTTSSTVEWALAELIRHPDVLKKLQHELDDVVGNGHLVTETDLPQLTFLAAVIKETFRLHPSTPLSLPRVAAEDCEVDGYRIPKDTTLLVNVWAIARDPASWGDDVLEFRPTRFLPGGLHESVDVKGGDYELIPFGAGRRICAGLSWGLRMVTLMTATLVHAFDWTLVDGMTPEKLDMEEAYGLTLQRAVPLMVQPVPRLLPSAYTM

SEQ ID NO: 20 - protein, 520 aa, Triticum aestivum
MDHDLLLLLLASLAAVAAAAVWHLRGAKSPKLPLPPGPRGWPVLGNLPQLGDKPHHTMAALARLHGPLFRLRFGSAEVVVAASAKVAAAFLRGHDANFSDRPPNSGAEHVAYNYQDLVFAPYGARWRALRKLCALHLFSARALDALRTVRQDETRLMVTRLLSSSSGSVSPAGLAVGQEANVCATNALARAAVGRRVFGDGVGEGAREFKDMVAELMQLAGVFNIGDFVPALRWLDPQGVVAKMKRLHRRYDRMMDGFISERGDRADGDGNDLLSVMLGMMRQSPPAAGEEDGIKFNETDIKALLLNLFTAGTDTTSSTVEWALAELIRHPNVLKKLQHELDDVVGNGRLVTESDLPQLTILAAVIKETFRLHPSTPLSLPRVTAEDCEVDGYRIPKDTTLLVNVWAIARDPASWGDDVLEFRPVRFLAGGSHETVDVKGGDYELIPFGAGRRICAGLSWGLRMVTLMTATLVHAFDWTLVDGMTPEKLDMEEAYGLTLQRAVPLMVQPVPRLLPSAYTV

SEQ ID NO: 21 - protein, 544 aa, Triticum aestivum
MPCARPTNKQTSPHPLPPSMTPAAMDHDLLLLLASLAAVIVAAVWHLRGHGSGARKPKLPLPPGPRGWPVLGNLPQLGDKPHHTMAALARLHGLLFRLRFGSAEVVVAASAKVAGSFLRAHDANFSDRPPNSGAEHVAYNYQDLVFAPYGARWRALRKLCAQHLFSARALDALRTVRQDEARLMVTRLLSSSDSPAGLAVGQEANVCATNALALAAVGRRVFGDGVGEGAREFKDMVVELMQLAGVFNIGDFVPALRWLDPQGVVAKMKRLHRRYDRMMDGFISERGDRADGDGNDLLSVMLGMMRQSPPAGGEEDGIKFNETDIKALLLNLFTAGTDTTSSTVEWALAELIRHPDVLKKLQHELDDVVGNGRLVTESDLPQLTFLAAVIKETFRLHPSTPLSLPRVAAEDCEVDGYRIPKDTTLLVNVWAIARDPASWGDDVLEFRPTRFLPGGSHESVDVKGGDYELIPFGAGRRICAGLSWGLRMVTLTTATLVHAFDWTLVDGMTPEKLDMEEAYGLTLQRAVPLMVQPVPRLLPSAYTM

SEQ ID NO: 22 - protein, 519 aa, Triticum aestivum
MDHDLLLLLASLAAVAVAAVCYLRSHGSGAKLPLPPGPRGWPVLGNLPQLGAKPHHTMAALARQHGPLFRLRFGSAELVVAASAKVAGSFLRAHDANFSDRPPNSGAEHVAYNYQDLVFAPYGARWRALRMLCALHLFSARALDALRSVRQDEARLMVTHLLSASSSPAQGVAIGQEANVCATNALARAAVGRRVVGDGVGESAREFKGMVVELMQLAGAFNIGDFVPALRWLDPQGVVAKMKHLHRRYDRIMDGFISEREHLAGEEEGKDLLSIMLAKMRQPLHADAGEDGIKFTETNIKALLLNLLTAGTDTTSSTVEWALAELIRHPDTLKQLQREVDDVVGTSRLVTEADLPRLTFLTAVIKETFRLHPSTPLSLPRVAAEDCEVDGYHVAKGTTLLVNVWAISRDPASWGADALEFRPARFLPGGSHETVDVKGGDYELIPFGAGRRMCAGLSWGLRIVTLMTATLVHAFDLSLVNGMTPDKLDMEEAYGLTLQRAVPLLVQPMPRLLPSAYAT

SEQ ID NO: 23 - protein, 521 aa, Triticum aestivum
MEIPLPLLLSTFAISVTICYVIIFFFRADKGRAPLPPGPRGWPVLGNLPQLGGKTHQTLHEMTRLYGPMLRLRFGSSLVVVAGSADVAKQFLRTHDAKFSSRPPNSGGEHMAYNYQDVVFAPYGPRWRAMRKVCAVNLFSARALDDLRAFREWEAALMVRCLADAAAAGMAVALAKTANVCTTNSLSRATVGLRVFDTAGSKLGAEEFNEIVLKLIEVGGVLNVGDFVPVLRWLDPQGVVAKMKKLHRRFDDMMNRIIAERRAGAFATTAGEEGGKDLLGLLLAMVQEDKSLTGAEENKITDTDVKALILNLLVAGTDTTSITVEWAMAELIRHPDIMKQAQEELDAVMGRERLVSESDLPRLTSLSAIIKETFRLHPSTPLSLPRMATEDCKVAGYCIPKGTELLVKVWGIARDPALWPDPLEFRPARFLPGGSHADVDVKGGDFGLIPFGAGRRICAGLSWGIRMVTVTTATLVHSFDWELPAGQTPDMEETFSLLLQLAVPLMVHPVPRLLPSAYQIA

SEQ ID NO: 24 - protein, 522 aa, Triticum aestivum
MEIPLPLLLSTFAISVTICYVIFFFFHADKGRAPLPPGPRGWPVLGNLPQLGGKTHRTLHEMTRLYGPMLRLRFGSSLVVVAGSADVAKQFLRTHDAKFSSRPPNSGGEHMAYNYQDVVFAPYGPRWRAMRKVCAVNLFSSRALDDLRGFREREAALMVRCLADSAATGGAVALAKAANVCTTNALSRATVGLRVFATAGSELGAEDFNEIVLKLIEVGGVLNVGDFVPALRWLDPQGVVAKMKKLHRRFDDMMNRIIAERRAGAIATKAGEEGGKDLLCLLLAMVQEDKSLTGGSEEDRMTDTDVKALILNLFVAGTDTTSITVEWAMAELIRHPDILKQAQKELDAVIGRDRLVLESDLPRLNFLNAIIKETFRLHPSTPLSLPRMATEECEVAGYRIPKGTELLVNVWGIARDPALWTDPLEFRPARFLPGGSHADIDIKGGDFGLIPFGAGRRICAGLSWGIRMVAVTTATLVHSFDWELPAGQMPDMEETFSLLLQLAVPLMVHPVPRLLPSAYQIA

SEQ ID NO: 25 - protein, 520 aa, Triticum aestivum
MEIPLPLLLSTFAISVTICYVILFFRADMGRAPLPPGPRGWPVLGNLPQLGGKTHKTLHEMARLYGPMLRLRFGSSLVVVAGSADVAKLFLRTHDAKFSSRPPNSGGEHMAYNYQDVVFAPYGPRWRAMRKVCAVNLFSARALDDLRAFREWEAALMVRCLADAAAAGMAVALGKAANVCTTNALSRATVGLRVFATAGSELGAEEFNEIVLKLIEVGGVLNVGDFVPALRWLDPQGVVAKMKKLHRRFDDMMNRIIAERRAGAFATTASEEGGKDLIGLLLAMVQEDKSLTGAEENKITDTEVKALILNLFVAGTDTTSITVEWAMAELIRHPDIMKQAQEELDAIVGRERLVSESDLPRLTFLSAIIKETFRLHPSTPLSLPRMTTEECEVAGYCIPKGTELLVNVWGIARDPALWPDPLEFRPARFLPGGSHADVDVKGGDFGLIPFGAGRRICAGLSWGIRMVAVTTATLVHSFDWELPAGQTPDMEERFSLLLQLAVPLMVHPVPRLLPSAYQIA

SEQ ID NO: 26 - protein, 218 aa, Triticum aestivum
MHSTCMQNLFVAGTDTTLIMVEWAMAELIRHPDTLKQAQEELDTIVGRERLISESHLPRLTFLSAVIKDTFRLHPSTPLLLLRMATEECETAGYRIPKGTELLVNVWGIAHDPALWPDSLEFRPAWFLPGGSHADVDVKGGDFGLIPFGAGRRICAGLSRGIRMVAVTTATLVHSFNWELPAGQTPDMEGTFSLLLQLAVPLMVHPVPRLLPSAYQIA

SEQ ID NO: 27 - protein, 544 aa, Triticum aestivum
MNTRAPAVLAYRSNATMHLVAMDIPLPLLLSTLAVAVGVCYVLATFFRADKGRAPLPPGPRGWPVLGNLPQLGGKTHQTMHEMSKVYGPVLRLRFGSSVVVVAGSAGAAEQFLRTHDAKFSSRPPNSGGEHMAYNYQDVVFAPYGPRWRAMRKVCAVNLFSARALDDLRGFREREAALMVRSLVDAAATGGVVAVGKAANVCTTNALSRAAVGLRVFAAAGAELGAKEFKEIVLEVMEVGGVLNVGDFVPALRWLDPQGVVARLKKLHRRFDDMMNGIIAERRAGGSTAGEEKEGKDLLGLLLAMVQEDKSLTGGEEDRITDTDVKALILNLFVAGTETTSTIVEWAVAELIRHPDMLKRAQEEMDAVVGRDRLVSESDLPRLTFLNAVIKETFRLHPSTPLSLPRMASEECEVAGYRIPKGTELLVNVWGIARDPALWPDPLEFRPARFLPGGTHADVDVKGGDFGLIPFGAGRRICAGLSWGLRVVTVTAATLVHSFDWELPAGQTPDKLNMEEAFSLLLQRAVPLMAHPVPRLLPSAYEIA

SEQ ID NO: 28 - protein, 528 aa, Triticum aestivum
MHLVAMGIPLPLLLSTLAIAVTICYVLATFFRADKGRAALPPGPRGWPVLGNLPQLGGKTHQTMHEMSKVYGPVLRLRFGSSVVVVAGSAAVAEQFLRTHDAKFSSRPPNSGGEHMAYNNQDVVFAPYGPRWRAMRKVCAVNLFSARALDDLRGFREREAALMVRSLVDAASGGGVVAVGKAANVCTTNALSRAAVGLRVFAAAGTELGAKEFKEIVLEVMEVGGVLNVGDFVPALRWLDPQGVVARLKKLHRRFDDMMNGIIAERRAGGSTAGEEKEGKDLLGLLLAMVQEDKSLTGGEEDRITDTDVKALILNLFVAGTETTSTIVEWAVAELIRHPDMLKRAQEEMDAVVGRDRLVSESDLPRLTFLNAVIKETFRLHPSTPLSLPRMASEECEVAGYRIPKGTELLVNVWGIARDPALWPDPLEFRPARFLPGGTHADVDVKGGDFGLIPFGAGRRICAGLSWGLRVVTVTAATLVHSFDWELPAGQTPDKLNMEEAFSLLLQRAVPLMVHPVPRLLPSAYQIA

SEQ ID NO: 29 - protein, 526 aa, Triticum aestivum
MHLVAMDIPLPLLLSTLAVAVTICYVLFFRADKGRAPLPPGPRGWPVLGNLPQLGGKTHQTMHEMSKVYGPVLRLRFGSSVVVVAGSAAVAEQFLRTHDAKFSSRPPNSGGEHMAYNYQDVVFAPYGPRWRAMRKVCAVNLFSARALDDLRGFREREAAFMVRSLADAASGGGLVAVGKAANVCTTNALSRAAVGLRVFAAAGTELGAKEFKEIVLEVMEVGGVLNVGDFVPALRWLDPQGVVARLKKLHRRFDDMMNGIIAERRAGAGTAGEEKEGKDLLGLLLAMVQEDKSLTGGEEDRITDTDVKALILNLFVAGTETTSTIVEWAVAELIRHPDMLKRAQEEMDAVVGRGRLVAESDLPRLTFLNAVIKETFRLHPSTPLSLPRMASEECEVAGYRIPKGTELLVNVWGIARDPALWPDPLEFRPARFLPGGTHADVDVKGGDFGLIPFGAGRRICAGLSWGLRVVTVTAATLVHSFDWELPTGQTPDKLNMEEAFSLLLQRAVPLMVHPVPRLLPSAYEIA

SEQ ID NO: 30 - protein, 535 aa, Zea mays
MELFVTTPDLPTPLLLSTLTIVSVVVCYVLFWKQQAAARRAPLPPGPRGWPVLGNLPQLGGKTHQTLHEMTKVYGPLLRLRFGSSTVVVAGSAAVAQQFLRAHDANFSSRPPNSGGELMAYNYQDVVFAPYGPRWRAMRKVCAVNLFSARALDDVRGVREREAALMVRSLAEQAHGGLDAPPAAVPVGKAINVCTTNALSRAAVGRRVFAAAGGDGGAREFKEIVLEVMQVGGVLNVGDFVPALRWLDPQGVAAKMKKLHRRFDDMMDEIIAGYREARRVAADGEESKDLLGLLLSMVDERPFDSGEEVRITETDVKALILNLFVAGTDTTSTIVEWSLAELIRHPEILRQAQEEMDAVAGRGRLVTESDLRSLTFFNAVIKETFRLHPSTPLSLPRMAAEECEVAGYRVPRGSELLVNVWGIARDPALWPDPLEFRPARFLPGGSHADVDVKGADFGLIPFGAGRRICAGLSWGLRMVTLTSATLVHAFDWELPAGQTPDKLNMEEAFTLLLQRAVPLVARPVPRLLPSAYEIA

SEQ ID NO: 31 - protein, 515 aa, Zea mays
MDVPLPLLLGSVAVSLVVWCLLLRRGGAGKGKRPLPPGPRGWPVLGNLPQVGAKPHHTMCAMAREYGPLFRLRFGSAEVVVAASARVAAQFLRAHDANFSNRPPNSGAEHVAYNYQDLVFAPYGSRWRALRKLCALHLFSAKALDDLRGVREGEVALMVRELARQGERGRAAVALGQVANVCATNTLARATVGRRVFAVDGGEGAREFKEMVVELMQLAGVFNVGDFVPALAWLDPQGVVGRMKRLHRRYDDMMNGIIRERKAAEEGKDLLSVLLARMREQQPLAEGDDTRFNETDIKALLLNLFTAGTDTTSSTVEWALAELIRHPDVLRKAQQELDAVVGRDRLVSESDLPRLTYLTAVIKETFRLHPSTPLSLPRVAAEECEVDGFRIPAGTTLLVNVWAIARDPEAWPEPLEFRPARFLPGGSHAGVDVKGSDFELIPFGAGRRICAGLSWGLRMVTLMTATLVHALDWDLADGMTADKLDMEEAYGLTLQRAVPLMVRPAPRLLPSAYAE

SEQ ID NO: 32 - protein, 855 aa, Zea mays
MDVPLPLLLGSLAVSVMVWCLVLRRGGDGKGKRPLPPGPRGWPVLGNLPQVGAKPHHTMCALAREYGPLFRLRFGSAEVVVAASARVAAQFLRAHDANFSNRPPNSGAEHVAYNYRDLVFAPYGSRWRALRKLCALHLFSAKALDDLRGVREGEVALMVRELARPRRGEGGRAAAVALGQVANVCATNTLARATVGRRVFAVDGGEGAREFKEMVVELMQLAGVFNVGDFVPALAWLDPQGVVGRMKRLHRRYDHMMNGIIRERKAAEEGKDLLSVLLARMRDQQQQPLAEGEDNRINETDVKALLLVSLLALTTSQRANGMDNCSGALRAVEESACDAEVVRPLSKLPNSSIGLYDSSFECDACGVGFVAELSGDYKHVTVNDTIEMLERMAHRGACGCEKNTGDGAGIMVALPHDFFKEVAKDAGIELPPLGEYAVAMFFMPTDEKRRKKGKAEFKKVAESLGHLYILRRLSIISVRASLNIKRGGERDFYMCSLSSRATVGMLLGVEDMHRFPVRSPWMDRGDLIRSPAARQIVSNYFSMFGPVQDVRIPYQQKRMFGFVTFVYAETVKVILSKGNPHFVCDARVLVKPYKEKGKVPGRFRKLQHTHHGGAEFVGCASPTGLLDSRDPYALLLLSGAQNLFTAGTDTTSSTVEWALAELIRHPDVLRKAQQELDAVVGRDRLVSESDLPRLTYLTAVIKETFRLHPSTPLSLPRVAAEECEVDGFRIPAGTTLLVNVWAIARDPEAWPEPLQFRPARFLPGGSHAGVDVKGSDFELIPFGAGRRICAGLSWGLRMVTLMTATLVHALEWDLADGVTAEKLDMEEAYGLTLQRAVPLMVRPAPRLLPSAYAAQ

SEQ ID NO: 33 - protein, 860 aa, Zea mays
MDVPLPLLLGSLAVSVMVWCLVLRRGGDGKGKRPLPPGPRGWPVLGNLPQVGAKPHHTMCALAREYGPLFRLRFGSAEVVVAASARVAAQFLRAHDANFSNRPPNSGAEHVAYNYRDLVFAPYGSRWRALRKLCALHLFSAKALDDLRGVREGEVALMVRELARPRRGEGGRAAAVALGQVANVCATNTLARATVGRRVFAVDGGEGAREFKEMVVELMQLAGVFNVGDFVPALAWLDPQGVVGRMKRLHRRYDHMMNGIIRERKAAEEGKDLLSVLLARMRDQQQQPLAEGEDNRINETDVKALLLASRVPNNHPAKRKPPPPPPPPPGRRRTSPTVAWKKEDKPRRRLEGGGQAPPPPPSHCQPLPTAPTPALQLPPRRMLPNALYNPGESRDGRMTVRVVMRYLVNKLGLEDDSQVNDTIEMLERMAHRGACGCEKNTGDGAGIMVALPHDFFKEVAKDAGIELPPLGEYAVAMFFMPTDEKRRKKGKAEFKKVGCRITGTWRQWACCSGVEDMHRFPVRSPWMDRGDLIRSPAARQIVSNYFSMFGPVQDVRIPYQQKRMFGFVTFVYAETVKVILSKGNPHFVCDARVLVKPYKEKGKVPGRFRKLQHTHHGGAEFVGCASPTGLLDSRDPYALLLLSGAQNLFTAGTDTTSSTVEWALAELIRHPDVLRKAQQELDAVVGRDRLVSESDLPRLTYLTAVIKETFRLHPSTPLSLPRVAAEECEVDGFRIPAGTTLLVNVWAIARDPEAWPEPLQFRPARFLPGGSHAGVDVKGSDFELIPFGAGRRICAGLSWGLRMVTLMTATLVHALEWDLADGVTAEKLDMEEAYGLTLQRAVPLMVRPAPRLLPSAYAAQ

SEQ ID NO: 34 - protein, 535 aa, Zea mays; identical to SEQ ID NO: 30

SEQ ID NO: 35 - protein, 515 aa, Zea mays; identical to SEQ ID NO: 31

SEQ ID NO: 36 - protein, 563 aa, Zea mays
MELFVTTPDLPTPLLLSTLTIVSVVVCYVLFWKQQAAARRAPLPPGPRGWPVLGNLPQLGGKTHQTLHEMTKVYGPLLRLRFGSSTVVVAGSAAVAQQFLRAHDANFSSRPPNSGGELMAYNYQDVVFAPYGPRWRAMRKVCAVNLFSARALDDVRGVREREAALMVRSLAEQAHGGLDAPPAAVPVGKAINVCTTNALSRAAVGRRVFAAAGGDGGAREFKEIVLEVMQVGGVLNVGDFVPALRWLDPQGVAAKMKKLHRRFDDMMDEIIAGYREARRVAADGEESKDLLGLLLSMVDERPFDSGEEVRITETDVKALILLTQQFRTKRISSFSLILSFMLARGHVRQNLFVAGTDTTSTIVEWSLAELIRHPEILRQAQEEMDAVAGRGRLVTESDLRSLTFFNAVIKETFRLHPSTPLSLPRMAAEECEVAGYRVPRGSELLVNVWGIARDPALWPDPLEFRPARFLPGGSHADVDVKGADFGLIPFGAGRRICAGLSWGLRMVTLTSATLVHAFDWELPAGQTPDKLNMEEAFTLLLQRAVPLVARPVPRLLPSAYEIA

SEQ ID NO: 37 - protein, 458 aa, Zea mays
  MOD_RES (134)..(141): Any amino acid
MDVPLPLLLGSLAVSVMVWCLVLRRGGDGKGKRPLPPGPRGWPVLGNLPQVGAKPHHTMCALAREYGPLFRLRFGSAEVVVAASARVAAQFLRAHDANFSNRPPNSGAEHVAYNYRDLVFAPYGSRWRALRKLXXXXXXXXDGGEGAREFKEMVVELMQLAGVFNVGDFVPALAWLDPQGVVGRMKRLHRRYDHMMNGIIRERKAAEEGKDLLSVLLARMRDQQQLAEGEDSRINETDVKALLLNLFTAGTDTTSSTVEWALAELIRHPDVLRKAQQELDAVVGRDRLVSESDLPRLTYLTAVIKETFRLHPSTPLSLPRVAAEECEVDGFRIPAGTTLLVNVWAIARDPEAWPEPLQFRPARFLPGGSHAGVDVKGSDFELIPFGAGRRICAGLTWGLRMVTLMTATLVHALDWDLADGVTAEKLDMEEAYGLTLQRAVPLMVRPAPRLLPSAYAAQ

SEQ ID NO: 38 - protein, 491 aa, Zea mays
MDVPLPLLLGSVAVSLVVWCLLLRRGGAGKGKRPLPPGPRGWPVLGNLPQVGAKPHHTMCALAREYGPLFRLRFGSAEVVVAASARVAAQFLRAHDANFSNRPPNSGAEHVAYNYQDLVFAPYGSRWRALRKLCALHLFSAKALDDLRGVREGEVALMVRELARQGERERAAVALGQVANVCATNTLARATVGRRVFAVDGGEGAREFKEMVVELMQLAGVFNVGDFVPALAWLDPQGVVGRMKRLHRRYDDMMNGIIRERKAAEEGKDLLSVLLARMREQQPLAEGDDTRFNETDIKALLLNLFTAGTDTTSSTVEWALAELIRHPDVLRKAQQELDAVVGRDRLVSESDLPRLTYLTAVIKETFRLHPSTPLSLPRVAAEECEAWPEPLEFRPGRFLPGGSHAGVDVKGSDFELIPFGAGRRICAGLSWGLRMVTLMTATLVHALDWDLADGMTADKLDMEEAYGLTLQRAVPLMVRPAPRLLPSAYAE

SEQ ID NO: 39 - protein, 535 aa, Zea mays
MELVLTTPDLPTPLLLSTLTIVSVVVCYVLFWKQQAAARRAPLPPGPRGWPVLGNLPQLGGKTHQTLHEMTKVYGPLLRLRFGSSTVVVAGSAAVAQQFLRAHDANFSSRPPNSGGELMAYNYQDVVFAPYGPRWRAMRKVCAVNLFSARALDDVRGVREREAALMVRSLAEQAHGGLDAPPAAVPVGKAINVCTTNALSRAAVGRRVFAAAAGDGGAREFKEIVLEVMQVGGVLNVGDFVPALRWLDPQGVAAKMKKLHRRFDDMMDEIIAGYREARRVAADGEESKDLLGLLLSMVDERPFDSGEEVRITETDVKALILNLFVAGTDTTSTIVEWSLAELIRHPEILRQAQEELDAVAGRGRLVSESDLRSLTFFNAVIKETFRLHPSTPLSLPRMAAEECEVAGYRVPRGSELLVNVWGIARDPALWPDPLEFRPARFLPGGSHADVDVKGADFGLIPFGAGRRICAGLSWGLRMVTLTSATLVHAFDWELPAGQTPDKLNMEEAFTLLLQRAVPLVARPVPRLLPSAYEIA

SEQ ID NO: 40 - protein, 118 aa, Triticum turgidum
MNVWAIARDPASWGPDPLEFRPVRFLPGGLHESADVKGGDYELIPFGAGRRICAGLGWGLRMVTLMTATLVHAFDWSLVDGTTPEKLNMEEAYGQTLQRAVPLVVQPVPRLLSSAYTV

SEQ ID NO: 41 - protein, 521 aa, Triticum turgidum
MDHDLLLLLLASLAAVVAATVWHLRGHGSGARKPKLPLPPGPRGWPVLGNLPQLGDKPHHTMAALARHHGPLFRLRFGSAEVVVAASAKVAGSFLRAHDANFSDRPPNSGAEHVAYNYQDLVFAPYGARWRALRKLCAQHLFSARALDALRQVRQDEARLMVTRLLSSSDSPAGLAVGQEANVCATNALALAAVGRRVFGDGVGEGAREFKDMVVELMQLAGVFNIGDFVPALRWLDPQGVVGKMKRLHRRYDLMMDGFISERGDRADGDGNDLLSVMLGMMRQSPPAAGEEDGIKFNETDIKALLLNLFTAGTDTTSSTVEWALAELIRHPDVLKKLQHELDDVVGNGHLVTETDLPQLTFLAAVIKETFRLHPSTPLSLPRVAAEDCEVDGYRIPKDTTLLVNVWAIARDPASWGDDVLEFRPTRFLPGGLHESVDVKGGDYELIPFGAGRRICAGLSWGLRMVTLMTATLVHAFDWTLVDGMTPEKLDMEEAYGLTLQRAVPLMVQPVPRLLPSAYTM

SEQ ID NO: 42 - protein, 523 aa, Triticum turgidum
MDHDLLLLLASLAAVAVAAVCYLRSHGSGAKLPLPPGPRGWPVLGNLPQLGAKPHHTMAALARQHGPLFRLRFGSAEVVVAASAKVAGSFLRAHDANFSDRPPNSGAEHVAYNYQDLVFAPYGARWRALRMLCALHLFSARALDALRSVRQDEARLMVTHLLSASSSPAQGVAIGQEANVCATNALARAAVGRRVVGDGVGESAREFKGMVVELMQLAGAFNIGDFVPALRWLDPQGVVAKMKHLHRRYDRIMDGFISEREHLAGEEEGKDLLSIMLAKMRQPLHADAGEDGIKFTETNIKALLLNLLTAGTDTTSSTVEWALVELIRHPDTLKQLQREVDDVVGTSRLVTEADLPRLTFLTAVIKETFRLHPSTPLSLPRVAAEDCEVDGYHVAKGTTLLVNVWAISRDPASWGADALEFRPARFLPGGSHETVDVKGGDYELIPFGAGRRMCAGLSWGLRIVTLMTATLVHAFDLSLVNGMTPDKLDMEEAYGLTLQRAVPLLVQPMPRLLPSAYATPCVN

SEQ ID NO: 43 - protein, 302 aa, Triticum turgidum
MEIPLTLLLSTFAISVTICYVIIFFFRADKGRAPLPPGPRGWPVLGNLPQLGGKTHQTLHEMTRLYGPMLRLRFGSSLVVVAGSADVAKQFLRTHDAKFSSRPPNSGGEHMAYNYQDVVFAPYGPRWRAMRKVCAVNLFSARALDDLRAFREWEALGAEEFNEIVLKLIEVGGVLNVGDFVPVLRWLDPQGVVAKMKKLHRRFDDMMNRIIAERRAGGFATTAGEEGGKDLLGLLLAMVQEDKSLTGAEENKITDTDVKALILLPAGQTPVMEETFSLLLQLAVPLMVHPVPRLLPSAYQIA

SEQ ID NO: 44 - protein, 305 aa, Triticum turgidum
MEIPLPLLLSTFAISVTICYVIFFFFHADKGRAPLPPGPRGWPVLGNLPQLSGKTHQTLHEMTKLYGPMLRLRFGSSLVVVAGSADVAKQFLRTHDARFSSRPPNSGGEHMAYNYQDVVFAPYGPRWRAMRKVCAVNLFSARALDDLRAFREREATEPGAVDFNEIVLKLIEVGGVLNVGDFVPALRWLDPQGVVAKMKKLHRRFDDMMNRIITERRTGAIAATAGEEDGKDLLGLLLAMVQEDKSLTGGSEEDRMTDTDVKALILLPAGKTPDMEETFSLLLQLAVPLMARPVPRLLPSAYQIA

SEQ ID NO: 45 - protein, 544 aa, Triticum turgidum
MNTRAPAVLAYRSNATMHLVAMDIPLPLLLSTLAVAVGVCYVLATFFRADKGRAPLPPGPRGWPVLGNLPQLGGKTHQTMHEMSKVYGPVLRLRFGSSVVVVAGSAGAAEQFLRTHDAKFSSRPPNSGGEHMAYNYQDVVFAPYGPRWRAMRKVCAVNLFSARALDDLRGFREREAALMVRSLVDAAATGGVVAVGKAANVCTTNALSRAAVGLRVFAAAGAELGAKEFKEIVLEVMEVGGVLNVGDFVPALRWLDPQGVVARLKKLHRRFDAMMNGIIAERRAGGSTAGEEKEGKDLLGLLLAMVQEDKSLTGGEEDRITDTDVKALILNLFVAGTETTSTIVEWAVAELIRHPDMLKRAQEEMDAVVGRDRLVSESDLPRLTFLNAVIKETFRLHPSTPLSLPRMASEECEVAGYRIPKGTELLVNVWGIARDPALWPDPLEFRPARFLPGGTHADVDVKGGDFGLIPFGAGRRICAGLSWGLRVVTVTAATLVHSFDWELPAGQTPDKLNMEEAFSLLLQRAVPLMAHPVPRLLPSAYEIA

SEQ ID NO: 46 - protein, 528 aa, Triticum turgidum; identical to SEQ ID NO: 28

SEQ ID NO: 47 - protein, 515 aa, Setaria italica
MDVPLPLLLGTVAVAAAAAWYLLLRRGGGGGKRPLPPGPRGWPVLGNLPQLGAKPHHTMAAMAREHGPLFRLRFGSAEVVVAASAAVAAQFLRAHDANFSNRPPNSGAEHVAYNYQDLVFAPYGARWRALRKLCALHLFSARALDDLRAVREGEVALMVRELARQRGPAVALGQAANVCATNTLARATVGRRVFAVDGGEGAREFKEMVVELMQLAGVFNVGDFVPALAWLDPQGVVGRMKRLHRRYDDMMDRIIREREAAGGDGNDLLGVLLTRMREHRPLADGEDGTINETDIKALLLNLFTAGTDTTSSTVEWALAELIRHPDVLAKAQQELDAVVGRGRLVSESDLPRLTYLTAVIKETFRLHPSTPLSLPRVAAEDCEVGGYLVPAGTTLLVNVWAIARDPDAWPEPLEFRPDRFLSGGPHAGVDVKGSDFELIPFGAGRRICAGLSWGLRMVTLMTAALVHGLDWHLAGGVDADKLDMEEAYGLTLQRAVPLMVRPEPRLLPSAYASVE

SEQ ID NO: 48 - protein, 472 aa, Setaria italica
MHMPCISFPRMSSDGKSMEIGRTMDIPTPLLLSTLAVSVVICYVLFWKQVATRKPAARPTPGGEHMAYNYQDVVFAPYGPRWRAMRKVCAVNLFSARALDDLRAVREREAALMVRSLAAAGQATAAVPLGRAVNVCTTNALSRAAVGRRVFAAGAGDDEGAREFKEIVLEVMQVGGVLNVGDFVPALRWLDPQGVVAKMKKLHRRFDDMMNGIIADRRKAGVTEEGKDLLGLLLAMVKDAGGEEDRITETNAKALILNLFVAGTDTTSTIVEWSLAELIRHPAILKQAQAELDAVVGRGRLLSESDLPRLTFFNAVIKETFRLHPSTPLSLPRMAAAECEVAGYRIPKGSELLVNVWGIARDPALWGPDPLEFRPARFLPGGSHADVDVKGGDFGLIPFGAGRRICAGLSWGLRMVTLASATLVHAFDWEMPAGQTPDELDMEEAFTLLLQRAVPLMVHPVPRLLPLAYEIA

SEQ ID NO: 49 - protein, 236 aa, Cenchrus americanus
MDLPLSLLLGTVAVAAVAAAWEAAGGDGPDLLGVLLARMREHQPLADGEDGTINETDMKALLLNLFTAGTDTTSSTVEWALAELLRHPDVLAKAQQELDAVVGRGRLVSESDLARLTYLTAVIKETFRLHPSTPLPDRFLPGGQHAGVDVKGSDFELIPFGAGRRICAGLSWGLRMVTLMTAALVHGLDWHLAGGVDADKLDMEEAYGLTLQRAVPLMVRPEPRLPPSAYAASSVE

SEQ ID NO: 50 - protein, 499 aa, Cenchrus americanus
  MOD_RES (153)..(212): Any amino acid
MDNIPTPLLLSTLAVSLVICYVLFWKQQAATRTKPQRAPLPPGPRGWPVLGNLPQLGGKTHQTLHEMTKVYGPLLRLRFGSSDVVVAGSAAVAEQFLRVHDANFSCRPPNSGGEHMAYNYQDVVFAPYGPRWRAMRKVCAVNLFSARALDDLXXXXXXXXXXXXXXXXXXXXXXXXXXXXXXXXXXXXXXXXXXXXXXXXXXXXXXXXXXXXTNALSRAAVGRRVFAAGGSGDDEGGAREFKEIVLEVMRVGGVLNVGDFVPALRWLDPQGVVAKMKKLHRRFDSMMNGIIADRRKAAGVTTEEGKDLLGLLLEMVKDERPLAGGEEDRITETDAKALILNLFVAGTDTTSTIVEWSLAELIRHPTILKQAQEELDAVVGRGRLVAESDLPRLTFFAAVFRPARFLPGGSHAGVDVKGGDFGLIPFGAGRRICAGLSWGLRMVTLASATLVHAFDWELPAGQTPDKLDMEEAFTLLLQRATPLMVQPVPRLLPSAYEIA

SEQ ID NO: 51 - protein, 517 aa, Sorghum bicolor
MDVPLPLLLGSLAVSVVVWCLLLRRGGDGKGKGKRPMPPGPRGWPVLGNLPQLGSHPHHTMCALAKKYGPLFRLRFGSAEVVVAASARVAAQFLRTHDANFSNRPPNSGAEHVAYNYQDMAFAPYGSRWRALRKLCALHLFSAKALDDLRSIREGEVALLVRELSRHQHQHAGVPLGQVANVCATNTLARATVGRRVFAVDGGEEAREFKDMVVELMQLAGVFNVGDFVPALARLDLQGVVGKMKRLHRRYDDMMNGIIRERKAAEEGKDLLSVLLARTREQQSIADGEDSRITETEIKALLLNLFTAGTDTTSSTVEWALAELIRHPDVLKKAQEELDAVVGRNRLVSELDLPRLTYLTAVIKETFRMHPSTPLSLPRIAAEECEVDGFRIPAGTTLLVNVWAIARDPEAWPEPLQFRPDRFLPGGSHAGVDVKGSDFELIPFGAGRRICAGLSWGLRMVTLMTATLVHALDWDLADGMTADKLDMEEAYGLTLQRAVPLKVRPAPRLLPSAYAAE

SEQ ID NO: 52 - protein, 479 aa, Sorghum bicolor
MDVPLPLLLGSLAVSVVVWCLLLRRGGDGKGKGKRPMPPGPRGWPVLGNLPQLGSHPHHTMCALAKKYGPLFRLRFGSAEVVVAASARVAAQFLRTHDANFSNRPPNSGAEHVAYNYQDMAFAPYGSRWRALRKLCALHLFSAKALDDLRSIREGEVALLVRELSRHQHQHAGVPLGQVANVCATNTLARATVGRRVFAVDGGEEAREFKDMVVELMQLAGVFNVGDFVPALARLDLQGVVGKMKRLHRRYDDMMNGIIRERKAAEEGKDLLSVLLARTREQQSIADGEDSRITETEIKALLLNLFTAGTDTTSSTVEWALAELIRHPDVLKKAQEELDAVVGRNRLVSELDLPRLTYLTAVIKETFRMHPSTPLSLPRIAAEECEVDGFRIPAGTTLLVNVWAIARDPEAWPEPLQFRPDRFLPGGSHAGVDVKGSDFELIPFGAGRRICAGLSWGLRMVTLMTATLVHALDWTSPTA

SEQ ID NO: 53 - protein, 517 aa, Sorghum bicolor
MHVPLLLGSLAVSVVVWCLLLRRGGDGKGKGNGKRPLPPGPRGWPVLGNLPQVGSHPHHTMYALAKEYGPLFRLRFGSADVVVAASARVAVQFLRAHDANFSNRPPNSGAEHMAYNYQDMVFAPYGSRWRALRKLCALHLFSAKALDDLRGVREGEVALMVRQLALHQHQHAGVPLGQVANVCATNTLARATVGRRVFAVDGGEEAREFKDMVVELMQLAGVFNVGDFVPALAWLDLQGVVGKMKRLHRRYDDMMNSIIRKRKAAEEGKDLLSVLLARMREQQSLADGEDSRINETGIKALLLDLFTAGTDTTSSTVEWALAELIRHPDVLKKAQEELDAVVGRDRLVSETDLPRLTYLTAVIKETFRLHPSTPLSLPRVAAEECEVDGFRIPAGTTLLVNVWAIARDPEAWPEPLQFRPDRFLPGGSHAGVDVKGSDFELIPFGAGRRICAGLSWGLRMVTLMTATLVHALDWDLADGMTADKLDMEEAYGLTLQRAVPLMVRPTPRLLPSAYAAE

SEQ ID NO: 54 - protein, 554 aa, Sorghum bicolor
MQVASVYIDEPLSLANHTRTTLSPTPSAPPVNRATQTMDVPLPLLLGSLAVSVVVWCLLLRRGGNGKGKGKRPLPPGPRGWPVLGNLPQVGSHPHHTMCALAKEYGPLFRLRFGSAEVVVAASARVAAQFLRAHDANFSNRPPNSGAEHVAYNYQDLVFAPYGSRWRALRKLCALHLFSAKALDDLRGVREGEVALMVRELARHQHQHAGVPLGQVANVCATNTLARATVGRRVFAVDGGEEAREFKDMVVELMQLAGVFNVGDFVPALAWLDLQGVVGKMKRLHRRYDDMMNGIIRERKAVEEGKDLLSVLLARMREQQSLADGEDSMINETDIKALLLNLFTAGTDTTSSTVEWALAELIRHPDVLKKAQEELDAVVGRDRLVSESDLPRLTYLTAVIKETFRLHPSTPLSLPRVAAEECEVDGFRIPAGTTLLVNVWAIARDPEAWPEPLQFRPDRFLPGGSHAGVDVKGSDFELIPFGAGRRICAGLSWGLRMVTLMTATLVHALDWDLADGMTANKLDMEEAYGLTLQRAVPLMVRPAPRLLPSAYAAE

SEQ ID NO: 55 - protein, 535 aa, Sorghum bicolor
MVMELVLATPDLPTPLLLSALTVAVSVAVCYVLFWKQQQAAARRAPLPPGPRGWPVLGNLPQLGGKTHQTLHELTKVYGPLLRLRFGSSDVVVAGSAAVAEQFLRVHDANFSCRPPNSGGELMAYNYQDVVFAPYGPRWRAMRKVCAVNLFSARALDDICDVREREAALMVRSLAEQAARDRNTPVALGKAVNVCTTNALSRAAVGRRVFAAAGAGDEGAREFKEIVLEVMEVGGVLNVGDFVPALRWLDPQGVVGRMKKLHRRFDDMMNGIIADSRKARATPADGEESKDLLGLLLSMVEDEGSDDEVRITETDVKALILNLFIAGTDTTSTIAEWSLAELIRHPDILKQAQEELDTVVGRGRLVTESDLRHLTFFNAVIKETFRLHPSTPLSLPRMAAEECEIAGYSIPKGCELLVNVWGIARDPALWPDPLEFRPARFLPGGSHSDVDVKGGNFGLIPFGAGRRICAGLSWGLRMVTLTSATLVHAFDWELPVGQTPDKLNMEEAFTLLLQRAVPLMAHPIPRLLPSAYEIA

SEQ ID NO: 56 - protein, 521 aa, Brachypodium distachyon
MEDMPLPLLIGSLFIILAMWYILFHHGSENNAKWSRLPLPPGPCGWPLLGNLPQLGAKPHHTMCALAWEHGPLFRLRLGSTEVVVASSAGIAMQFLRHHDANFSNRPPNSGAEHIAYNYQDLVFASYGTRWRALRKLCALHLFSAKALNNLRNVREGEVRLMVRELAWAAAGPAPAVALGQQANMCVTNTLARATIGRRVFAVDTAREFKEMVVELMQLAGVFNLGDFVPALRWLDPQGVVAKMKRLHRRYDNMMNGFIKEREPACLSAGAEAKDLLSVMLVKMREQQPLYHEEGKLTNTDIKALLLNLFTAGTDTASSTVEWALAELIRHPDVLKQVQRELDVVVGNDRLVSESDLPGLTFLPAVIKETFRLHPPTPLSLPRVAAEECEVNGYHIPKGTTLLVNVWAIARDPASWPDHPLEFRPVRFLPGGSHESLDVKGSDYELIPFGAGRRICAGLGWGIQMVTLMTTTLVHAFDWSLVDGMTPDKLDMEEAYGLTLQRAMPLFVQPVPRLLPSAYAM

SEQ ID NO: 57 - protein, 542 aa, Brachypodium distachyon
MLAFCMSKRSNSWRATAEACMELIGALDVPLRLPWLVSALAISVTVCYILFFSRAGKGNGKGLPPGPRGWPVLGNLPQLGGKTHQTLHELTKVYGPVLRLRLGSSVAVVAGTAGTAEQFLRAHDAQFRDRPPNSGGEHMAYNVFGPYGPRWRAMRKVCAVNLFSARALDGLRGFREREAALMVKSLAAAAASAAEPVALGKAANVCTTNALSRAAVGRRVFDEMGGSAGGELKEIVLEVIDVGGVLNVGDFVPALRWLDPQGVVARMKKLHRRFDDMMNGIIGERLQGTDAAGEKDLLGLLLDAMMKEDKSLSGGEELTHTDIKALILNLFVAGTDTTSSIVEWAMSELIRHPDLLQQAQEELDAVVGRARLVSESDMSRLPFLTAVIKETFRPHPSTPLSLPRMASEECFVAGYRIPKGTELVVNVWGIARDPALWPDPLEFRPARFLIGGSNSVVDLKGSNFELIPFGAGRRICAGLSWGLRIVMIAVATLVHAFDWKLPVGQTPDELNMEEALSLLLLRAVPLMVHPAPRLLPSAYEIA

SEQ ID NO: 58 - protein, 527 aa, Brachypodium distachyon
MDDFLLVAGSLALALTVCYYFIIHDNNNKAKKLPLPLPPGPRGWPVLGNLPQLGAAPHQTMRALAAEHGPLFRLRFGSAEVVVAASASVAARFLRGHDANFGDRPPNSGAEHVAYNYRDLVFAPYGARWRALRKLLALHLFSAKAIDALRGVRELEVALMVKGLRVSSSAPAGVAVGQEANVCATNALARAAVGRRVFFSGGGGGADSREFKEMVVELMQLAGVFNLGDFIPALRWLDPQGVVAKMKKLHRRYDDMMNGFIKERDAGAGAEQGKDLLSVMLGKMRELGGDDNNGGEEGEFTEVDIKALLLNLFTAGTDTTSSTVEWALAELIRHPDVLRQLQQELDAVVGKDRLVSESDLPRLAFLAAVIKETFRLHPSTPLSLPRLAAEECEVDGYRIPKGTTLLVNVWAIARDPASWADPLEFRPARFLPGGSHEGVDVKGGDYELIPFGAGRRICAGLSWGLRMVTLMTATLVHGFDWALVNGMTPDKLDMEEAYGLTLQRAVPLMVQPVPRLLPSAYAVQCDG

SEQ ID NO: 59 - protein, 530 aa, Brachypodium distachyon
MELLDGLDVPLLPALLSALAISLTICYVLFFSRAGKGLPPGPRGWPVLGNLPQLGGKTHQTLHEMSKLYGPVLRLRFGSSVVVVAGSAGAAEQFLRTNDAKFSNRPPNSGGEHMAYNYQDVVFGPYGPRWRAMRKVCAVNLFSARALDDLRGFREREASLMVKSLADAAAASGAGPVVALGKAANVCTTNALSRAAVGRRVFAAAGGEGAREFKEIVLEVMEVGGVLNVGDFVPALRWLDPQGVVARMKKLHRRFDDMMNGIIAEREGGCGMAPGEDGKEKDLLGLLLGMMQEEKSLTGGEEDDKITHTDIKALVLNLFVAGTETTSTIVEWAVAELIRHPDLLQQAQEELDAVVGRARVVSEADLPRLPFFTAVIKETFRLHPSTPLSLPRMASEECFVAGYRIPKGTELLVNIWGIARDPALWPDPLEFRPSRFLAGGSHADVDLKGADFGLIPFGAGRRICAGLSWGLRMVTITAATLVHAFDWELPAGQTPDKLNMEEAFSLLLQRAMPLMVHPVRRLLPSAYEIV

SEQ ID NO: 60 - protein, 515 aa, Hordeum vulgare
MEIPLPLLLSTLAISVTICYVIFFFFRSDKGCAPLPPGPRGWPVLGNLPQLGGKTHQTLHEMTRLYGPMFRLWFGSSLVVVAGSADMAKLFLRTHDAKFSSRPPNSGGEHMAYNYQDVVFAPYGPRWRAMRKVCAVNLFSARALDDLHSFREREAALMVRCLADSAAVGRVVALAKAANVCTTNALSRATVGLRVFATAGSELGAEDFNEIVLKLIEVGGILNVGDFVPALRWLDPQGVVAKMKKLHRRFDDMMNRIIAQRRAVSTTAGKDLLALLLAMVQEDKSLTGVEEDKIRDTDVKALILNLFVAGTDTTSITVEWAMAELIRHPHILKQAQEELDAVVGRDRLVLESDLPHLTFLNAVIKETFRLHPSTPLSLPRMAIEECEVAGHRIPKGTQLLVNVWGIARDPTLWPDPLEFRPARFLPGGSHAGVDVKGGDFGLIPFGAGRRICAGLSWGIRMVTVTTATLVHSFDWEMSAGQMPDMEETFSLLLQLAVPLMVHPVPRLLPSAYEIT

SEQ ID NO: 61 - protein, 510 aa, Gossypium sp.
MASFVLYSILSAVFLYFVFITSRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKISSLHLFSGKALDDFRQIREEEIRVLVRALASAKTKVNLGQLLNVCTVNALGQVMMGKRVFGDGSGGSDPEADEFKSMVVELMQLAGVFNIGDFIPALEWLDLQGVQAKMKKLHNRFDRFLSAILEEHKTKARQSNGQVKHKDFLSTLISLENVDGAEGGKLSDTEIKALLLNMFTAGTDTSSSTVEWAMAELIRHPNIMAQVRKELDSVVGRDRLVSDLDLPNLTYFQAVIKETFRIHPSTPLSLPRMASDSCDINGYHIPKGATLLVNVWAISRDPNEWNNPLEFRPERFLPGGERPNADVRGNDFEVIPFGAGRRICAGMSLGLRMVQLLTATLAHAFEWELADGLMPEKLDMEEAYGLTLQRAAPLMVHPRPRLSKHAY

SEQ ID NO: 62 - protein, 510 aa, Gossypium sp.
MPSFDTILLRDLVAAACLFFITRYFIRRLLSNPKRTLPPGPKGWPIVGALPLLGSMPHVELAKLAKKYGPVMYLKMGTCNMVVASTPDAARAFLKTLDLNFSNRPSNAGATHIAYNSQDMVFAEYGPRWKLLRKLSNLHMLGGKALEDWSQVRAVELGHMLRAMCESSRKGEPVVVPEMLTYAMANMIGQVILSRRVFVTKGSESNEFKDMVVELMTSAGLFNIGDFIPSIAWMDLQGIEGEMKKLHNRWDVLLTKMMKEHEETAYERKGKPDFLDIIMDNRENSAGERLSLTNVKALLLNLFTAGTDTSSSIIEWALAEILKNPKILNKAHEEMDKVIGRNRRLEESDIPKLPYLQAICKETFRKHPSTPLNLPRVSTQACEINGYYIPKNTRLSVNIWAIGRDPDVWGNPLDFTPERFLSGRFAKIDPRGNDFELIPFGAGRRICAGTRMGIVLVEYILGTLLHSFDWMLPPGNGELNMDEAFGLALQKAVPLSAMVRPRLAPTAYVS

SEQ ID NO: 63 - protein, 422 aa, Gossypium sp.
MASFVLYSILSAVFLYFVFITSRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKISSLHLFSGKALDDFRQIREEEIRVLVRALASAKTKVNLGQLLNVCTVNALGQVMMGKRVFGDGSGGSDPEADEFKSMVVELMQLAGVFNIGDFIPALEWLDLQGVQAKMKKLHNRFDRFLSAILEEHKTKARQSNGQVKHKDFLSTLISLENVDGAEGGKLSDTEIKALLLNMFTAGTDTSSSTVEWAMAELIRHPNIMAQVRKELDSVVGRDRLVSDLDLPNLTYFQAVIKETFRIHPSTPLSLPRMASDSCDINGYHIPKGSCPAAKGRTLMLGAMILRSYRSAPGVESVPE

SEQ ID NO: 64 - protein, 510 aa, Gossypium sp.
MATPSWFSYLTPWLATLALILFSLRLCRRRKLNLPPGPKPWPIIGNLNLIGSLPHQSIHALSRKYGPIMQLKFGSFPVVVASSVEMAKAVLKTNDVIFTDRPKTAAGKYTTYNYSDITWSPYGPYWRQARKICLTELFNAKRLESYQYIRREEMNLFLKRLYESSGTQIVLKDHLSSLSLNVISRMVFGKKYTEGSGENEIVTPNEFKEMLDELFLLNGVLDIGDSIPWLSFLDLQGYIKRMKALSKRFDRFLEHVLDEHNARREGAEDYVAKDMVDVLLQLSEDPNLEVKLERHGVKAFTQDMIAGGTESSAVTVEWAISELLKKPEILAKATEELDMVIGRERWVEEKDVVSLPYIDSIAKETMRLHPVAPMLVPRVARQDCEIAGYDIPKGTRAFVNVWTIGRDPSLWDNPNEFWPDRFMGKSIDVKGHDFELLPFGAGRRMCPGYPLGIKVIQASLANVLHGFTWKLPNNTTKEDLNMEEIFGLSTPKKYPLEAIAEPRLPLHMYS

SEQ ID NO: 65 - protein, 511 aa, Gossypium sp.
MESPSWVSYLTAWLATLALILLSLRFRPRRKLNFPPGPKPWPVIGNLDLICSLPHRSIHALSQKYGPLMQLKFGSFPVVVASSVEMAKAFLKTHDVIFAGRPKIAAGEYTTYNYSDITWSPYGPYWRQARKMCMTELFSAKRLESYEYIRREEMKLLLKGLYESSGVPIVLKDRLSDLSLNVISRMVFGKKYTEGTGENEIVTPKEFKEMLDELFLLNGVLDIGDSIPWLRFLDLQGNIKRMKALSKKFDKFLEHVLDEHNARRRDVKDYVAKDMVDVLLQLADDPNLDVKLERHGVKAFSQDMIAGGTESSAVTVEWAISEMLKKPEIFAKATEELDRVIGRERWVEERDIENLPYIDSIAKETMRLHPVAPMLVPRMTREDCQVDGYDILKGTRALVNVWTIGRDPTVWDNPNEFCPERFIDKTIDVKGHDFQLLPFGAGRRMCPGYPLGIKVIQASLANLLHGFTWKLPGNMTKEDLDMEEIFGLSTPKKCPLQAVAVPKLPLHLYSH

SEQ ID NO: 66 - protein, 510 aa, Gossypium hirsutum
MASFVLYSILSAVFLYFVFITSRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKMSSLHLFSGKALDDFRQIREEEIRVLVRALASAKTKVNLGQLLNVCTVNALGQVMMGKRVFGDGSGGSDPEADEFKSMVVELMQLAGVFNIGDFIPALEWLDLQGVQAKMKKLHNKFDRFLSAILEEHKTKARQSNGQVKHKDFLSTLISLENVDGAEGGKLSDTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKGATLLVNVWAISRDPNEWNNPLEFRPERFLPGGERPNADVRGNDFEVIPFGAGRRICAGMSLGLRMVQLLTATLAHAFEWELADGLMPEKLDMEEAYGLTLQRAAPLMVHPRPRLSKHAY

SEQ ID NO: 67 - protein, 510 aa, Gossypium hirsutum
MASFVLYSILSTVFLYFVFIISRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKISSVHLFSGKALDDFRHIREEEIRVLVRALASAKTKVNLGQLLNVCTVNALGQVMMGKRVFGDGSGGADPEADEFKSMVVELMQLAGVFNIGDFIPALEWLDLQGVQAKMKKLHNRFDRFLSGILEEHKTKARQSNGQVKHKDLLSTLISLENADGAEGGKLSDTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKDATLLVNVWAISRDPNEWNNPLEFRPERFLPGGERPNADVRGNDFEVIPFGAGRRICAGMSLGLHMVQLLTATLAHAFDWELADGLMPEKLDMEEAYGLTLQRAAPLMVHPRPRLSKHAY

SEQ ID NO: 68 - protein, 510 aa, Gossypium hirsutum
MAPFVLYSILSAVFLYFVFITSRKRRRLPLPPGPKPWPSIGNLPHMSPVPHQGLAAMAKVYGPLMHLRLGFVDVVVAASASMAAQFLKVHDSNFSSRPPNAGAKYVAYNYQDLVFAPYGPRWRLLRKMSSLHLFSGKALDDFRQIREEEIRVLVRALASAKTKVNLGQLLNVCTVNALGQVMMGKRVFGDGSGGSDPEADEFKSMVVELMQLAGVFNIGDFIPALEWLDLQGVQAKTKKLHNKFDRFLSAILEEHKTKARQSNGQVKHKDFLSTLISLENVDGAEGGKLSDTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKGATLLVNVWAISRDPNEWNNPLEFRPERFLPGGERPNADVRGNDFEVIPFGAGRRICAGMSLGLRMVQLLTATLAHAFEWELADGLMPEKLDMEEAYGLTLQRAAPLMVHPRPRLSKHAY

SEQ ID NO: 69 - protein, 80 aa, Gossypium hirsutum
MPSFDTILLRDLVAAACLFFITRYFIRRLLSNPKRTLPPGPKGWPIVGALPLLGSMPHVELAKLAKKYGPVMYLKMGTCN

SEQ ID NO: 70 - protein, 510 aa, Gossypium hirsutum
MPSFDTILLRDLVAAACLFFITRYFIRRLLSNPKRTLPPGPKGWPVVGALPLLGSMPHVELAKLAKKYGPVMYLKMGTCNMVVASTPDTARAFLKTLDLNFSNRPSNAGATHIAYNSQDMVFAEYGPRWKLLRKLSNLHMLGGKALEDWSQVRAVELGHMLRAMWESSRKGEPVVVPEMLTYAMANMIGQVILSRRVFVTKGSESNEFKDMVVELMTSAGLFNIGDFIPSIAWMDLQGIEGEMKKLHNRWDVLLTKMMKEHEETAYERKGKPDFLDIIMDNRENSAGERLSLTNVKALLLNLFTAGTDTSSSIIEWALAEILKNPKILNKAHEEMDKVIGRNRRLEESDIPKLPYLQAICKETFRKHPSTPLNLPRVSTQPCEINGYYIPKNTRLSVNIWAIGRDPDVWGNPLDFTPERFLSGRFAKIDPRGNDFELIPFGAGRRICAGTRMGIVLVEYILGTLLHSFDWMLPPGTGELNMDESFGLALQKTVPLSAMVRPRLAPTAYVS

SEQ ID NO: 71 - protein, 510 aa, Gossypium hirsutum
MPSFDTILLRDLVAAACLFFITRYFIRRLLSNPKRTLPPGPKGWPVVGALPLLGSMPHVELAKLAKKYGPVMYLKMGTCNMVVASTPDTARAFLKTLDLNFSNRPSNAGATHIAYNSQDMVFAEYGPRWKLLRKLSNLHMLGGEALEDWSQVRAVELGHMLRAMWESSRKGEPVVVPEMLTYAMANMIGQVILSRRVFVTKGSESNEFKDMVVELMTSAGLFNIGDFIPSIAWMDLQGIEGEMKKLHNRWDVLLTKMMKGHEETAYERKGKPDFLDIIMDNRENSAGERLSLTNVKALLLNLFTAGTDTSSSIIEWALAEILKNPKILNKAHEEMDRVIGRNRRLEESDIPKLPYLQAICKETFRKHPSTPLNLPRVSTQACEINGYYIPKNTRLSVNIWAIGRDPDVWGNPLDFTPERFLSGRFAKIDPRGNDFELIPFGAGRRICAGTRMGIVLVEYILGTLLHSFDWMLPPGTGELNMDEAFGLALQKAVPLSAMVRPRLAPTAYVS

SEQ ID NO: 72 - protein, 511 aa, Gossypium hirsutum
MESPSWVSYLIAWLATLALILLSLRFRPRRKLNLPPGPKPWPVIGNLDLIGSLPHRSIHSLSQKYGPLMQLKFGSFPVVVASSVEMAKAFLKTHDVIFAGRPKIAAGEYTTYNYSDITWSPYGPYWRQARKMCMTELFSAKRLESYEYIRREEMKLLLKGFYESSGVPIVLKDHLSDLSLNVISRMVFGKKYTEGTGENEIVTPKEFKEMLDELFLLNGVLDIGDSIPWLRFLDLQGNIKRMKALSKKFDKFLEHVLDEHNARRRDVKDYVAKDMVDVLLQLADDPNLDVKLERHGVKAFSQDLIAGGTESSAVTVEWAISEMLKKPEIFAKATEELDRVIGRERWVEERDIVNLPYIDSIAKETMRLHPVAPMLVPRMTREDCQVDGYDILKGTRALVNVWTIGRDPTVWDNPNEFFPERFIDKTIDVKGHDFQLLPFGAGRRMCPGYPLGIKVIQASLANLLHGFNWKLPGNMTKDDLDMEEIFGLSTPKKCPLQAVAVPKLPLHMYSH

SEQ ID NO: 73 - protein, 511 aa, Gossypium hirsutum
MESPSWVSYLTAWLATLALILLSLRFRPRRKLNFPPGPKPWPVIGNLDLIGSLPHRSIHALSQKYGPLMQLKFGSFPVVVASSVEMAKAFLKTHDVIFAGRPKIAAGEYTTYNYSDITWSPYGPYWRQARKMCMTELFSAKRLESYEYIRREEMKLLLKGLYESSGVPIVLKDRLSDLSLNVISRMVFGKKYTEGTGENEIVTPKEFKEMLDELFLLNGVLDIGDSIPWLRFLDLQGNIKRMKALSKKFDKFLEHVLDEHNARRRDVKDYAAKDMVDVLLQLADDPNLDVKLERHGVKAFSQDLIAGGTESSAVTVEWAISEMLKKPEIFAKATEELDRVIGRERWVEERDTVNLPYIDSIAKETMRLHPVAPMLVPRMTREDCQVDGYDILKGTRALVNVWTIGRDPTVWDNPNEFCPERFIDKTIDVKGHDFQLLPFGAGRRMCPGYPLGIKVIQASLANLLHGFTWKLPGNMTKENLDMEEIFGLSTPKKCPLQAVAVPKLPLHLYSH

SEQ ID NO: 74 - protein, 497 aa, Gossypium hirsutum
MTQQAILLSLRFRPRRKLNFPPGPKPWPVIGNLDLIGSLPHRSIHALSQKYGPLMQLKFGSFPVVVASSVEMAKAFLKTHDVIFAGRPKIAAGEYTTYNYSDITWSPYGPYWRQARKMCMTELFSAKRLESYEYIRREEMKLLLKGLYESSGVPIVLKDRLSDLSLNVISRMVFGKKYTEGTGENEIVTPKEFKEMLDELFLLNGVLDIGDSIPWLRFLDLQGNIKRMKALSKKFDKFLEHVLDEHNARRRDVKDYAAKDMVDVLLQLADDPNLDVKLERHGVKAFSQDLIAGGTESSAVTVEWAISEMLKKPEIFAKATGELDRVIGRERWVEERDTVNLPYIDSIAKETMRLHPVAPMLVPRMTREDCQVDGYDILKGTRALVNVWTIGRDPTVWDNPNEFCPERFIDKTIDVKGHDFQLLPFGAGRRMCPGYPLGIKVIQASLANLLHGFTWKLPGNMTKENLDMEEIFGLSTPKKCPLQAVAVPKLPLHLYSH

SEQ ID NO: 75 - protein, 510 aa, Gossypium barbadense
MTSFVLYSILSTVFLYFVFIISRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKISSVHLFSGKALDDFRHIREEEIRVLVRALASAKTKVNLGQLLNVCTVNALGQVMMGKRVFGDGSGGADPEADEFKSMVVELMQLAGVFNIGDFIPALEWLDLQGVQAKMKKLHNRFDRFLSGILEEHKTKARQSNGQVKHKDLLSTLISLENADGAEGGKLSNTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKGATLLVNVWAISRDPDEWNNPLEFRPERFLPGGERPNADVRGNDFEVIPFGAGRRICAGMSLGLRMVQLLTATLAHAFDWELADGLMPEKLDMEEAYGLTLQRAAPLMVHPRPRLSKHAY

SEQ ID NO: 76 - protein, 510 aa, Gossypium barbadense
MASFVLYSILSAVFLYFVFITSRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKMSSLHLFSGKALDDFRQIREEEIRVLVRALASAKTKVNLGQLLNVCTVNALGQVMMGKRVFGDGSGGSDPEADEFKSMVVELMQLAGVFNIGDFIPALEWLDLQGVQAKMKKLHNKFDRFLSAILEEHKTKARQSNGQVKHKDFLSTLISLENVDGAEGGKLSDTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKGATLLVNVWAISRDPNEWNNPLEFRPERFLPGGERPNADVRGNDFEVIPFGAGRRICAGMSLGLRMVQLLTATLAHAFEWELADGLMPDKLDMEEAYGLTLQRAAPLMVHPRPRLSKHAY

SEQ ID NO: 77 - protein, 510 aa, Gossypium barbadense
MASFVLYSILSAVFLYFVFITSRKRRRLPLPPGPKPWPIIGNLPHMSPVPHQGLAAMAKVYGPLMHLRLGFVDVVVAASASMAAQFLKVHDSNFSSRPPNAGAKYVAYNYQDLVFAPYGPRWRLLRKMSSLHLFSGKALDDFRQIREEEIRVLVRALASAKTKVNLGQLLNVCTVNALGQVMMGKRVFGDGSGGSDPEADEFKSMVVELMQLAGVFNIGDFIPALEWLDLQGVQAKMKKLHNRFDRFLSGILEEHKTKARQSNGQVKHKDLLSTLISLENADGAEGGKLSNTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKGATLLVNVWAISRDPNEWNNPLEFRPERFLPGGERPNADVRGNDFEVIPFGAGRRICAGMSLGLRMVQLLTATLAHAFDWELADGLMPEKLDMEEAYGLTLQRAAPLMVHPRPRLSKHAY

SEQ ID NO: 78 - protein, 430 aa, Gossypium barbadense
MLVPHQGLAAMAKVYGPLMHLRLGFVDVVVAASASMAAQFLKVHDSNFSSRPPNAGAKYVAYNYQDLEEIRVLVRALASAKTKVNLGQLLNVCTVNALGQVMMGKRVFGDGSGGADPEADEFKSMVVELMQLAGVFNIGDFIPALEWLDLQGVQAKMKKLHNRFDRFLSGILEEHKTKARQSNGQVKHKDLLSTLISLENADGAEGGKLSNTEIKALLLNMFTAGTDTSSSTVEWAMAELIRHPNIMAQVRKELDSVVGRDRLVSDLDLPNLTYFQAVIKETFRLHPSTPLSLPRMASDSCDINGYHIPKGATLLVNVWAISRDPDEWNNPLEFRPERFLPGGERPNADVRGNDFEVIPFGAGRRICAGMSLGLRMVQLLTATLAHAFDWELADGLMPEKLDMEEAYGLTLQRAAPLMVHPRPRLSKHAY

SEQ ID NO: 79 - protein, 510 aa, Gossypium barbadense
MPSFDTILLRDLVAAACLFFITRYFIRRLLSNPKRTLPPGPKGWPIVGALPLLGSMPHVELAKLAKKYGPVMYLKMGTCNMVVASTPDAARAFLKTLDLNFSNRPSNAGATHIAYNSQDMVFAEYGPRWKLLRKLSNLHMLGGKALEDWSQVRAVELGHMLRAMCESSRKGEPVVVPEMLTYAMANMIGQVILSRRVFVTKGSESNEFKDMVVELMTSAGLFNVGDFIPSIAWMDLQGIEGEMKKLHNRWDVLLTKMMKEHEETAYERKGKPDFLDIIMDNRENSAGERLSLTNVKALLLNLFTAGTDTSSSIIEWALAEILKNPKILNKAHEEMDKVIGRNRRLEESDIPKLPYLQAICKETFRKHPSTPLNLPRVSTQACEINGYYIPKNTRLSVNIWAIGRDPDVWGNPLDFTPERFLSGRFAKIDPRGNDFELIPFGAGRRICAGTRMGIVLVEYILGTLLHSFDWMLPPGTGELNMDEAFGLALQKAVPLSAMVRPRLAPTAYVS

SEQ ID NO: 80 - protein, 510 aa, Gossypium barbadense
MPSFDTILLRDLVAAACLFFITRYFIRRLLSNPKRTLPPGPKGWPVVGALPLLGSMPHVELAKLAKKYGPVMYLKMGTCNMVVASTPDTARAFLKTLDLNFSNRPSNAGATHIAYNSQDMVFAEYGPRWKLLRKLSNLHMLGGKALEDWSQVRAVELGHMLRAMWESSRKGEPVVVPEMLTYAMANMIGQVILSRRVFVTKGSESNEFKDMVVELMTSAGLFNIGDFIPSIAWMDLQGIEGEMKKLHKRWDVLLTKMMKEHEETAYERKGKPDFLDIIMENRENSAGERLSLTNVKALLLNLFTAGTDTSSSIIEWALAEILKNPKILNKAHEEMDKVIGRNRRLEESDIPKLPYLQAICKETFRKHPSTPLNLPRVSTQPCEINGYYIPKNTRLSVNIWAIGRDPDVWGNPLDFTPERFLSGRFAKIDPRGNDFELIPFGAGRRICAGTRMGIVLVEYILGTLLHSFDWMLPPGTGELNMDESFGLALQKTVPLSAMVRPRLAPTAYVS

SEQ ID NO: 81 - protein, 510 aa, Gossypium barbadense
MPSFDTILLRDLVAAAFLFFITRYFIRRILSNPKRILPPGPNGWPVVGALPLLGSMPHVELAKLAKKYGPVMYLKMGTCNMVVASTPDAARAFLKTLDLNFSNRPSNAGATHIAYDSQDMVFAEYGPRWKLLRKLSNLHMLGGRALEDWSQVRAVELGHMLRAMCESSRKGEPVVVPEMLTYAMANMIGQVILSRRVFVTKGSESNEFKDMVVELMTSAGLFNIGDFIPSIAWMDLQGIEGEMKKLHKRWDVLLTKMMKEHEETAYERKGKPDFLDIIMDNRENSAGERLSLTNVKALLLNLFTAGTDTSSSIIEWALAEILKNPKILNKAHEEMDKVIGRNRRLEESDVLKLPYLQAICKETFRKHPSTPLNLPRVSTQPCEINGYYIPKNTRLSVNIWAIGRDPDVWGNPLDFTPERFLSGRFAKIDPRGNDFELIPFGAGRRICAGTRMGIVLVEYILGTLLHSFDWMLPPGTGELNMDESFGLALQKTVPLSAMVRPRLAPTAYVS

SEQ ID NO: 82 - protein, 320 aa, Gossypium barbadense
MESPSWVSYLIAWLATLALILLSLRFRPRRKLNLPPGPKPWPVIGNLDLIGSLPHRSIHSLSQKYGPLMQLKFGSFPVVVASSVEMAKAFLKTHDVIFAGRPKIAAGEYTTYNYSDITWSPYGPYWRQARKMCMTELFSAKRLESYEYIRREEMKLLLKGFYESSGVPIVLKDHLSDLSLNVISRMVFGKKYTEGTGENEIVTPKEFKEMLDELFLLNGVLDIGDSIPWLRFLDLQGNIKRMKALSKKFDKFLEHVLDEHNARRRDVKDYVAKDMVDVLLQLADDPNLDVKLERHGVKAFSQDLIAGGTESSAVTVEWAI

SEQ ID NO: 83 - protein, 510 aa, Gossypium barbadense
MATPSWFSYLTPWLATLALILFSLRLCRRRKLNLPPGPKPWPIIGNLNLVGSLPHQSIHALSRKYGPIMQLKFGSFPVVVASSVEMAKAVLKTNDVIFTDRPKTAAGKYTTYNYSDITWSPYGPYWRQARKICLTELFNAKRLESYQYIRREEMNLFLKRLYESSGTQIVLKDHLSSLSLNVISRMVFGKKYTEGSGENEIVTPNEFKEMLDELFLLNGVLDIGDSIPWLSFLDLQGYIKRMKALSKRLDRFLEHVLDEHNARREGAEDYVAKDMVDVLLQLSEDPNLEVKLERHGVKAFTQDMIAGGTESSAVTVEWAISELLKKPEILAKATEELDMVIGRERWVEEKDVVSLPYIDSIAKETMRLHPVAPMLVPRVARQDCEIAGYDIPKGTRAFVNVWTIGRDPSLWDNPNEFWPDRFMGKSIDVKGHDFELLPFGAGRRMCPGYPLGIKVIQASLANVLHGFTWKLPNNTTKDDLNMEEIFGLSTPKKYPLEAIAEPRLPLHMYS

SEQ ID NO: 84 - protein, 717 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHEAMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHETMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQAVIKENFRLHPPTPLSLPHIASESCEINGYHIPKGSTLLTNIWAIARDPDQWTDPLSFRPERFLPGGEKAGVDVKGNDFELIPFGAGRRICAGLSLGLRTIQLLTATLVHGFEWELAGGVTPEKLNMEETYGITLQRAVPLVVHPKPRLDMSAYGLGSA

SEQ ID NO: 85 - protein, 511 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHEAMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQAVIKENFRLHPPTPLSLPHIASESCEINGYHIPKGSTLLTNIWAIARDPDQWSDPLTFRPERFLPGGEKAGVDVKGNDFELIPFGAGRRICAGLSLGLRTIQLLTATLVHGFEWELAGGVTPEKLNMEETYGITLQRAVPLVVHPKLRLDMSAYGLGSA

SEQ ID NO: 86 - protein, 511 aa, Brassica napus; identical to SEQ ID NO: 85

SEQ ID NO: 87 - protein, 511 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHETMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQAVIKENFRLHPPTPLSLPHIASESCEINGYHIPKGSTLLTNIWAIARDPDQWTDPLSFRPERFLPGGEKAGVDVKGNDFELIPFGAGRRICAGLSLGLRTIQLLTATLVHGFEWELAGGVTPEKLNMEETYGITLQRAVPLVVHPKPRLDMSAYGLGSA

SEQ ID NO: 88 - protein, 511 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHEAMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQAVIKENFRLHPPTPLSLPHIASESCEINGYHIPKGSTLLTNIWAIARDPDQWSDPLTFRPERFLPGGEKAGVDVKGNDFELIPFGAGRRICAGLSLGLRTIQLLTATLVHGFEWELAGGVTPEKLNMEETYGITLQRAVPLVVHPKPRLDRSAYGLGSA

SEQ ID NO: 89 - protein, 511 aa, Brassica napus; identical to SEQ ID NO: 87

SEQ ID NO: 90 - protein, 511 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHEAMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQAVIKENFRLHPPTPLSLPHIASESCEINGYHIPKGSTLLTNIWAIARDPDQWTDPLSFRPERFLPGGEKAGVDVKGNDFELIPFGAGRRICAGLSLGLRTIQLLTATLVHGFEWELAGGVTPEKLNMEETYGITLQRAVPLVVHPKLRLDMSAYGLGSA

SEQ ID NO: 91 - protein, 511 aa, Brassica napus; identical to SEQ ID NO: 87

SEQ ID NO: 92 - protein, 511 aa, Brassica napus; identical to SEQ ID NO: 85

SEQ ID NO: 93 - protein, 511 aa, Brassica napus; identical to SEQ ID NO: 87

SEQ ID NO: 94 - protein, 363 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHEAMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQVTRTGNSDCFG

SEQ ID NO: 95 - protein, 363 aa, Brassica napus
MTNLYLTILLPTFIFLIVLVLSRRRNNRLPPGPNPWPIIGNLPHMGPKPHQTLAAMVTTYGPILHLRLGFADVVVAASKSVAEQFLKVHDANFASRPPNSGAKHMAYNYQDLVFAPYGQRWRMLRKISSVHLFSAKALEDFKHVRQEEVGTLMRELARANTKPVNLGQLVNMCVLNALGREMIGRRLFGADADHKAEEFRSMVTEMMALAGVFNIGDFVPALDCLDLQGVAGKMKRLHKRFDAFLSSILEEHETMKNGQDQKHTDMLSTLISLKGTDFDGEGGTLTDTEIKALLLNMFTAGTDTSASTVDWAIAELIRHPEIMRKAQEELDSVVGRGRPINESDLSQLPYLQVTRTENSDCFG

SEQ ID NO: 96 - protein, 511 aa, Brassica napus; identical to SEQ ID NO: 90

SEQ ID NO: 97 - protein, 511 aa, Brassica napus; identical to SEQ ID NO: 87

SEQ ID NO: 98 - protein, 363 aa, Brassica napus; identical to SEQ ID NO: 94

SEQ ID NO: 99 - protein, 363 aa, Brassica napus; identical to SEQ ID NO: 95

SEQ ID NO: 100 - protein, 519 aa, Saccharum sp.
MELPTWASFLGVVLATVMLLKAILGRRRRVYNLPPGPKPWPIIGNLNLMGALPHRSIHELSRKYGPLMQLRFGSFPVVVGSSVDMAKFFLKTHDVVFTDRPKTAAGKYTTYNYRDITWSPYGAYWRQARKMCLTELFSAKRLESYEYIRAAEVRALLRDLHSASGSGRAVMLKDHLSTVSLNVITRMVLGKKYLDKDEVASAGSVTMTTPEEFKWMLDELFLLNGVLNIGDSIPWLDWMDLQGYIKRMKKLSKMFDRFLEHVVEEHNQRRLREGKDFVAKDMVDVLLQIADDPTLEVELNRESVKAFTQDLIAGGTESSAVTVEWAISELLKKPEVIVKATEELDRVIGRGRWVTEKDIPSLPYVDAIVKETMRLHPVAPMLVPRLSREDTTVAGYDIPAGTRVLVSVWSIGRDPALWDAPEEFMPERFLSSRLDVKGQDYELLPFGSGRRMCPGYSLGLKVIQVSLANLLHGFSWSLPYGVTKEELSMEEIFGLSTPRKFPLEAVVEPKLPAHLYAEP

SEQ ID NO: 101 - protein, 507 aa, Saccharum sp.
MELSAWASVFAVVFTTVVYLGAVHARRRRACNSLPGPKPWPIIGNFNLLGALPHRSLDALSKRHGPLMRVQFGSFPVVIASSVDMAKFFLKTHDSVFIDRPKMAAGKYTTYNYSNIAWSPYGAYWRQARKICADELFSARRLESFEHVRQEEVHALLRTLHGTAGQVVPLKECLSTMSLNIITRMVLGRKCVDKEVVASGGGSVTTWKEFRWMLDELFLLNGVLNIGDWIPWLSWLDLQGYVRRMKRVGRMFNQFMENVVEEHNERRLREGDAFVPQDMVDRLLQLADDPSLDVKLTRDSVKAFTQSAAVIVEWAISELLKNPDVFAKATEELDGVIGRDRWVTEKDIPHLPYMDAIVKETMRLHMVVPLLSPRLSREDTSVGGYDIPAGTRVLINAWTISRDPALWDAPEEFRPERFVGSKIDVKGQDFELLPFGSGRRMCPGYSLGLKVIQVTLVNLLHGFAWRLPDGMTEEELSMEEVFGLSTPRKFPLQAVVEPKLPARMYTA

SEQ ID NO: 102 - protein, 516 aa, Saccharum sp.
MDATQDSPLFLFPAAATLLSPLLAVLLVVLSLLWLYPGGPAWALIISRSRATPPPGTPGVVTALAGPAAHRTLASLSQSLPGGGSALLAFSVGLTRLVVASQPDTARELLASAAFADRPVKDAARGLLFHRAMGFAPSGDYWRALRRISSAYLFSPRSVSATAPRRVAIGERMLRDLSAAATGGGGGGEVVMRRVLHAASLDHVMATVFGARYDADSAEGAELEEMVKEGYDLLGLFNWGDHLPLLRWLDLQGVRRRCRSLVSRVNVFVARIIEEHRQKKKDDAANGESAAGDFVDVLLGLEGEEKLSDSDMIAVLWEMIFRGTDTVAILLEWVMARMVLHPGIQSKAQAELDAVVGRGRAVSDADVARLPYLQRVVKETLRVHPPGPLLSWARLAVHDAVVGGHLVPAGTTAMVNMWAIAHDPVVWAEPSAFRPERFEEEDVSVLGGDLRLAPFGAGRRVCPGKTLALATVHLWLAQLLHRFQWAPADGGVDLAERLGMSLEMEKPLVCKPTPRW

SEQ ID NO: 103 - protein, 516 aa, Saccharum sp.; identical to SEQ ID NO: 102

SEQ ID NO: 104 - protein, 341 aa, Saccharum sp.
MELPTWASFLGVVLATVMLLKAILGRRRRVYNLPPGPKPWPIIGNLNLMGALPHRSIHELSRKYGPLMQLRFGSFPVVVGSSVDMAKFFLKTHDVVFTDRPKTAAGKYTTYNYRDITWSPYGAYWRQARKMCLTELFSAKRLESYEYIRAAEVRALLRDLHSASGSGRAVMLKDHLSTVSLNVITRMVLGKKYLDKDEVASAGSVTMTTPEEFKWMLDELFLLNGVLNIGDSIPWLDWMDLQGYIKRMKKLSKMFDRFLEHVVEEHNQRRLREGKDFVAKDMVDVLMQIADDPTLEVELDRESVKAFTQDLIAGGTESSAVTVEWAISELLKKPEVIAKAT

SEQ ID NO: 105 - protein, 479 aa, Saccharum sp.
MSAGYFKNKHSLGARSVPVHAGSCYASSQGPLWFLVVPLMLELLPFICRRLHHRPNAGDDDRKRSKPLLPSPPGRLPVIGHLHLIGDLPHVSLRDLATKHDHGGGLMLLQLGTVPILVVSSPHAAQAVLRTHDHVFASRPAPKVLHNFLYGSSTIAFGPYGEHWRKVRKLVTTRLFTVKKVRQVMAKLKKAMATGMAVEMSETMNTFANEIMCRVLSGKFFKEDSRNKTFRELIEMNVALYAGFSLENYFPGLVNSLGIFTRMVSRKADETHERWDDVLENIISDHERRAEQEESADFVDLMLSVQQEYDLFDAGTGTSYLTLELAMAELMRHPHIMTKLQAEVRNKIPNGQEMVREEDLASMAYLRAVVKETLRLHPPAPLFLPYQSMVDCEIDGYTIPSGTRVIINSWAVCRHVESWEKAEEFMPERFMDGGSAAAVDFKGNDFQFIPFGAGRRMCPGINFGLAIVEIMLANLIVLF

SEQ ID NO: 106 - protein, 452 aa, Saccharum sp.
MDEFLYQSLLLSVVALVKLAFIKRRPRLPPGPWKLPVIGSMHHLINVLPHRALRDLAAVHGPLMMLQLGQTALVVASSKETARAVLKTHDTNFATRPKLLAGQIVGYEWVDILFAPSGDYWRKLRQLCAAEILSPKRVLSFRHIREDEVMLRVEEIRAAGPSTPVNLSVMFHSITNSVVSRAATRAATKAVVGLASGFNIPDLFPGWTTVLAKLTGMTCSLQDIHKTVHTILEEIIQERKAIRDEKISSGAEDIDENLVDVLLGLQEKGGFGFQLNNSIIKAIILDMFAGGTGTSGSAMEWGMSELMRNPEVMKKLQPAGADQGGSIEECELDGYTIPAKSRVIINAWAIGRDPRYWEAADEFKPERFEDGARDFTGSSYEFLPFGSGRRMCPGFNYGLASMELAFVGLLYHFDWSLPDGVEEVDMGEAPGLGVRRRTPLLLCATPFVPVDA

SEQ ID NO: 107 - protein, 408 aa, Saccharum sp.
MLLQLGTVSNLVVSSPRAARAVLRTHDHVFASRPTTKVLHNFLYGSSTIAFGPYGEHWRKVRKLVTTHLFTVKKVNSFCHARQEEVRLVMAKLKKAMATGMEVDMSETMNTFANDIMCCVVSGKLFREDGRNKTFRELIEMNSALYAGFSLENYFPRLVNSLGIFTRFVSRKADKTHERWDEVLENIISDHERQSFNYRHGDRAEQEEGTDFVDVMLSVQQEYGISRDHIKAVLMDMFDAGTVTSSLVLELAMAELMRHPHLMSKLQAEVRNKTPNGEEMVKQENLASMSYLRAVVKETLRHLESWEKAEKFMPERFMDGGSAATIDLKGNDFQFIPFGAGRRMCPGINFGLVTVEIMLANLMYCFDWGLPAGMDKKDIDMTEVFGLTVHRKEKLMLVPKLPGTASYA

SEQ ID NO: 108 - protein, 543 aa, Saccharum sp.
MSMHQPTSAAATQLHHAAMEASLMSLSFLQLAFTAVAAIAALAVAVAVTRYNRRYMGLRLPPGPPVWPVVGNLFQVAFSGKLFIHYIRDLRKEYGPILTLRMGERTLVIISSAELAHEALVEKGREFASRPRENTTRNIFSSNKFTVNSAVYGAEWRSLRRNMVSGMLSTSRLREFAHARRRAMDRFVSRMRAEALASPDGASVWVLRNARFAVFCILLDMTFGLLDLHEEHIVHIDAVMKRVLLAVGVRMDDYLPFLRPFFWRHQRRALAVRREQVDTLLPLISRRRAILRDMKSSSPPDPNVAAPFSYLDSVLDLHIEGRDGTPTDDELVTLCAELINGGTDTTATAIEWGMARIVDNPSIQARLHEEIMQQVGDARPVDDKDTDAMPYLQAFVKELLRKHPPTYFSLTHAAVQPGSKLAGYDVPVDANLDIFLPTISEDPKLWDRPTEFDPDRFVSGGEMGDMTGSGGIRMIPFGAGRRICPGLAMGTTHIALMVARMVQAFEWRAHPSQPPLDFKDKVEFTVVMDRPLLAAVKPRNLSF

SEQ ID NO: 109 - protein, 542 aa, Saccharum sp.
MSMHQPTSAAATQLHHAAMEASLMSLSFLQLAFTAVAAIAALAVAVAVTRYNRRYMGLRLPPGPPVWPVVGNLFQVAFSGKLFIHYIRDLRKEYGPILTLRMGERTLVIISSAELAHEALVEKGQEFASRPRENTTRNIFSSNKFTVNSAVYGAEWRSLRRNMVSGMLSTSRLREFAHARRRAMDRFVSRMRAEAAASPDGASVWVLRNARFAVFCILLDMTFGLLDLHEEHIVHIDAVMKRVLLAVGVRMDDYLPFLRPFFWRHQRRALAVRREQVDTLLPLISRRRAILRDMKSSSPPDPNVAAPFSYLDSVLDLHIEGRDGAPTDDELVTLCAELINGGTDTTATAIEWGLARIVDNPSIQARLHEEIMHQVGDARPVDDKDTDAMPYLQAFVKELLRKHPPTYFSLTHAAVQPGSKLAGYDVPVDANLDIFLPTISEDPKLWDRPTEFDPDRFVSGGEMGDMTGSGGIRMIPFGAGRRICPGLAMGTTHIALMVARMVQAFEWRAHPSQPPLDFKDKVEFTVVMDRPLLAAVKPRNLS

SEQ ID NO: 110 - protein, 304 aa, Saccharum sp.
MAKLKKAMATGMEVDMSETMNTFANDIMCCVVSGKLFREDGRNKTFRELIEMNSALYAGFSLENYFPRLVNSLGIFTRFVSRKADKTHERWDEVLENIISDHERQSFNYRHGDRAEQEEGTDFVDVMLSVQQEYGISRDHIKAVLMDMFDAGTVTSSLVLELAMAELMRHPHLMSKLQAEVRNKTPNGEEMVKQENLASMSYLRAVVKETLRFMDGGSAATIDLKGNDFQFIPFGAGRRMCPGINFGLVTVEIMLANLMYCFDWGLPAGMDKKDIDMTEVFGLTVHRKEKLMLVPKLPGTASYA

SEQ ID NO: 111 - protein, 439 aa, Saccharum sp.
MGKITPQTQNSQTWISLIFDEMSDMSMMTASDRVAPYIHASLSLHWYGPIFKTNLVGQPMVVSADPEVNRFIFQQEGKLFRSWYPETANIIIGEKTIDEFNGPTQKFVRNIISRLFGLEYLKQDLIPELEKDIRDTFAEWTTKPSIDVHDSTPDVIFVLVAKKMLGLHPSESRELRKNYSSFLQGLISFPIYFPGTTFYQCMQGKNNMLNLMSNLLRKRLSMPEKHGDILDLMVEELQSENPTIDDKFATDTLSAILFTSFVTLSPNLTLAFKFLSDNPAVLDALKEEHDTILRNRKDSSSGFTWEEYKSLTFTTMVINELMRMSNPTPGIFRKTLTDVQVNGYTIPAGWMVMMSPMAVHLNPAFFEDPLDFNPWRWLDESKRNAQKNFVPFGLGTRACPAAEFSKLFIALFLHVLVTKYRLLLAHDKSIYTFVMLAAL

SEQ ID NO: 112 - protein, 515 aa, Saccharum officinarum
METLHAHDELFSCVVLVLVTTITILYLKQLLLAAFERRAGSPSLPCPRGLPLIGNLHQLGTAPHDSLAALAAKHAAPLMLLRLGSVPTLVVSTADALRAVFQPNDRAMSGRPALYAATRITYGLQDIVFSPPDGAFWRAARRASLSELLSAPRVRSFRDVREGEAAALVAAITDMSGSGSPVNLSEEVMATSNKILRRVAFGDGGGEESIEAGKVLDETQKLLGGFFVADYMPWLGWLDALRGLRRRLERNFHELDAFYEKVIDDHLSKRGAGADASKGEDLVDVLLRLHGDPAYQSTFNSRDQIKGILTDMFIAGTDTAAATVEWTMTELVRHPDILAKAQKEVRAAVVGKDIVLESDLPRLKYLKQVIRESMRVHPPVPLLVPRETIEPCTVYGCEIPARTRVFVNAKAIGQDPDAWGPDAARFVPERHEEIADLSDHKPWHDSFSLVPFGVGRRSCPGVHFATSVVELLLANLLFCFDWRAPHGEVDLEQETGLTVHRKNPLVLVAERRGVL

SEQ ID NO: 113 - protein, 539 aa, Saccharum officinarum
MARALVAMALRFLRDYVRASDLAVAAAVLFVCSAVRSRLSSRPGEPMLWPVVGIIPTLFAHLAIGDVYDWGAVVLSRCRGTFPYRGTWGGGSSGVITSVPANVEHVLKDNFDNYPKGPYYRERFAELLGDGIFNADGDSWRAQRKAASAEMHSARFLRFSAATIERLVCGRLVPLLETLSERGHSVDLQDVLLRFAFDNICAAAFGVEAGCLADGLPDVPFARAFERATELSLTRFYTPPFIWKPKRLLCVGSERALVEAARAVREFAERTVADRRAELCKIGDLAGRCDLLSRLMSSSPPPADAGAGLAAGYSDEFLRDFCISFILAGRDTSSVALTWFFWLLASHPDVEARVLDDIARVGGGDVGAMDYLHAALTESMRLYPPVPVDFKEALEDDVLPDGTLVRARQRVIYFTYAMGRDKATWGPDCLEFRPERWLNKSGAFAGGAESPYKYVVFNAGPRLCVGKRFAYTQMKTVAAAVLARFRVEVVPGQEVKPKLNTTLYMKSGLMVRFVAREQRHELGHPVPAAADDAGGCSLH

SEQ ID NO: 114 - protein, 539 aa, Saccharum officinarum
MARALVAMALRFLRDYVRASDLAVAAAVLFVCSAVRSRLSSRPGEPMLWPVVGIIPTLFEHLAIGDVYDWGAAVLSRCRGTFPYRGTWGGGSSGVITSVPANVEHVLKDNFDNYPKGPYYRERFAELLGDGIFNADGDSWRAQRKAASAEMHSARFLRFSAATIERLVRGRLVPLLETLSERGHSVDLQDVLLRFAFDNICAAAFGVEAGCLADGLPDVPFARAFERATELSLTRFYTPPFIWKPKRLLCVGSERALVEAARAVREFAERTVADRRAELCKIGDLAGRCDLLSRLMSSSPPPADAGAGLAAGYSDEFLRDFCISFILAGRDTSSVALTWFFWLLASHPDVEARVLDDIARVGGGDVGAMDCLHAALTESMRLYPPVPVDFKEALEDDVLPDGTLVRARQRVIYFTYAMGRDKATWGPDCLEFRPERWLNKSGAFAGGAESPYKYVVFNAGPRLCVGKRFAYTQMKTVAAAVLARFRVEVVPGQEVKPKLNTTLYMKSGLMVRFVAREQRHELGHPVPAAADDAGGCSLH

SEQ ID NO: 115 - protein, 541 aa, Saccharum officinarum
MARALVAMALRFLRDYVRASDLAVAAAVLFVCSAARSRLSSRPGEPMLWPVVGIIPTLFAHLAIGDVYDWGAAVLSRCRGTFPYRGTWGGGSSGVITSVPANVEHVLKANFDNYPKGPYYRERFAELLGDGIFNADGDSWRVQRKAASSEMHSARFLQFSAATIERLVRGRLVPLLETLSERGADDAVVDLQDVLLRFAFDNICAAAFGVEAGCLADGLPDVPFAHAFERATELSLTRFYTPPFIWKPKRLLCVGSERALVEAARAVREFAERTVADRRAELRKVGDLAGRCDLLSRLMSSSPPPADAGAGLAAGYSDEFLRDFCISFILAGRDTSSVALTWFFWLLAFHPDVEARVLDDIALAGGDVGATDYLHAALTESMRLYPPVPVDFKEALEDDVLPDGTLVRARQRVIYFTYAMGRDKATWGPDCLEFCPERWLNKSGAFAGGAESPYKYVVFNAGPRLCVGKRFAYTQMKTVAAAVLARFRVEVVPGQEVKPKLNTTLYMKSGLMVRFVAREQRHELGHPVPAAADDAGGCSLH

SEQ ID NO: 116 - protein, 513 aa, Glycine max
MSPLIVALATIAAAILIYRIIKFITRPSLPLPPGPKPWPIVGNLPHMGPVPHHSLAALARIHGPLMHLRLGFVDVVVAASASVAEQFLKIHDSNFSSRPPNAGAKYIAYNYQDLVFAPYGPRWRLLRKLTSVHLFSGKAMNEFRHLRQEEVARLTCNLASSDTKAVNLGQLLNVCTTNALARAMIGRRVFNDGNGGCDPRADEFKAMVMEVMVLAGVFNIGDFIPSLEWLDLQGVQAKMKKLHKRFDAFLTSIIEEHNNSSSKNENHKNFLSILLSLKDVRDDHGNHLTDTEIKALLLNMFTAGTDTSSSTTEWAIAELIKNPQILAKLQQELDTVVGRDRSVKEEDLAHLPYLQAVIKETFRLHPSTPLSVPRAAAESCEIFGYHIPKGATLLVNIWAIARDPKEWNDPLEFRPERFLLGGEKADVDVRGNDFEVIPFGAGRRICAGLSLGLQMVQLLTAALAHSFDWELEDCMNPEKLNMDEAYGLTLQRAVPLSVHPRPRLAPHVYSMSS

SEQ ID NO: 117 - protein, 513 aa, Glycine max
MSTWVIGFATIIAAVLIYRVLKPISRPSSSLPLPPGPRPWPIVGNLPHMGPAPHQGLANLAQTHGPLMHLRLGFVDVVVAASASVAEQFLKIHDANFCSRPLNFRTTYLAYNKQDLVFAPYGPKWRFLRKLTTVHMFSAKAMDDFSQLRQEEVARLTCKLARSSSKAVNLRQLLNVCTTNALTRIMIGRRIFNDDSSGCDPKADEFKSMVGELMTLFGVFNIGDFIPALDWLDLQGVKAKTKKLHKKVDAFLTTILEEHKSFENDKHQGLLSALLSLTKDPQEGHTIVEPEIKAILANMLVAGTDTSSSTIEWAIAELIKNSRIMVQVQQELNVVVGQDRLVTELDLPHLPYLQAVVKETLRLHPPTPLSLPRFAENSCEIFNYHIPKGATLLVNVWAIGRDPKEWIDPLEFKPERFLPGNEKVDVDVKGNNFELIPFGAGRRICVGMSLGLKIVQLLIATLAHSFDWELENGTDPKRLNMDETYGITLQKAMPLSVHPHPRLSQHVYSSSSL

SEQ ID NO: 118 - protein, 512 aa, Glycine max
MSAWVIAFATVVAATLIYRLFKLITVPSLPLPPGPRPWPIVGNLPHMGPAPHQGLAALAQTHGPLMHLRLGFVDVVVASSASVAEQFLKIHDANFCSRPCNSRTTYLTYNQQDLVFAPYGPRWRFLRKLSTVHMFSAKAMDDFRELRQEEVERLTCNLARSSSKVVNLRQLLNVCTTNILARIMIGRRIFSDNSSNCDPRADEFKSMVVDLMVLAGVFNIGDFIPCLDWLDLQGVKPKTKKLYERFDKFLTSILEEHKISKNEKHQDLLSVFLSLKETPQGEHQLIESEIKAVLGDMFTAGTDTSSSTVEWAITELIKNPRIMIQVQQELNVVVGQDRLVTELDLPHLPYLQAVVKETLRLHPPTPLSLPRFAENSCEIFNYHIPKGATLLVNVWAIGRDPKEWIDPLEFKPERFFPGGEKDDVDVKGNNFELIPFGAGRRICVGMSLGLKVVQLLIATLAHSFDWELENGADPKRLNMDETYGITLQKALPLFVHPHPRLSQHVYSSSSSL

SEQ ID NO: 119 - protein, 511 aa, Glycine max
MSPWVIAVATIVAAILIYRVLKHIAGPSLPLPPGPRPWPIVGNLPHMGPAPHQGLAALAKTHGPLMHLRLGFVHVVVAASAAVAEQFLKVHDANFCNRPYNFRTTYMTYNKKDIAFYPYGPRWRFLRKICTVHMFSGKAMDNFSQLRQEEVERLACNLTRSNSKAVNLRQLLNVCITNIMARITIGRRIFNDDSCNCDPRADEFKSMVEEHMALLGVFNIGDFIPPLDWLDLQGLKTKTKKLHKRFDILLSSILEEHKISKNAKHQDLLSVLLSLKETPQEGHELVEEEIKSILGDMFTAGTDTSLSTIEWAIAELIKNPKIMIKVQQELTTIVGQNRLVTELDLPHLPYLNAVVKETLRLHPPTPLSLPRVAEESCEIFNYHIPKGATLLVNVWAIGRDPKEWLDPLEFKPERFLPGGEKADVDIRGNNFEVIPFGAGRRICVGMSLGIKVVQLLIASLAHAFDWELENGYDPKKLNMDEAYGLTLQRAVPLSIHTHPRLSQHVYSSLSL

SEQ ID NO: 120 - protein, 452 aa, Glycine max
MYLRLGFVDVVVAASASVAEQFLKVHDANFSSRPLNSMTTYMTYNQKDLAFAPYGPRWRFLRKISSVHMFSVKALDDFRQLRQEEVERLTSNLASSGSTAVNLGQLVNVCTTNTLARVMIGRRLFNDSRSSWDAKADEFKSMVVELMVLNRVFNIGDFIPILDRLDLQGVKSKTKKLHKRFDTFLTSILEEHKIFKNEKHQDLYLTTLLSLKEAPQEGYKLDESEIKAILLDMFTAGTDTSSSTIEWAIAELIRNPRVMVRVQQEMDIVVGRDRRVTELDLPQLPYLQAVVKETFRLHPPTPLSLPRVATESCEIFDYHIPKGTTLLVNIWAIGRDPNEWIDPLEFKPERFLLGGEKAGVDVMGTNFEVIPFGAGRRICVGMGLGLKVVQLLTATLAHTFVWELENGLDPKNLNMDEAHGFILQREMPLFVHPYPRLSRHVYSSSSSPSSSS

SEQ ID NO: 121 - protein, 354 aa, Oryza sativa
MASLMEVQVPLLGMGTTMGALALALVVVVVVHVAVNAFGRRRLPPSPASLPVIGHLHLLRPPVHRTFHELAARLGPLMHVRLGSTHCVVASSAEVAAELIRSHEAKISERPLTAVARQFAYESAGFAFAPYSPHWRFMKRLCMSELLGPRTVEQLRPVRRAGLVSLLRHVLSQPEAEAVDLTRELIRMSNTSIIRMAASTVPSSVTEEAQELVKVVAELVGAFNADDYIALCRGWDLQGLGRRAADVHKRFDALLEEMIRPERFLAGGGGEGVEPRGQHFQFMPFGSGRRGCPGMGLALQSVPAVVAALLQCFDWQCMDNKLIDMEEADGLVCARKHRLLLHAHPRLHPFPPLL

SEQ ID NO: 122 - protein, 516 aa, Oryza sativa
MASLMEVQVPLLGMGTTMGALALALVVVVVVHVAVNAFGRRRLPPSPASLPVIGHLHLLRPPVHRTFHELAARLGPLMHVRLGSTHCVVASSAEVAAELIRSHEAKISERPLTAVARQFAYESAGFAFAPYSPHWRFMKRLCMSELLGPRTVEQLRPVRRAGLVSLLRHVLSQPEAEAVDLTRELIRMSNTSIIRMAASTVPGSVTEEAQELVKVVAELVGAFNADDYIALCRGWDLQGLGRRAADVHKRFDALLEEMIRHKEEARMRKKTDTDVGSKDLLDILLDKAEDGAAEVKLTRDNIKAFIIDVVTAGSDTSAAMVEWMVAELMNHPEALRKVREEIEAVVGRDRIAGEGDLPRLPYLQAAYKETLRLRPAAPIAHRQSTEEIQIRGFRVPAQTAVFINVWAIGRDPAYWEEPLEFRPERFLAGGGGEGVEPRGQHFQFMPFGSGRRGCPGMGLALQSVPAVVAALLQCFDWQCMDNKLIDMEEADGLVCARKHRLLLHAHPRLHPFPPLL

SEQ ID NO: 123 - protein, 549 aa, Brachypodium distachyon
MAMAASSMEQLLQVDPAMATYSILAIALVTAVLVLINRIGGNGAGKQRRHGLPPSPRRLPVIGHLHLLRPPVHRTFQELASGLGAPLMHIRLGSTHCLVASSAAAATELIRSHEGKISERPLTAVARQFAYGSDSGFAFAPYGPHWRAMKRLCMSELLGPRTVELLRPVRRAGLVSLLHTVIRKSPEPVDLTAELIRMSNASIIRMMASTVPGSVTEEAQALVKAVAELVGAFNVEDYIAVCRGWDLQGLGKRAADVHRRFDALLEDMIAHKEEARAAKKAIRGEDDQEPETKKTMAESKDLIDILLDKMEDENAAEETKLTREKIKAFTIDVVTAGSDTSAAMVEWMLAELMNHPECLRKVRDEIDAVVGSNRITGEADIANLPYLQAAYKETLRLRPAAPIAHRQSTEDMELATGGCFTVPVGTAVFINLWAIGRDPEHWGQTALEFRPERFMLGGESEKLEPRGQHFQYLPFGSGRRGCPGMGLALQSVPAVVAALVQCFHWTVVPKAGEEKAVIDMEESDGLVRARKHPLLLRASPRLNPFPAVV

SEQ ID NO: 124 - protein, 563 aa, Triticum aestivum
MCSPEVAGGTLAAMATASSMQQPALLLLRQLTQDPVTASLLAVALATAVLMIAALSRGGGRKPRLPPSPRGFPVIGHLHLVRPPVHRTFHDLAARLGPLMHIRLGSTHCVVASSAGVAAELIRTHEGKISERPLTAVARQFAYGDDGFAFAPYGPHWRSMKRLCMSELLGPRTVEQLRPVRRAGLVSLLQSVLHQASGAEAVDLTAALIRLSNTSIIRMMASTVPGSVTGEAQALVKAVAELVGAFNVEDYIAVCRGWDLQGLGRRAADVHRRFDALLEQMIRHKEEAREARKMRGGAEGETPEKKTATGTTTESSKDLLDILLDKLEDDAAAEVKLTRKKIKAFVIDVVTAGSDTSAAMVEWMLAELMNHPECLRKVREEIDAVVGRDRIAGEGDVASLPYLQAAYKETLRLRPAAPIAHRQSTEEMVVTAAGGVGGFTVPAGTAVFMNLWSIARDPANWDAPLEFRPERFMAGGRNEALDPRGQHFQYLPFGSGRRGCPGMGLALQSVPAVVAALVQCFDWAVDGDAKKIDMEEADGLVCARKHPLLLRPSPRLSPFPAVV

SEQ ID NO: 125 - protein, 551 aa, Triticum aestivum
MATASSMQQPALLLLRQLMQDPVIASLLAVALATVIMLIGAVSRGGGRKPRLPPSPRGFPVIGHLHLVRPPVHRTFHDLAARLGPLMHIRLGSTHCVVASSAGVAAELIRTHEGKISERPLTAVARQFAYGDDGFAFAPYGPHWRSMKRLCMSELLGPRTVEQLRPVRRAGLVSLLQSVLHQASGAEAVDLTAALIRLSNTSIIRMMASTVPGSVTEEAQALVKAVAELVGAFNVEDYIAVCRGWDLQGLRRRAADVHRRFDALLEEMIRHKEEAREARKMRGGGEGETPEKKTATGTTTESSKDLLDILLDKLEDDAAAEVKLTRKKIKAFVIDVVTAGSDTSAAMVEWMLAELMNHPECLRKVRAEIDAVVGRDRIAGEGDVASLPYLQAAYKETLRLRPAAPIAHRQSTEEMVISAAGGGVGGFTVPAGTAVFMNLWSIARDPANWDAPLEFRPERFMAGGRNEALDPRGQHFQYLPFGSGRRGCPGMGLALQSVPAVVAALVQCFDWAVDGDGKKIDMEEADGLVCARKHPLLLRPSPRLSPFPAVV

SEQ ID NO: 126 - protein, 549 aa, Triticum aestivum
MAMASSMQQPALLLLRQLTQDPVTASLLAAALATAVLMIAAVRRGGGRKPRLPPSPRGFPVIGHLHLVRPPVHRTFHDLAARLGPLMHIRLGSTHCVVASSAGVAAELIRTHEGKISERPLTAVARQFAYGDDGFAFAPYGPHWRSMKRLCMSELLGPRTVEQLRPVRRAGLVSLLQSVLHQASGAEAVDLTAALIRLSNTSIIRMMASTVPGSVTEEAQELVKAVAELVGAFNVEDYIAVCRGWDLQGLGRRAADVHRRFDALLEEMIRHKEEAREARRMRGGGEGETPEKKTATGTTTESSKDLLDILLDKLEDDAAAEVKLTRKKIKAFVIDVVTAGSDTSAAMVEWMLAELMNHPECLRKVRSEIDAVVGRDRIAGEGDVASLPYLQAAYKETLRLRPAAPIAHRQSTEEMVVTAAGGFTVPAGTAVFINLWSIARDPANWDAPLEFRPERFLAGGRNEALDPRGQHFQYLPFGSGRRGCPGMGLALQSVPAVVAALVQCFDWAVPADIDGKKIDMEEADGLVCARKHPLLLRPSPRLSPFPAVV

SEQ ID NO: 127 - protein, 559 aa, Triticum turgidum
MCDPEVAGATLAAMATASSMQQPALLLRQLTQDPVTASLLAAALATAVLMIAAVSRGGGRKPRLPPSPRGFPVIGHLHLVRPPVHRTFHDLAARLGPLMHIRLGSTHCVVASSAGVAAELIRAHEGKISERPLTAVARQFAYGDDGFAFAPYGPHWRSMKRLCMSELLGPRTVEQLRPVRRAGLVSLLQSVLHRASGAEAVDLTAALIRLSNTSIIRMMASTVPGSVTEEAQALVKAVAELVGAFNVEDYIAVCRGWDLQGLRRRAADVHRRFDALLEEMIRHKEEAREARKMRGGAEGETPEKKTATGTTTESSKDLLDILLDKLEDDAAAEVKLTRKKIKAFVIDVVTAGSDTSAAMVEWMLAELMNHPECLRKVRAEIDAVVGRDRIAGEGDVASLPYLQAAYKETLRLRPAAPIAHRQSAEEMVISAAGGFTVPAGTAVFINLWSIARDPANWDAPLEFRPERFMAGGRNEALDPRGQHFQYLPFGSGRRGCPGMGLALQSVPAVVAALVQCFDWAVDGDAEKIDMEEADGLVCARKHPLLLRPSPRLSPFPAVV

SEQ ID NO: 128 - protein, 549 aa, Triticum turgidum; identical to SEQ ID NO: 126

SEQ ID NO: 129 - protein, 543 aa, Setaria italica
MAMETEQPLPILLSADSVAVLAVGTLLALALNHLVSSWRSARRLPPSPPGLPVIGHLHLLRPPAHRTFHELAGKLGPLMHIRLGSTHCVVAGSADVARELIHRHDAAISGRPVTALARLFSYSSAGFAFTPYSPRWRFLRRLCVSEVLSPRTVEQLRPVRRAALAPLLRAVLAASERGEAADVTGELVRFANASIIRMVASDAPGSVADEAQGLVKAVTELIGAFNVEDYVPLCRGWDLQGLRSTAAGVHRRFDALLEQMIRHKEEARERGRSCGAIYELEHEQEDEKGSAPATRKRNKDLLDILLEKAEDEAAEVKLTRENIKAFITDVVTAGSDSSAATVEWMLAELVNHPEVMRKVREEIDAVVTGDCRIVGEADLPRLPYLQAAFKETLRLHPGAPIAHRVSTAEISVRGFMVPPRTAVFINVWAIGRDPAFWEDPTAFRPERFMPGGAAAGLEPQPRGHHFQFMPFGGGRRGCPGVGLAQQSVPAVLAALVQCFDWAVADGETGLVDMEESDVGLVCARKHPLLLRPTPRLNPFPSVV

SEQ ID NO: 130 - protein, 538 aa, Cenchrus americanus
  MOD_RES (384)..(468): Any amino acid
MEMEQPLPMLLSADTVAIMAVVTFLALAVNHLVSSWLSSPRRRLPPSPPGLPVIGHLHLLRLPAHRTFHELAGKLGPLMHLRLGSTHCVVASSADVARELILRHDAAISGRPVTALARLFSYGSVGFAFTPYSPRWRFLRRLCVSELVRFASASIIRMVASDAPGNVSDEAQGLVKSVTELIGAFNVEDYVPLCRGWDLQGLRRTADGVHRRFDALLEQMIRHKEEARERARSDMAEHEQHDKKDASASAAPTTRKRNKDLLDILLEKAEDDEAEVKLTRENIKAFITDVVTAGSDSSAATVEWMLAELVNHPEAMRKVREEIDAAVGEDSRIVSEADLPRLPYLQAAFKETLRLHPGAPIAHRVSSAEEMAVGGFTVPPRTAXXXXXXXXXXXXXXXXXXXXXXXXXXXXXXXXXXXXXXXXXXXXXXXXXXXXXXXXXXXXXXXXXXXXXXXXXXXXXXXXXXXXXPGVGLAQQSVPAVLAALVQCFDWAAVVDGEMSPTGSLVNMEESDVGLVCARKHSLLLRPTARLNPFPAVV

SEQ ID NO: 131 - protein, 337 aa, Cenchrus americanus
MVASTVPGRVADEAQELVKDVAELVGAFNADDYIALCRGWDLQGLRRRAADVHRRFDALLEEILRHKEDAREARKLLMLDGGDGARKKKEAATATTAHKDLLDILMDKAEDKTAEANLTRDNIKAFIIDVVTAGSDTSAAMVEWMLAELMNHPEALRKVVAEIDGVVGGERIAGEADLPQLPYLMAAYKETLRLHPAAPIAHRQSSEEMVLRGFTVPPQTAVFINIWAIGRDPAFWEDPLAFRPERFMPGGAAESLEPRGQHFHFMPFGSGRRGCPGMGLALQSVPAVLAALVQCFDWATAAGEPIDMDESDGLVCARKHPLLLRPTPRLNPFPAVV

SEQ ID NO: 132 - protein, 552 aa, Sorghum bicolor
MAMDQPAMPMLMSTDSAAAVMVLLSVATLLLALNHHLLSSWRRRSSRRLPPSPPRLPVIGHLHLLRPPVHRTFHELATRLGAPLMHIRLGSTHCVVAGTADVARELIRDHDAAISGRPVSVLSRLFSYGSAGFAFTPNSRHWRFLRRLCVSEVLGTRTVEQLRHVRRGSLAELLRAVRASSARGDAVDVTRELIRFSNTAIIRMVASDAAVTDEAQELVKAVTELLGAFNLEDYVPLCRGWDLQGLRRKATVVHRRFDAVLEQMIRHKEAARDMERRRRGGSGTLEDKRVEGPPATTCKQRNKDLLDILLDKAEDETAEVKLTRENMKAFIIDVVTAGSDSSAVTVEWMLAELMNHPEALGKVRDEIDAVVGGGDGRIVGEADLARLPYLQATFKETLRLHPGAPIAHRQSTTEMVVRGFTVPPETAVYINLWAIGRDPSFWEDPLAFRPERFMPGGAAEGLEPRGGGGGGQQFQFMPFGSGRRGCPGMGLAQQSVPAVLAALVQCFDWAAADDGETAAIGMDESDVGLVCARKHPLVLRPTARLNPFPAVV

SEQ ID NO: 133 - protein, 555 aa, Sorghum bicolor
MAAMEEQPLSSSSTSMAIVLSLLKNNPADAVLLALVAVVALRHYLISSWRQQEQARRLPPGPRRLPVIGHLHLLRPPVHRTFQELASRMGPLMHIQLGSTHCVVASSPEVASELIRGHEGSISERPLTAVARQFAYDSAGFAFAPYNTHWRFMKRLCMSELLGPRTVEQLRPIRRAGTVSLLGDLLLAAASSETETETVVDLTRHLIRLSNTSIIRMVASTVPGSVTDEAQELVKAVAELVGAFNADDYIAVIRGWDLQGLRRRAADVHRRFDALLEDILKHKEEARAARRRLDDDDGHRVSKKQATAPHSKDLLDILMDKAEDPAAEVKLTRENIKAFIIDVVTAGSDTSAAMVEWMLAELLNHPETLRKVVEEIDAVVGGDRIASEADLPQLPYLMAAYKETLRLHPAAPIAHRQSTDEMVVRGFTVPPQTAVFINVWAIGRDPAYWEEPLAFRPERFMPGGAADSLEPRGQHFQYMPFGSGRRGCPGMGLALQSVPAVLAALVQCFHWATVDGDGDGDSKIDMSESDGLVCARKKPLLLRPTPRLSPFPAVV

SEQ ID NO: 134 - protein, 538 aa, Zea mays
MAMDLLAMPVLLSADSAAAVLVLLSVATVVALKHLLSSWRRSPRRRLPPSPTPLPVIGHLHLLRPPVHRTFHELATRLGAPLMHIRLGSTHCVVVGSADVARELIHDHDATISGRPVSVLSRLFSYGSAGFAFTPYSPHWRFLRRLCVSEVLGPRTVEQLRHVRRGSLVSLLRSVLASSARGDNKVDLTRELIRFSTTSIIRMVASDVGVTDEAQELVKGVAELLGAFNLEDYVPLCRGWDLQGLRRKANGVHRRFDAVLEQMIRHKEEARDRERGRGGAAQEDKKGWPATCKQRNKDLLDILLDMAENETAEVKLTRENMKAFIVDVVTAGSDSSAATVEWMLAELMNHPEALRKVRAEIDAVVGADRIVGEEDLPRLPYLQATFKETLRLHPGAPIAHRESTGEMVVRGFTVPPRTAVFFNLWAIGRDPSCWEEPLAFRPERFMPGGASEGLPPRGQQFQFIPFGSGRRACPGMGLAQQSVPTVLAALVQCFDWAAVDGETAAMGMDESDGGLVCARKHPLVLRPTARLNPFPAVV

SEQ ID NO: 135 - protein, 544 aa, Zea mays
MEEQQPRPRPSIMFVLSSLAKNNPESVLALIAVLTVVALRHLISSWRQQAPLPPSPTSLPVIGHLHLLRPPVHRTFQELASRIGPLMHIRLGSTHCVVASSPEVASELIRGHEGSISERPLTAVARQFAYDSAGFAFAPYNTHWRFMKRLCMSELLGPRTVEQLRPIRRAGTVSLLADLLASSARGETVDLTRHLIRLSNTSIIRMVASTVPGSVTDEAQEVVKDVAELVGAFNVDDYISLVRGWDLQGLRRRAAGVHRRFDALLEDILRHKEEARAARRLDQDDDGRGSSKQDKKQATHSKDLLDILMDKADDPAAEIKLTRENIKAFIIDVVTAGSDTSAAMVEWMLAELMNHPETLRKVAEEIDAVVGGDRIASEADLPQLPYLMAAYKETLRLHPAAPIAHRQSSEEMVVRGFTVPPQTAVFINVWAIGRDPAYWEEPLAFRPERFMPGGAAESLEPRGQHFQYMPFGSGRRGCPGMGLALQSVPAVLAALVQCFHWATVDGDGGVNKIDMSESDGLVCARKKPLLLRPTPRLTPFPAVV

SEQ ID NO: 136 - protein, 314 aa, Zea mays
  MOD_RES (78)..(78): Any amino acid
MKEQQPRPRPSIMFVLSSLAKNNPEAVLALIAVVTVVALRHLISSWRQQAPLPPSPTSLPVIGHLHLLRPPVHRTFQXWTRRRTRRRRSSSPRENIKAFIIDVVTAGSDTSAAMVEWMLAELMNHPETLRKVVEEIDAVVGGDRIASEADLPRLPYLMAAYKETLRLHPAAPIAHRQSSAEMVVRGFTVPPQTAVFINVWAIGRDPAYWEEPLAFRPERFMPGGAAENLEPRGQHFQYMPFGSGRRGCPGMGLALQSVPAVLAALVQCFHWATVDGDGGVNKIDMSESDGLVCARKKPLLLRATPRLTPFPAVV

SEQ ID NO: 137 - protein, 532 aa, Zea mays
MEEQQLRARPNMMVLSSLAKNNPEAVLALIAFVTVVALRQLISSWRQHGRLPPGPTSLPVIGHLHLLRPPVHRTLQELASRIGPLMHIRLGSTNCVVASSPEVVSELIRGHEGSISARPFTAVARQFSYDSAGFVFEPYNTHWRFMKRLCMSELLGPRTVEQLRPVRRAVTVSLVSDLLASSARGETVDITRHLIRLTNTSIIRMVASTVSGSVTDEAHELAKAVIEVVGAFNVDDYIAVVRGWDLQGLGRKAADVHRRFDALLEDILRHKEEARAARRLDDGHGKQATHSKDLLDILMDKAEDPAAEVKLTRENIKAFVIDVVTSGSDTSAAMAEWMLAELMNHPETLRKVVEEIDAVVGGGRIASEADLPQLPYLMAVYKETLRLHPAGPIAHRQSTEEMVVHGFTVPPQSTVLIHVWAIGRDPAYWEEPLLFRPERFMPGGAAESLEPRGKHFQYIPFGSGRRGCPGMGLAMQSVPAVVAALVQCFYWATVDGGVNKIDMSESDGLVCARKKPLLLRPTSRLTPFPPVV

SEQ ID NO: 138 - protein, 477 aa, Zea mays
MAMDLLAMPVLLSADSAAAVLVLLSVATVVALKHLLSSWRRSPRRRLPPSPTPLPVIGHLHLLRPPVHRTFHELATRLGAPLMHIRLGSTHCVVVGSADVARELIHDHDATISGRPVSVLSRLFSYGSAGFAFTPYSPHWRFLRRLCVSEVLGPRTVEQLRHVRRGSLVSLLRSVLASSARGDNKVDLTRELIRFSTTSIIRMVASDVGVTDEAQELVKGVAELLGAFNLEDYVPLCRGWDLQGLRRKANGVHRRFDAVLEQMIRHKEEARDRERGRGGAAQEDKKGWPATCKQRNKDLLDILLDMAENETAEVKLTRENMKAFIVETLRLHPGAPIAHRESTGEMVVRGFTVPPRTAVFFNLWAIGRDPSCWEEPLAFRPERFMPGGASEGLPPRGQQFQFIPFGSGRRACPGMGLAQQSVPTVLAALVQCFDWAAVDGETAAMGMDESDGGLVCARKHPLVLRPTARLNPFPAVV

SEQ ID NO: 139 - protein, 532 aa, Zea mays
MFVLSSLAKNNPESVLALIAVLTVVALRHLISSWRQQARLPPSPTSLPVIGHLHLLRPPVHRTFQELASRIGPLMHIRLGSTHCVVASTPEVASELIRGHEGSISERPLTAVARQFAYDSAGFAFAPYNTHWRFMKRLCMSELLGPRTVEQLRPVRRAGTVSLLADLLASSARGETVDLTRHLIRLSNTSIIRMVASTVPGSVTDEAQEVVKDVAELVGAFNVDDYISLVRGWDLQGLRRRAAGVHRRFDALLEDILRHKEEARAARRLDQDDDGRGSSKQDKKQATHSKDLLDILMDKADDPAAEIKLTRENIKAFIIDVVTAGSDTSAAMVEWMLAELMNHPETLRKVAEEIDAVVGGDRIASEADLPQLPYLMAAYKETLRLHPAAPIAHRQSSEEMVVRGFTVPPQTAVFINVWAIGRDPAYWEEPLAFRPERFMPGGAAESLEPRGQHFQYMPFGSGRRGCPGMGLALQSVPAVLAALVQCFHWATVDGDGGVNKIDMSESDGLVCARKKPLLLRPTPRLTPFPAVV

SEQ ID NO: 140 - protein, 544 aa, Zea mays
MKEQQPRPRPSIMFVLSSLAKNNPEAVLALIAVVTVVALRHLISSWRQQAPLPPSPTSLPVIGHLHLLRPPVHRTFQELASRIGPLMHIRLGSTHCVVASSPEVASELIRGHEGSISERPLTAVARQFAYDSAGFAFAPYNTHWRFMKRLCMSELLGPRTVEQLRPIRRAGTVSLLGDLLASSARGETVDLTRHLIRLSNTSIIRMVASTVPGSVTDEAQKVVKDVAELVGAFNVDDYIAVVRGWDLQGLRRRAADVHRRFDALLEDILRHKEEARAARRLDQDDGQGISSKQDKKQATHSKDLLDILMDKAEDQAAEVKLTRENIKAFIIDVVTAGSDTSAAMVEWMLAELMNHQETLRKVVEEIDAVVGGDRIASEADLPRLPYLMAAYKETLRLHPAAPIAHRQSSEEMVVRGFTVPPQTAVFINVWAIGRDPAYWEEPLAFRPERFMPGGAAESLEPRGQHFQYMPFGSGRRGCPGMGLALQSVPAVLAALVQCFHWATVDGDGGVNKIDMSESDGLVCARKKPLLLRPTPRLTPFPAVV

SEQ ID NO: 141 - protein, 427 aa, Zea mays
MKEQQPRPRPSIMFVLSSLAKNNPEAVLALIAVVTVVALRHLISSWRQQAPLPPSPTSLPVIGHLHLLRPPVHRTFQELASRIGPLMHIRLGSTHCVVASSPEKVVKDVAELVGAFNVDDYIAVVRGWDLQGLRRRAADVHRRFDALLEDILRHKEEARAARRLDQDDGQGISSKQDKKQATHSKDLLDILMDKAEDQAAEVKLTRENIKAFIIDVVTAGSDTSAAMVEWMLAELMNHPETLRKVVEEIDAVVGGDRIASEADLPRLPYLMAAYKETLRLHPAAPIAHRQSSAEMVVRGFTVPPQTAVFINVWAIGRDPAYWEEPLAFRPERFMPGGAAENLEPRGQHFQYMPFGSGRRGCPGMGLALQSVPAVLAALVQCFHWATVDGDGGVNKIDMSESDGLVCARKKPLLLRATPRLTPFPAVV

SEQ ID NO: 142 - protein, 521 aa, Zea mays
MMVLSSLAKNNPEAVLALIAFVTVVALRHLISSWRQHGRLPPGPTSLPVIGHLHLLRPPVHRTLQELASRIGPLMHIRLGSTNCVVASSPEVASELIRGHEGSISARPFTAVARKFSYDSAGFVFEPYNTHWRFMKRLCMSELLGPRTVEQLRPVRRAVTVSLVSDLLASSARGETVDITRHLIRLTNTSIIRMVASTVSGSVTDEAHELAKAVIEVVGAFNVDDYIAVVRGWDFQGLGRKAADVHRRFDALLEDILRHKEEARAARRLDDGHGKQATHSKDLLDILMDKAEDPAAEVKLTRENIKAFVIDVVTSGSDTSAAMAEWMLAELMNHPETLRKVVEEIDAVVGGGRIASEADLPQLPYLMAVYKETLRLHPAGPIAHRQSTEEMVVHGFTVPPQSTVLIHVWAIGRDPAYWEEPLLFRPERFMPGGAAESLEPRGKHFQYIPFGSGRRGCPGMGLAMQSVPAVVAALVQCFHWSTVDGGMDKIDMSESDGLVCARKKPLLLRPTSRLTPFPPVV

SEQ ID NO: 143 - protein, 481 aa, Zea mays
MAMDLLAMPVLLSADSAAAVLVLLSVATVVALKHLLSSWRRSPRRRLPPSPTPLPVIGHLHLLRPPVHRTFHELATRLGAPLMHIRLGSTHCVVVGSADVARELIHDHDATISGRPVSVLSRLFSYGSAGFAFTPYSPHWRFLRRLCVSEVLGPRTVEQLRHVRRGSLVSLLRSVLASSARGDNKVDLTRELIRFSTTSIIRMVASDVGVTDEAQELVKGVAELLGAFNLEDYVPLCRGWDLQGLRRKANGVHRRFDAVLEQMIRHKEEARDRERSRGGAAQEDKKGWPATCKQRNKDLLDILLDMAENETAEVKLTRENMKAFIVATFKETLRLHPGAPIAHRESTGEMVVRGFTVPPRTAVFFNLWAIGRDPSCWEEPLAFRPERFMPGGASEGLPPRGQQFQFIPFGSGRRACPGMGLAQQSVPTVLAALVQCFDWAAVDGETAAMGMDESDGGLVCARKHPLVLRPTARLNPFPAVV

SEQ ID NO: 144 - protein, 544 aa, Zea mays
MEEQQPRPRPSIMFVLSSLAKNNPESVLALIAVLTVVALRHLISSWRQQARLPPSPTSLPVIGHLHLLRPPVHRTFQELASRIGPLMHIRLGSTHCVVASTPEVASELIRGHEGSISERPLTAVARQFAYDSAGFAFAPYNTHWRFMKRLCMSELLGPRTVEQLRPVRRAGTVSLLADLLASSARGETVDLTRHLIRLSNTSIIRMVASTVPGSVTDEAQEVVKDVAELVGAFNVDDYISLVRGWDLQGLRRRAAGVHRRFDALLEDILRHKEEARAARRLDQDDDGRGSSKQDKKQATHSKDLLDILMDKADDPAAEIKLTRENIKAFIIDVVTAGSDTSAAMVEWMLAELMNHPETLRKVAEEIDAVVGGDRIASEADLPQLPYLMAAYKETLRLHPAAPIAHRQSSEEMVVRGFTVPPQTAVFINVWAIGRDPAYWEEPLAFRPERFMPGGAAESLEPRGQHFQYMPFGSGRRGCPGMGLALQSVPAVLAALVQCFHWATVDGDGGVNKIDMSESDGLVCARKKPLLLRPTPRLTPFPAVV

SEQ ID NO: 145 - protein, 532 aa, Zea mays
MEEQQLRARPNMMVLSSLAKNNPEAVLALIAFVTVVALRHLISSWRQHGRLPPGPTSLPVIGHLHLLRPPVHRTLQELASRIGPLMHIRLGSTNCVVASSPEVASELIRGHEGSISARPFTAVARKFSYDSAGFVFEPYNTHWRFMKRLCMSELLGPRTVEQLRPVRRAVTVSLVSDLLASSARGETVDITRHLIRLTNTSIIRMVASTVSGSVTDEAHELAKAVIEVVGAFNVDDYIAVVRGWDFQGLGRKAADVHRRFDALLEDILRHKEEARAARRLDDGHGKQATHSKDLLDILMDKAEDPAAEVKLTRENIKAFVIDVVTSGSDTSAAMAEWMLAELMNHPETLRKVVEEIDAVVGGGRIASEADLPQLPYLMAVYKETLRLHPAGPIAHRQSTEEMVVHGFTVPPQSTVLIHVWAIGRDPAYWEEPLLFRPERFMPGGAAESLEPRGKHFQYIPFGSGRRGCPGMGLAMQSVPAVVAALVQCFYWATVDGGVDKIDMSESDGLVCARKKPLLLRPTSRLTPFPPVV

SEQ ID NO: 146 - DNA, 33 bp, Oryza sativa
gggtggcccgtgctgggcaacctgccgcagctc

SEQ ID NO: 147 - DNA, 26 bp, Oryza sativa
gcccgctgttccggctccggttcggc

SEQ ID NO: 148 - DNA, 29 bp, Oryza sativa
gggacttcgtgccggcgctccggtggctc

SEQ ID NO: 149 - DNA, 33 bp, Oryza sativa
ggatggccggtgctgggcaacctgccgcagctg

SEQ ID NO: 150 - DNA, 26 bp, Oryza sativa
gcccgctgatccggctccggttcggg

SEQ ID NO: 151 - DNA, 29 bp, Oryza sativa
gcgacttcgtgccggcgctccggtggctg

(19) United States
(12) Patent Application Publication
(10) Pub. No.: US 2023/0257758 A1
(43) Pub. Date: Aug. 17, 2023

(54) PLANT METABOLITE-MEDIATED INDUCTION OF BIOFILM FORMATION IN SOIL BACTERIA TO INCREASE BIOLOGICAL NITROGEN FIXATION AND PLANT NITROGEN ASSIMILATION

(71) Applicant: The Regents of the University of California, Oakland, CA (US)

(72) Inventors: Eduardo BLUMWALD, Davis, CA (US); Dawei YAN, Davis, CA (US); Howard-Yana SHAPIRO, Davis, CA (US)

(21) Appl. No.: 18/015,229

(22) PCT Filed: Jul. 13, 2021
(86) PCT No.: PCT/US2021/041482

§ 371 Date: Jan. 9, 2023

Related U.S. Application Data
(60) Provisional application No. 63/051,267, filed on Jul. 13, 2020.

(51) Int. Cl. C12N 15/82; C12N 9/22

(52) U.S. Cl. CPC C12N 15/8243; C12N 15/8261; C12N 9/22; C12N 2310/20

(57) ABSTRACT

The present disclosure provides methods for increasing the yield of grain crops grown under reduced inorganic nitrogen conditions.

### CROSS-REFERENCE TO RELATED APPLICATIONS

This application is a US National Phase application Under 371 of PCT/US2021/041482 filed Jul. 13, 2021, which claims priority to U.S. Provisional Patent Application No. 63/051,267, filed Jul. 13, 2020, each of which is incorporated herein by reference in its entirety.

### REFERENCE TO AN ELECTRONIC SEQUENCE LISTING

The contents of the electronic sequence listing (081906-1252964_SL.txt; Size: 610,655 bytes; and Date of Creation: Dec. 2, 2022) is herein incorporated by reference in its entirety.

### BACKGROUND OF THE INVENTION

In the soil, plants are constantly exposed to a microbe-rich environment that can be beneficial or detrimental to plant growth. When potentially compatible bacterial partners sense plant (host) signals, an extensive, multiple stage, chemical communication is established to develop a successful plant-microbe interaction (1, 2). By contrast, plants have unique defense mechanisms to fight pathogen infections, and the arms race between host plants and pathogens rapidly drives the coevolution of plant resistance genes and pathogen avirulence effectors (3, 4). The adaptation of plants to such environments involves shaping their microbiota through the action of root exudates (5). It was estimated that plants extrude up to 20% of their fixed carbon in exchange for benefits such as acquisition of phosphorus and nitrogen, defense against biotic and abiotic stresses (6, 7).

The best-characterized example of symbiosis between plant and bacteria is the association of legumes and nitrogen fixation rhizobia, with the characteristic formation of root nodules. The nodule is the main organ for nitrogen fixation and its formation requires common symbiotic pathways (1, 2). In the soil, Rhizobia sense the host chemical signals (for example, flavonoids) and further activate the expression of nod genes through the nodD-flavonoids interaction. Nod gene-encoded lipochitooligosaccharides (LCDs) can be recognized by the LysM receptor kinase, located at the plasma membrane of the legume root, and calcium spiking can be triggered in the nucleus. The calcium signal is decoded by Ca²⁺/CaM-dependent protein kinases (CCaMK) and the phosphorylation of the transcription factor CYCLOPS. A set of other transcription factors is then activated for the regulation of the curling of the host's root hairs and the growth of an infection thread, leading to the development of nodules (2, 8).

The legume-rhizobium symbiosis has a very strict specificity, such that each legume can interact with only a specific group of rhizobia and vice versa (9). This narrowed host range restricts the application of rhizobia to other important non-leguminous crops such as rice, wheat, or corn. On the other hand, non-leguminous crops may form mutualistic relationships with other plant growth promoting bacteria (PGPB) and benefit from their partners for their nitrogen needs. Nitrogen derived from air (Ndfa), estimated by ¹⁵N enrichment experiments, showed that biological nitrogen fixation (BNF) can contribute between 1.5˜21.0% of the total nitrogen requirement of rice, depending on the genotypes (10). Interestingly, the common symbiotic pathway seems to not be required for such interactions, at least for the case of Azoarcus sp.-rice interactions (11). How such mutualistic relationships are established or regulated remain to be investigated.

Biofilms are essential for optimal colonization of host plant and contribute to nitrogen fixation. Biofilms are often seeded by “aggregates” that are embedded in a self-produced matrix of extracellular polymeric substances (EPS) containing polysaccharides, proteins, lipids, and extracellular DNA (12). The matrix provides shelter and nutrients for the bacteria, and it contributes to tolerance/resistance toward antimicrobial compounds. In addition, biofilms enable effective interactions by chemical communication (quorum sensing) to remodel the soil bacterial community dynamically, making biofilms one of the most successful modes of life on earth (13). In some cases, biofilm formation is indispensable for a successful bacterial colonization. For example, the Gluconacetobacter diazotrophicus mutant MGD, which is defective in polysaccharide production, cannot form biofilm (does not produce EPS) and cannot attach to plant root surfaces nor colonize endophytically the roots (14).

The formation of the EPS matrix of biofilms also generates heterogeneity, including the establishment of stable gradients of nutrients, pH, and redox conditions. More importantly, because of the decreased oxygen diffusion across bacterial biofilms, free-living nitrogen-fixing bacteria (Azospirillum brasilense, Pseudomonas stutzeri, etc) are able to fix nitrogen under natural aerobic conditions (15), since the bacterial nitrogenase is protected from oxygen-induced damage due to the low oxygen concentration at the bacterial surface.

Flavonoids are a group of metabolites associated with cell signaling pathways, responses to microorganisms, and, in general, are correlated with the response of plants to oxidants. Flavonoids consist of benzene rings connected by a short carbon chain (3-4 carbons). Flavonoids comprise six major subtypes, including chalcones, flavones, isoflavonoids, flavanones, anthoxanthins, and anthocyanins (often responsible for the red/violet color of certain plant organs).

There is a need for new methods for developing crop plants with increased ability to fix atmospheric nitrogen, e.g., to allow them to grow under reduced inorganic nitrogen conditions. The present disclosure satisfies this need and provides other advantages as well.

### BRIEF SUMMARY OF THE INVENTION

The present disclosure provides methods and compositions for increasing the ability of plants to assimilate atmospheric nitrogen, in particular by modifying the plants such that they produce increased levels of flavones. The flavones can be exuded by the roots of the plant, inducing increased biofilm formation and N-fixation by bacteria in the soil.

In one aspect, the present disclosure provides a method of increasing the ability of a crop plant to assimilate atmospheric nitrogen, the method comprising modifying the expression of a gene involved in flavone biosynthesis or degradation in one or more cells of the plant such that the plant produces an increased amount of one or more flavones, wherein the one or more flavones are exuded from the plant's roots.

In some embodiments of the method, the one or more flavones induces biofilm formation in N-fixing bacteria present in the soil in proximity to the plant's roots. In some embodiments, the biofilm formation leads to an increase in the ability of the bacteria to fix atmospheric nitrogen, and wherein the fixed atmospheric nitrogen is assimilated by the plant. In some embodiments, the at least one of the one or more flavones are glycosylated. In some embodiments, the one or more flavones comprise apigenin, apigenin-7-glucoside, or luteolin.

In some embodiments, the expression of the gene in the one or more cells of the plant is modified by editing an endogenous copy of the gene. In some such embodiments, the endogenous copy of the gene is modified by introducing into one or more cells of the plant a guide RNA targeting the gene and an RNA-guided nuclease. In some embodiments, the method further comprises introducing into the one or more cells a donor template comprising sequences homologous to the genomic region surrounding the target site of the guide RNA, wherein the RNA-guided nuclease cleaves the DNA at the target site and the DNA is repaired using the donor template. In some embodiments, the RNA-guided nuclease is Cas9 or Cpf1.

In some embodiments, the endogenous copy of the gene is modified so as to reduce or eliminate its expression. In some such embodiments, the endogenous copy of the gene is deleted. In some embodiments, the gene is CYP 75B3 or CYP 75B4, or a homolog or ortholog thereof. In some embodiments, the gene comprises a nucleotide sequence that is substantially identical (sharing at least about 50%, 55%, 60%, 65%, 70%, 75%, 80%, 85%, 90%, 95%, 96%, 97%, 98%, 99% or more identity) to any one of SEQ ID NOS: 2, 4, 6 or 8, or encodes a polypeptide comprising an amino acid sequence that is substantially identical (sharing at least about 50%, 55%, 60%, 65%, 70%, 75%, 80%, 85%, 90%, 95%, 96%, 97%, 98%, 99% or more identity) to any one of SEQ ID NOS: 1, 3, 5, 7, or 14-120.

In some embodiments, the guide RNA comprises a target sequence that is substantially identical (e.g., comprising 0, 1, 2, or 3 mismatches) to any one of SEQ ID NOS: 11-13. In some embodiments, the guide RNA comprises a target sequence that is substantially identical (e.g., comprising 0, 1, 2, or 3 mismatches) to a sequence within SEQ ID NO: 9 or SEQ ID NO:10.

In some embodiments, the endogenous copy of the gene is modified so as to increase its expression. In some such embodiments, the endogenous copy of the gene is modified by replacing the endogenous promoter with a heterologous promoter. In some embodiments, the heterologous promoter is an inducible promoter. In some embodiments, the heterologous promoter is a constitutive promoter. In some embodiments, the heterologous promoter is a tissue-specific promoter. In some embodiments, the heterologous promoter is a root-specific promoter. In some embodiments, the gene is CYP 93G1 or a homolog or ortholog thereof. In some embodiments, the gene encodes a polypeptide comprising an amino acid sequence that is substantially identical (sharing at least about 50%, 55%, 60%, 65%, 70%, 75%, 80%, 85%, 90%, 95%, 96%, 97%, 98%, 99% or more identity) to any one of SEQ ID NOS: 121-145.

In some embodiments, the method further comprises generating a stable plant line from the one or more cells of the plant. In some embodiments, the crop plant is a grain crop. In some embodiments, the grain crop is rice. In some embodiments, the crop plant is selected from the group consisting of corn, wheat, rice, soy, cotton, canola, and sugarcane.

In another aspect, the present disclosure provides a genetically modified crop plant produced using the method of any one of the herein-described methods.

In another aspect, the present disclosure provides a genetically modified plant comprising: i) a mutation or deletion in a CYP75B3 or CYP75B4 gene, or homolog or ortholog thereof, that causes a reduced amount of CYP75B3 or CYP75B4 enzyme and/or enzymatic activity compared to a wild-type plant without the mutation or deletion in the CYP75B3 or CYP75B4 gene; or ii) an expression cassette comprising a polynucleotide encoding a CYP 93G1 gene, or a homolog or ortholog thereof, operably linked to a promoter, such that the plant comprises an increased amount of CYP93G1 enzyme and/or enzymatic activity compared to a wild-type plant without the expression cassette; wherein the genetically modified crop plant produces an increased amount of one or more flavones as compared to a wild-type plant that is not genetically modified, wherein the one or more flavones are exuded from the genetically modified crop plant's roots.

In some embodiments, the plant is selected from the group consisting of corn, wheat, rice, soy, cotton, canola, and sugarcane.

In another aspect, the present disclosure provides a method of increasing the assimilation of atmospheric nitrogen in a grain crop plant grown under reduced inorganic nitrogen conditions, the method comprising: providing a genetically modified crop plant in which the expression of a gene involved in flavone biosynthesis or degradation has been modified in one or more cells such that the roots of the plant exude increased amounts of one or more flavones as compared to a wild-type plant; and growing the plant in soil comprising an amount of inorganic nitrogen that is lower than a standard or recommended amount for the crop plant.

In some embodiments of the method, the crop plant is rice, and the amount of inorganic nitrogen in the soil is less than 50 ppm. In some such embodiments, the amount of inorganic nitrogen in the soil is about 25 ppm. In some embodiments, the genetically modified plant is any of the herein-described plants. In some embodiments, N₂-fixing bacteria in the soil in which the genetically modified plant is grown show greater biofilm formation than control N₂-fixing bacteria in soil in which a wild-type plant is grown. In some embodiments, N₂-fixing bacteria in the soil in which the genetically modified plant is grown show greater adherence to the root surface and/or inside the root tissue of the plant than control N₂-fixing bacteria in soil in which a wild-type plant is grown. In some embodiments, the crop plant is a grain crop, and wherein the number of tillers, tassels, or spikes in the genetically modified plant grown in the soil comprising the reduced amount of inorganic nitrogen is at least 30% greater than in a wild-type plant grown in equivalent soil. In some embodiments, the number of grain or seed-bearing organs and/or the seed yield in the genetically modified plant grown in the soil comprising the reduced amount of inorganic nitrogen is at least 30% greater than in a wild-type plant grown in equivalent soil. In some embodiments, the genetically modified plant grown in the soil comprising the reduced amount of inorganic nitrogen assimilates at least twice the amount of atmospheric nitrogen than the amount assimilated by a wild-type plant grown in equivalent soil.

### BRIEF DESCRIPTION OF THE DRAWINGS

FIG. 1. Workflow for chemical screening.

FIG. 2. Biofilm formation of Glucanoacetobacter diazotrophicus incubated with wild type rice (Oryza sativa Kitaake) root exudates supplemented with FL-500 chemical library.

FIG. 3. Chemical screening identifies apigenin and luteolin as biofilm inducers for the nitrogen fixation bacteria Gluconacetobacter diazotrophicus. Biofilm formation of Glucanoacetobacter diazotrophicus was assessed incubated with wild-type rice (Oryza sativa Kitaake) root exudates supplemented with 2 μl of each of 500 flavonoid and derivated compounds of a chemical library (FL-500, TimTec) and 700 compounds (natural and synthetic) (NPDepo library). Chemical screening was performed in a 96-well plate with each well containing: 198 μL of the Kitaake exudate and 2 μL of the 10 mM compound from the chemical libraries. An equal volume (2 μL) of DMSO was added to each well and served as the negative control. Gluconacetobacter diazotrophicus was added to the final OD600=0.01 to each well and incubated in a shaker at 150 rpm, 28° C. for 3 days before biofilm quantification by crystal violet staining. The value of each well in biofilm quantification was normalized to that of the DMSO control in each plate (DMSO=1). The heatmap was generated by the mean value of 3 biological replicates for each compound.

FIG. 4. Chemical structures and hierarchical clustering of the top 21 positive regulators of biofilm based on pairwise compound similarities defined using the Atom Pair descriptors and Tanimoto coefficiency (chemmine.ucr.edu/). The chemicals are also clustered into 3 groups with different colors by the K-Means algorithm. MW: molecular weight.

FIGS. 5A-5C. Effects of the addition of luteolin or apigenin to biofilm formation in Glucanoacetobacter diazotrophicus. FIG. 5A: Effects of the addition of luteolin to biofilm formation in a Glucanoacetobacter diazotrophicus suspension. FIG. 5B: Effects of the addition of the aglycone or the 0-glucoside of apigenin to biofilm formation in a Glucanoacetobacter diazotrophicus suspension FIG. 5C: Apigenin and apigenin-7-O-glucoside promote nitrogen fixation in Glucanoacetobacter diazotrophicus as demonstrated by the acetylene reduction assay (ARA).

FIG. 6. Biosynthetic pathways of flavonoids in rice

FIG. 7. Effect of natural flavonoids on biofilm formation in Glucanoacetobacter diazotrophicus. Induction of biofilm production in Gluconacetobacter diazotrophicus exposed to Oryza sativa root exudates supplemented with 100 mM of the indicated compounds. Controls are exudates without compound and exudates with DMSO.

FIG. 8. Induction of biofilm production in facultative N₂-fixing bacteria.

FIGS. 9A-9C. Effect of luteolin on biofilm production in Azoarcus sp. CIB (FIG. 9A), Azoarcus communis (FIG. 9B), and Burkholderia vietnamensis (FIG. 9C).

FIG. 10. Biosynthetic pathways of flavone-derived metabolites in rice. Apigenin, Luteolin, and chrysoeriol are synthesized from Naringenin. Apigenin and Luteolin are conjugated to their -5-O- and -7-0-glycosylated forms.

FIG. 11. Effects of Naringenin, Apigenin, Apigenin-7-Glucoside, and Luteolin on biofilm formation on Gluconacetobacter diazotrophicus. Values are the Mean±SD (n=6).

FIGS. 12A-12C. Effects of flavones (Naringenin, Apigenin, Apigenin-7-Glucoside) on bacterium N₂-fixation. FIG. 12A: Activity was assessed by measuring the conversion of acetylene to ethylene by Gas Chromatography. FIG. 12B: Assimilation of Nitrogen by Kitaake rice plants, incubated with Glucanoacetobacter in the absence (DMSO) or presence of Apigenin. Nitrogen assimilation was assessed by feeding ¹⁵N₂ and measuring assimilated inorganic ¹⁵N in leaf tissues after 2 weeks, using Mass Spectroscopy. FIG. 12C: Kitaake rice roots incubated with Glucanoacetobacter in the absence (DMSO) or presence of Apigenin. Adherence of bacteria to the root surface and inside the root tissue can be seen in the presence of Apigenin (Bacteria constitutively expressing a fluorescent marker).

FIG. 13. Glucanoacetobacter detected in the intracellular space of rice roots.

FIG. 14. Silencing of CYP75B3/B4 (Os10g17260/Os16974) would decrease the synthesis of Luteolin, increasing the concentration of apigenin and Apigenin-glucoside derivatives.

FIGS. 15A-15C. Apigenin and apigenin-conjugates contents in roots and root exudates of wild-type (Kitaake) and cyp75b3/b4 homozygous knockouts (CRISPR lines #87 and #104). FIG. 15A: Relative gene expression, as measured by qRT-PCR, of genes encoding CYP75B3 and CYP75B4 in wild-type (Kitaake) and T1 homozygous CRISPR/Cas9-silenced cyp75bB3/and cyp75bB4 lines (CRISPR lines #87 and #104). FIG. 15B: Amount of Apigeninapigenin, Apigeninapigenin-7-Glucoronide and Apigeninapigenin-7-Glucoside in root extracts of wild-type and cyp75b3/b4 lines. FIG. 15C: Amount of apigenin, apigenin-7-Glucoronide and apigenin-7-Glucoside in root exudates of wild-type and cyp75b3/b4 lines. Values are the Mean±S.E (n=5). *P<0.05, **P<0.01 and ***P<0.001 (Student t-test compared with Kitaake control).

FIGS. 16A-16D. cyp75b3/b4-silenced lines induce enhanced biofilm production in bacteria and induce nitrogen fixation in rice plants. Root extracts (FIG. 16A) and root exudates (FIG. 16B) from cyp75b3/b4-silenced rice lines (CRISPR) generate enhanced biofilm production in Gluconacetobacter diazotrophicus. Values are the Mean±S.D. (n=4-6). ** and *** indicate P<0.01 and ***P<0.001, respectively (Student t-test compared with Kitaake control). Root exudate of the CRISPR line induced higher expression of the gumD gene (responsible for the first step in exopolysaccharide (EPS) production of biofilm in Gluconacetobacter diazotrophicus). FIG. 16C: The Gluconacetobacter diazotrophicus was double-labelled by a constitutive expressed mcherry (genpro::mcherry) and the promoter of the gumD gene-driven GFP (gumDpro::GFP). FIG. 16D: The CRISPR line incorporated more nitrogen from the air (delta ¹⁵N) when grown in the greenhouse at both 8 weeks and 16 weeks after germination. Kitaake control and the CRISPR lines were grown in soil for the indicated time. A 10 ml-segment of the root (5 cm below the root-shoot junction) was harvested, after shaking off the loosely attached soil, and sealed in a 20 ml glass tube. Soil from the pots was sampled as bulk soil control. Ten ml of the air was then replaced by ¹⁵N₂ and the tube with each individual sample was incubated at 28° C. for three days. Material from the tubes was dried at 60° C. for seven days before ¹⁵N analysis at UC Davis Stable Isotope Facility. * and *** indicate P<0.05 and P<0.001, respectively (Student t-test compared with Kitaake control).

FIGS. 17A-17D. Wild Type Kitaake rice and cyp75b3/b4 knockout lines were grown in the greenhouse and supplemented with only 30% of the Nitrogen (25 ppm) needed to attain full growth. FIG. 17A: Knockout plants displayed enhanced growth and seed yield. Although the knockout plants were somewhat shorter than the wild-type plants (FIG. 17B), they displayed an increased number of panicles/plant (FIG. 17C) and increased seeds/plant (FIG. 17D).

FIG. 18. Chromosome region of CYP75B3 and the (gRNA) target sequences. Figure discloses SEQ ID NOS 146-151, 11, 13 and 12, respectively, in order of appearance.

### DETAILED DESCRIPTION OF THE INVENTION

### 1. Introduction

The present disclosure provides methods for generating and using genetically modified plants to induce biofilm formation in N-fixing bacteria, increasing their ability to fix atmospheric nitrogen that is then assimilated by the plants, and thereby allowing them to grow efficiently under reduced inorganic nitrogen conditions. The disclosure is based on the surprising discovery that increasing the production of flavones such as apigenin in the roots of the plants allows for the enhanced growth of the plants under such reduced nitrogen conditions. Without being bound by the following theory, it is believed that the flavones produced by the present plants are secreted into the soil and enhance biofilm formation by N-fixing bacteria in the soil. It is believed that the increased biofilm formation allows the enhanced interaction of the plant roots with the N-fixing bacteria, allowing nitrogen uptake by the plant and efficient growth even in the presence of reduced inorganic nitrogen in the soil.

### 2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The “CRISPR-Cas” system refers to a class of bacterial systems for defense against foreign nucleic acids. CRISPR-Cas systems are found in a wide range of eubacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9. Endogenous systems function with two RNAs transcribed from the CRISPR locus: crRNA, which includes the spacer sequences and which determines the target specificity of the system, and the transactivating tracrRNA. Exogenous systems, however, can function which a single chimeric guide RNA that incorporates both the crRNA and tracrRNA components. In addition, modified systems have been developed with entirely or partially catalytically inactive Cas proteins that are still capable of, e.g., specifically binding to nucleic acid targets as directed by the guide RNA, but which lack endonuclease activity entirely, or which only cleave a single strand, and which are thus useful for, e.g., nucleic acid labeling purposes or for enhanced targeting specificity. Any of these endogenous or exogenous CRISPR-Cas system, of any class, type, or subtype, or with any type of modification, can be utilized in the present methods. In particular, “Cas” proteins can be any member of the Cas protein family, including, inter alia, Cas3, Cas5, Cas6, Cas7, Cas8, Cas9, Cas10, Cas12 (including Cas12a, or Cpf1), Cas13, Cse1, Cse2, Csy1, Csy2, Csy3, GSU0054, Csm2, Cmr5, Csx11, Csx10, Csf1, Csn2, Cas4, C2c1, C2c3, C2c2, and others. In particular embodiments, Cas proteins with endonuclease activity are used, e.g., Cas3, Cas9, or Cas12a (Cpf1).

“Flavones” are a class of molecules in the flavonoid family comprising a backbone of 2-phenylchromen-4-one. Any flavone produced by a grain crop plant used in the invention is encompassed by the term, including derivatives such as glycosylated forms of the flavones. Flavones of the invention include, but are not limited to, apigenin, luteolin, tricin, chrysoeriaol, apigenin-5-O-glucoside, apigenin-7-O-glucoside, luteolin-5-O-glucoside, or luteolin-7-O-glucoside.

“CYP75B3” and “CYP75B4” refer to genes, and homologs, orthologs, variants, derivatives, and fragments thereof, that encode the flavonoid 3′-monooxygenase CYP75B3 and CYP75B4 enzymes, which catalyze, e.g., the 3′ hydroxylation of the flavonoid B-ring to the 3′,4′-hydroxylated state, the 3′ hydroxylation of apigenin to form luteolin, the conversion of naringenin to eriodictyol, the conversion of kaempferol to quercetin, and other reactions. See, e.g., UniProt Refs Q7G602 and Q8LM92, the entire disclosures of which are herein incorporated by reference.

“CYP93G1” refers to a gene, and homologs, orthologs, variants, derivatives, and fragments thereof, that encodes cytochrome P450 93G1, an enzyme that functions as a flavone synthase II (FNSII) that catalyzes the direct conversion of flavanones to flavones. See, e.g., UniProt Ref Q0JFI2, the entire disclosure of which is herein incorporated by reference.

The term “nucleic acid sequence encoding a polypeptide” refers to a segment of DNA, which in some embodiments may be a gene or a portion thereof, that is involved in producing a polypeptide chain (e.g., an RNA-guided nuclease such as Cas9). A gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either mRNA or other RNA (e.g. sgRNA) or protein expressed from a particular gene.

The terms “expression” and “expressed” refer to the production of a transcriptional and/or translational product, e.g., of a nucleic acid sequence encoding a protein (e.g., a guide RNA or RNA-guided nuclease). In some embodiments, the term refers to the production of a transcriptional and/or translational product encoded by a gene (e.g., a gene encoding a protein) or a portion thereof. The level of expression of a DNA molecule in a cell may be assessed on the basis of either the amount of corresponding mRNA that is present within the cell or the amount of protein encoded by that DNA produced by the cell.

The term “recombinant” when used with reference, e.g., to a polynucleotide, protein, vector, or cell, indicates that the polynucleotide, protein, vector, or cell has been modified by the introduction of a heterologous nucleic acid or protein or the alteration of a native nucleic acid or protein, or that the cell is derived from a cell so modified. For example, recombinant polynucleotides contain nucleic acid sequences that are not found within the native (non-recombinant) form of the polynucleotide.

As used herein, the terms “polynucleotide,” “nucleic acid,” and “nucleotide,” refer to deoxyribonucleic acids (DNA) or ribonucleic acids (RNA) and polymers thereof. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid. Unless otherwise indicated, a particular nucleic acid sequence also implicitly encompasses conservatively modified variants thereof (e.g., degenerate codon substitutions), homologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “vector” and “expression vector” refer to a nucleic acid construct, e.g., plasmid or viral vector, generated recombinantly or synthetically, with a series of specified nucleic acid elements that permit transcription of a particular nucleic acid sequence (e.g., a guide RNA and/or RNA-guided nuclease) in a cell. In some embodiments, a vector includes a polynucleotide to be transcribed, operably linked to a promoter, e.g., a constitutive or inducible promoter. Other elements that may be present in a vector include those that enhance transcription (e.g., enhancers), those that terminate transcription (e.g., terminators), those that confer certain binding affinity or antigenicity to a protein (e.g., recombinant protein) produced from the vector, and those that enable replication of the vector and its packaging (e.g., into a viral particle). In some embodiments, the vector is a viral vector (i.e., a viral genome or a portion thereof).

The terms “polypeptide,” “peptide,” and “protein” are used interchangeably herein to refer to a polymer of amino acid residues. All three terms apply to amino acid polymers in which one or more amino acid residues are an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

### 3. Generating Crop Plants with Increased N₂ Assimilation

### Plants

The present methods can be used to modify any plant, including monocots and dicots, grains, trees, and vegetable crops, in order to increase its ability to interact with nitrogen-fixing bacteria in the soil. In particular embodiments, the plant is a crop species such as corn, wheat, rice, soy, cotton, canola, or sugarcane. In particular embodiments, the crop plant is a grain crop. Crops that can be used include, but are not limited to, cereals, oilseeds, pulses, hays, and others. A non-limiting list of cereals that can be used includes rice (e.g., Oryza, Zizani spp.), wheat (e.g., Triticum aestivum), barley (e.g., Hordeum vulgare), oat (e.g., Avena sativa), rye (e.g., Secale cereal), triticale (e.g., Triticosecale spp.), corn (e.g., Zea mays), sorghum Sorghum spp., millet (e.g., Digitaria, Echinochloa, Eleusine, Panicum, Setaria, Pennisetum, spp.), canary seed (e.g., Phalaris canariensis), teff (e.g., Eragrostis abyssinica), and Job's Tears (e.g., Coix lacryma-jobi). In particular embodiments, the plant is rice, e.g., Oryza sativa. A non-limiting list of oilseeds includes soybeans (e.g., Glycine spp.), peanuts (e.g., Arachis hypogaea), canola and mustard (e.g., Brassica spp., Brassica napus), sunflower, (e.g., Helianthus annuus), safflower (e.g., Carthamus spp., and flax (e.g., Linum spp.). A non-limiting list of pulses include pinto beans (e.g., Phaseolus vulgaris), lima beans (e.g., Phaseolus lunatus), mungo beans (e.g., Phaseolus mung), adzuki beans (e.g., Phaseolus angularis), chickpeas (e.g., Cicer arietinum), field, green and yellow peas (e.g., Pisum spp.), lentils (e.g., Lens spp.), fava beans (e.g., Vicia faba), and others including Dolichos, Cajanus, Vigna, Pachyrhizus, Tetragonolobus, spp. A non-limiting list of hay and pasture plants includes grasses such as Meadow Foxtail (e.g., Alopecurus pratensis), Brome (e.g., Bromus spp.), Orchard Grass (e.g., Dactylis glomerata), Fescue (e.g., Festuca spp.), rye grass (e.g., Lolium spp.), reed canary grass (e.g., Phalaris arundinacea), Kentucky blue grass (e.g., Poa pratensis), Timothy (e.g., Phleum pretense), and redtop (e.g., Agropyron spp.), as well as legumes such as alfalfa and yellow trefoil (e.g., Medicago spp., Medicago sativa), clovers (Trifolium spp.), birdsgoot trefoil (e.g., Lotus corniculatus), and vetch (e.g., Vicia spp.). Other plants that can used includes buckwheat, tobacco, hemp, sugar beets, and amaranth. In some embodiments, the plant is a shrub such as cotton (e.g., Gossypium hirsutum, Gossypium barbadense.) In some embodiments, the plant is a grass such as sugarcane (e.g., Saccharum officinarum). A non-limiting list of plants that can be used is shown, e.g., in Tables 1 and 2.

In some embodiments, the plant is a tree. Any tree can be modified using the present methods, including angiosperms and gymnosperms. A non-limiting list of trees includes, e.g., cycads, ginkgo, conifers (e.g., araucarias, cedars, cypresses, Douglas firs, firs, hemlocks, junipers, larches, pines, podocarps, redwoods, spruces, yews), monocotyledonous trees (e.g., palms, agaves, aloes, dracaenas, screw pines, yuccas) and dicotyledons (e.g., birches, elms, hollies, magnolias, maples, oaks, poplars, ashes, and willows). In a particular embodiment, the tree is a poplar (e.g., cottonwood, aspen, balsam poplar), e.g., Populus alba, Populus grandidentata, Populus tremula, Populus tremuloides, Populus deltoids, Populus fremontii, Populus nigra, Populus angustifolia, Populus balsamifera, Populus trichocarpa, or Populus heterophylla.

In some embodiments, the plant is a vegetable. Vegetables that can be used include, but are not limited to, Arugula (Eruca sativa), Beet (Beta vulgaris vulgaris), Bok choy (Brassica rapa), Broccoli (Brassica oleracea), Brussels sprouts (Brassica oleracea), Cabbage (Brassica oleracea), Celery (Apium graveolens), Chicory (Cichorium intybus), Chinese mallow (Malva verticillata), Garland Chrysanthemum (Chrysanthemum coronarium), Collard greens (Brassica oleracea), Common purslane (Portulaca oleracea), Corn salad (Valerianella locusta), Cress (Lepidium sativum), Dandelion (Taraxacum officinale), Dill (Anethum graveolens), Endive (Cichorium endivia), Grape (Vitis), Greater plantain (Plantago major), Kale (Brassica oleracea), Lamb's lettuce (Valerianella locusta), Land cress (Barbarea verna), Lettuce (Lactuca sativa), Mustard (Sinapis alba), Napa cabbage (Brassica rapa), New Zealand Spinach (Tetragonia tetragonioides), Pea (Pisum sativum), Poke (Phytolacca Americana), Radicchio (Cichorium intybus), Sorrel (Rumex acetosa), Sour cabbage (Brassica oleracea), Spinach (Spinacia oleracea), Summer purslane (Portulaca oleracea), Swiss chard (Beta vulgaris cicla), Turnip greens (Brassica rapa), Watercress (Nasturtium officinale), Water spinach (Ipomoea aquatic), and Yarrow (Achillea millefolium). Also included are fruits and flowers such as gourds, squashes, Pumpkins, Avocado, Bell pepper, Cucumber, Eggplant, Sweet pepper, Tomato, Vanilla, Zucchini, Artichoke, Broccoli, Caper, and Cauliflower.

### Modifying Flavone Production

In the present methods, the plants are modified to increase the production of one or more flavones, in particular in the roots of the plant. Any flavone that increases biofilm formation in facultative N₂-fixing bacteria can be used. In some embodiments, the flavones increased in the plants include apigenin, luteolin, tricin, chrysoeriaol, apigenin-5-O-glucoside, apigenin-7-O-glucoside, luteolin-5-O-glucoside, or luteolin-7-O-glucoside, or combinations thereof. In particular embodiments, the flavone increased in the plant is apigenin, apigenin-5-O-glucoside, or apigenin-7-O-glucoside.

It will be appreciated that, in addition to flavones, other plant molecules can be identified using the herein-described assays that have biofilm-inducing activity, and plants can be generated that produce elevated levels of the molecules. For example, heterooctacyclic compounds, anthraquinones, or other flavonoids can be used. Methods to increase the production of such non-flavone molecules, as described herein for flavones, can be carried out in combination with, or in place of, the present methods to increase the production of flavones, with the effects of the molecules on biofilm formation and/or atmospheric nitrogen fixation assessed, e.g., using any of the methods for detecting and/or quantifying biofilm formation or nitrogen fixation described herein.

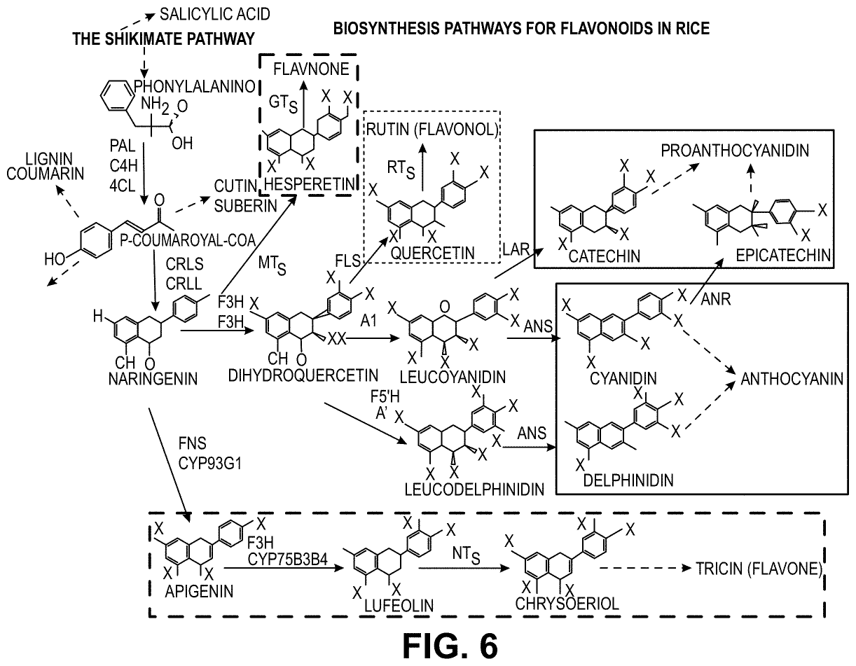

In particular embodiments, the modification of the plants involves the upregulation or downregulation of one or more genes encoding enzymes involved in flavone biosynthesis or degradation. The enzymes can be any enzyme that affects the production or degradation of one or more flavones. Some such enzymes, in rice and other plants, are indicated, for example, in FIGS. 6, 10, and 14.

### Flavone Synthase (e.g., CYP93G1) Upregulation

In some embodiments, a flavone synthase (e.g., a flavone synthase I or flavone synthase II such as CYP 93G1 (CYP93G1) in rice, or an equivalent flavone synthase, e.g., another CYP 93 or CYP 93G enzyme, or a homolog or ortholog thereof, in another plant species) is upregulated so as to increase the synthesis of, e.g., apigenin from naringenin (see, e.g., Lam et al. (2014) Plant Physiol. 165(3):1315-1327; Du et al. (2009) J. Exper. Bot. 61(4):983-994; Du et al. (2016) PlosOne doi.org/10.1371/journalpone.0165020; the entire disclosure of each of which is herein incorporated by reference in its entirety). CYP93G1 sequences can be found, e.g., at NCBI accession nos. AK100972.1 and UniProt Q0JFI2, and additional information, including information useful for identifying homologs in other species, can be found, e.g., at the Plant Metabolic Network (PMN, plantcyc.org) entry for CYP93G1. In addition, sequences of suitable CYP93G1 enzymes in diverse species are presented herein as SEQ ID NOS: 121-145.

Such enzymes can be upregulated in any of a number of ways, as described in more detail elsewhere herein. For example, the enzymes can be upregulated by introducing a transgene into the plant encoding any of the herein-described CYP93G1 enzymes, or homologs or orthologs thereof, or derivatives, variants, analogs, or fragments of any of the enzymes, homologs, or orthologs. In some embodiments, a transgene is introduced that encodes any one of SEQ ID NOS:121-145 or a fragment of any one of SEQ ID NOS:121-145, or encodes a polypeptide having at least about 50%, 55%, 60%. 65%. 70%, 75%, 80%, 85%, 90%, 95%, 96%, 97%, 98%, 99% or more identity to any one of SEQ ID NOS:121-145 or a fragment of any one of SEQ ID NOS:121-145, or any of the genes listed in Table 2. As described in more detail herein, the transgene can be introduced using any of a number of suitable methods, including, e.g., CRISPR-mediated genetic modification. In particular embodiments, the transgene is introduced as an expression cassette, e.g., a coding sequence as described herein, operably linked to a promoter, e.g., a constitutive, inducible, or organ/tissue-specific promoter. A non-limiting list of suitable promoters includes promoters from, e.g., CaMV 35S, Ubi-1, CAM19, MMV, SVBV, nos, ocs, Act1, HSP18.2, Rd29, adh, rbcS-3A, Chn48, PvSR2, cgmt1, HVADhn45, PtDr102, CaPrx, R2329, R2184, OsNAC6, PPP, Zmglp1, PnGLP, PDX1, and others. In particular embodiments, a root-specific promoter is used, including, but not limited to, promoters from TobRB7, rolD, SIREO, CaPrx, 0503g01700, 0502g37190, EgTIP2, ET304, and others.

### Hydroxylase (e.g., CYP75B3/B4) Inhibition

In some embodiments, an enzyme, or gene encoding an enzyme, that converts a flavone to another flavone is inhibited. For example, in particular embodiments, apigenin levels are increased by inhibiting a hydroxylase such as CYP 75B3 (or CYP75B3) and/or CYP 75B4 (or CYP75B4) in rice, or an equivalent enzyme, e.g., homolog or ortholog, in another species, which are involved in the conversion of, e.g., apigenin to luteolin (see, e.g., Lam et al. (2019) New Phyt. doi.org/10.1111/nph.15795; Shih et al. (2008) Planta 228:1043-1054; Lam et al. (2015) Plant. Phys. 175:1527-1536; Park et al. (2016) Int. J. Mol. Sci. 17:e1549; the entire disclosure of each of which is herein incorporated by reference in its entirety). The enzymes can be inhibited in any of a number of ways. In some embodiments, the enzymes are inhibited by generating transgenic plants: i) with a deletion or mutation in the CYP75B3/B4 gene that causes decreased or abolished expression of the enzyme; ii) that express an inhibitor of CYP75B3/B4 gene expression (e.g., siRNA, miRNA), or iii) that express an inhibitor of CYP75B3/B4 enzymatic activity (e.g., peptide inhibitor, antibody). In some embodiments, the enzymes are inhibited through the application of an inhibitor, e.g., small molecule inhibitor, to the plants.

The sequence of an exemplary CYP75B3 from Oryza sativa Japonica can be found, e.g., at NCBI accession no. AK064736 and UniProt Q7G602, and additional information, including for identifying homologs in other species can be found, e.g., at the Plant Metabolic Network (PMN) entry for CYP75B3. The sequence of an exemplary CYP75B4 from Oryza sativa Japonica can be found, e.g., at NCBI accession nos. AK070442 and UniProt Q8LM92, and additional information, including information useful for identifying homologs in other species, can be found, e.g., at the Plant Metabolic Network (PMN, plantcyc.org) entry for CYP75B4. Suitable amino acid sequences for CYP75B3/B4 from Oryza sativa japonica and indica are also shown as SEQ ID NOS: 1, 3, 5, 7, and suitable nucleotide sequences are also shown as SEQ ID NOS: 2, 4, 6, and 8. Exemplary amino acid sequences for orthologs in other species are shown, e.g., as SEQ ID NOS: 14-120. Any polypeptide from any plant species comprising at least about 50%, 55%, 60%. 65%. 70%. 75%, 80%, 85%, 90%, 95%, 96%, 97%, 98%, 99%, or more identity to any one of SEQ ID NOS:1, 3, 5, 7, 14-120, or a fragment thereof, or any polynucleotide from any plant species comprising at least about 50%, 55%, 60%. 65%. 70%, 75%, 80%, 85%, 90%, 95%, 96%, 97%, 98%, 99%, or more identity to SEQ ID NO:2, 4, 6, or 8, or a fragment thereof, or encoding any one of SEQ ID NOS:1, 3, 5, 7, 14-120, or a fragment thereof, can be used (e.g., targeted for inhibition) in the present methods, as can any of the orthologs listed in Table 1.

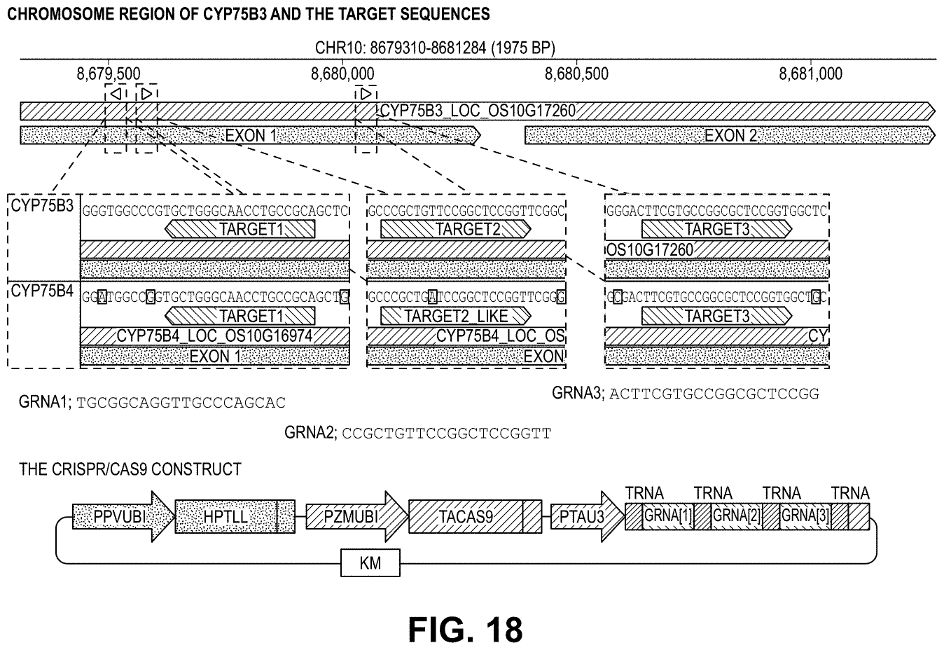

In particular methods, the gene or encoded protein is inhibited using a CRISPR-Cas system, e.g., by introducing a guide RNA targeting the gene of interest (e.g., a CYP75B3/B4 gene), a Cas enzyme such as Cas9 or Cpf1, and a homologous template, in order to inactivate the gene by deleting or mutating it. For example, a CYP75B3 and/or CYP75B4 gene can be targeted by using a guide RNA with a target sequence falling within the genomic locus encoding the enzyme. For example, the guide RNA can have a target sequence comprising any of the sequences, or fragments thereof, shown in FIG. 18 or presented as SEQ ID NOS: 11-13, or having about 50%, 55%, 60%. 65%. 70%, 75%, 80%, 85%, 90%, 95%, 96%, 97%, 98%, 99%, or more identity to any of the sequences, or fragments thereof, shown in FIG. 18 or presented as SEQ ID NOS: 11-13.

In some embodiments, a CYP75B3 and/or CYP75B4 gene is targeted using a guide RNA with a target sequence located within a genomic sequence shown as SEQ ID NO: 9 or SEQ ID NO:10, located within a genomic sequence corresponding to any of the Gene ID numbers shown in Table 1, or comprising at least about 50%, 55%, 60%. 65%. 70%, 75%, 80%, 85%, 90%, 95%, 96%, 97%, 98%, 99% to any subsequence within SEQ ID NOS: 9 or SEQ ID NO:10 or any of the genomic sequences corresponding to any of the Gene ID numbers shown in Table 1.

A non-limiting list of orthologs from various species, any of which can be inhibited using any of the herein-described methods, can be found, e.g., in the website: bioinformatics.psb.ugent.be/plaza/versions/plaza_v4_5_monocots/gene_families/view/ORTH O04x5M002123, the entire contents of which are herein incorporated by reference. This website provides, e.g., sequence and other genetic information about 119 genes in the ORTHO04x5M002123 family in 32 spermatophyte species, any of which can be inhibited using the present methods. In particular, a non-limiting list of exemplary orthologs that can be inhibited in the present methods is shown in Table 1.

TABLE 1
A non-limiting list of CYP75B3/B4 orthologs from other species.
Sequences and other information for each of the genes can be found,
e.g., at the website: bioinformatics.psb.ugent.be/plaza/versions/
plaza_v4_5_monocots/gene_families/view/ORTHO04x5M002123 and
elsewhere herein, and as SEQ ID NOS: 14-120.

| Species | Gene ID |
|---|---|
| Oryza sativa ssp. indica | OsR498G1018420100 |
| Oryza sativa ssp. indica | OsR498G1018427100 |
| Oryza sativa ssp. japonica | LOC_Os10g16974 |
| Oryza sativa ssp. japonica | LOC_Os10g17260 |
| Triticum aestivum | TraesCS1A02G442200 |
| Triticum aestivum | TraesCS1A02G442300 |
| Triticum aestivum | TraesCS1B02G476400 |
| Triticum aestivum | TraesCS1D02G450100 |
| Triticum aestivum | TraesCS2B02G613200 |
| Triticum aestivum | TraesCS6A02G012600 |
| Triticum aestivum | TraesCS6B02G018800 |
| Triticum aestivum | TraesCS6D02G015200 |
| Triticum aestivum | TraesCS6D02G015300 |
| Triticum aestivum | TraesCS7A02G411700 |
| Triticum aestivum | TraesCS7B02G310900 |
| Triticum aestivum | TraesCS7D02G404900 |
| Zea mays B73 | Zm00001d010521 |
| Zea mays B73 | Zm00001d017077 |
| Zea mays B73 | Zm00001d050955 |
| Zea mays B104 | Zm00007a00002679 |
| Zea mays B104 | Zm00007a00006475 |
| Zea mays B104 | Zm00007a00021951 |
| Zea mays B104 | Zm00007a00044616 |
| Zea mays PH207 | Zm00008a016611 |
| Zea mays PH207 | Zm00008a022212 |
| Zea mays PH207 | Zm00008a031477 |
| Triticum turgidum | TRITD1Av1G229990 |
| Triticum turgidum | TRITD1Av1G230000 |
| Triticum turgidum | TRITD2Bv1G262360 |
| Triticum turgidum | TRITD6Av1G001970 |
| Triticum turgidum | TRITD6Bv1G003180 |
| Triticum turgidum | TRITD7Av1G223010 |
| Triticum turgidum | TRITD7Bv1G170910 |
| Setaria italica | Seita.9G242900 |
| Setaria italica | Seita.9G244600 |
| Cenchrus americanus | Pgl_GLEAN_10033465 |
| Cenchrus americanus | Pgl_GLEAN_10033479 |
| Sorghum bicolor | Sobic.004G200800 |
| Sorghum bicolor | Sobic.004G200833 |
| Sorghum bicolor | Sobic.004G200900 |
| Sorghum bicolor | Sobic.004G201100 |
| Sorghum bicolor | Sobic.009G162500 |
| Brachypodium distachyon | Bradi1g17180 |
| Brachypodium distachyon | Bradi1g24840 |
| Brachypodium distachyon | Bradi3g04750 |
| Brachypodium distachyon | Bradi4g16560 |
| Hordeum vulgare | HORVU6Hr1G002400 |
| Gossypium raimondii (the putative | XP_012438857 |
| contributor of the D subgenome to | |
| the economically important fiber- | |
| producing cotton species | |
| Gossypium hirsutum and | |
| Gossypium barbadense.) | |
| Gossypium raimondii | XP_012478317 |
| Gossypium raimondii | KJB51033 |
| Gossypium raimondii | XP_012454458 |
| Gossypium raimondii | XP_012490769 |
| Gossypium hirsutum(90% of the | NP_001314443 |
| world's cotton production) | |
| Gossypium hirsutum | XP_016741685 |
| Gossypium hirsutum | ACY06905 |
| Gossypium hirsutum | NP_001314550 |
| Gossypium hirsutum | NP_001314530 |
| Gossypium hirsutum | ACY06904 |
| Gossypium hirsutum | XP_016710494 |
| Gossypium hirsutum | KAG4120389 |
| Gossypium hirsutum | NP_001314163.1 |
| Gossypium barbadense(5% of the | KAB2053485 |
| world's cotton production) | |
| Gossypium barbadense | KAB1669149 |
| Gossypium barbadense | PPD88185 |
| Gossypium barbadense | PPR81792 |
| Gossypium barbadense | KAB2021362 |
| Gossypium barbadense | KAB2074130 |
| Gossypium barbadense | KAB2074128 |
| Gossypium barbadense | KAB2057053 |
| Gossypium barbadense | KAB2007859 |
| Brassica napus cultivar Darmor_v5 | BnaC09g47980D |
| Brassica napus cultivar Darmor_v5 | BnaA10g23330D |
| Brassica napus cultivar ZS11 | BnaA10G0256900ZS |
| Brassica napus cultivar ZS11 | BnaC09G0570900ZS |
| Brassica napus cultivar Gangan | BnaA10G0251000GG |
| Brassica napus cultivar Gangan | BnaC09G0516100GG |
| Brassica napus cultivar Quinta | BnaA10G0248800QU |
| Brassica napus cultivar Quinta | BnaC09G0534300QU |
| Brassica napus cultivar Shengli | BnaA10G0220400SL |
| Brassica napus cultivar Shengli | BnaC09G0396500SL |
| Brassica napus cultivar Tapidor | BnaA10G0249900TA |
| Brassica napus cultivar Tapidor | BnaC09G0550200TA |
| Brassica napus cultivar Westar | BnaA10G0251800WE |
| Brassica napus cultivar Westar | BnaC09G0543700WE |
| Brassica napus cultivar Zheyou7 | BnaA10G0234400ZY |
| Brassica napus cultivar Zheyou7 | BnaC09G0517700ZY |
| Saccharum hybrid cultivar R570 | AGT17103 |
| Saccharum hybrid cultivar R570 | AGT17101 |
| Saccharum hybrid cultivar R570 | AGT16621 |
| Saccharum hybrid cultivar R570 | AGT16132 |
| Saccharum hybrid cultivar R570 | AGT17102 |
| Saccharum hybrid cultivar R570 | AGT16178 |
| Saccharum hybrid cultivar R570 | AGT16989 |
| Saccharum hybrid cultivar R570 | AGT16177 |
| Saccharum hybrid cultivar R570 | AGT16905 |
| Saccharum hybrid cultivar R570 | AGT16500 |
| Saccharum hybrid cultivar R570 | AGT16853 |
| Saccharum hybrid cultivar R570 | AGT17443 |
| Saccharum officinarum | AWA44852 |
| Saccharum officinarum | AWA44857 |
| Saccharum officinarum | AWA44838 |
| Saccharum officinarum | AWA44954 |
| Glycine max | Glyma.06G202300 |
| Glycine max | Glyma.05G021800 |
| Glycine max | Glyma.05G021900 |
| Glycine max | Glyma.05G022100 |
| Glycine max | Glyma.17G077700 |

TABLE 2
A non-limiting list of CYP93G1 orthologs
from other species. Sequences and other information
for each of the genes can be found, e.g., at the
website: bioinformatics.psb.ugent.be/plaza/versions/
plaza_v4_5_monocots/gene_families/view/and as
SEQ ID NOS: 121 to 145.

| Species | Gene ID |
|---|---|
| Oryza sativa ssp. japonica | LOC_Os04g01140 |
| Oryza sativa ssp. indica | OsR498G0407413200 |
| Brachypodium distachyon | Bradi5g02460 |
| Triticum aestivum | TraesCS2D02G043500 |
| Triticum aestivum | TraesCS2A02G044900 |
| Triticum aestivum | TraesCS2B02G057100 |
| Triticum turgidum | TRITD2Av1G010200 |
| Triticum turgidum | TRITD2Bv1G013440 |
| Setaria italica | Seita.1G019400 |
| Cenchrus americanus | Pgl_GLEAN_10038007 |
| Cenchrus americanus | Pgl_GLEAN_10012559 |
| Sorghum bicolor | Sobic.004G108200 |
| Sorghum bicolor | Sobic.006G001000 |
| Zea mays B104 | Zm00007a00042926 |
| Zea mays B104 | Zm00007a00044196 |
| Zea mays B104 | Zm00007a00044088 |
| Zea mays B104 | Zm00007a00049351 |
| Zea mays PH207 | Zm00008a021549 |
| Zea mays PH207 | Zm00008a037571 |
| Zea mays PH207 | Zm00001d004555 |
| Zea mays PH207 | Zm00008a008017 |
| Zea mays PH207 | Zm00008a037570 |
| Zea mays B73 | Zm00001d016151 |
| Zea mays B73 | Zm00001d024946 |
| Zea mays B73 | Zm00001d024943 |

### Other Modifications

In some embodiments, the level of glycosylation of one or more flavones is modified by upregulating or downregulating an enzyme such as a UDP-dependent glycosyltransferase (UGT) such as UGT 707A2-A5 or UGT 706D1-E1 (see, e.g., Peng et al. (2017) Nature Comm. 8: 1975; the entire disclosure of which is herein incorporated by reference), e.g., OsUGT707A2 in rice, or an equivalent enzyme in another species. Sequence and other information about OsUGT707A2, including information useful for identifying homologs in other species, can be found, e.g., at the Rice Genome Annotation Project (rice.plantbiology.msu.edu) entry for LOC/Os07g32060. Sequence and other information about OsUGT706D1, including information useful for identifying homologs in other species, can be found, e.g., at the Rice Genome Annotation Project (rice.plantbiology.msu.edu) entry for LOC/Os01g53460.

It will be appreciated that more than one modification in gene expression, or an alteration in enzyme activity or stability, can be made in a single plant, e.g., upregulating a flavone synthase (such as CYP 93G1) to increase the level of multiple flavones and simultaneously inhibiting an enzyme (such as CYP 73B3 or CYP 73B4) to increase the level of a specific flavone such as apigenin, and/or modulating the expression of a glycosyltransferase to alter the glycosylation of one or more flavones.

### Methods of Altering Expression or Activity

The expression of the genes can be modified in any of a number of ways. For example, to increase the level of expression of a gene, the endogenous promoter can be replaced with a heterologous promoter capable of overexpressing the gene. The heterologous promoter can be inducible or constitutive, and can be ubiquitous or tissue specific (e.g., expressed particularly in the roots). Any promoter capable of driving overexpression of the gene in plant cells can be used, e.g., a CaMV35S promoter, an Act1 promoter, an Adh1 promoter, a ScBV promoter, or a Ubi1 promoter. Examples of inducible promoters that can be used include, but are not limited to, EST (induced by estrogen) and DEX (induced by dexamethasone). In some embodiments, instead of modifying the endogenous gene, a transgene is introduced comprising a coding sequence for the gene, operably linked to a promoter. In some embodiments, the expression of a gene is inhibited or silenced, e.g., by disrupting or deleting an endogenous copy of the gene. In some embodiments, an inhibitor of the enzyme or its expression is expressed, e.g., by RNAi, e.g., siRNA, miRNA, peptide inhibitors, antibody inhibitors, etc.

It will be appreciated that the inhibition of genes involved in flavone biosynthesis or degradation, e.g., CYP73B3 or CYP73B4, can be achieved not only by deleting or otherwise silencing the gene through, e.g., CRISPR-mediated genomic editing or through expression of an inhibitor such as RNAi, but also by other standard means, e.g., through the application of molecules to the plants that inhibit the enzymatic activity or decrease the stability of the enzymes, e.g., the products of CYP73B3 and/or CYP73B4, or that decrease the stability or translation of mRNA transcribed from the genes.

In typical embodiments, the plants are genetically modified using an RNA-guided nuclease, e.g. endonuclease. In particular embodiments, a CRISPR-Cas system is used to modify one or more target genes involved in the synthesis or degradation of one or more flavones. Other methods can also be used, e.g. transcription activator-like effector nucleases (TALENs), zinc-finger nucleases (ZFNs), and others. Any type of genetic modification can be performed, including insertions of one or more sequences into the genome (e.g., to introduce a transgene or regulatory element), deletions of one or more sequences in the genome (e.g., to inactivate an gene), replacement of one or more sequences in the genome (e.g., to replace an endogenous promoter with a heterologous promoter), and alteration of one or more nucleotides in the genome (e.g., to modify the regulation and/or the expression level of a gene).

In particular embodiments of the disclosure, a CRISPR-Cas system is used, e.g., Type II CRISPR-Cas system. The CRISPR-Cas system includes a guide RNA, e.g., sgRNA, that targets the genomic sequence to be altered, and a nuclease that interacts with the guide RNA and cleaves or binds to the targeted genomic sequence. The guide RNA can take any form, including as a single guide RNA, or sgRNA (e.g., a single RNA comprising both crRNA and tracrRNA elements) or as separate crRNA and tracrRNA elements. Standard methods can be used for the design of suitable guide RNAs, e.g., sgRNAs, e.g., as described in Cui et al. (2018) Interdisc. Sci.: Comp. Life Sci. 10(2):455-465; Bauer et al. (2018) Front. Pharmacol: 12 Jul. 2018, doi.org/10.3389/fphar.2018.00749; Mohr et al. (2016) FEBS J., doi.org/10.1111/febs.13777, the entire disclosures of which are herein incorporated by reference.

Any CRISPR nuclease can be used in the present methods, including, but not limited to, Cas9, Cas12a/Cpf1, or Cas3, and the nuclease can be from any source, e.g., Streptococcus pyogenes (e.g. SpCas9), Staphylococcus aureus (SaCas9), Streptococcus thermophiles (StCas9), Neisseria meningitides (NmCas9), Francisella novicida (FnCas9), and Campylobacter jejuni (CjCas9). The guide RNA and nuclease can be used in various ways to effect genomic modifications in the cells. For example, two guide RNAs can be used that flank an undesired gene or genomic sequence, and cleavage of the two target sites leads to the deletion of the gene or genomic sequence. In some embodiments, a guide RNA targeting a gene or genomic sequence of interest is used, and the cleavage of the gene or genomic sequence of interest and subsequent repair by the cell leads to the generation of an insertion, deletion, or mutation of nucleotides at the site of cleavage. In some embodiments, one or more additional polynucleotides are introduced into the cells together with the guide RNA and nuclease, e.g., a donor template comprising regions sharing homology to the targeted genomic sequence (e.g., homology to both sides of the guide RNA target site), with sequences present between the homologous regions effecting a deletion, insertion, or alteration of the genomic sequence via homologous recombination. In particular embodiments, the guide RNA used comprises a target sequence that is substantially identical (e.g., with 0, 1, 2, or 3 mismatches) to any one of SEQ ID NOS:11-13, or that falls within any of the genomic sequences shown as SEQ ID NOS: 9-10 or as listed in Table 1 or Table 2.

In particular embodiments, one or more polynucleotides are introduced into cells of the plant encoding a guide RNA and encoding the RNA-guided nuclease, e.g., Cas9. For example, a vector, e.g., a viral vector, plasmid vector, or Agrobacterium vector, encoding one or more guide RNAs and an RNA-guided nuclease is introduced into plant cells, e.g., by transfection, wherein the one or more guide RNAs and the RNA-guided nuclease are expressed in the cells. In some embodiments, one or more guide RNAs are preassembled with RNA-guided nucleases as ribonucleoproteins (RNPs), and the assembled ribonucleoproteins are introduced into plant cells.

The elements of the CRISPR-Cas system can be introduced in any of a number of ways. In some embodiments, the elements are introduced using polyethylene glycol (PEG), e.g., polyethylene glycol-calcium (PEG-Cat). In some embodiments, the elements are introduced using electroporation. Other suitable methods include microinjection, DEAE-dextran treatment, lipofection, nanoparticle-mediated transfection, protein transduction domain-mediated transfection, and biolistic bombardment. Methods for introducing RNA-guided nucleases into plant cells to effect genetic modifications that can be used include those disclosed in, e.g., Toda et al. (2019) Nature Plants 5(4):363-368; Osakabe et al. (2018) Nat Protoc 13(12):2844-2863; Soda et al. (2018) Plant Physiol Biochem 131:2-11; WO2017061806A1; Mishra et al. (2018) Frontiers Plant Sci. 19, doi.org/10.3389/fpls.2018.03161; the entire disclosures of which are herein incorporated by reference.

Using the present methods, plant lines can be generated (e.g., generated from transfected cells or protoplasts) comprising the genetic modification and producing one or more flavones at higher levels than in wild-type plants. For example, plant lines can be generated by introducing guide RNA, an RNA-guided nuclease, and optionally a template DNA into isolated plant cells or protoplasts, and generating plants from the cells using standard methods.

### Assessing Compounds and Plants

Any of a number of assays can be used to assess plants generated using the present methods, as well as to assess candidate plant molecules (e.g., other flavones) for their ability to upregulate biofilm production and assimilation of N₂-fixing bacteria. For example, to confirm that the plants are exuding increased levels of the one or more flavones, root exudates from the plants can be isolated and the quantities and identities of the flavones determined, e.g., using mass spectrometry. In addition, the exudates (or other candidate biofilm-inducing molecules) can be incubated with N₂-fixing bacteria, e.g., Glucanoacetobacter diazotrophicus, and the biofilm produced by the bacteria assessed. The biofilm can be quantified, e.g., by incubating the exudate (or candidate molecule or molecules) and bacteria in the wells of a microtiter plate, removing the cultures from the plate, washing the wells, adding a solution of crystal violet, rinsing and drying the plate, and then adding ethanol and measuring absorbance at, e.g., 540 nm. See, e.g. Example 1 and www.jove.com/video/2437/microtiter-dish-biofilm-formation-assay, the entire disclosure of which is herein incorporated by reference.

The activity of the exudate or of candidate molecules can also be assessed in vivo, e.g., by using transgenic N₂-fixing bacteria such as Glucanoacetobacter diazotrophicus that constitutively express a label such as mCherry. The bacteria can also express labeled components of biofilms, e.g., in bacteria transformed with gumDpro::GFP. The double labeling in such bacteria allows the visualization of the bacteria and, independently, the development of biofilm in the presence or absence of the exudate or candidate molecule.

The N₂-fixing activity of the bacteria can be assessed, e.g., using an acetylene reduction assay (ARA), in which bacteria are cultured in the presence of acetylene gas, and the conversion of acetylene to ethylene measured by, e.g., gas chromatography.

As noted above, the present assays can be used both to assess the presence and biofilm-inducing activity of flavones in plant exudates, as well as to assess the relative biofilm-inducing activities of different flavones or other molecules. For example, the assays can be used to determine which flavones or other molecules, or combinations of flavones and/or other molecules, have the greatest biofilm-inducing activity. The identification of such molecules or combinations of molecules can guide the selection of plant gene or genes to be upregulated or downregulated using the present methods.

The genetically modified plants themselves can also be assessed in any of a number of ways. For example, plants can be grown in the presence of fluorescently labeled N₂-fixing bacteria, and the adherence of the bacteria to the plant root hairs, either attached to the root surface or present inside the plant tissues, can be determined. The plants can also be assessed by determining the number of tillers and/or the seed yield. In some embodiments, the assimilation of N₂ fixed by bacteria in the soil is assessed by, e.g., growing the plants in the presence of ¹⁵N₂ gas, and then measuring the level of ¹⁵N assimilated in the plant leaves, e.g., using Mass spectroscopy.

In some embodiments, plants generated using the present methods show an increase in the amount of one or more flavones exuded of at least 5%, 10%, 15%, 20%, 25%, 30%, 40%, 50%, 60%, 70%, 80%, 90% or more as compared to the amount exuded in a wild-type plant. In some embodiments, plants generated using the present methods show an increase of at least 5%, 10%, 15%, 20%, 25%, 30%, 40%, 50%, 60%, or more in the number of tillers/tassels/spikes and/or in the seed yield as compared to in wild-type plants. In some embodiments, plants generated using the present methods, or exudates from said plants, induce an increase of at least about 0.1 (i.e., an increase of about 10%), 0.2, 0.3, 0.4, 0.5, 0.6, 0.7, 0.8, 0.9, 1-fold, 2-fold, 3-fold, 4-fold, or more, in biofilm formation in Glucanoacetobacter diazotrophicus or other N₂-fixing bacteria as compared to wild-type plants, or exudates from wild-type plants. In some embodiments, plants generated using the present methods induce an increase of at least about 0.1, 0.2, 0.3, 0.4, 0.5, 0.6, 0.7, 0.8, 0.9, 1-fold, 2-fold, 3-fold, 4-fold, or more, of nitrogen assimilation when grown under low nitrogen conditions as compared to wild-type plants.

Because of the increased assimilation of N₂-fixing bacteria by the plants as enabled by the present methods, the present plants can assimilate sufficient nitrogen to produce high yields even when inorganic nitrogen levels in the soil are low. As used herein, “reduced” or “low” or “minimal” inorganic “nitrogen conditions” or “nitrogen levels” refers to conditions in which the level of inorganic nitrogen, e.g., the level resulting from the introduction of fertilizer, is lower than the level that would normally be used for the crop plant, or which is recommended for the crop plant. For example, for rice plants, a level of inorganic nitrogen of less than 50 ppm can be used, e.g. about 25 ppm. In some embodiments, the level of inorganic nitrogen is at least about 10%, 20%, 30%, 40%, 50%, 60%, 70%, or 80% lower than the normal or recommended level.

### 4. Kits

In another aspect, kits are provided herein. In some embodiments, the kit comprises one or more element for producing genetically modified grain crop plants according to the present invention. The kit can comprise, e.g., one or more elements described herein for practicing the present methods, e.g., a guide RNA, an RNA-guided nuclease, a polynucleotide encoding an RNA-guided nuclease, a CRISPR-Cas RNP, culture medium, transfection reagents, etc.

Kits of the present invention can be packaged in a way that allows for safe or convenient storage or use (e.g., in a box or other container having a lid). Typically, kits of the present invention include one or more containers, each container storing a particular kit component such as a reagent, and so on. The choice of container will depend on the particular form of its contents, e.g., a kit component that is in liquid form, powder form, etc. Furthermore, containers can be made of materials that are designed to maximize the shelf-life of the kit components. As a non-limiting example, kit components that are light-sensitive can be stored in containers that are opaque.

In some embodiments, the kit contains one or more containers or devices, e.g. petri dish, flask, syringe, for practicing the present methods. In yet other embodiments, the kit further comprises instructions for use, e.g., containing directions (i.e., protocols) for the practice of the methods of this invention (e.g., instructions for using the kit for generating and using plants with increased flavone production). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### 5. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. Plant Metabolite-Mediated Induction of Biofilm Formation in Soil Bacteria Increases Biological Nitrogen Fixation of Crop Plants

We hypothesized that under low Nitrogen soil content conditions, the induction of biofilm formation in N₂-fixing bacteria by plant metabolites will decrease the Oxygen concentration in the vicinity of the bacterial cell, eliminating the inhibition of bacterial Nitrogenase by Oxygen and thereby the increasing bacterial atmospheric N₂ fixation activity. As a consequence, the soil N-fertilization required to attain agricultural yield production of non-leguminous crops will decrease. This not only will reduce the costs associated with the fertilization of agricultural lands, but will also significantly contribute to reducing the environmental burden generated by nitrates leaching into water aquifers, with a concomitant increase in nitrate concentrations and negative consequences for human health (15).

Our strategy is based on the following steps: (1) Screen the effects of different compounds on their ability to promote the formation of biofilms in N₂-fixing bacteria; (2) Identify plant metabolites—secreted by the plant roots—that increase —N₂-fixing bacteria biofilm production; and (3) Manipulate plant metabolic pathways (for example, via CRISPR/Cas9-mediated silencing) to increase the production (and secretion by the plant roots) of the metabolites identified.

We also hypothesized that these compounds that selectively induce biofilm formation will also benefit overall plant fitness in the soil and rhizosphere, thereby contributing to an efficient mutualistic relationship with the host plants.

### Chemical Screening of Biofilm Inducers

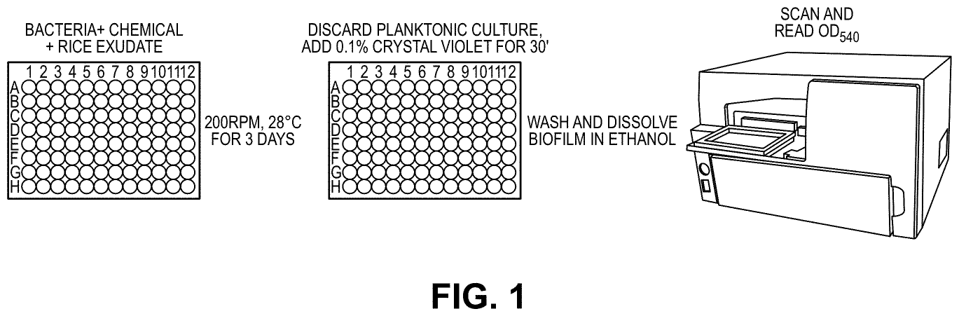

To assess the effect(s) of different chemicals on biofilm formation in N₂-fixing bacteria, we used a published protocol (www.jove.com/video/2437/microtiter-dish-biofilm-formation-assay). Basically, bacteria were grown in a 96-well plate in a rich-nutrient medium at 28° C. The compound to be tested was added and the culture was grown overnight. Plant exudates and 2 μl of the compound were added to the well and the bacteria grown for 3 days under shaking (200 rpm). After 3 days, the planktonic bacterial cultures were discarded and the wells were thoroughly washed with water. A solution of 1% of crystal violet was added to each well of the plate and the plate shaken for 10-15 min at 200 rpm. The plates are rinsed 3-4 times with water (by submerging the plants in a tub of water), shaken vigorously and blotted on a stack of paper towels (to eliminate excess of cells and dye), the microliter plate was placed upside down and air dried. To quantify the amount of biofilm that adhered to the well walls, 200 μl of ethanol were added to each well, the plates shacked at 200 rpm, at 28° C. for 10-15 min. The absorbance of the solution was measured at 540 nm, using ethanol as a blank (FIG. 1).

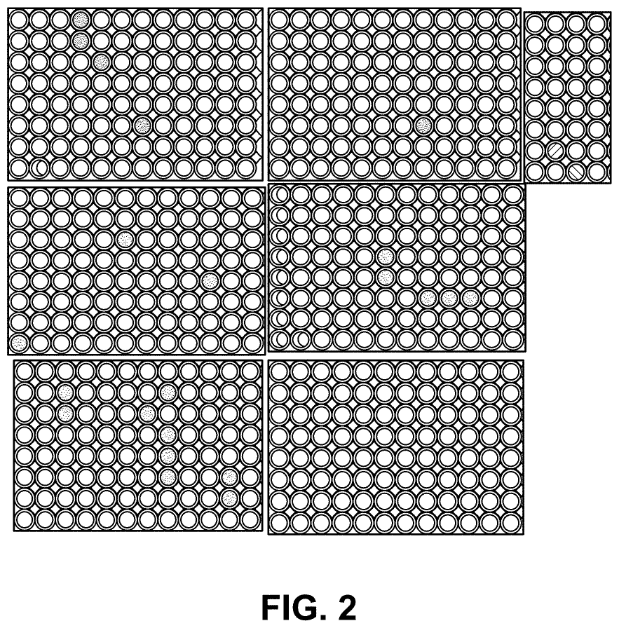

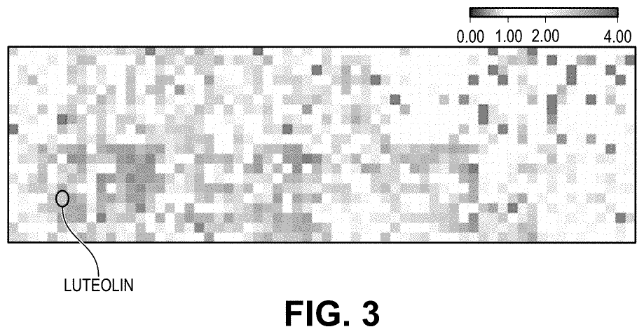

Flavonoids secreted by soybean roots have been shown to play roles in attracting rhizobia and in inducing the expression of rhizobial nod genes. In order to assess whether flavonoids could play some role in the induction of biofilm formation in N₂-fixing bacteria, we screened a chemical library comprised of 500 flavonoid derivatives of different origin (bacteria, plant and animal) (TimTec, Tampa, Fla., USA). Using the protocol described above, we tested biofilm synthesis using Glucanoacetobacter diazotrophicus as a representative of N₂-fixing bacteria. Several compounds enhanced biofilm production (FIGS. 2 and 3).

Characterization of Some Compounds Inducing Biofilm Formation in G. diazotrophicus.

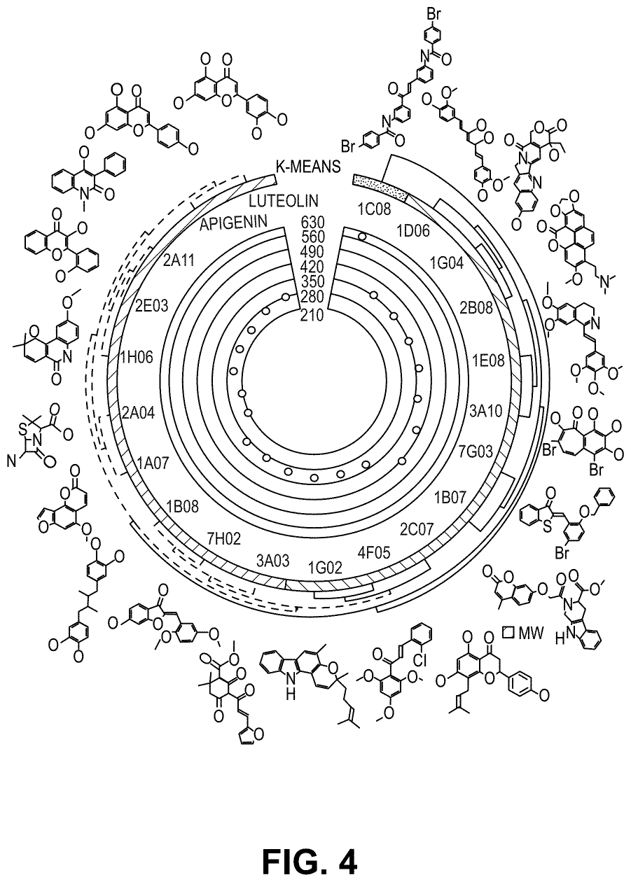

In order to assess structure-function of the different compounds, we performed a hierarchical clustering of the 20 compounds (chosen per their ability to induce biofilm formation in Glucanoacetobacter and other bacteria. To obtain the clustering we used Workbench Tools, an online service useful for the analysis and clustering of small molecules by structural similarities and physicochemical properties (ChemMine.ucr.edu/tools) (FIG. 4).

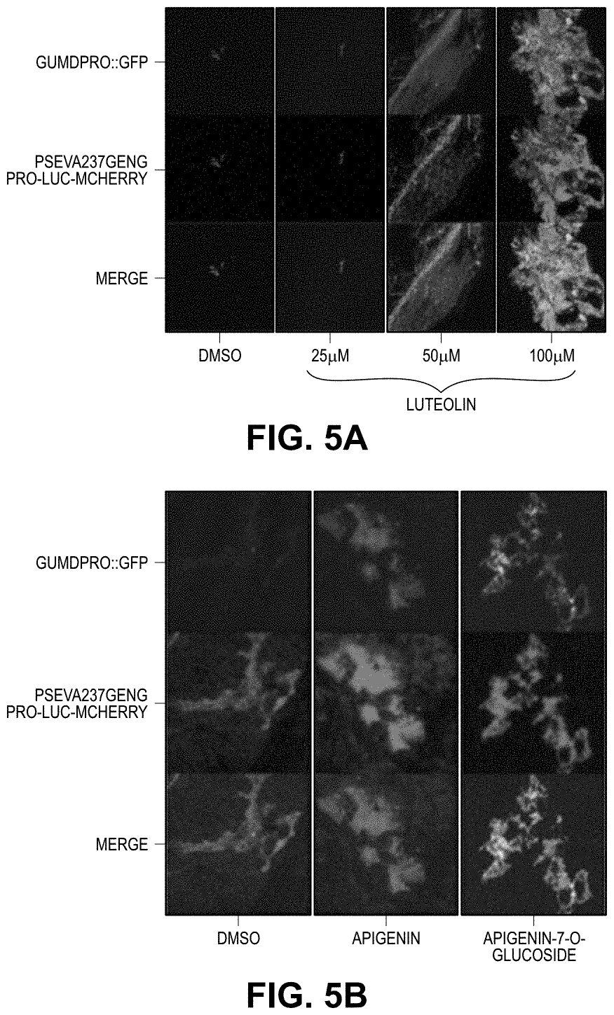

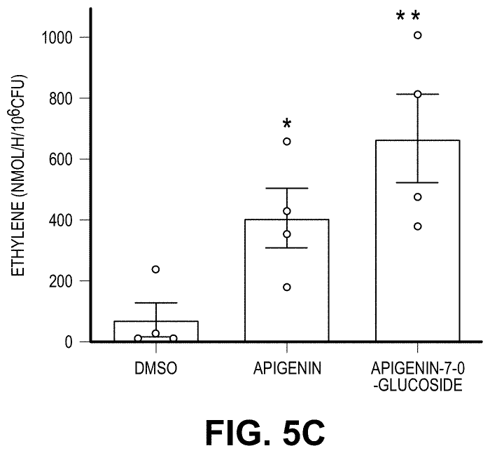

Our results indicated clustering among common moieties, particularly among heterooctacyclic compounds (e.g., Staurosporine) and flavonols (e.g., luteolin, apigenin) and anthraquinones (e.g. 2H03 and 4G03—Papaverine). (FIG. 4). Interestingly, flavonoids and flavonols have been shown to play essential roles in legume-rhizobium interaction for nodule formation (8). Therefore we assessed the effects of luteolin and apigenin in vivo. First we assessed the formation of chemical-induced formation of biofilm in bacterial cultures. For this, we generated transgenic Glucanoacetobacter constitutively expressing mCherry (transformed with pSEVAGeng-Luc-mCherry) in order to visualize mCherry fluorescent bacteria. Then we transformed the mCherry expressing bacteria with gumDpro::GFP. GumD encodes for components of the bacterial Exopolysacharides (EPS)] in order to visualize GFP-labelled biofilms. Thus the double labelling allowed as to follow the development of biofilm while visualizing the bacteria. The addition of luteolin to a suspension of Glucanoacetobacter showed the induction of biofilm formation by increasing amounts of luteolin (FIG. 5A). The addition of apigenin or its conjugate apigenin 7-O-glucoside showed the induction of biofilm formation (FIG. 5B).

Flavonoids perform several functions; pigments producing colors, inhibitors of cell cycle and also chemical messengers. Secretion of flavonoids was shown to aid symbiotic relationships between rhizobia and plants. Some flavonoids are associated with the response of plants to plant diseases. A representation of the different biosynthetic pathways in rice is shown in FIG. 6.

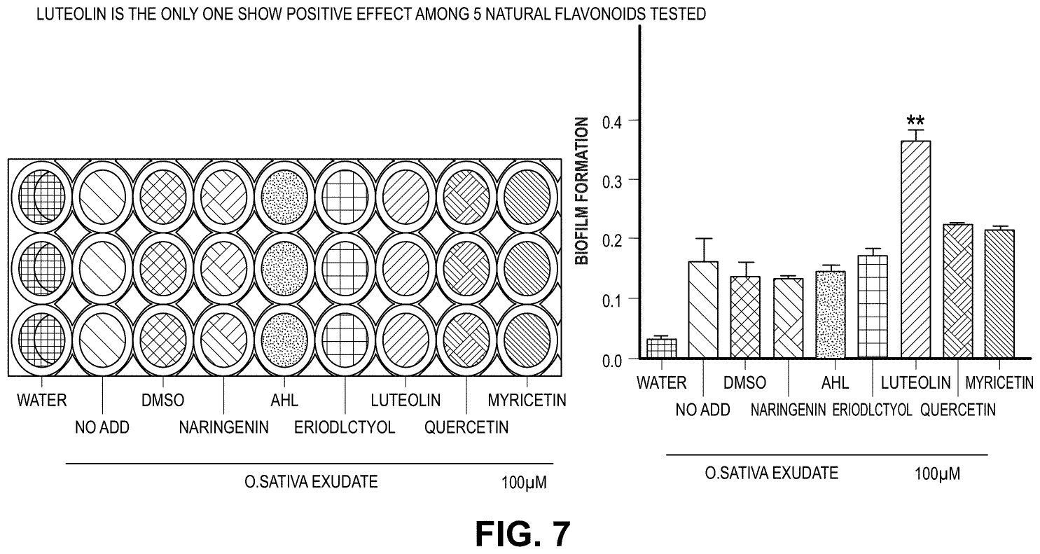

In order to assess the effect of compounds representing different group of flavonoids, we evaluated the formation of biofilm in Gluconacetobacter diazotrophicus exposed for 3 days to root exudates from Oryza sativa supplemented with Naringenin or Eriodictyol or Luteolin or Quercetin or Myricetin or AHL (Acyl Homoserine Lactone), a well-known compound shown to mediate interaction of bacteria and plant roots. Only luteolin induced a significant increase in biofilm production in Glucanoacetobacter (FIG. 7).

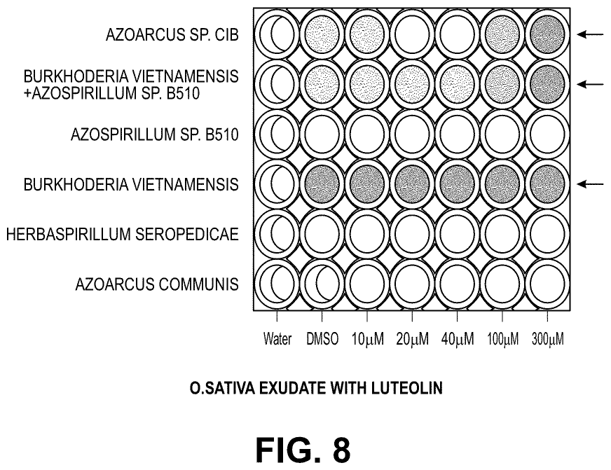

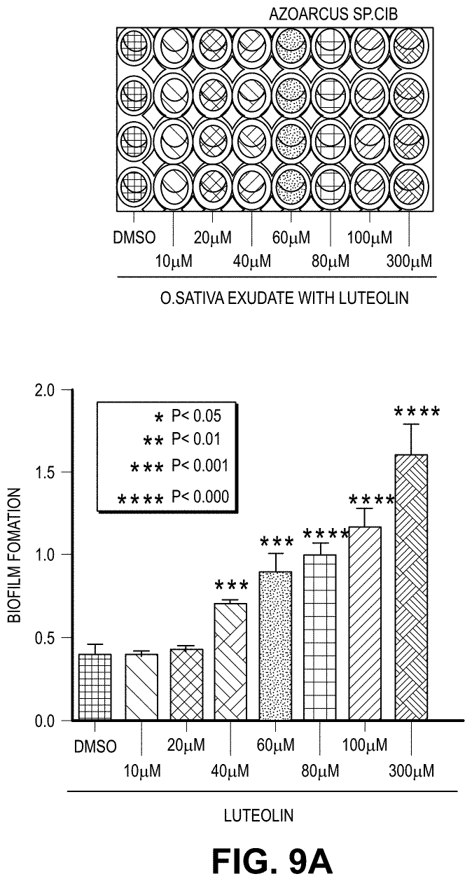

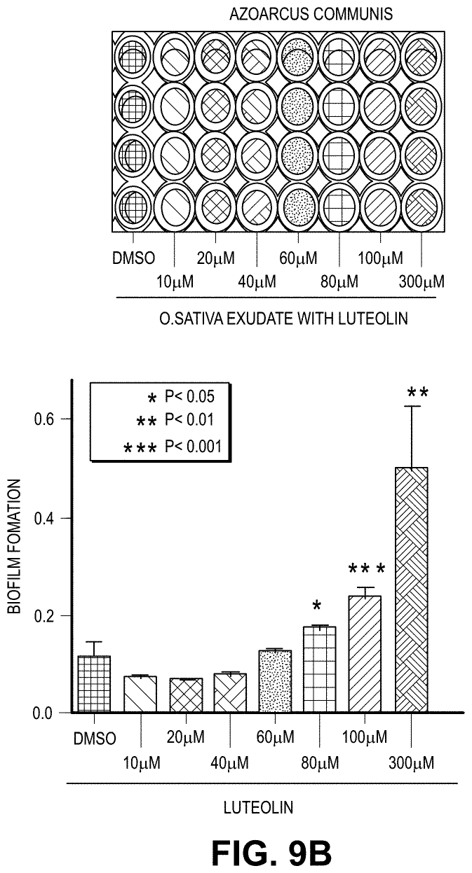

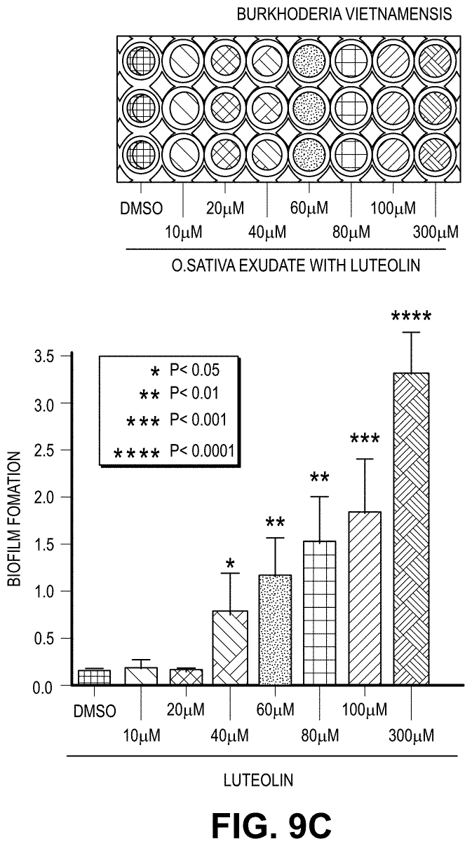

The effects of luteolin on the induction of biofilm production was tested in a number of N₂-fixing bacteria (FIG. 8). While Burkholderia vietnamensis and Azoarcus sp. CIB displayed a luteolin-induced biofilm synthesis, Azospirillum sp. 8510, Azoarcus communis, and Herbaspirillum seropedicae did not show an enhanced biofilm production. Also the response of the bacteria to luteolin was not uniform; Azoarcus sp. CIB displayed a lesser response to luteolin than Burkholderia vietnamensis. These results suggested a variety-specific differences on the synthesis of biofilms in response to flavonoids (see FIG. 9).

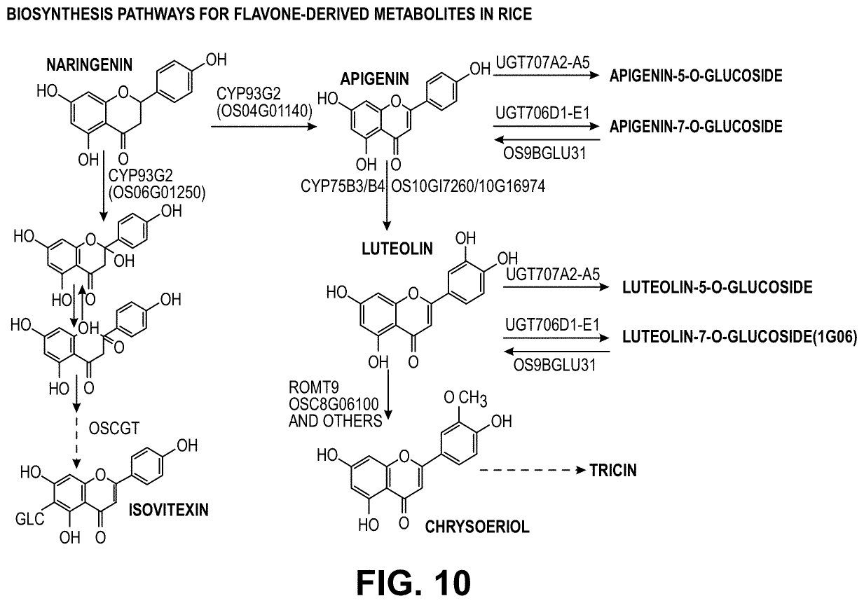

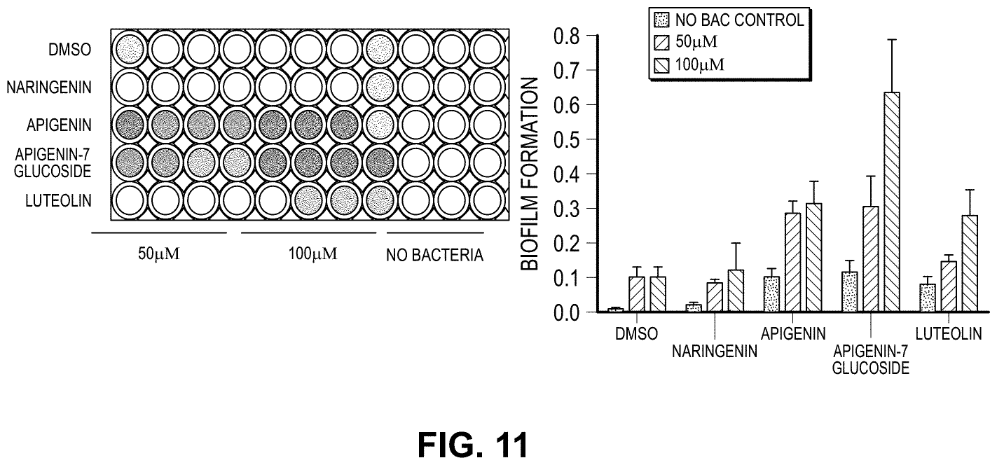

Flavones are a class of flavonoids synthesized directly from flavanones (i.e., Naringenin) (FIG. 10). Flavone formation is catalyzed by a flavone synthase which belongs to the plant cytochrome P450 superfamily. Most flavonoids, including flavones such as Apigenin and Luteolin, occur as glycosides. Glycosylation increases the chemical stability, bioavailability, and bioactivity of flavonoids. Glycosylation of Apigenin and Luteolin are catalyzed by flavonoid-glucosyltransferases. We tested the effects of Naringenin, Luteolin, Apigenin and Apigenin-7-glucoside on biofilm formation of Glucanoacetobacter diazotrophicus. The bacteria was incubated with 3 days with Oryza sativa root exudates supplemented with indicated concentrations of flavone-compounds (FIG. 11). The results clearly indicated the strongest biofilm induction in the bacteria incubated with apigenin and apigenin-7-glucoside, followed by Luteolin and Naringenin.

We investigated whether the increased flavone-induced bacterial biofilm production (elicited by the addition of the flavones Naringenin, Apigenin or Apigenin-7-Glucoside) increased bacteria N₂-fixation. Also, we tested whether the plant took up the nitrogen assimilated by the bacteria. It should be noted that we used Apigenin instead of Luteolin for 2 reasons: a) Apigenin induced a larger biofilm production than Luteolin (FIG. 11); b) Apigenin and its glucoside-derivative are less expensive than Luteolin.

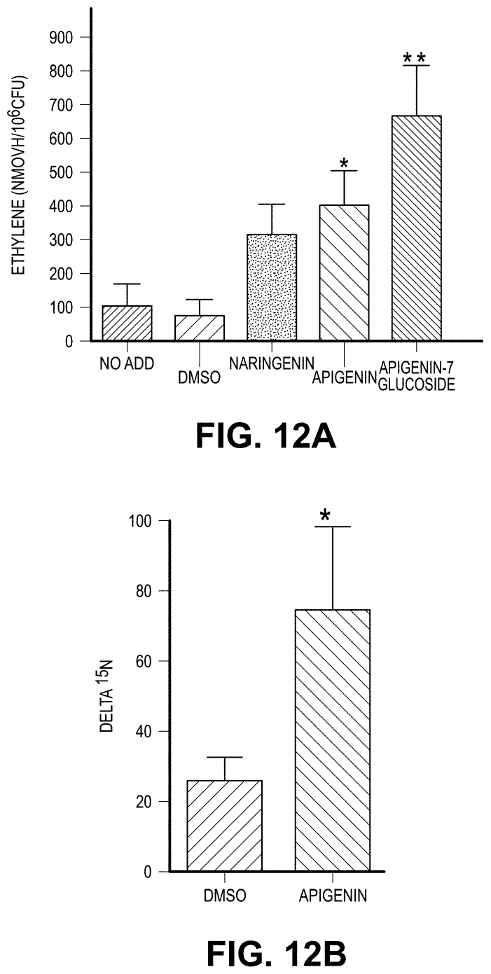

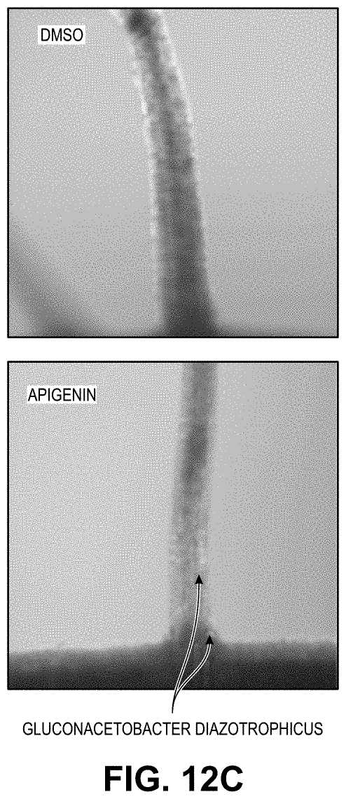

To assess the effects of the flavones on bacteria N₂ fixation, we used the acetylene reduction assay (ARA), where gas acetylene is added, and the resulting ethylene is measured by Gas Chromatography. The Bacterium was grown in tubes with Kitaake rice root exudates and 100 μM, shaken for 3 days at 28° C. Ten % of the air in the tube was replaced by acetylene, the cells incubated for 4 days and ethylene was measured by gas chromatography (FIG. 12A). We also assessed whether the N₂ fixed by the bacteria is assimilated by the plant. Rice seedlings were grown in soil in the presence of bacteria and Apigenin or DMSO (control). ¹⁵N₂ gas was added, the tubes closed and plants were incubated for 2 days. Following incubation, the leaves were cut and dried and the ¹⁵N-assimilated in the leaves was measured. Our results showed that the plants incubated with Apigenin displayed a significant increase in ¹⁵N into Nitrogen compounds, indicating that the bacteria fixed the ¹⁵N₂ and the resulting ammonium was assimilated by the plants (FIG. 12B).

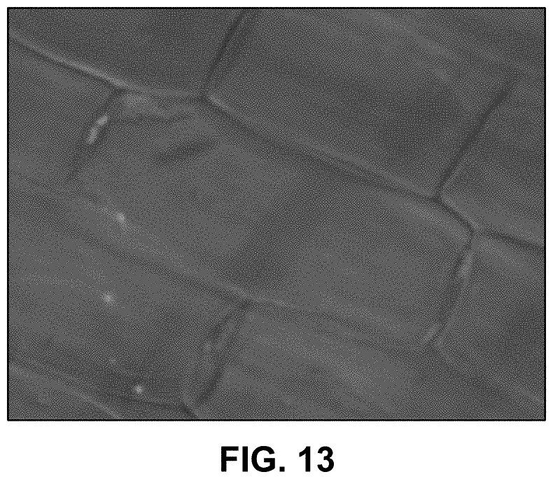

Microscopic observation of the rice root hairs showed extensive adherence of the bacteria (labelled with a fluorescence marker) to the biofilm (FIG. 12C). No bacteria was seen on the control treatments. Initial Confocal measurements would indicate that the bacteria also colonized the intracellular spaces of the rice roots. Quantitative experiments are underway to quantitate number of bacterial cells inside the plant tissues (FIG. 13) and number of cells adhered to the roots. However, clearly, the bulk of the bacteria is in the attached to the root surface (not shown).

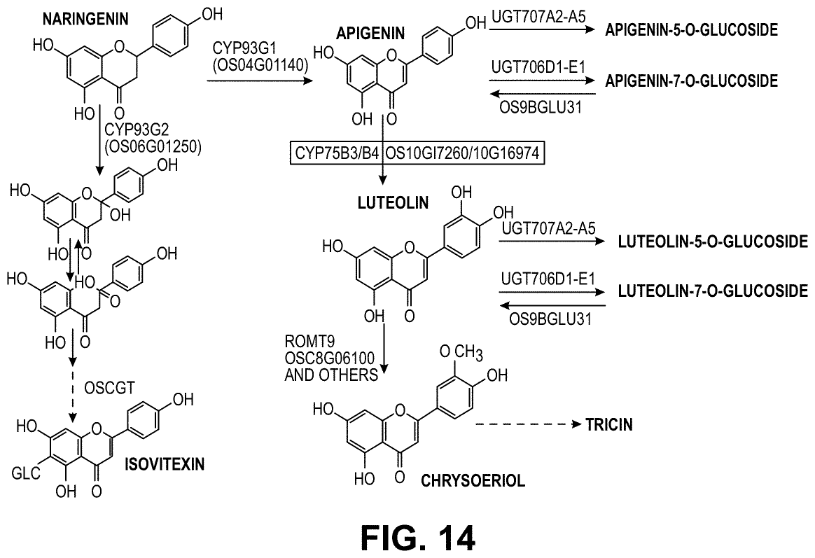

Our results showed that flavones and their glucoside derivatives induced biofilm formation in the N₂-fixing bacteria. The development of a biofilm, with its low permeability to Oxygen, provides a protection to the bacterial Nitrogenase from oxidative damage, thus allowing N₂-fixation by the free-living bacteria. Our hypothesis is that it is possible to increase N-assimilation in crop plants, if the plants can produce more flavones (which will be extruded to the soil by the roots). Interestingly, the larger effect of the flavone-glycoside derivatives on bacterial biofilm formation, would make feasible to alter the flavones (for example, Apigenin) biosynthetic pathway (including its glucosylation). An analysis of the flavone-derived metabolites in rice (and in most crops) (see FIG. 14) would indicate that changing the expression of genes encoding enzymes associated with flavone biosynthesis/degradation (whether overexpression with inducible promoters or gene silencing) could be used to increase flavone concentrations. For example, silencing Os10g17260/Os10g16974 encoding the cyt P450 CYP75B3/75B4, would generate an excess of Apigenin (since its conversion to Luteolin would be inhibited) and part of the Apigenin could be converted to Apigenin 5-O-glucoside and/or Apigenin 7-O-glucoside. (see FIG. 14), and larger amounts of Apigenin and its glucoside derivative(s) would be exuded by the roots to the soil with the concomitant effect on biofilm formation and N₂-fixation.

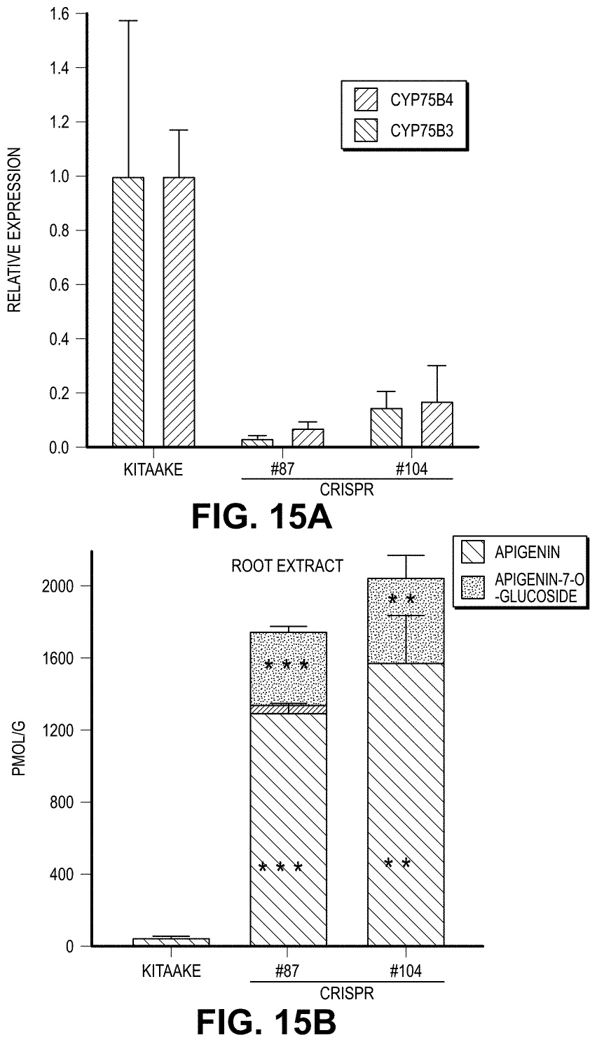

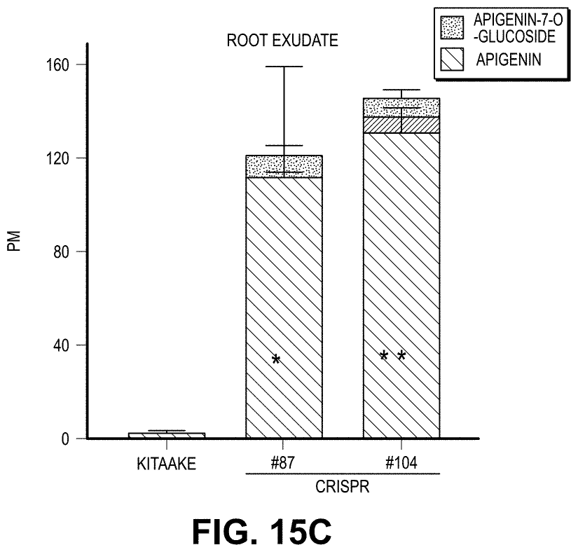

We generated CRISPR/Cas9 constructs, transformed rice plants and obtained plant lines with decreased expression of cyp75B3 and cyp75B4 (FIG. 15A). We obtained a number of transgenic homozygous lines and measured their flavone contents. The silencing of Os10g17260/Os10g16974 resulted in a reduction of the Cyt P450 (CYP75B3/75B4), mediating the formation of Luteolin from Apigenin, and induced a significant increase in Apigenin and its derivative, Apigenin-7-Glucoside in both roots (FIG. 15B) and root exudates (FIG. 15C).

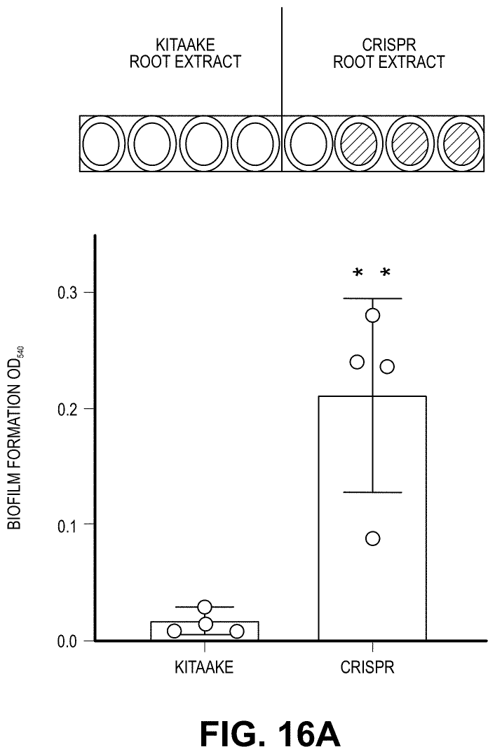

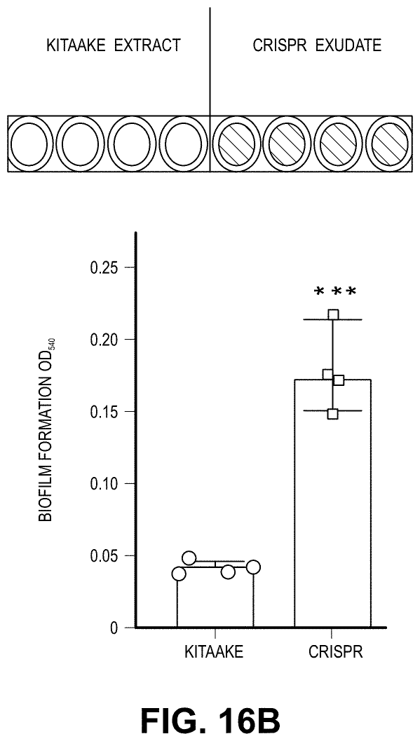

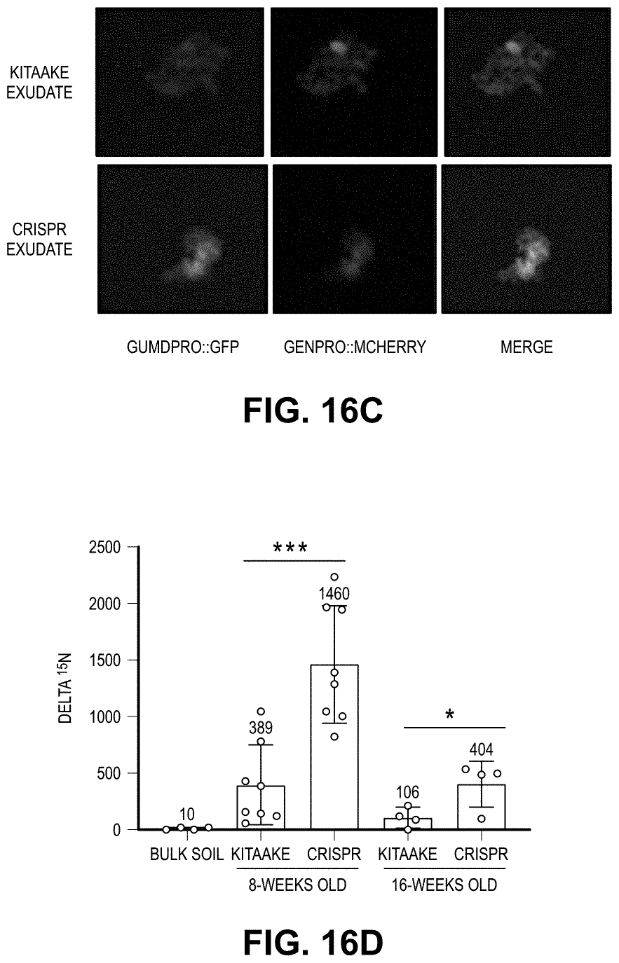

Root extracts and root exudates, obtained from cyp75b3/cyp75b4 (Os10g17260/Os10g16974) CRISPR/Cas9 knockout plants, increased biofilm production in Glucanoacetobacter diazotrophicus suspension (FIGS. 16A, 16B). The root exudate of the CRISPR line induced higher expression of the gumD gene, which is responsible for the first step in exopolysaccharide (EPS) production of biofilm in Gluconacetobacter diazotrophicus (FIG. 16C). The CRISPR/Cas9 rice lines incorporated more nitrogen from air (delta ¹⁵N) when grown in the greenhouse at both 8 weeks and 16 weeks of germination (FIG. 16D).

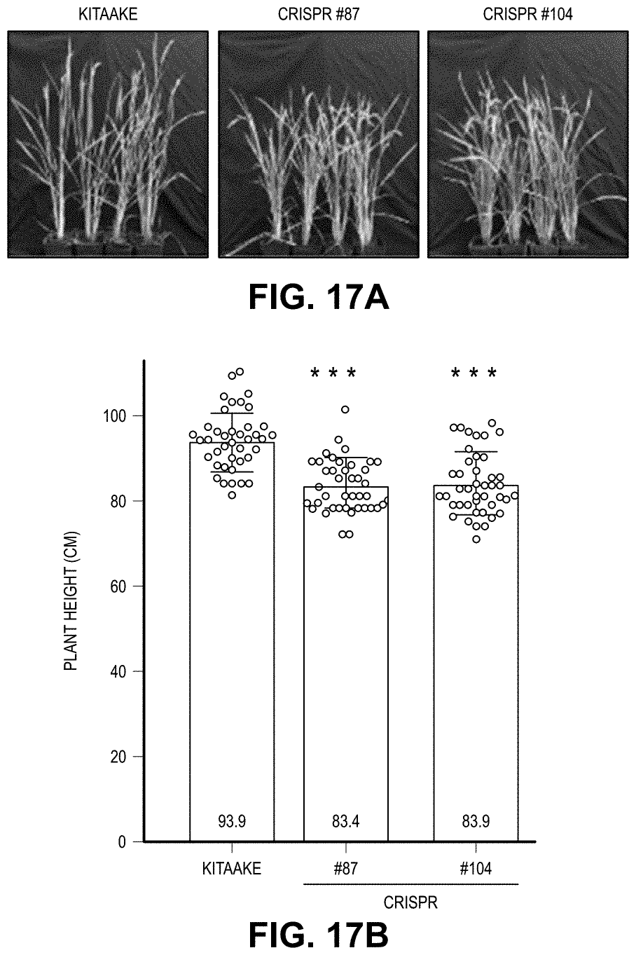

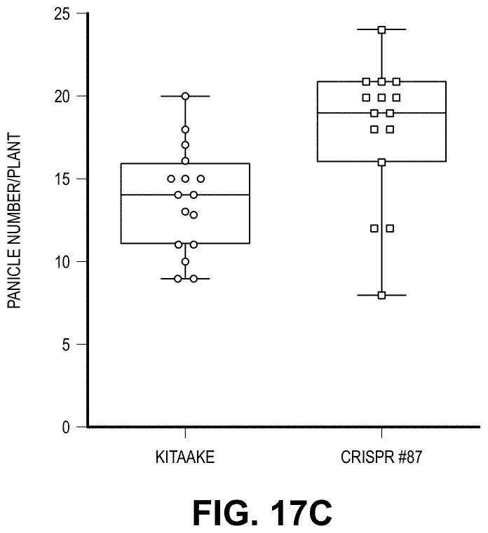

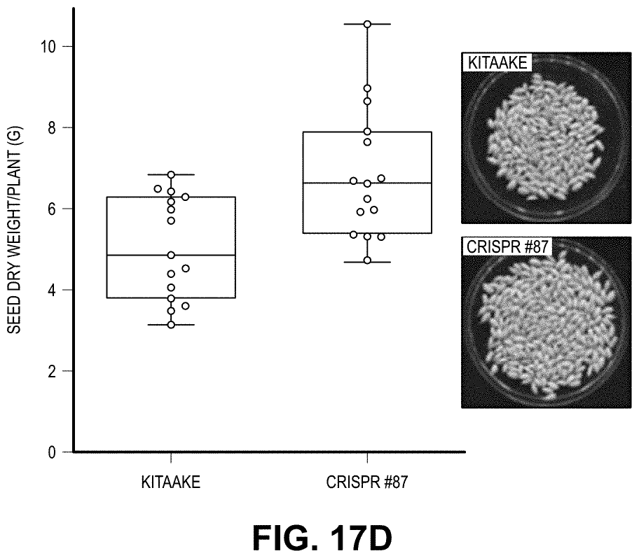

Kitaake wild-type and Crispr #87 and Crispr #104 silenced lines were grown in the greenhouse at standard growth conditions, the plants were fertilized, but the Nitrogen levels were kept at only 30% of the concentration recommended (25 ppm N). Notably, the silenced plants were somewhat shorter (FIG. 17B) but displayed a 40% increase in tiller number (FIG. 17C).

Plants were grown to maturity and seeds were harvested, dried and weighed. The silenced plants displayed a 40% yield increase as compared to the wild type plants grown at the same conditions (FIG. 17D).

Our results suggest the generation of Nitrogen-fixation in rice and other grain crops. The strategy involves the silencing of pathways associated with the catabolism of flavones (Apigenin, Luteolin, etc.). This strategy induced the accumulation of these metabolites inside the plant and the exudation of the flavones from the roots into the soil, where they activated the biofilm synthesis in the N₂-fixing bacteria. If plants are grown under minimal (deficient) inorganic N-conditions, the biofilm synthesis in the bacteria facilitates their N₂-fixation. The colonization of the plant roots by the N₂-fixing bacteria and its concomitant N₂-fixation will allow the reduction of agronomical operational costs (by reducing N-input) and also will provide an important tool to reduce nitrate contamination of groundwater, reducing its leaching into the water supplies.

### REFERENCES

1. Martin F M, Uroz S, Barker D G. Ancestral alliances: Plant mutualistic symbioses with fungi and bacteria. Science 356, (2017).
2. Zipfel C, Oldroyd G E D. Plant signalling in symbiosis and immunity. Nature 543, 328-336 (2017).
3. Khan M, Subramaniam R, Desveaux D. Of guards, decoys, baits and traps: pathogen perception in plants by type III effector sensors. Current opinion in microbiology 29, 49-55 (2016).
4. Bialas A, et al. Lessons in Effector and NLR Biology of Plant-Microbe Systems. Molecular plant-microbe interactions: MPMI 31, 34-45 (2018).
5. Sasse J, Martinoia E, Northen T. Feed Your Friends: Do Plant Exudates Shape the Root Microbiome? Trends in plant science 23, 25-41 (2018).
6. Haichar F E Z, Heulin T, Guyonnet J P, Achouak W. Stable isotope probing of carbon flow in the plant holobiont. Current opinion in biotechnology 41, 9-13 (2016).
7. Backer R, et al. Plant Growth-Promoting Rhizobacteria: Context, Mechanisms of Action, and Roadmap to Commercialization of Biostimulants for Sustainable Agriculture. Front Plant Sci 9, 1473 (2018).
8. Oldroyd G E, Murray J D, Poole P S, Downie J A. The rules of engagement in the legume-rhizobial symbiosis. Annual review of genetics 45, 119-144 (2011).
9. Wang D, Yang S, Tang F, Zhu H. Symbiosis specificity in the legume: rhizobial mutualism. Cellular microbiology 14, 334-342 (2012).
10. Shrestha R K, Ladha J K. Genotypic Variation in Promotion of Rice Dinitrogen Fixation as Determined by Nitrogen-15 Dilution. Soil Sci Soc Am J 60, 1815-1821 (1996).
11. Chen X, et al. Rice responds to endophytic colonization which is independent of the common symbiotic signaling pathway. The New phytologist 208, 531-543 (2015).
12. Flemming H C, Wingender J. The biofilm matrix. Nature reviews Microbiology 8, 623-633 (2010).
13. Flemming H C, Wingender J, Szewzyk U, Steinberg P, Rice S A, Kjelleberg S. Biofilms: an emergent form of bacterial life. Nature reviews Microbiology 14, 563-575 (2016).
14. Meneses C H, Rouws L F, Simoes-Araujo J L, Vidal M S, Baldani J I. Exopolysaccharide production is required for biofilm formation and plant colonization by the nitrogen-fixing endophyte Gluconacetobacter diazotrophicus. Molecular plant-microbe interactions: MPMI 24, 1448-1458 (2011).
15. Ward M H, Jones R R, Brender J D, de Kok T M, Weyer P J, Nolan B T, Villanueva C M and van Breda S G. Int. J. Environm. Res. Public Health 15, 1557 (2018)
16. Wang D, Xu A, Elmerich C, Ma L Z. Biofilm formation enables free-living nitrogen-fixing rhizobacteria to fix nitrogen under aerobic conditions. The ISME journal 11, 1602-1613 (2017).
17. Voges M, Bai Y, Schulze-Lefert P, Sanely E S. Plant-derived coumarins shape the composition of an Arabidopsis synthetic root microbiome. Proceedings of the National Academy of Sciences of the United States of America 116, 12558-12565 (2019).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

INFORMAL SEQUENCE LISTING

SEQ ID NO: 1
Oryza sativa ssp. Indica (OsR498G1018420100.01)
Amino acid sequence
MEVAAMEISTSLLLTTVALSVIVCYALVFSRAGKARAPLPLPPGPRGWPVLGNLPQL
GGKTHQTLHEMTKVYGPLIRLRFGSSDVVVAGSAPVAAQFLRTHDANFSSRPRNSG
GEHMAYNGRDVVFGPYGPRWRAMRKICAVNLFSARALDDLRAFREREAVLMVRSL
AEASAAPGSSSPAAVVLGKEVNVCTTNALSRAAVGRRVFAAGAGEGAREFKEIVLE
VMEVGGVLNVGDFVPALRWLDPQGVVARMKKLHHRFDDMMNAIIAERRAGSLLK
PTDSREEGKDLLGLLLAMVQEQEWLAAGEDDRITDTEIKALILNLFVAGTDTTSTIVE
WTMAELIRHPDILKQAQEELDVVVGRDRLLLESDLSHLTFFHAIIKETFRLHPSTPLSL
PRMASEECEIAGYRIPKGAELLVNVWGIARDPAIWPDPLEYKPSRFLPGGTHTDVDV
KGNDFGLIPFGAGRRICAGLSWGLRMVTMTAATLVHAFDWQLPADQTPDKLNMDE
AFTLLLQRAEPLVVHPVPRLLPSAYNIA
SEQ ID NO: 2
Oryza sativa ssp. Indica (OsR498G1018420100.01)
Nucleotide sequence
ATGGAGGTCGCCGCCATGGAGATCTCTACCTCATTGCTCCTCACCACCGTGGCTC
TCTCCGTCATCGTGTGCTACGCCCTGGTCTTCTCCCGCGCCGGGAAGGCGCGTGC
GCCGCTGCCGCTGCCGCCTGGCCCCAGGGGATGGCCGGTGCTGGGCAACCTGCC
GCAGCTGGGCGGGAAGACGCACCAGACGCTGCACGAGATGACCAAGGTGTACG
GCCCGCTGATCCGGCTCCGGTTCGGGAGCTCCGACGTGGTGGTCGCCGGCTCGGC
GCCGGTGGCGGCGCAGTTCCTCCGCACCCACGATGCCAACTTCAGCAGCCGGCC
ACGCAACTCCGGCGGCGAGCACATGGCGTACAACGGCCGGGACGTCGTGTTCGG
GCCGTACGGGCCGCGGTGGCGCGCCATGCGGAAGATTTGCGCCGTCAACCTCTTC
TCCGCGCGCGCGCTCGACGACCTGCGCGCTTTCCGGGAGCGGGAGGCCGTGCTG
ATGGTTAGGTCGCTGGCGGAGGCGAGCGCCGCCCCTGGGTCGTCGTCTCCAGCG
GCGGTGGTCCTGGGAAAGGAGGTGAATGTCTGCACGACGAACGCGCTGTCGCGC
GCCGCGGTCGGGCGCCGCGTGTTCGCCGCCGGCGCGGGCGAGGGCGCGAGGGAG
TTCAAAGAGATCGTGCTGGAGGTGATGGAGGTGGGTGGTGTGCTGAACGTCGGC
GACTTCGTGCCGGCGCTCCGGTGGCTGGACCCGCAGGGCGTGGTAGCGAGGATG
AAAAAGCTGCACCACCGGTTCGACGACATGATGAACGCGATCATCGCGGAGAGG
AGGGCCGGATCACTACTCAAACCAACCGACAGTCGTGAGGAAGGTAAGGACTTG
CTTGGCTTGCTCCTGGCTATGGTGCAGGAGCAGGAGTGGCTCGCCGCCGGCGAG
GACGACAGGATCACCGACACGGAAATCAAGGCCCTTATCCTGAATCTATTCGTG
GCGGGCACAGACACAACATCAACCATAGTTGAGTGGACAATGGCAGAGCTGATT
CGACACCCAGATATCCTCAAGCAGGCCCAAGAGGAGCTAGATGTTGTTGTGGGT
CGTGATAGGCTCCTCTTAGAGTCGGATCTATCACATCTCACCTTCTTCCATGCTAT
CATCAAGGAGACATTCCGTCTTCATCCATCAACCCCGCTCTCGCTGCCACGCATG
GCATCTGAGGAGTGTGAGATCGCAGGCTACCGTATCCCCAAGGGTGCAGAGTTG
CTGGTCAATGTGTGGGGGATCGCCCGTGACCCAGCCATATGGCCTGACCCACTAG
AGTACAAGCCCTCTCGGTTCCTCCCCGGTGGGACGCACACTGATGTGGATGTCAA
GGGAAATGATTTCGGACTTATACCATTCGGTGCAGGGCGAAGGATATGCGCCGG
CCTCAGTTGGGGCCTGCGGATGGTCACCATGACAGCGGCCACGCTGGTGCATGC
ATTCGACTGGCAGCTACCAGCGGACCAGACGCCAGACAAGCTCAATATGGATGA
GGCGTTTACCCTCCTGCTGCAAAGGGCAGAGCCATTGGTGGTTCACCCGGTACCA
AGGCTTCTCCCATCCGCTTACAATATTGCATAA
SEQ ID NO: 3
Oryza sativa ssp. Indica (OsR498G1018427100.01)
Amino acid sequence
MEVAAMEISTSLLLTTVALSVIVCYALVFSRAGKARAPLPLPPGPRGWPVLGNLPQL
GGKTHQTLHEMTKVYGPLIRLRFGSSDVVVAGSAPVAAQFLRTHDANFSSRPRNSG
GEHMAYNGRDVVFGPYGPRWRAMRKICAVNLFSARALDDLRAFREREAVLMVRSL
AEASAAPGSSSPAAVVLGKEVNVCTTNALSRAAVGRRVFAAGAGEGAREFKEIVLE
VMEVGGVLNVGDFVPALRWLDPQGVVARMKKLHHRFDDMMNAIIAERRAGSLLK
PTDSREEGKDLLGLLLAMVQEQEWLAAGEDDRITDTEIKALILNLFVAGTDTTSTIVE
WTMAELIRHPDILKQAQEELDVVVGRDRLLLESDLSHLTFFHAIIKETFRLHPSTPLSL
PRMASEECEIAGYRIPKGAELLVNVWGIARDPAIWPDPLEYKPSRFLPGGTHTDVDV
KGNDFGLIPFGAGRRICAGLSWGLRMVTMTAATLVHAFDWQLPADQTPDKLNMDE
AFTLLLQRAEPLVVHPVPRLLPSAYNIA
SEQ ID NO: 4
Oryza sativa ssp. Indica (OsR498G1018427100.01)
Nucleotide sequence
ATGGAGGTCGCCGCCATGGAGATCTCTACCTCATTGCTCCTCACCACCGTGGCTC
TCTCCGTCATCGTGTGCTACGCCCTGGTCTTCTCCCGCGCCGGGAAGGCGCGTGC
GCCGCTGCCGCTGCCGCCTGGCCCCAGGGGATGGCCGGTGCTGGGCAACCTGCC
GCAGCTGGGCGGGAAGACGCACCAGACGCTGCACGAGATGACCAAGGTGTACG
GCCCGCTGATCCGGCTCCGGTTCGGGAGCTCCGACGTGGTGGTCGCCGGCTCGGC
GCCGGTGGCGGCGCAGTTCCTCCGCACCCACGATGCCAACTTCAGCAGCCGGCC
ACGCAACTCCGGCGGCGAGCACATGGCGTACAACGGCCGGGACGTCGTGTTCGG
GCCGTACGGGCCGCGGTGGCGCGCCATGCGGAAGATTTGCGCCGTCAACCTCTTC
TCCGCGCGCGCGCTCGACGACCTGCGCGCTTTCCGGGAGCGGGAGGCCGTGCTG
ATGGTTAGGTCGCTGGCGGAGGCGAGCGCCGCCCCTGGGTCGTCGTCTCCAGCG
GCGGTGGTCCTGGGAAAGGAGGTGAATGTCTGCACGACGAACGCGCTGTCGCGC
GCCGCGGTCGGGCGCCGCGTGTTCGCCGCCGGCGCGGGCGAGGGCGCGAGGGAG
TTCAAAGAGATCGTGCTGGAGGTGATGGAGGTGGGTGGTGTGCTGAACGTCGGC
GACTTCGTGCCGGCGCTCCGGTGGCTGGACCCGCAGGGCGTGGTAGCGAGGATG
AAAAAGCTGCACCACCGGTTCGACGACATGATGAACGCGATCATCGCGGAGAGG
AGGGCCGGATCACTACTCAAACCAACCGACAGTCGTGAGGAAGGTAAGGACTTG
CTTGGCTTGCTCCTGGCTATGGTGCAGGAGCAGGAGTGGCTCGCCGCCGGCGAG
GACGACAGGATCACCGACACGGAAATCAAGGCCCTTATCCTGAATCTATTCGTG
GCGGGCACAGACACAACATCAACCATAGTTGAGTGGACAATGGCAGAGCTGATT
CGACACCCAGATATCCTCAAGCAGGCCCAAGAGGAGCTAGATGTTGTTGTGGGT
CGTGATAGGCTCCTCTTAGAGTCGGATCTATCACATCTCACCTTCTTCCATGCTAT
CATCAAGGAGACATTCCGTCTTCATCCATCAACCCCGCTCTCGCTGCCACGCATG
GCATCTGAGGAGTGTGAGATCGCAGGCTACCGTATCCCCAAGGGTGCAGAGTTG
CTGGTCAATGTGTGGGGGATCGCCCGTGACCCAGCCATATGGCCTGACCCACTAG
AGTACAAGCCCTCTCGGTTCCTCCCCGGTGGGACGCACACTGATGTGGATGTCAA
GGGAAATGATTTCGGACTTATACCATTCGGTGCAGGGCGAAGGATATGCGCCGG
CCTCAGTTGGGGCCTGCGGATGGTCACCATGACAGCGGCCACGCTGGTGCATGC
ATTCGACTGGCAGCTACCAGCGGACCAGACGCCAGACAAGCTCAATATGGATGA
GGCGTTTACCCTCCTGCTGCAAAGGGCAGAGCCATTGGTGGTTCACCCGGTACCA
AGGCTTCTCCCATCCGCTTACAATATTGCATAA
SEQ ID NO: 5
Oryza sativa ssp. Japonica (LOC_Os10g16974)
Amino acid sequence
MEVAAMEISTSLLLTTVALSVIVCYALVFSRAGKARAPLPLPPGPRGWPVLGNLPQL
GGKTHQTLHEMTKVYGPLIRLRFGSSDVVVAGSAPVAAQFLRTHDANFSSRPRNSG
GEHMAYNGRDVVFGPYGPRWRAMRKICAVNLFSARALDDLRAFREREAVLMVRSL
AEASAAPGSSSPAAVVLGKEVNVCTTNALSRAAVGRRVFAAGAGEGAREFKEIVLE
VMEVGGVLNVGDFVPALRWLDPQGVVARMKKLHRRFDDMMNAIIAERRAGSLLKP
TDSREEGKDLLGLLLAMVQEQEWLAAGEDDRITDTEIKALILNLFVAGTDTTSTIVE
WTMAELIRHPDILKHAQEELDVVVGRDRLLSESDLSHLTFFHAIIKETFRLHPSTPLSL
PRMASEECEIAGYRIPKGAELLVNVWGIARDPAIWPDPLEYKPSRFLPGGTHTDVDV
KGNDFGLIPFGAGRRICAGLSWGLRMVTMTAATLVHAFDWQLPADQTPDKLNMDE
AFTLLLQRAEPLVVHPVPRLLPSAYNIA
SEQ ID NO: 6
Oryza sativa ssp. Japonica (LOC_Os10g16974)
Nucleotide sequence
ATGGAGGTCGCCGCCATGGAGATCTCTACCTCATTGCTCCTCACCACCGTGGCTC
TCTCCGTCATCGTGTGCTACGCCCTGGTCTTCTCCCGCGCCGGGAAGGCGCGTGC
GCCGCTGCCGCTGCCGCCTGGCCCCAGGGGATGGCCGGTGCTGGGCAACCTGCC
GCAGCTGGGCGGGAAGACGCACCAGACGCTGCACGAGATGACCAAGGTGTACG
GCCCGCTGATCCGGCTCCGGTTCGGGAGCTCCGACGTGGTGGTCGCCGGCTCGGC
GCCGGTGGCGGCGCAGTTCCTCCGCACCCACGATGCCAACTTCAGCAGCCGGCC
ACGCAACTCCGGCGGCGAGCACATGGCGTACAACGGCCGGGACGTCGTGTTCGG
GCCGTACGGGCCGCGGTGGCGCGCCATGCGGAAGATTTGCGCCGTCAACCTCTTC
TCCGCGCGCGCGCTCGACGACCTGCGCGCTTTCCGGGAGCGGGAGGCCGTGCTG
ATGGTTAGGTCGCTGGCGGAGGCGAGCGCCGCCCCTGGGTCGTCGTCTCCAGCG
GCGGTGGTCCTGGGAAAGGAGGTGAATGTCTGCACGACGAACGCGCTGTCGCGC
GCCGCGGTCGGGCGCCGCGTGTTCGCCGCCGGCGCGGGCGAGGGCGCGAGGGAG
TTCAAAGAGATCGTGCTGGAGGTGATGGAGGTGGGTGGTGTGCTGAACGTCGGC
GACTTCGTGCCGGCGCTCCGGTGGCTGGACCCGCAGGGCGTGGTAGCGAGGATG
AAAAAGCTGCACCGCCGGTTCGACGACATGATGAACGCGATCATCGCGGAGAGG
AGGGCCGGATCACTACTCAAACCAACCGACAGTCGTGAGGAAGGTAAGGACTTG
CTTGGCTTGCTCCTGGCTATGGTGCAGGAGCAGGAGTGGCTCGCCGCCGGCGAG
GACGACAGGATCACCGACACGGAAATCAAGGCCCTTATCCTGAATCTATTCGTG
GCGGGCACAGACACAACATCAACCATAGTTGAGTGGACAATGGCAGAGCTGATT
CGACACCCAGATATCCTCAAGCACGCCCAAGAGGAGCTAGATGTTGTTGTGGGT
CGTGATAGGCTCCTCTCAGAGTCGGATCTATCACATCTCACCTTCTTCCATGCTAT
CATCAAGGAGACATTCCGTCTACATCCATCAACACCGCTCTCGCTGCCACGCATG
GCATCTGAGGAGTGTGAGATCGCAGGCTACCGTATCCCCAAGGGTGCAGAGTTG
CTGGTCAATGTGTGGGGGATCGCCCGTGACCCAGCCATATGGCCTGACCCACTAG
AGTACAAGCCCTCTCGGTTCCTCCCCGGTGGGACGCACACTGATGTGGATGTCAA
GGGAAATGATTTCGGACTTATACCATTCGGTGCAGGGCGAAGGATATGCGCCGG
CCTCAGTTGGGGCCTGCGGATGGTCACCATGACAGCGGCCACGCTGGTGCATGC
ATTCGACTGGCAGCTACCAGCGGACCAGACGCCAGACAAGCTCAATATGGATGA
GGCGTTTACCCTCCTGCTGCAAAGGGCAGAGCCATTGGTGGTTCACCCGGTACCA
AGGCTTCTCCCATCCGCTTACAATATTGCATAA
SEQ ID NO: 7
Oryza sativa ssp. Japonica (LOC_Os10g17260)
Amino acid sequence
MDVVPLPLLLGSLAVSAAVWYLVYFLRGGSGGDAARKRRPLPPGPRGWPVLGNLP
QLGDKPHHTMCALARQYGPLFRLRFGCAEVVVAASAPVAAQFLRGHDANFSNRPPN
SGAEHVAYNYQDLVFAPYGARWRALRKLCALHLFSAKALDDLRAVREGEVALMV
RNLARQQAASVALGQEANVCATNTLARATIGHRVFAVDGGEGAREFKEMVVELMQ
LAGVFNVGDFVPALRWLDPQGVVAKMKRLHRRYDNMMNGFINERKAGAQPDGVA
AGEHGNDLLSVLLARMQEEQKLDGDGEKITETDIKALLLNLFTAGTDTTSSTVEWAL
AELIRHPDVLKEAQHELDTVVGRGRLVSESDLPRLPYLTAVIKETFRLHPSTPLSLPRE
AAEECEVDGYRIPKGATLLVNVWAIARDPTQWPDPLQYQPSRFLPGRMHADVDVK
GADFGLIPFGAGRRICAGLSWGLRMVTLMTATLVHGFDWTLANGATPDKLNMEEA
YGLTLQRAVPLMVQPVPRLLPSAYGV
SEQ ID NO: 8
Oryza sativa ssp. Japonica
Nucleotide sequence
AAACCCGCATTTCCCATCGTACAACGAGCGAGCGGATCATACGGTCATGGACGTT
GTGCCTCTCCCGCTGCTGCTCGGCTCCCTGGCCGTGTCCGCCGCCGTGTGGTACCT
TGTGTACTTCCTCCGCGGCGGCAGCGGCGGCGACGCGGCGAGGAAGCGGCGGCC
TTTGCCACCCGGGCCACGCGGGTGGCCCGTGCTGGGCAACCTGCCGCAGCTCGGC
GACAAGCCGCACCACACCATGTGCGCCCTGGCGCGGCAGTACGGCCCGCTGTTC
CGGCTCCGGTTCGGCTGCGCCGAGGTGGTGGTGGCCGCGTCGGCGCCCGTGGCTG
CGCAGTTCCTGCGCGGGCACGATGCCAACTTCAGCAACCGCCCGCCCAACTCGG
GCGCCGAGCACGTCGCGTACAACTACCAGGACCTCGTCTTCGCGCCCTACGGTGC
TCGCTGGCGCGCCCTGCGGAAGCTGTGCGCGCTCCACCTCTTCTCGGCCAAGGCG
CTCGACGACCTCCGAGCAGTCCGGGAGGGCGAGGTCGCGCTCATGGTGAGGAAC
CTCGCTCGGCAGCAGGCGGCGTCAGTGGCGCTGGGGCAGGAAGCGAACGTCTGC
GCCACGAACACGCTGGCCCGCGCCACCATCGGTCACCGGGTGTTCGCCGTCGAC
GGCGGGGAAGGCGCAAGGGAGTTCAAGGAGATGGTTGTGGAGCTGATGCAGCTC
GCCGGCGTTTTCAACGTCGGGGACTTCGTGCCGGCGCTCCGGTGGCTCGACCCGC
AGGGCGTCGTGGCAAAGATGAAGAGGCTGCACCGTCGGTACGACAACATGATGA
ACGGATTCATCAACGAAAGGAAGGCCGGGGCGCAGCCCGACGGGGTCGCCGCTG
GCGAGCACGGCAACGACCTTCTAAGCGTGCTGCTGGCGAGGATGCAGGAGGAGC
AGAAGCTGGACGGCGACGGCGAAAAGATCACCGAAACTGACATCAAAGCTCTGC
TCCTGAACCTATTCACTGCGGGGACGGATACGACATCGAGCACGGTGGAGTGGG
CACTGGCGGAGCTGATCCGGCACCCGGACGTCCTCAAGGAGGCCCAGCATGAGC
TTGACACCGTCGTCGGTAGGGGTCGTCTCGTGTCCGAGTCTGACCTTCCACGCCT
CCCCTACCTCACCGCGGTGATCAAGGAGACGTTTCGGCTTCACCCGTCAACGCCG
CTCTCACTGCCTCGGGAGGCTGCAGAGGAGTGTGAGGTGGACGGCTACCGTATC
CCCAAGGGCGCTACCCTCCTAGTCAACGTCTGGGCTATAGCCCGTGACCCGACCC
AATGGCCCGACCCGCTACAGTACCAGCCTTCTCGGTTTCTCCCCGGCAGGATGCA
TGCAGACGTGGATGTCAAGGGTGCTGATTTCGGCCTGATACCATTCGGAGCAGG
ACGGAGAATATGCGCTGGCCTTAGTTGGGGCTTGCGGATGGTCACACTGATGACT
GCCACGCTAGTGCACGGGTTCGACTGGACCTTGGCTAACGGCGCGACTCCGGAC
AAGCTCAACATGGAGGAGGCCTATGGGCTCACCTTGCAGAGGGCCGTGCCGTTG
ATGGTCCAGCCCGTGCCAAGGCTGCTTCCATCGGCTTATGGAGTATAAAACCGGT
CTACTTACTAGTACCACTTTAAATTAAGGTCAGAAATCGGTGGAGACTACTTGCA
GTGTTGGCCGCATTATATGACGTATTATTTTGTTTTGTTTGTTGGTGGAAAAATAA
AGTAGTCTATCTCAGTGTTATCTGGCACTAAAGGAACTCTAGAAATGGTGGCAAA
ATAGAGTACTATCGTGGAATCATAAAAAAGGATTATTTGGTGTATAATACAGAA
AAATTTATG
SEQ ID NO: 9
Genomic sequence, rice CYP75B3
Exons, Target sequences, PAM (NGG), Target-like sequence
>CYP75B3_LOC_Os10g17260_chr10_8679310-8681284
TAAACCCGCATTTCCCATCGTACAACGAGCGAGCGGATCATACGGTCATGGACGTTGTGCCT
CTCCCGCTGCTGCTCGGCTCCCTGGCCGTGTCCGCCGCCGTGTGGTACCTTGTGTACTTCCT
CCgcggcggcagcggcggcgacgcggcgaggaagcggcggcCTTTGCCACCCGGGCCACGCG
GGTGGCCCGTGCTGGGCAACCTGCCGCAGCTCGGCGACAAGCCGCACCACACCATGTGCGCC
CTGGCGCGGCAGTACGGCCCGCTGTTCCGGCTCCGGTTCGGCTGCGCCGAGGTGGTGGTGGC
CGCGTCGGCGCCCGTGGCTGCGCAGTTCCTGCGCGGGCACGATGCCAACTTCAGCAACCGCC
CGCCCAACTCGGGCGCCGAGCACGTCGCGTACAACTACCAGGACCTCGTCTTCGCGCCCTAC
GGTGCTCGCTGGCGCGCCCTGCGGAAGCTGTGCGCGCTCCACCTCTTCTCGGCCAAGGCGCT
CGACGACCTCCGAGCAGTCCGGGAGGGCGAGGTCGCGCTCATGGTGAGGAACCTCGCTCGGC
AGCAGGCGGCGTCAGTGGCGCTGGGGCAGGAAGCGAACGTCTGCGCCACGAACACGCTGGCC
CGCGCCACCATCGGTCACCGGGTGTTCGCCGTCGACGGCGGGGAAGGCGCAAGGGAGTTCAA
GGAGATGGTTGTGGAGCTGATGCAGCTCGCCGGCGTTTTCAACGTCGGGGACTTCGTGCCGG
CGCTCCGGTGGCTCGACCCGCAGGGCGTCGTGGCAAAGATGAAGAGGCTGCACCGTCGGTAC
GACAACATGATGAACGGATTCATCAACGAAAGGAAGGCCGGGGCGCAGCCCGACGGGGTCGC
CGCTGGCGAGCACGGCAACGACCTTCTAAGCGTGCTGCTGGCGAGGATGCAGGAGGAGCAGA
AGCTGGACGGCGACGGCGAAAAGATCACCGAAACTGACATCAAAGCTCTGCTCCTGGTAAGT
TCCTGATGACCGTGCCTTTTCAGATTATCGCAACACCACTTCCATGTTGACATGATCTTTCT
TCTTTCTTTTTGTGGATCGTGATAGAACCTATTCACTGCGGGGACGGATACGACATCGAGCA
CGGTGGAGTGGGCACTGGCGGAGCTGATCCGGCACCCGGACGTCCTCAAGGAGGCCCAGCAT
GAGCTTCACACCGTCGTCGGTAGGGGTCGTCTCGTGTCCGAGTCTGACCTTCCACGCCTCCC
CTACCTCACCGCGGTGATCAAGGAGACGTTTCGGCTTCACCCGTCAACGCCGCTCTCACTGC
CTCGGGAGGCTGCAGAGGAGTGTGAGGTGGACGGCTACCGTATCCCCAAGGGCGCTACCCTC
CTAGTCAACGTCTGGGCTATAGCCCGTGACCCGACCCAATGGCCCGACCCGCTACAGTACCA
GCCTTCTCGGTTTCTCCCCGGCAGGATGCATGCAGACGTGGATGTCAAGGGTGCTGATTTCG
GCCTGATACCATTCGGAGCAGGACGGAGAATATGCGCTGGCCTTAGTTGGGGCTTGCGGATG
GTCACACTGATGACTGCCACGCTAGTGCACGGGTTCGACTGGACCTTGGCTAACGGCGCGAC
TCCGGACAAGCTCAACATGGAGGAGGCCTATGGGCTCACCTTGCAGAGGGCCGTGCCGTTGA
TGGTCCAGCCCGTGCCAAGGCTGCTTCCATCGGCTTATGGAGTATAAAACCGGTCTACTTAC
TAGTACCACTTTAAATTAAGGTCAGAAATCGGTGGAGACTACTTGCAGTGTTGGCCGCATTA
TATGACGTATTATTTTGTTTTGTTTGTTGGTGGAAAAATAAAGTAGTCTATCTCAGTGTTAT
CTGGCACTAAAGGAACTCTAGAAATGGTGGCAAAATAGAGTACTATCGTGGAATCATAAAAA
AGGATTATTTGGTGTATAATACAGAAAAATTTATGAACACGCTGGTATATATG
SEQ ID NO: 10
>CYP75B4_LOC_Os10g16974_chr10_8494248-8504329
GGTGGTAGGTAAGGGATCTCAGGATGGGACCTGGCACCCATATCCACCAACCACTGTTGTCC
CTGAGATAATAGACGCGTGCTTTGCAGAGTGATCCAAAGCTAGCTAGTCCTACCAACAATGG
AGGTCGCCGCCATGGAGATCTCTACCTCATTGCTCCTCACCACCGTGGCTCTCTCCGTCATC
GTGTGCTACGCCCTGGTCTTCTCCCGCGCCGGGAAGGCGCGTGCGCCGCTGCCGCTGCCGCC
TGGCCCCAGGGGATGGCCGGTGCTGGGCAACCTGCCGCAGCTGGGCGGGAAGACGCACCAGA
CGCTGCACGAGATGACCAAGGTGTACGGCCCGCTGATCCGGCTCCGGTTCGGGAGCTCCGAC
GTGGTGGTCGCCGGCTCGGCGCCGGTGGCGGCGCAGTTCCTCCGCACCCACGATGCCAACTT
CAGCAGCCGGCCACGCAACTCCGGCGGCGAGCACATGGCGTACAACGGCCGGGACGTCGTGT
TCGGGCCGTACGGGCCGCGGTGGCGCGCCATGCGGAAGATTTGCGCCGTCAACCTCTTCTCC
GCGCGCGCGCTCGACGACCTGCGCGCTTTCCGGGAGCGGGAGGCCGTGCTGATGGTTAGGTC
GCTGGCGGAGGCGAGCGCCGCCCCTGGGTCGTCGTCTCCAGCGGCGGTGGTCCTGGGAAAGG
AGGTGAATGTCTGCACGACGAAcgcgctgtcgcgcgccgcggtcgggcgccgcgtgttcgcc
gccggcgcgggcgagggcgcgAGGGAGTTCAAAGAGATCGTGCTGGAGGTGATGGAGGTGGG
TGGTGTGCTGAACGTCGGCGACTTCGTGCCGGCGCTCCGGTGGCTGGACCCGCAGGGCGTGG
TAGCGAGGATGAAAAAGCTGCACCGCCGGTTCGACGACATGATGAACGCGATCATCGCGGAG
AGGAGGGCCGGATCACTACTCAAACCAACCGACAGTCGTGAGGAAGGTAAGGACTTGCTTGG
CTTGCTCCTGGCTATGGTGCAGGAGCAGGAGTGGCTCGCCGCCGGCGAGGACGACAGGATCA
CCGACACGGAAATCAAGGCCCTTATCCTGGTTCGTGATTCATGCTCTGATTTAGTAGATAGA
CACTCACTCGTTCATTGCatattaactaagtagctataattttttaagaaaaataataaaat
atattagtatataatatattactttacaaacatataaattaaattaaatttgatttttataa
ataacatataGATTATAAGATCCACGTTAAATGAGTCTACATCCACTCAATTATTAGAATCT
GTCCCCTGAACTTTTTTACTGTTGTCTGTCTACGTCATGTCCAAAACACTAGTAATGTTTGT
TTCTCCTTTGTTGAGAATCTGTTTTTCCCACGTCGATGTGCTTTCTGTTCGGAATTTGGTTT
GGGAATTTGGGGATATGCGTCGTTTTGCGCACCAGAAGACCAGAACACGTACTTGTCGTCAT
CACCACTCATTTGGGTAACAGATTATCAAGTAGACTGGTTGTCGGGCTTCCAATAGTAAAAT
CTAATTCGAGAGCCCTCCTGTTTTAGCGCTATCATTCGACGCTGGCAGAGCCGTCTGATCGC
TCGCGTCATTATCGAGTCCATCTGCGTAGGCCTCGCAGTCTACtggtaattcttacgatcac
agataaaatccgcaagcgcacgggtatacagatgtagcacttcccctacggagtattccaaa
gggtatcgaatccaaggaaacatgtgtggtcagttcttcctccggttcatccaagaacacca
agcaaaggatagggcgggatagcgaggattcactggtgagaaatagtgtctaggaaagttta
agtttaatcctaacgtaatacttcaggcactggtaacccgctattcccagatgttgctctac
tacgtacccggacagggaagacttaagtgatctcgagggctgtcaccacctctacacctacc
tcaaacgtactgtgggatacacagtaattactggataacaattacctaaacaccacgtctaa
gcaattaatatctactttagtatttataactcaccaaagcaatctctatatttcagttgatt
atagtgaacgataatcccgtatgctatttaggaactaaccaagagataattctcacaagata
aatctaaattactcaggaagaatattatattgaaatcagagtaatgaacaaaataaaagaaa
tgagagaagattaccgacaactccagaattcttccgacttcttctactctactctcttccta
ttctagtatacaatatagtacaatagagcctcttataatttagctcaatcttggaagtgtgt
gtaagagtgaaggagtgaaactccttatatagaggtaggtatgactgttacacgatgcgaat
tgtcggaaatgccccgcaaccgccatcaggagatgatcaggaccatccacgccaaaccccag
cctgaacggctgagattttggttcggccgaaccaaggggttcggccgaacgtgggctaggcc
cacctggcctggccttcggcccatctcctccgctggtcctcctttatccatttctcggagtt
ttgagctgagtctttgatattttgatgatcacaacccatccttgtatgaatacacgtttctc
ctcactttagtctgattttactcccaacttcggggttcaacacctgcatacaaatgaacacc
aacactagtggaatatgtgagattaaacacctatcgctatattgaatgtgttattatctgga
ctttatgcagaggttggcggtatagaatcagcatttaacagccgccaacaTAGTCGTAGGCA
TGGCTTTCTGGATAAGGATCGAGATGAAACCACTCCAACCACACGTCACACGCAAATCCTGC
TTTAGAGAGTCACAGAGAGAGAGGTTACAGTTTCTCCCCcatgtccttttcctcttcaaaca
atgttttctctcaaattacctatccgatcaacaatccgattacaccattgtgttcgttacaa
ttaaatcatcacaacaagctctgacatgattatattacgatgaaaaaatatttatatttata
aattacttttattatatatgtaagttacttttatcatacatataagttgcttttagatttga
ctaaattacttttatatttgacATAtaaaagtaaatttaataaagtctgaaagtagtttaca
tatattataaaattaacttaaaacaaaacggaagtaactttgtcacgacattagaagtaaat
tcgttgtagggataaaaatataactttatctaaaattaaatttaattagcacaaatcaACAC
ACGTAAGTTTAACAATTTTTAAAAGTAACTTCAATGTTAATTGGAAGTAACTTTTGCATGGT
TCAAGTTGAAAGGAGATACTAGATGGAGTACAAACTAGCTACCACTAGCTGTGTAGTCACGT
CCAATGAAAAAATGCATTAATTAaagttactttctttttgttatagttacttctataatata
tttaaattacttttaggctttattgaatttacttttatatgtctaagaagtaatttagtgaa
atctaaaaataatttagatatattataaaagtaatttattatttttcatcaaaatataatca
tgtgagatcttgttataaagatttaattgttacaaacacaacgatataatcggatcgtagat
cagataactagtttaagagaaaattgtatTTGAAATATAGGTGGACAATGTCTCTCATATAT
GGAGTGGTAGCTGGTTTAGACAAATCAGCTACCACTTGCTGTGTAGTCACGTTCCCAATCGT
AAAATCCTCCTTTTCCGTGGGAATAGGATAATGCACCACCTAAATTATTTTAATGCTACCTT
ACTCTAAACTTCGGATATCCGCACGGTCTTCAAAGCAGTGACAAATCTAGAATTTTTATTTT
GGTGTGCCCACACAATCCTCAAAAGCCAAATCAGAGTCACTCATATACACAAATACAGTAAA
AGTTCtttttttaaaggaacacaacaagggagggccccattgctaaaagattattattaaaa
aagaaagaaggttacaaagcaaattacaacaaaaggatccaatctctgactacatgttgatc
actcactttcaacctaaacagcaaaaggaggaagtctcctttaagaagttgcctccaagaaa
aaaacaaaggtaatctgttctcaaaaatgaaaccattcctttctttccaaatattccaagcc
cctaaaaggaacttttctataaagcattgcccattgtaaacatgtttagccttcatgatcat
attgctcaaactgagagaactatcccacaagatgcctaaataagtgcaacaagtcaccgaga
aagggcacttaaagaaaagatgcattaaagtgtcagtacatctctaccgacaaagaacacaa
aacaaatcataatctggtggagcacagtgtttgtgatccagcataaattagtgttcaatcta
tccatgaaaacaagccagctaaatactttaaccttggagaaaaccttactcttccaaagcca
cacaaagtgctgagggggctgcaagtgcctaaaattcagagcataaaacttctgggaagtat
atttcaaaccaccccaaatataggaccaaatatatggctatctcactctcctgagaaagatc
aatagtgctaataaaatctgaaagagcctgaaactcagtataagcttggtgagacaaaggca
gctgaaaattgtctgacatttgagctgacagatagccatgcacaaatgatattttgttggca
gcaaatgaataaagtctaggcattgagaaactgagagggggattatcagaccaaatatcctc
ccaaaaagaacatgttaaaccattccccacattgcatctagcaatccctctatagaaatcca
tcagcttataaatatctctacaccaaaaagaccctttagagatgacaaatgaggggccactt
gatcataataagaggaccagataagatcaacccatggcacattccttctattatagaatttg
tccaaaaatttcaccaaaagagcttgattccgaatagaaaggttgataacaccaagcccccc
ttttactttaggtttacaaactttgtcccaagctgctaaattttctgcttgaattcacatat
gaacatctccaaagacaatgtttccttgcactatcaatattatcaattactgtcttaggcaa
cattatagtgcacatgtaattggttggcaaggaggagaaaactgaattaactagagtaagcc
tattaccatataacaagaaatctgagtttgcagacaatctcctttcaattccttcaactaga
ggagcaaaatcaaccactctaggcttagttgttcccaaaggcagaccaagataagtaaaggg
gagtgaaccaattttgcaaccagaaaccctagcaagcatctcatcttttccatcactcaagt
ttttagggattaaacatgatttttgatagttaacctttaatctagtaccttgagcaaaagat
tgaagcagaacttttagagtaaaaagctccttcccacaatccttaacatataaaagagtatc
atctgcatattggaccactgggaagttattgtcctgactatagggcaatggcttggataaca
aatacaaatggtgtgctatgtttattaaagattgcagaagatcagctcccacaacaaacaac
ggcggtgaaaggggtcttacaccccccctccccccccccccgcctacaatgaaaattctttc
taggaactccattcagaagaactgaagagaagcagaagaaaagatcttctgaatccagttta
accattttctaaaaaccccatagctttcatcaccaacaaaatagcttcatgttcaatagtat
caaaggctttctcaaagtccaactttaaaagaacgatttcccttcttgagtggtgacattga
tgaagaaactcaaaattccaagcaaggcaatcctgaatagttctcccttttataaaactata
ttgatttggatgtattactgataggattaccatctgcaatctgccagccaacaacttcgtca
aaatcttcagagaaatccccataagagaaataggcctatagtgatttatagtctcaggacac
aattttttaggcaccaaagtgatgaaagagtgattcaaaggattcaagtctagagagcctca
atgaaaatcagcgcataatttataataatcttgacatataatgggccagcatttcttaataa
aaaggccattaaaaccatccgaatcaggagctctatcaatagggagattcttaataagctta
tcaacctcattcttagaaaagggggcatccaggatttccaaaccgtcttgtgaagggattaa
agaagggagatcaaaaggcatacattcaaaacaagaaattcccatcctcatcttaaaagcat
tccaagcagtatgggctttagcctcatgatcagagagggttgacccatccacatcaaccaaa
atagctatagaattcttcctgtatgattcagttgccattgcatggaaaaaatttgtgttttc
ccctccaaatttagcccatctaatagtacaccttttcttccagtatgtttgcttatatttct
aagtatgtaagcaacttagatctgacaaaaactctgaaattccattccaacacagtgagatc
tctatattcctcaatgatatcaaaaaaaaaaagatcacaacattacattttgtgatgagttt
ctgaagcttacatagattcctactccattttataagcccctttcttaaagctttgagtttag
atgaaatatttctagcagcatccaaaaaactgccatgattagcccaaatagattggactaaa
tcaaaaaacctttcatgctcaacctataaattctcaaacctgaacacatttgatctaggaat
aacaactgcacaatacaggcggtatgatcagaagttgacctagctaaaggcttgaataaagt
attaggaaaagaggaagaccaactagtagaagtgaaaaaccagtccaattgctcaagttgag
ggtgattttgcatgttactccaggtaaaggccatgcccttaatagggacttccactaaaccc
aaattactgatgacctcattaaaaagaaaaatatcattcatatccgctcctggcttatttct
atttagaactgaacgatagaagttaaaatcaccaagcaacagccaaaaatcaccaacaggaa
tttggagactataaagccaagccaaaaaaattagacctttcaacaccagtacagggctcata
aatagtaaccattgtccaagataaactagaatcattagaagtaaaagtgagctggacaacaa
aactttcaattgtgatattcataacagagaagacataactattccagcaaaccaaaataccc
ccaaatgcacccctagaaggggaaaaaagcaaatttgtcaaaacgcttaggggtgaacttcc
taatgaaggaatgatcaaaattacttctcttagtttcatgtaaacaaaaaaaaatgcacatc
cgctttcctctatcttattcctaatagccaaccacccatcatcagaatttatgcctcttaca
ttctaacacagaacattccagtcctaaaagcaccattcatttTAAACAGTGATAGTAGTGTA
GATCATATCTTACATAACACAAACAAAGAAATGAAACAATGTTAATGAAACACTTTTTTAAG
CTTAGAAATGCTTGCACGCACCATCATTAATGATTCATTGTAACTTATTGATTTCACATTTC
ACAAATATGCATCATCCAAACAAAGTAAAATTGTAAAAGCTAGAAAATTAGCTGTGCTACGA
GCCTACTAACTTGCCTGATTACCCCCTATACTGGTTAGGATAATGGTTATAATagccgtaac
cagcaaatcgagactaaagatctcgatctttagtaccggttgaaataacaagtactaaagat
gcataccaagtcAAAAAACTATATGTGGGATGTGGGACTCAAACTTACGATCTCTCACCCCA
ATCCTCACGTGCGTTACCATCCCACCTAGTACACACATCTGACTTAGATAAATATGCTTTCT
TTTTAATCTAATCGTAAAGACATCTTTAGtaaaaatatcattagtccaagttagtattacca
acagagaataaagatcctccagcattatttttggttggtgataccaaccggtactaaagaag
tatttttagtaccgattagtaacattaaccatgagtaaaactgtttctagggagttagactt
ttagaatcggtactaaagaatcttataccagttcttaatccaaccaagatattttttatttt
ggATACCACAATAAAAAGATCAGTTATATAGTAAATGTTGGTGCTACTTGACTGGTTGGCGG
TGACAACTACCGTCGGCCATAGCGTGCCTGCGTGTCGTGGGCTGCACCACTACGCTGGACTC
CATCTCACTGCCCGTTGTGCAGCGCTCGCCGCTTCGCATGTGCCACAAGACGAGAGATGAGA
TACAGAGAAAACCCTATACACGCACGTGCACGCCCGATAGATTCGTGTGCTGGTGGAGTCCA
GACAATGCAGCGGGGCCTTGCACTCTATTTCGGCATTTATTTATTTATTGTGACCAGGCACC
AACCGATCATTTAGGCCTATTGCTGCATACTGCTTTTTAAAATTATTTTGCAAATTTTGGGT
ATGCTGCCAGTCCATACCGGGAAGGCAGGAACCCGTCCGCCCCTCTTTGAAGTACTACTAAT
ACATGTTACATTATCATTTTTAATAATATGTTTATAAAAATATTGAAAACATTGCCACATGA
ATGTGtttatattataaatatattattatataccatttttcatatattaaattttagttatt
tttatataGACCCATCCTATGATGTCATATGTCTTTCTGCTGACTATGCAGAATCTATTCGT
GGCGGGCACAGACACAACATCAACCATAGTTGAGTGGACAATGGCAGAGCTGATTCGACACC
CAGATATCCTCAAGCACGCCCAAGAGGAGCTAGATGTTGTTGTGGGTCGTGATAGGCTCCTC
TCAGAGTCGGATCTATCACATCTCACCTTCTTCCATGCTATCATCAAGGAGACATTCCGTCT
ACATCCATCAACACCGCTCTCGCTGCCACGCATGGCATCTGAGGAGTGTGAGATCGCAGGCT
ACCGTATCCCCAAGGGTGCAGAGTTGCTGGTCAATGTGTGGGGGATCGCCCGTGACCCAGCC
ATATGGCCTGACCCACTAGAGTACAAGCCCTCTCGGTTCCTCCCCGGTGGGACGCACACTGA
TGTGGATGTCAAGGGAAATGATTTCGGACTTATACCATTCGGTGCAGGGCGAAGGATATGCG
CCGGCCTCAGTTGGGGCCTGCGGATGGTCACCATGACAGCGGCCACGCTGGTGCATGCATTC
GACTGGCAGCTACCAGCGGACCAGACGCCAGACAAGCTCAATATGGATGAGGCGTTTACCCT
CCTGCTGCAAAGGGCAGAGCCATTGGTGGTTCACCCGGTACCAAGGCTTCTCCCATCCGCTT
ACAATATTGCATAAAGATTTACGAGTTGAATATAATTAACGAAAAGTTATTTCCGTGTGTGT
GGCATCAAATAAATAGAGGGTATGAACTTTTGTCATGGTGTTGCATCATTGTTGTATGTTGG
TAGATTGGTTTTTCACGAGTATCTATACTCCTTATAAAAAGGAGTAGTGGTGATGATTCTGC
TACCACCCCACTACCAACTCTTATCtttttttAAGGACATCTAGGATTAGTGGGCCCATATG
TCATTACTCTCACCAACTTTTATTCTTGTGAAAGGTTATTACCGTGCGAATAAATAGTGGGT
TTGAACTGTCGTTGTGTTATATCATTCGATGTATGTTGATTGGTTTTGTTTTTCACAAGGAG
TATACATATATTAAGGGACAGAATAATTGTCAGTCGCT
SEQ ID NO: 11
GRNA1;
TGCGGCAGGTTGCCCAGCAC
SEQ ID NO: 12
GRNA2;
CCGCTGTTCCGGCTCCGGTT
SEQ ID NO: 13
GRNA3;
ACTTCGTGCCGGCGCTCCGG
CYP75B3/B4 SEQUENCES
SEQ ID NO: 14
Oryza sativa ssp. indica
OsR498G1018420100
MEVAAMEISTSLLLTTVALSVIVCYALVFSRAGKARAPLPLPPGPRGWPVLGNLPQL
GGKTHQTLHEMTKVYGPLIRLRFGSSDVVVAGSAPVAAQFLRTHDANFSSRPRNSG
GEHMAYNGRDVVFGPYGPRWRAMRKICAVNLFSARALDDLRAFREREAVLMVRSL
AEASAAPGSSSPAAVVLGKEVNVCTTNALSRAAVGRRVFAAGAGEGAREFKEIVLE
VMEVGGVLNVGDFVPALRWLDPQGVVARMKKLHHRFDDMMNAIIAERRAGSLLK
PTDSREEGKDLLGLLLAMVQEQEWLAAGEDDRITDTEIKALILNLFVAGTDTTSTIVE
WTMAELIRHPDILKQAQEELDVVVGRDRLLLESDLSHLTFFHAIIKETFRLHPSTPLSL
PRMASEECEIAGYRIPKGAELLVNVWGIARDPAIWPDPLEYKPSRFLPGGTHTDVDV
KGNDFGLIPFGAGRRICAGLSWGLRMVTMTAATLVHAFDWQLPADQTPDKLNMDE
AFTLLLQRAEPLVVHPVPRLLPSAYNIA*
SEQ ID NO: 15
Oryza sativa ssp. indica
OsR498G1018427100
MDVVPLPLLLGSLAVSAAVWYLVYFLRGGSGGDAARKRRPLPPGPRGWPVLGNLP
QLGDKPHHTMCALARQYGPLFRLRFGCAEVVVAASAPVAAQFLRGHDANFSNRPPN
SGAEHVAYNYQDLVFAPYGARWRALRKLCALHLFSAKALDDLRAVREGEVALMV
RNLARQQAASVALGQEANVCATNTLARATIGHRVFAVDGGEGAREFKEMVVELMQ
LAGVFNVGDFVPALRWLDPQGVVAKMKRLHRRYDNMMNGFINERKAGAQPDGVA
AGEHGNDLLSVLLARMQEEQKLDGDGEKITETDIKALLLNLFTAGTDTTSSTVEWAL
AELIRHPDVLKEAQHELDTVVGRGRLVSESDLPRLPYLTAVIKETFRLHPSTPLSLPRE
AAEECEVDGYRIPKGATLLVNVWAIARDPTQWPDPLQYQPSRFLPGRMHADVDVK
GADFGLIPFGAGRRICAGLSWGLRMVTLMTATLVHGFDWTLANGATPDKLNMEEA
YGLTLQRAVPLMVQPVPRLLPSAYGV*
SEQ ID NO: 16
Oryza sativa ssp. japonica
LOC_Os10g16974
MEVAAMEISTSLLLTTVALSVIVCYALVFSRAGKARAPLPLPPGPRGWPVLGNLPQL
GGKTHQTLHEMTKVYGPLIRLRFGSSDVVVAGSAPVAAQFLRTHDANFSSRPRNSG
GEHMAYNGRDVVFGPYGPRWRAMRKICAVNLFSARALDDLRAFREREAVLMVRSL
AEASAAPGSSSPAAVVLGKEVNVCTTNALSRAAVGRRVFAAGAGEGAREFKEIVLE
VMEVGGVLNVGDFVPALRWLDPQGVVARMKKLHRRFDDMMNAIIAERRAGSLLKP
TDSREEGKDLLGLLLAMVQEQEWLAAGEDDRITDTEIKALILNLFVAGTDTTSTIVE
WTMAELIRHPDILKHAQEELDVVVGRDRLLSESDLSHLTFFHAIIKETFRLHPSTPLSL
PRMASEECEIAGYRIPKGAELLVNVWGIARDPAIWPDPLEYKPSRFLPGGTHTDVDV
KGNDFGLIPFGAGRRICAGLSWGLRMVTMTAATLVHAFDWQLPADQTPDKLNMDE
AFTLLLQRAEPLVVHPVPRLLPSAYNIA*
SEQ ID NO: 17
Oryza sativa ssp. japonica
LOC_Os10g17260
MDVVPLPLLLGSLAVSAAVWYLVYFLRGGSGGDAARKRRPLPPGPRGWPVLGNLP
QLGDKPHHTMCALARQYGPLFRLRFGCAEVVVAASAPVAAQFLRGHDANFSNRPPN
SGAEHVAYNYQDLVFAPYGARWRALRKLCALHLFSAKALDDLRAVREGEVALMV
RNLARQQAASVALGQEANVCATNTLARATIGHRVFAVDGGEGAREFKEMVVELMQ
AGEHGNDLLSVLLARMQEEQKLDGDGEKITETDIKALLLNLFTAGTDTTSSTVEWAL
AELIRHPDVLKEAQHELDTVVGRGRLVSESDLPRLPYLTAVIKETFRLHPSTPLSLPRE
AAEECEVDGYRIPKGATLLVNVWAIARDPTQWPDPLQYQPSRFLPGRMHADVDVK
GADFGLIPFGAGRRICAGLSWGLRMVTLMTATLVHGFDWTLANGATPDKLNMEEA
YGLTLQRAVPLMVQPVPRLLPSAYGV*
SEQ ID NO: 18
Triticum aestivum
TraesCS1A02G442200
MDHSLLLLLASLAAVAVAAVWHLRSHGRRTKLPLPPGPRGWPVLGNLPQLGAMPH
HTMAALARQHGPLFRLRFGSVEVVVTASAKVARSFLRAHDTNFSDRPPTSGAEHLA
YNYQDLVFAPYGARWCALRKLCALHLFSARALDALRTIRQDEARLMVTHLLSSSSP
AGVAVNLCAINVRATNALARAAIGGRMFGDGVGEGAREFKDMVVELMQLAGVLNI
GDFVPALRWLDPQGVVAKMKRLHRRYDRMMDGFISERGQHAGEMEGNDLLSVML
ATIRWQSPADAGEEDGIKFTEIDIKALLLNLFTAGTDTTSSTVEWALAELIRDPCILKQ
LQHELDGVETFRLHPATPLSLPRVAAEDCEVDGYHVSKGTTLIMNVWAIARDPASW
GPDPLEFRPVRFLPGGLHESADVKGGDYELIPFGAGRRICAGLGWGLRMVTLMTATL
VHAFDWSLVDGTMPEKLNMEEAYGQTLQRAVPLVVQPVPRLLSSAYTV*
SEQ ID NO: 19
Triticum aestivum
TraesCS1A02G442300
MDHDLLLLLLASLVAVVAATVWHLRGHGSGARKPKLPLPPGPRGWPVLGNLPQLG
DKPHHTMAALARHHGPLFRLRFGSAEVVVAASAKVAGSFLRAHDANFSDRPPNSGA
EHVAYNYQDLVFAPYGARWRALRKLCAQHLFSARALDALRQVRQDEARLMVTRLL
SSSDSPAGLAVGQEANVCATNALALAAVGRRVFGDGVGEGAREFKDMVVELMQLA
GVFNIGDFVPALRWLDPQGVVGKMKRLHRRYDLMMDGFISERGDRADGDGNDLLS
VMLGMMRQSPPAAGEEDGIKFNETDIKALLLNLFTAGTDTTSSTVEWALAELIRHPD
VLKKLQHELDDVVGNGHLVTETDLPQLTFLAAVIKETFRLHPSTPLSLPRVAAEDCE
VDGYRIPKDTTLLVNVWAIARDPASWGDDVLEFRPTRFLPGGLHESVDVKGGDYELI
PFGAGRRICAGLSWGLRMVTLMTATLVHAFDWTLVDGMTPEKLDMEEAYGLTLQR
AVPLMVQPVPRLLPSAYTM*
SEQ ID NO: 20
Triticum aestivum
TraesCS1B02G476400
MDHDLLLLLLASLAAVAAAAVWHLRGAKSPKLPLPPGPRGWPVLGNLPQLGDKPH
HTMAALARLHGPLFRLRFGSAEVVVAASAKVAAAFLRGHDANFSDRPPNSGAEHVA
YNYQDLVFAPYGARWRALRKLCALHLFSARALDALRTVRQDETRLMVTRLLSSSSG
SVSPAGLAVGQEANVCATNALARAAVGRRVFGDGVGEGAREFKDMVAELMQLAG
VFNIGDFVPALRWLDPQGVVAKMKRLHRRYDRMMDGFISERGDRADGDGNDLLSV
MLGMMRQSPPAAGEEDGIKFNETDIKALLLNLFTAGTDTTSSTVEWALAELIRHPNV
LKKLQHELDDVVGNGRLVTESDLPQLTILAAVIKETFRLHPSTPLSLPRVTAEDCEVD
GYRIPKDTTLLVNVWAIARDPASWGDDVLEFRPVRFLAGGSHETVDVKGGDYELIPF
GAGRRICAGLSWGLRMVTLMTATLVHAFDWTLVDGMTPEKLDMEEAYGLTLQRA
VPLMVQPVPRLLPSAYTV*
SEQ ID NO: 21
Triticum aestivum
TraesCS1D02G450100
MPCARPTNKQTSPHPLPPSMTPAAMDHDLLLLLASLAAVIVAAVWHLRGHGSGARK
PKLPLPPGPRGWPVLGNLPQLGDKPHHTMAALARLHGLLFRLRFGSAEVVVAASAK
VAGSFLRAHDANFSDRPPNSGAEHVAYNYQDLVFAPYGARWRALRKLCAQHLFSA
RALDALRTVRQDEARLMVTRLLSSSDSPAGLAVGQEANVCATNALALAAVGRRVF
GDGVGEGAREFKDMVVELMQLAGVFNIGDFVPALRWLDPQGVVAKMKRLHRRYD
RMMDGFISERGDRADGDGNDLLSVMLGMMRQSPPAGGEEDGIKFNETDIKALLLNL
FTAGTDTTSSTVEWALAELIRHPDVLKKLQHELDDVVGNGRLVTESDLPQLTFLAAV
IKETFRLHPSTPLSLPRVAAEDCEVDGYRIPKDTTLLVNVWAIARDPASWGDDVLEFR
PTRFLPGGSHESVDVKGGDYELIPFGAGRRICAGLSWGLRMVTLTTATLVHAFDWTL
VDGMTPEKLDMEEAYGLTLQRAVPLMVQPVPRLLPSAYTM*
SEQ ID NO: 22
Triticum aestivum
TraesCS2B02G613200
MDHDLLLLLASLAAVAVAAVCYLRSHGSGAKLPLPPGPRGWPVLGNLPQLGAKPH
HTMAALARQHGPLFRLRFGSAELVVAASAKVAGSFLRAHDANFSDRPPNSGAEHVA
YNYQDLVFAPYGARWRALRMLCALHLFSARALDALRSVRQDEARLMVTHLLSASS
SPAQGVAIGQEANVCATNALARAAVGRRVVGDGVGESAREFKGMVVELMQLAGA
FNIGDFVPALRWLDPQGVVAKMKHLHRRYDRIMDGFISEREHLAGEEEGKDLLSIML
AKMRQPLHADAGEDGIKFTETNIKALLLNLLTAGTDTTSSTVEWALAELIRHPDTLK
QLQREVDDVVGTSRLVTEADLPRLTFLTAVIKETFRLHPSTPLSLPRVAAEDCEVDGY
HVAKGTTLLVNVWAISRDPASWGADALEFRPARFLPGGSHETVDVKGGDYELIPFG
AGRRMCAGLSWGLRIVTLMTATLVHAFDLSLVNGMTPDKLDMEEAYGLTLQRAVP
LLVQPMPRLLPSAYAT*
SEQ ID NO: 23
Triticum aestivum
TraesCS6A02G012600
MEIPLPLLLSTFAISVTICYVIIFFFRADKGRAPLPPGPRGWPVLGNLPQLGGKTHQTL
HEMTRLYGPMLRLRFGSSLVVVAGSADVAKQFLRTHDAKFSSRPPNSGGEHMAYN
YQDVVFAPYGPRWRAMRKVCAVNLFSARALDDLRAFREWEAALMVRCLADAAAA
GMAVALAKTANVCTTNSLSRATVGLRVFDTAGSKLGAEEFNEIVLKLIEVGGVLNV
GDFVPVLRWLDPQGVVAKMKKLHRRFDDMMNRIIAERRAGAFATTAGEEGGKDLL
GLLLAMVQEDKSLTGAEENKITDTDVKALILNLLVAGTDTTSITVEWAMAELIRHPDI
MKQAQEELDAVMGRERLVSESDLPRLTSLSAIIKETFRLHPSTPLSLPRMATEDCKVA
GYCIPKGTELLVKVWGIARDPALWPDPLEFRPARFLPGGSHADVDVKGGDFGLIPFG
AGRRICAGLSWGIRMVTVTTATLVHSFDWELPAGQTPDMEETFSLLLQLAVPLMVH
PVPRLLPSAYQIA*
SEQ ID NO: 24
Triticum aestivum
TraesCS6B02G018800
MEIPLPLLLSTFAISVTICYVIFFFFHADKGRAPLPPGPRGWPVLGNLPQLGGKTHRTL
HEMTRLYGPMLRLRFGSSLVVVAGSADVAKQFLRTHDAKFSSRPPNSGGEHMAYN
YQDVVFAPYGPRWRAMRKVCAVNLFSSRALDDLRGFREREAALMVRCLADSAATG
GAVALAKAANVCTTNALSRATVGLRVFATAGSELGAEDFNEIVLKLIEVGGVLNVG
DFVPALRWLDPQGVVAKMKKLHRRFDDMMNRIIAERRAGAIATKAGEEGGKDLLC
LLLAMVQEDKSLTGGSEEDRMTDTDVKALILNLFVAGTDTTSITVEWAMAELIRHPD
ILKQAQKELDAVIGRDRLVLESDLPRLNFLNAIIKETFRLHPSTPLSLPRMATEECEVA
GYRIPKGTELLVNVWGIARDPALWTDPLEFRPARFLPGGSHADIDIKGGDFGLIPFGA
GRRICAGLSWGIRMVAVTTATLVHSFDWELPAGQMPDMEETFSLLLQLAVPLMVHP
VPRLLPSAYQIA*
SEQ ID NO: 25
Triticum aestivum
TraesCS6D02G015200
MEIPLPLLLSTFAISVTICYVILFFRADMGRAPLPPGPRGWPVLGNLPQLGGKTHKTLH
EMARLYGPMLRLRFGSSLVVVAGSADVAKLFLRTHDAKFSSRPPNSGGEHMAYNY
QDVVFAPYGPRWRAMRKVCAVNLFSARALDDLRAFREWEAALMVRCLADAAAAG
MAVALGKAANVCTTNALSRATVGLRVFATAGSELGAEEFNEIVLKLIEVGGVLNVG
DFVPALRWLDPQGVVAKMKKLHRRFDDMMNRIIAERRAGAFATTASEEGGKDLIGL
LLAMVQEDKSLTGAEENKITDTEVKALILNLFVAGTDTTSITVEWAMAELIRHPDIM
KQAQEELDAIVGRERLVSESDLPRLTFLSAIIKETFRLHPSTPLSLPRMTTEECEVAGY
CIPKGTELLVNVWGIARDPALWPDPLEFRPARFLPGGSHADVDVKGGDFGLIPFGAG
PRLLPSAYQIA*
SEQ ID NO: 26
Triticum aestivum
TraesCS6D02G015300
MHSTCMQNLFVAGTDTTLIMVEWAMAELIRHPDTLKQAQEELDTIVGRERLISESHL
PRLTFLSAVIKDTFRLHPSTPLLLLRMATEECETAGYRIPKGTELLVNVWGIAHDPAL
WPDSLEFRPAWFLPGGSHADVDVKGGDFGLIPFGAGRRICAGLSRGIRMVAVTTATL
VHSFNWELPAGQTPDMEGTFSLLLQLAVPLMVHPVPRLLPSAYQIA*
SEQ ID NO: 27
Triticum aestivum
TraesCS7A02G411700
MNTRAPAVLAYRSNATMHLVAMDIPLPLLLSTLAVAVGVCYVLATFFRADKGRAPL
PPGPRGWPVLGNLPQLGGKTHQTMHEMSKVYGPVLRLRFGSSVVVVAGSAGAAEQ
FLRTHDAKFSSRPPNSGGEHMAYNYQDVVFAPYGPRWRAMRKVCAVNLFSARALD
DLRGFREREAALMVRSLVDAAATGGVVAVGKAANVCTTNALSRAAVGLRVFAAA
GAELGAKEFKEIVLEVMEVGGVLNVGDFVPALRWLDPQGVVARLKKLHRRFDDM
MNGIIAERRAGGSTAGEEKEGKDLLGLLLAMVQEDKSLTGGEEDRITDTDVKALILN
LFVAGTETTSTIVEWAVAELIRHPDMLKRAQEEMDAVVGRDRLVSESDLPRLTFLNA
VIKETFRLHPSTPLSLPRMASEECEVAGYRIPKGTELLVNVWGIARDPALWPDPLEFR
PARFLPGGTHADVDVKGGDFGLIPFGAGRRICAGLSWGLRVVTVTAATLVHSFDWE
LPAGQTPDKLNMEEAFSLLLQRAVPLMAHPVPRLLPSAYEIA*
SEQ ID NO: 28
Triticum aestivum
TraesCS7B02G310900
MHLVAMGIPLPLLLSTLAIAVTICYVLATFFRADKGRAALPPGPRGWPVLGNLPQLG
GKTHQTMHEMSKVYGPVLRLRFGSSVVVVAGSAAVAEQFLRTHDAKFSSRPPNSGG
EHMAYNNQDVVFAPYGPRWRAMRKVCAVNLFSARALDDLRGFREREAALMVRSL
VDAASGGGVVAVGKAANVCTTNALSRAAVGLRVFAAAGTELGAKEFKEIVLEVME
VGGVLNVGDFVPALRWLDPQGVVARLKKLHRRFDDMMNGIIAERRAGGSTAGEEK
EGKDLLGLLLAMVQEDKSLTGGEEDRITDTDVKALILNLFVAGTETTSTIVEWAVAE
LIRHPDMLKRAQEEMDAVVGRDRLVSESDLPRLTFLNAVIKETFRLHPSTPLSLPRMA
SEECEVAGYRIPKGTELLVNVWGIARDPALWPDPLEFRPARFLPGGTHADVDVKGG
LQRAVPLMVHPVPRLLPSAYQIA*
SEQ ID NO: 29
Triticum aestivum
TraesCS7D02G404900
MHLVAMDIPLPLLLSTLAVAVTICYVLFFRADKGRAPLPPGPRGWPVLGNLPQLGGK
THQTMHEMSKVYGPVLRLRFGSSVVVVAGSAAVAEQFLRTHDAKFSSRPPNSGGEH
MAYNYQDVVFAPYGPRWRAMRKVCAVNLFSARALDDLRGFREREAAFMVRSLAD
AASGGGLVAVGKAANVCTTNALSRAAVGLRVFAAAGTELGAKEFKEIVLEVMEVG
GVLNVGDFVPALRWLDPQGVVARLKKLHRRFDDMMNGIIAERRAGAGTAGEEKEG
KDLLGLLLAMVQEDKSLTGGEEDRITDTDVKALILNLFVAGTETTSTIVEWAVAELIR
HPDMLKRAQEEMDAVVGRGRLVAESDLPRLTFLNAVIKETFRLHPSTPLSLPRMASE
ECEVAGYRIPKGTELLVNVWGIARDPALWPDPLEFRPARFLPGGTHADVDVKGGDF
GLIPFGAGRRICAGLSWGLRVVTVTAATLVHSFDWELPTGQTPDKLNMEEAFSLLLQ
RAVPLMVHPVPRLLPSAYEIA*
SEQ ID NO: 30
Zea mays B73
Zm00001d010521
MELFVTTPDLPTPLLLSTLTIVSVVVCYVLFWKQQAAARRAPLPPGPRGWPVLGNLP
QLGGKTHQTLHEMTKVYGPLLRLRFGSSTVVVAGSAAVAQQFLRAHDANFSSRPPN
SGGELMAYNYQDVVFAPYGPRWRAMRKVCAVNLFSARALDDVRGVREREAALMV
RSLAEQAHGGLDAPPAAVPVGKAINVCTTNALSRAAVGRRVFAAAGGDGGAREFK
EIVLEVMQVGGVLNVGDFVPALRWLDPQGVAAKMKKLHRRFDDMMDEIIAGYREA
RRVAADGEESKDLLGLLLSMVDERPFDSGEEVRITETDVKALILNLFVAGTDTTSTIV
EWSLAELIRHPEILRQAQEEMDAVAGRGRLVTESDLRSLTFFNAVIKETFRLHPSTPLS
LPRMAAEECEVAGYRVPRGSELLVNVWGIARDPALWPDPLEFRPARFLPGGSHADV
EAFTLLLQRAVPLVARPVPRLLPSAYEIA*
SEQ ID NO: 31
Zea mays B73
Zm00001d017077
MDVPLPLLLGSVAVSLVVWCLLLRRGGAGKGKRPLPPGPRGWPVLGNLPQVGAKP
HHTMCAMAREYGPLFRLRFGSAEVVVAASARVAAQFLRAHDANFSNRPPNSGAEH
VAYNYQDLVFAPYGSRWRALRKLCALHLFSAKALDDLRGVREGEVALMVRELARQ
GERGRAAVALGQVANVCATNTLARATVGRRVFAVDGGEGAREFKEMVVELMQLA
GVFNVGDFVPALAWLDPQGVVGRMKRLHRRYDDMMNGIIRERKAAEEGKDLLSVL
LARMREQQPLAEGDDTRFNETDIKALLLNLFTAGTDTTSSTVEWALAELIRHPDVLR
KAQQELDAVVGRDRLVSESDLPRLTYLTAVIKETFRLHPSTPLSLPRVAAEECEVDGF
RIPAGTTLLVNVWAIARDPEAWPEPLEFRPARFLPGGSHAGVDVKGSDFELIPFGAGR
RICAGLSWGLRMVTLMTATLVHALDWDLADGMTADKLDMEEAYGLTLQRAVPLM
VRPAPRLLPSAYAE*
SEQ ID NO: 32
Zea mays B73
Zm00001d050955
MDVPLPLLLGSLAVSVMVWCLVLRRGGDGKGKRPLPPGPRGWPVLGNLPQVGAKP
HHTMCALAREYGPLFRLRFGSAEVVVAASARVAAQFLRAHDANFSNRPPNSGAEHV
AYNYRDLVFAPYGSRWRALRKLCALHLFSAKALDDLRGVREGEVALMVRELARPR
RGEGGRAAAVALGQVANVCATNTLARATVGRRVFAVDGGEGAREFKEMVVELMQ
LAGVFNVGDFVPALAWLDPQGVVGRMKRLHRRYDHMMNGIIRERKAAEEGKDLLS
VLLARMRDQQQQPLAEGEDNRINETDVKALLLVSLLALTTSQRANGMDNCSGALRA
VEESACDAEVVRPLSKLPNSSIGLYDSSFECDACGVGFVAELSGDYKHVTVNDTIEM
LERMAHRGACGCEKNTGDGAGIMVALPHDFFKEVAKDAGIELPPLGEYAVAMFFM
PTDEKRRKKGKAEFKKVAESLGHLYILRRLSIISVRASLNIKRGGERDFYMCSLSSRA
TVGMLLGVEDMHRFPVRSPWMDRGDLIRSPAARQIVSNYFSMFGPVQDVRIPYQQK
RMFGFVTFVYAETVKVILSKGNPHFVCDARVLVKPYKEKGKVPGRFRKLQHTHHGG
AEFVGCASPTGLLDSRDPYALLLLSGAQNLFTAGTDTTSSTVEWALAELIRHPDVLR
KAQQELDAVVGRDRLVSESDLPRLTYLTAVIKETFRLHPSTPLSLPRVAAEECEVDGF
RIPAGTTLLVNVWAIARDPEAWPEPLQFRPARFLPGGSHAGVDVKGSDFELIPFGAGR
RICAGLSWGLRMVTLMTATLVHALEWDLADGVTAEKLDMEEAYGLTLQRAVPLM
VRPAPRLLPSAYAAQ*
SEQ ID NO: 33
Zea mays B104
Zm00007a00002679
MDVPLPLLLGSLAVSVMVWCLVLRRGGDGKGKRPLPPGPRGWPVLGNLPQVGAKP
HHTMCALAREYGPLFRLRFGSAEVVVAASARVAAQFLRAHDANFSNRPPNSGAEHV
AYNYRDLVFAPYGSRWRALRKLCALHLFSAKALDDLRGVREGEVALMVRELARPR
RGEGGRAAAVALGQVANVCATNTLARATVGRRVFAVDGGEGAREFKEMVVELMQ
LAGVFNVGDFVPALAWLDPQGVVGRMKRLHRRYDHMMNGIIRERKAAEEGKDLLS
VLLARMRDQQQQPLAEGEDNRINETDVKALLLASRVPNNHPAKRKPPPPPPPPPGRR
RTSPTVAWKKEDKPRRRLEGGGQAPPPPPSHCQPLPTAPTPALQLPPRRMLPNALYN
PGESRDGRMTVRVVMRYLVNKLGLEDDSQVNDTIEMLERMAHRGACGCEKNTGD
GAGIMVALPHDFFKEVAKDAGIELPPLGEYAVAMFFMPTDEKRRKKGKAEFKKVGC
RITGTWRQWACCSGVEDMHRFPVRSPWMDRGDLIRSPAARQIVSNYFSMFGPVQDV
RIPYQQKRMFGFVTFVYAETVKVILSKGNPHFVCDARVLVKPYKEKGKVPGRFRKL
QHTHHGGAEFVGCASPTGLLDSRDPYALLLLSGAQNLFTAGTDTTSSTVEWALAELI
RHPDVLRKAQQELDAVVGRDRLVSESDLPRLTYLTAVIKETFRLHPSTPLSLPRVAAE
ECEVDGFRIPAGTTLLVNVWAIARDPEAWPEPLQFRPARFLPGGSHAGVDVKGSDFE
LIPFGAGRRICAGLSWGLRMVTLMTATLVHALEWDLADGVTAEKLDMEEAYGLTL
QRAVPLMVRPAPRLLPSAYAAQ*
SEQ ID NO: 34
Zea mays B104
Zm00007a00006475
MELFVTTPDLPTPLLLSTLTIVSVVVCYVLFWKQQAAARRAPLPPGPRGWPVLGNLP
QLGGKTHQTLHEMTKVYGPLLRLRFGSSTVVVAGSAAVAQQFLRAHDANFSSRPPN
SGGELMAYNYQDVVFAPYGPRWRAMRKVCAVNLFSARALDDVRGVREREAALMV
RSLAEQAHGGLDAPPAAVPVGKAINVCTTNALSRAAVGRRVFAAAGGDGGAREFK
EIVLEVMQVGGVLNVGDFVPALRWLDPQGVAAKMKKLHRRFDDMMDEIIAGYREA
RRVAADGEESKDLLGLLLSMVDERPFDSGEEVRITETDVKALILNLFVAGTDTTSTIV
EWSLAELIRHPEILRQAQEEMDAVAGRGRLVTESDLRSLTFFNAVIKETFRLHPSTPLS
LPRMAAEECEVAGYRVPRGSELLVNVWGIARDPALWPDPLEFRPARFLPGGSHADV
DVKGADFGLIPFGAGRRICAGLSWGLRMVTLTSATLVHAFDWELPAGQTPDKLNME
EAFTLLLQRAVPLVARPVPRLLPSAYEIA*
SEQ ID NO: 35
Zea mays B104
Zm00007a00021951
MDVPLPLLLGSVAVSLVVWCLLLRRGGAGKGKRPLPPGPRGWPVLGNLPQVGAKP
HHTMCAMAREYGPLFRLRFGSAEVVVAASARVAAQFLRAHDANFSNRPPNSGAEH
VAYNYQDLVFAPYGSRWRALRKLCALHLFSAKALDDLRGVREGEVALMVRELARQ
GERGRAAVALGQVANVCATNTLARATVGRRVFAVDGGEGAREFKEMVVELMQLA
GVFNVGDFVPALAWLDPQGVVGRMKRLHRRYDDMMNGIIRERKAAEEGKDLLSVL
LARMREQQPLAEGDDTRFNETDIKALLLNLFTAGTDTTSSTVEWALAELIRHPDVLR
KAQQELDAVVGRDRLVSESDLPRLTYLTAVIKETFRLHPSTPLSLPRVAAEECEVDGF
RIPAGTTLLVNVWAIARDPEAWPEPLEFRPARFLPGGSHAGVDVKGSDFELIPFGAGR
RICAGLSWGLRMVTLMTATLVHALDWDLADGMTADKLDMEEAYGLTLQRAVPLM
VRPAPRLLPSAYAE*
SEQ ID NO: 36
Zea mays B104
Zm00007a00044616
MELFVTTPDLPTPLLLSTLTIVSVVVCYVLFWKQQAAARRAPLPPGPRGWPVLGNLP
QLGGKTHQTLHEMTKVYGPLLRLRFGSSTVVVAGSAAVAQQFLRAHDANFSSRPPN
SGGELMAYNYQDVVFAPYGPRWRAMRKVCAVNLFSARALDDVRGVREREAALMV
RSLAEQAHGGLDAPPAAVPVGKAINVCTTNALSRAAVGRRVFAAAGGDGGAREFK
EIVLEVMQVGGVLNVGDFVPALRWLDPQGVAAKMKKLHRRFDDMMDEIIAGYREA
RRVAADGEESKDLLGLLLSMVDERPFDSGEEVRITETDVKALILLTQQFRTKRISSFSL
ILSFMLARGHVRQNLFVAGTDTTSTIVEWSLAELIRHPEILRQAQEEMDAVAGRGRL
VTESDLRSLTFFNAVIKETFRLHPSTPLSLPRMAAEECEVAGYRVPRGSELLVNVWGI
ARDPALWPDPLEFRPARFLPGGSHADVDVKGADFGLIPFGAGRRICAGLSWGLRMV
SEQ ID NO: 37
Zea mays PH207
Zm00008a016611
MDVPLPLLLGSLAVSVMVWCLVLRRGGDGKGKRPLPPGPRGWPVLGNLPQVGAKP
HHTMCALAREYGPLFRLRFGSAEVVVAASARVAAQFLRAHDANFSNRPPNSGAEHV
AYNYRDLVFAPYGSRWRALRKLXXXXXXXXDGGEGAREFKEMVVELMQLAGVFN
VGDFVPALAWLDPQGVVGRMKRLHRRYDHMMNGIIRERKAAEEGKDLLSVLLAR
MRDQQQLAEGEDSRINETDVKALLLNLFTAGTDTTSSTVEWALAELIRHPDVLRKAQ
QELDAVVGRDRLVSESDLPRLTYLTAVIKETFRLHPSTPLSLPRVAAEECEVDGFRIPA
GTTLLVNVWAIARDPEAWPEPLQFRPARFLPGGSHAGVDVKGSDFELIPFGAGRRIC
AGLTWGLRMVTLMTATLVHALDWDLADGVTAEKLDMEEAYGLTLQRAVPLMVRP
APRLLPSAYAAQ*
SEQ ID NO: 38
Zea mays PH207
Zm00008a022212
MDVPLPLLLGSVAVSLVVWCLLLRRGGAGKGKRPLPPGPRGWPVLGNLPQVGAKP
HHTMCALAREYGPLFRLRFGSAEVVVAASARVAAQFLRAHDANFSNRPPNSGAEHV
AYNYQDLVFAPYGSRWRALRKLCALHLFSAKALDDLRGVREGEVALMVRELARQG
ERERAAVALGQVANVCATNTLARATVGRRVFAVDGGEGAREFKEMVVELMQLAG
VFNVGDFVPALAWLDPQGVVGRMKRLHRRYDDMMNGIIRERKAAEEGKDLLSVLL
ARMREQQPLAEGDDTRFNETDIKALLLNLFTAGTDTTSSTVEWALAELIRHPDVLRK
AQQELDAVVGRDRLVSESDLPRLTYLTAVIKETFRLHPSTPLSLPRVAAEECEAWPEP
LEFRPGRFLPGGSHAGVDVKGSDFELIPFGAGRRICAGLSWGLRMVTLMTATLVHAL
DWDLADGMTADKLDMEEAYGLTLQRAVPLMVRPAPRLLPSAYAE*
SEQ ID NO: 39
Zea mays PH207
Zm00008a031477
MELVLTTPDLPTPLLLSTLTIVSVVVCYVLFWKQQAAARRAPLPPGPRGWPVLGNLP
QLGGKTHQTLHEMTKVYGPLLRLRFGSSTVVVAGSAAVAQQFLRAHDANFSSRPPN
SGGELMAYNYQDVVFAPYGPRWRAMRKVCAVNLFSARALDDVRGVREREAALMV
RSLAEQAHGGLDAPPAAVPVGKAINVCTTNALSRAAVGRRVFAAAAGDGGAREFK
EIVLEVMQVGGVLNVGDFVPALRWLDPQGVAAKMKKLHRRFDDMMDEIIAGYREA
RRVAADGEESKDLLGLLLSMVDERPFDSGEEVRITETDVKALILNLFVAGTDTTSTIV
EWSLAELIRHPEILRQAQEELDAVAGRGRLVSESDLRSLTFFNAVIKETFRLHPSTPLS
LPRMAAEECEVAGYRVPRGSELLVNVWGIARDPALWPDPLEFRPARFLPGGSHADV
DVKGADFGLIPFGAGRRICAGLSWGLRMVTLTSATLVHAFDWELPAGQTPDKLNME
EAFTLLLQRAVPLVARPVPRLLPSAYEIA*
SEQ ID NO: 40
Triticum turgidum
TRITD1Av1G229990
MNVWAIARDPASWGPDPLEFRPVRFLPGGLHESADVKGGDYELIPFGAGRRICAGLG
WGLRMVTLMTATLVHAFDWSLVDGTTPEKLNMEEAYGQTLQRAVPLVVQPVPRLL
SSAYTV*
SEQ ID NO: 41
Triticum turgidum
TRITD1Av1G230000
MDHDLLLLLLASLAAVVAATVWHLRGHGSGARKPKLPLPPGPRGWPVLGNLPQLG
DKPHHTMAALARHHGPLFRLRFGSAEVVVAASAKVAGSFLRAHDANFSDRPPNSGA
EHVAYNYQDLVFAPYGARWRALRKLCAQHLFSARALDALRQVRQDEARLMVTRLL
SSSDSPAGLAVGQEANVCATNALALAAVGRRVFGDGVGEGAREFKDMVVELMQLA
GVFNIGDFVPALRWLDPQGVVGKMKRLHRRYDLMMDGFISERGDRADGDGNDLLS
VMLGMMRQSPPAAGEEDGIKFNETDIKALLLNLFTAGTDTTSSTVEWALAELIRHPD
VLKKLQHELDDVVGNGHLVTETDLPQLTFLAAVIKETFRLHPSTPLSLPRVAAEDCE
VDGYRIPKDTTLLVNVWAIARDPASWGDDVLEFRPTRFLPGGLHESVDVKGGDYELI
PFGAGRRICAGLSWGLRMVTLMTATLVHAFDWTLVDGMTPEKLDMEEAYGLTLQR
AVPLMVQPVPRLLPSAYTM*
SEQ ID NO: 42
Triticum turgidum
TRITD2Bv1G262360
MDHDLLLLLASLAAVAVAAVCYLRSHGSGAKLPLPPGPRGWPVLGNLPQLGAKPH
HTMAALARQHGPLFRLRFGSAEVVVAASAKVAGSFLRAHDANFSDRPPNSGAEHVA
YNYQDLVFAPYGARWRALRMLCALHLFSARALDALRSVRQDEARLMVTHLLSASS
SPAQGVAIGQEANVCATNALARAAVGRRVVGDGVGESAREFKGMVVELMQLAGA
FNIGDFVPALRWLDPQGVVAKMKHLHRRYDRIMDGFISEREHLAGEEEGKDLLSIML
AKMRQPLHADAGEDGIKFTETNIKALLLNLLTAGTDTTSSTVEWALVELIRHPDTLK
QLQREVDDVVGTSRLVTEADLPRLTFLTAVIKETFRLHPSTPLSLPRVAAEDCEVDGY
HVAKGTTLLVNVWAISRDPASWGADALEFRPARFLPGGSHETVDVKGGDYELIPFG
AGRRMCAGLSWGLRIVTLMTATLVHAFDLSLVNGMTPDKLDMEEAYGLTLQRAVP
LLVQPMPRLLPSAYATPCVN*
SEQ ID NO: 43
Triticum turgidum
TRITD6Av1G001970
MEIPLTLLLSTFAISVTICYVIIFFFRADKGRAPLPPGPRGWPVLGNLPQLGGKTHQTL
HEMTRLYGPMLRLRFGSSLVVVAGSADVAKQFLRTHDAKFSSRPPNSGGEHMAYN
YQDVVFAPYGPRWRAMRKVCAVNLFSARALDDLRAFREWEALGAEEFNEIVLKLIE
VGGVLNVGDFVPVLRWLDPQGVVAKMKKLHRRFDDMMNRIIAERRAGGFATTAGE
EGGKDLLGLLLAMVQEDKSLTGAEENKITDTDVKALILLPAGQTPVMEETFSLLLQL
AVPLMVHPVPRLLPSAYQIA*
SEQ ID NO: 44
Triticum turgidum
TRITD6Bv1G003180
MEIPLPLLLSTFAISVTICYVIFFFFHADKGRAPLPPGPRGWPVLGNLPQLSGKTHQTL
HEMTKLYGPMLRLRFGSSLVVVAGSADVAKQFLRTHDARFSSRPPNSGGEHMAYN
YQDVVFAPYGPRWRAMRKVCAVNLFSARALDDLRAFREREATEPGAVDFNEIVLKL
IEVGGVLNVGDFVPALRWLDPQGVVAKMKKLHRRFDDMMNRIITERRTGAIAATAG
EEDGKDLLGLLLAMVQEDKSLTGGSEEDRMTDTDVKALILLPAGKTPDMEETFSLLL
QLAVPLMARPVPRLLPSAYQIA*
SEQ ID NO: 45
Triticum turgidum
TRITD7Av1G223010
MNTRAPAVLAYRSNATMHLVAMDIPLPLLLSTLAVAVGVCYVLATFFRADKGRAPL
PPGPRGWPVLGNLPQLGGKTHQTMHEMSKVYGPVLRLRFGSSVVVVAGSAGAAEQ
FLRTHDAKFSSRPPNSGGEHMAYNYQDVVFAPYGPRWRAMRKVCAVNLFSARALD
DLRGFREREAALMVRSLVDAAATGGVVAVGKAANVCTTNALSRAAVGLRVFAAA
GAELGAKEFKEIVLEVMEVGGVLNVGDFVPALRWLDPQGVVARLKKLHRRFDAM
MNGIIAERRAGGSTAGEEKEGKDLLGLLLAMVQEDKSLTGGEEDRITDTDVKALILN
LFVAGTETTSTIVEWAVAELIRHPDMLKRAQEEMDAVVGRDRLVSESDLPRLTFLNA
VIKETFRLHPSTPLSLPRMASEECEVAGYRIPKGTELLVNVWGIARDPALWPDPLEFR
PARFLPGGTHADVDVKGGDFGLIPFGAGRRICAGLSWGLRVVTVTAATLVHSFDWE
LPAGQTPDKLNMEEAFSLLLQRAVPLMAHPVPRLLPSAYEIA*
SEQ ID NO: 46
Triticum turgidum
TRITD7Bv1G170910
MHLVAMGIPLPLLLSTLAIAVTICYVLATFFRADKGRAALPPGPRGWPVLGNLPQLG
GKTHQTMHEMSKVYGPVLRLRFGSSVVVVAGSAAVAEQFLRTHDAKFSSRPPNSGG
EHMAYNNQDVVFAPYGPRWRAMRKVCAVNLFSARALDDLRGFREREAALMVRSL
VDAASGGGVVAVGKAANVCTTNALSRAAVGLRVFAAAGTELGAKEFKEIVLEVME
VGGVLNVGDFVPALRWLDPQGVVARLKKLHRRFDDMMNGIIAERRAGGSTAGEEK
EGKDLLGLLLAMVQEDKSLTGGEEDRITDTDVKALILNLFVAGTETTSTIVEWAVAE
LIRHPDMLKRAQEEMDAVVGRDRLVSESDLPRLTFLNAVIKETFRLHPSTPLSLPRMA
SEECEVAGYRIPKGTELLVNVWGIARDPALWPDPLEFRPARFLPGGTHADVDVKGG
DFGLIPFGAGRRICAGLSWGLRVVTVTAATLVHSFDWELPAGQTPDKLNMEEAFSLL
LQRAVPLMVHPVPRLLPSAYQIA*
SEQ ID NO: 47
Setaria italica
Seita.9G242900
MDVPLPLLLGTVAVAAAAAWYLLLRRGGGGGKRPLPPGPRGWPVLGNLPQLGAKP
HHTMAAMAREHGPLFRLRFGSAEVVVAASAAVAAQFLRAHDANFSNRPPNSGAEH
VAYNYQDLVFAPYGARWRALRKLCALHLFSARALDDLRAVREGEVALMVRELARQ
RGPAVALGQAANVCATNTLARATVGRRVFAVDGGEGAREFKEMVVELMQLAGVF
NVGDFVPALAWLDPQGVVGRMKRLHRRYDDMMDRIIREREAAGGDGNDLLGVLL
TRMREHRPLADGEDGTINETDIKALLLNLFTAGTDTTSSTVEWALAELIRHPDVLAK
AQQELDAVVGRGRLVSESDLPRLTYLTAVIKETFRLHPSTPLSLPRVAAEDCEVGGY
LVPAGTTLLVNVWAIARDPDAWPEPLEFRPDRFLSGGPHAGVDVKGSDFELIPFGAG
RRICAGLSWGLRMVTLMTAALVHGLDWHLAGGVDADKLDMEEAYGLTLQRAVPL
MVRPEPRLLPSAYASVE*
SEQ ID NO: 48
Setaria italica
SEITA.9G244600
MHMPCISFPRMSSDGKSMEIGRTMDIPTPLLLSTLAVSVVICYVLFWKQVATRKPAA
RPTPGGEHMAYNYQDVVFAPYGPRWRAMRKVCAVNLFSARALDDLRAVREREAA
LMVRSLAAAGQATAAVPLGRAVNVCTTNALSRAAVGRRVFAAGAGDDEGAREFKE
IVLEVMQVGGVLNVGDFVPALRWLDPQGVVAKMKKLHRRFDDMMNGIIADRRKA
GVTEEGKDLLGLLLAMVKDAGGEEDRITETNAKALILNLFVAGTDTTSTIVEWSLAE
LIRHPAILKQAQAELDAVVGRGRLLSESDLPRLTFFNAVIKETFRLHPSTPLSLPRMAA
AECEVAGYRIPKGSELLVNVWGIARDPALWGPDPLEFRPARFLPGGSHADVDVKGG
DFGLIPFGAGRRICAGLSWGLRMVTLASATLVHAFDWEMPAGQTPDELDMEEAFTL
LLQRAVPLMVHPVPRLLPLAYEIA*
SEQ ID NO: 49
Cenchrus americanus
Pgl_GLEAN_10033465
MDLPLSLLLGTVAVAAVAAAWEAAGGDGPDLLGVLLARMREHQPLADGEDGTINE
TDMKALLLNLFTAGTDTTSSTVEWALAELLRHPDVLAKAQQELDAVVGRGRLVSES
DLARLTYLTAVIKETFRLHPSTPLPDRFLPGGQHAGVDVKGSDFELIPFGAGRRICAG
LSWGLRMVTLMTAALVHGLDWHLAGGVDADKLDMEEAYGLTLQRAVPLMVRPEP
RLPPSAYAASSVE*
SEQ ID NO: 50
Cenchrus americanus
Pgl_GLEAN_10033479
MDNIPTPLLLSTLAVSLVICYVLFWKQQAATRTKPQRAPLPPGPRGWPVLGNLPQLG
GKTHQTLHEMTKVYGPLLRLRFGSSDVVVAGSAAVAEQFLRVHDANFSCRPPNSGG
AAVGRRVFAAGGSGDDEGGAREFKEIVLEVMRVGGVLNVGDFVPALRWLDPQGVV
AKMKKLHRRFDSMMNGIIADRRKAAGVTTEEGKDLLGLLLEMVKDERPLAGGEED
RITETDAKALILNLFVAGTDTTSTIVEWSLAELIRHPTILKQAQEELDAVVGRGRLVA
ESDLPRLTFFAAVFRPARFLPGGSHAGVDVKGGDFGLIPFGAGRRICAGLSWGLRMV
TLASATLVHAFDWELPAGQTPDKLDMEEAFTLLLQRATPLMVQPVPRLLPSAYEIA*
SEQ ID NO: 51
Sorghum bicolor
Sobic.004G200800
MDVPLPLLLGSLAVSVVVWCLLLRRGGDGKGKGKRPMPPGPRGWPVLGNLPQLGS
HPHHTMCALAKKYGPLFRLRFGSAEVVVAASARVAAQFLRTHDANFSNRPPNSGAE
HVAYNYQDMAFAPYGSRWRALRKLCALHLFSAKALDDLRSIREGEVALLVRELSRH
QHQHAGVPLGQVANVCATNTLARATVGRRVFAVDGGEEAREFKDMVVELMQLAG
VFNVGDFVPALARLDLQGVVGKMKRLHRRYDDMMNGIIRERKAAEEGKDLLSVLL
ARTREQQSIADGEDSRITETEIKALLLNLFTAGTDTTSSTVEWALAELIRHPDVLKKA
QEELDAVVGRNRLVSELDLPRLTYLTAVIKETFRMHPSTPLSLPRIAAEECEVDGFRIP
AGTTLLVNVWAIARDPEAWPEPLQFRPDRFLPGGSHAGVDVKGSDFELIPFGAGRRI
CAGLSWGLRMVTLMTATLVHALDWDLADGMTADKLDMEEAYGLTLQRAVPLKV
RPAPRLLPSAYAAE*
SEQ ID NO: 52
Sorghum bicolor
Sobic.004G200833
MDVPLPLLLGSLAVSVVVWCLLLRRGGDGKGKGKRPMPPGPRGWPVLGNLPQLGS
HPHHTMCALAKKYGPLFRLRFGSAEVVVAASARVAAQFLRTHDANFSNRPPNSGAE
HVAYNYQDMAFAPYGSRWRALRKLCALHLFSAKALDDLRSIREGEVALLVRELSRH
QHQHAGVPLGQVANVCATNTLARATVGRRVFAVDGGEEAREFKDMVVELMQLAG
VFNVGDFVPALARLDLQGVVGKMKRLHRRYDDMMNGIIRERKAAEEGKDLLSVLL
ARTREQQSIADGEDSRITETEIKALLLNLFTAGTDTTSSTVEWALAELIRHPDVLKKA
QEELDAVVGRNRLVSELDLPRLTYLTAVIKETFRMHPSTPLSLPRIAAEECEVDGFRIP
AGTTLLVNVWAIARDPEAWPEPLQFRPDRFLPGGSHAGVDVKGSDFELIPFGAGRRI
CAGLSWGLRMVTLMTATLVHALDWTSPTA*
SEQ ID NO: 53
Sorghum bicolor
Sobic.004G200900
MHVPLLLGSLAVSVVVWCLLLRRGGDGKGKGNGKRPLPPGPRGWPVLGNLPQVGS
HPHHTMYALAKEYGPLFRLRFGSADVVVAASARVAVQFLRAHDANFSNRPPNSGAE
HMAYNYQDMVFAPYGSRWRALRKLCALHLFSAKALDDLRGVREGEVALMVRQLA
LHQHQHAGVPLGQVANVCATNTLARATVGRRVFAVDGGEEAREFKDMVVELMQL
AGVFNVGDFVPALAWLDLQGVVGKMKRLHRRYDDMMNSIIRKRKAAEEGKDLLS
VLLARMREQQSLADGEDSRINETGIKALLLDLFTAGTDTTSSTVEWALAELIRHPDVL
KKAQEELDAVVGRDRLVSETDLPRLTYLTAVIKETFRLHPSTPLSLPRVAAEECEVDG
FRIPAGTTLLVNVWAIARDPEAWPEPLQFRPDRFLPGGSHAGVDVKGSDFELIPFGAG
RRICAGLSWGLRMVTLMTATLVHALDWDLADGMTADKLDMEEAYGLTLQRAVPL
MVRPTPRLLPSAYAAE*
SEQ ID NO: 54
Sorghum bicolor
Sobic.004G201100
MQVASVYIDEPLSLANHTRTTLSPTPSAPPVNRATQTMDVPLPLLLGSLAVSVVVWC
LLLRRGGNGKGKGKRPLPPGPRGWPVLGNLPQVGSHPHHTMCALAKEYGPLFRLRF
GSAEVVVAASARVAAQFLRAHDANFSNRPPNSGAEHVAYNYQDLVFAPYGSRWRA
LRKLCALHLFSAKALDDLRGVREGEVALMVRELARHQHQHAGVPLGQVANVCATN
TLARATVGRRVFAVDGGEEAREFKDMVVELMQLAGVFNVGDFVPALAWLDLQGV
VGKMKRLHRRYDDMMNGIIRERKAVEEGKDLLSVLLARMREQQSLADGEDSMINE
TDIKALLLNLFTAGTDTTSSTVEWALAELIRHPDVLKKAQEELDAVVGRDRLVSESD
LPRLTYLTAVIKETFRLHPSTPLSLPRVAAEECEVDGFRIPAGTTLLVNVWAIARDPEA
WPEPLQFRPDRFLPGGSHAGVDVKGSDFELIPFGAGRRICAGLSWGLRMVTLMTATL
VHALDWDLADGMTANKLDMEEAYGLTLQRAVPLMVRPAPRLLPSAYAAE*
SEQ ID NO: 55
Sorghum bicolor
Sobic.009G162500
MVMELVLATPDLPTPLLLSALTVAVSVAVCYVLFWKQQQAAARRAPLPPGPRGWP
VLGNLPQLGGKTHQTLHELTKVYGPLLRLRFGSSDVVVAGSAAVAEQFLRVHDANF
SCRPPNSGGELMAYNYQDVVFAPYGPRWRAMRKVCAVNLFSARALDDICDVRERE
AALMVRSLAEQAARDRNTPVALGKAVNVCTTNALSRAAVGRRVFAAAGAGDEGA
REFKEIVLEVMEVGGVLNVGDFVPALRWLDPQGVVGRMKKLHRRFDDMMNGIIAD
SRKARATPADGEESKDLLGLLLSMVEDEGSDDEVRITETDVKALILNLFIAGTDTTSTI
AEWSLAELIRHPDILKQAQEELDTVVGRGRLVTESDLRHLTFFNAVIKETFRLHPSTP
LSLPRMAAEECEIAGYSIPKGCELLVNVWGIARDPALWPDPLEFRPARFLPGGSHSDV
DVKGGNFGLIPFGAGRRICAGLSWGLRMVTLTSATLVHAFDWELPVGQTPDKLNME
EAFTLLLQRAVPLMAHPIPRLLPSAYEIA*
SEQ ID NO: 56
Brachypodium distachyon
Bradi1g17180
MEDMPLPLLIGSLFIILAMWYILFHHGSENNAKWSRLPLPPGPCGWPLLGNLPQLGA
KPHHTMCALAWEHGPLFRLRLGSTEVVVASSAGIAMQFLRHHDANFSNRPPNSGAE
HIAYNYQDLVFASYGTRWRALRKLCALHLFSAKALNNLRNVREGEVRLMVRELAW
AAAGPAPAVALGQQANMCVTNTLARATIGRRVFAVDTAREFKEMVVELMQLAGVF
NLGDFVPALRWLDPQGVVAKMKRLHRRYDNMMNGFIKEREPACLSAGAEAKDLLS
VMLVKMREQQPLYHEEGKLTNTDIKALLLNLFTAGTDTASSTVEWALAELIRHPDV
LKQVQRELDVVVGNDRLVSESDLPGLTFLPAVIKETFRLHPPTPLSLPRVAAEECEVN
GYHIPKGTTLLVNVWAIARDPASWPDHPLEFRPVRFLPGGSHESLDVKGSDYELIPFG
AGRRICAGLGWGIQMVTLMTTTLVHAFDWSLVDGMTPDKLDMEEAYGLTLQRAM
PLFVQPVPRLLPSAYAM*
SEQ ID NO: 57
Brachypodium distachyon
Bradi1g24840
MLAFCMSKRSNSWRATAEACMELIGALDVPLRLPWLVSALAISVTVCYILFFSRAGK
GNGKGLPPGPRGWPVLGNLPQLGGKTHQTLHELTKVYGPVLRLRLGSSVAVVAGTA
GTAEQFLRAHDAQFRDRPPNSGGEHMAYNVFGPYGPRWRAMRKVCAVNLFSARAL
DGLRGFREREAALMVKSLAAAAASAAEPVALGKAANVCTTNALSRAAVGRRVFDE
MGGSAGGELKEIVLEVIDVGGVLNVGDFVPALRWLDPQGVVARMKKLHRRFDDM
MNGIIGERLQGTDAAGEKDLLGLLLDAMMKEDKSLSGGEELTHTDIKALILNLFVAG
TDTTSSIVEWAMSELIRHPDLLQQAQEELDAVVGRARLVSESDMSRLPFLTAVIKETF
RPHPSTPLSLPRMASEECFVAGYRIPKGTELVVNVWGIARDPALWPDPLEFRPARFLI
GGSNSVVDLKGSNFELIPFGAGRRICAGLSWGLRIVMIAVATLVHAFDWKLPVGQTP
DELNMEEALSLLLLRAVPLMVHPAPRLLPSAYEIA*
SEQ ID NO: 58
Brachypodium distachyon
Bradi3g04750
MDDFLLVAGSLALALTVCYYFIIHDNNNKAKKLPLPLPPGPRGWPVLGNLPQLGAAP
HQTMRALAAEHGPLFRLRFGSAEVVVAASASVAARFLRGHDANFGDRPPNSGAEHV
AYNYRDLVFAPYGARWRALRKLLALHLFSAKAIDALRGVRELEVALMVKGLRVSSS
APAGVAVGQEANVCATNALARAAVGRRVFFSGGGGGADSREFKEMVVELMQLAG
VFNLGDFIPALRWLDPQGVVAKMKKLHRRYDDMMNGFIKERDAGAGAEQGKDLLS
VMLGKMRELGGDDNNGGEEGEFTEVDIKALLLNLFTAGTDTTSSTVEWALAELIRH
PDVLRQLQQELDAVVGKDRLVSESDLPRLAFLAAVIKETFRLHPSTPLSLPRLAAEEC
EVDGYRIPKGTTLLVNVWAIARDPASWADPLEFRPARFLPGGSHEGVDVKGGDYELI
PFGAGRRICAGLSWGLRMVTLMTATLVHGFDWALVNGMTPDKLDMEEAYGLTLQ
RAVPLMVQPVPRLLPSAYAVQCDG*
SEQ ID NO: 59
Brachypodium distachyon
Bradi4g16560
MELLDGLDVPLLPALLSALAISLTICYVLFFSRAGKGLPPGPRGWPVLGNLPQLGGKT
HQTLHEMSKLYGPVLRLRFGSSVVVVAGSAGAAEQFLRTNDAKFSNRPPNSGGEHM
AYNYQDVVFGPYGPRWRAMRKVCAVNLFSARALDDLRGFREREASLMVKSLADA
AAASGAGPVVALGKAANVCTTNALSRAAVGRRVFAAAGGEGAREFKEIVLEVMEV
GGVLNVGDFVPALRWLDPQGVVARMKKLHRRFDDMMNGIIAEREGGCGMAPGED
GKEKDLLGLLLGMMQEEKSLTGGEEDDKITHTDIKALVLNLFVAGTETTSTIVEWAV
AELIRHPDLLQQAQEELDAVVGRARVVSEADLPRLPFFTAVIKETFRLHPSTPLSLPR
MASEECFVAGYRIPKGTELLVNIWGIARDPALWPDPLEFRPSRFLAGGSHADVDLKG
LLQRAMPLMVHPVRRLLPSAYEIV*
SEQ ID NO: 60
Hordeum vulgare
HORVU6Hr1G002400
MEIPLPLLLSTLAISVTICYVIFFFFRSDKGCAPLPPGPRGWPVLGNLPQLGGKTHQTL
HEMTRLYGPMFRLWFGSSLVVVAGSADMAKLFLRTHDAKFSSRPPNSGGEHMAYN
YQDVVFAPYGPRWRAMRKVCAVNLFSARALDDLHSFREREAALMVRCLADSAAV
GRVVALAKAANVCTTNALSRATVGLRVFATAGSELGAEDFNEIVLKLIEVGGILNVG
DFVPALRWLDPQGVVAKMKKLHRRFDDMMNRIIAQRRAVSTTAGKDLLALLLAMV
QEDKSLTGVEEDKIRDTDVKALILNLFVAGTDTTSITVEWAMAELIRHPHILKQAQEE
LDAVVGRDRLVLESDLPHLTFLNAVIKETFRLHPSTPLSLPRMAIEECEVAGHRIPKGT
QLLVNVWGIARDPTLWPDPLEFRPARFLPGGSHAGVDVKGGDFGLIPFGAGRRICAG
LSWGIRMVTVTTATLVHSFDWEMSAGQMPDMEETFSLLLQLAVPLMVHPVPRLLPS
AYEIT*
SEQ ID NO: 61
Gossypium raimondii (the putative contributor of the D subgenome to the
economically important fiber-producing cotton species Gossypium hirsutum
and Gossypium barbadense.) XP_012438857
MASFVLYSILSAVFLYFVFITSRKRRRLPLPPGPKPWPIIGNLPHMSPVPHQGLAAMA
KVYGPLMHLRLGFVDVVVAASASMAAQFLKVHDSNFSSRPPNAGAKYVAYNYQDL
VFAPYGPRWRLLRKISSLHLFSGKALDDFRQIREEEIRVLVRALASAKTKVNLGQLLN
VCTVNALGQVMMGKRVFGDGSGGSDPEADEFKSMVVELMQLAGVFNIGDFIPALE
WLDLQGVQAKMKKLHNRFDRFLSAILEEHKTKARQSNGQVKHKDFLSTLISLENVD
GAEGGKLSDTEIKALLLNMFTAGTDTSSSTVEWAMAELIRHPNIMAQVRKELDSVV
GRDRLVSDLDLPNLTYFQAVIKETFRIHPSTPLSLPRMASDSCDINGYHIPKGATLLVN
VWAISRDPNEWNNPLEFRPERFLPGGERPNADVRGNDFEVIPFGAGRRICAGMSLGL
RMVQLLTATLAHAFEWELADGLMPEKLDMEEAYGLTLQRAAPLMVHPRPRLSKHA
Y
SEQ ID NO: 62
Gossypium raimondii
XP_012478317
MPSFDTILLRDLVAAACLFFITRYFIRRLLSNPKRTLPPGPKGWPIVGALPLLGSMPHV
ELAKLAKKYGPVMYLKMGTCNMVVASTPDAARAFLKTLDLNFSNRPSNAGATHIA
YNSQDMVFAEYGPRWKLLRKLSNLHMLGGKALEDWSQVRAVELGHMLRAMCESS
RKGEPVVVPEMLTYAMANMIGQVILSRRVFVTKGSESNEFKDMVVELMTSAGLFNI
GDFIPSIAWMDLQGIEGEMKKLHNRWDVLLTKMMKEHEETAYERKGKPDFLDIIMD
NRENSAGERLSLTNVKALLLNLFTAGTDTSSSIIEWALAEILKNPKILNKAHEEMDKV
IGRNRRLEESDIPKLPYLQAICKETFRKHPSTPLNLPRVSTQACEINGYYIPKNTRLSVN
IWAIGRDPDVWGNPLDFTPERFLSGRFAKIDPRGNDFELIPFGAGRRICAGTRMGIVL
VEYILGTLLHSFDWMLPPGNGELNMDEAFGLALQKAVPLSAMVRPRLAPTAYVS
SEQ ID NO: 63
Gossypium raimondii
KJB51033
MASFVLYSILSAVFLYFVFITSRKRRRLPLPPGPKPWPIIGNLPHMSPVPHQGLAAMA
KVYGPLMHLRLGFVDVVVAASASMAAQFLKVHDSNFSSRPPNAGAKYVAYNYQDL
VFAPYGPRWRLLRKISSLHLFSGKALDDFRQIREEEIRVLVRALASAKTKVNLGQLLN
VCTVNALGQVMMGKRVFGDGSGGSDPEADEFKSMVVELMQLAGVFNIGDFIPALE
WLDLQGVQAKMKKLHNRFDRFLSAILEEHKTKARQSNGQVKHKDFLSTLISLENVD
GAEGGKLSDTEIKALLLNMFTAGTDTSSSTVEWAMAELIRHPNIMAQVRKELDSVV
GRDRLVSDLDLPNLTYFQAVIKETFRIHPSTPLSLPRMASDSCDINGYHIPKGSCPAAK
GRTLMLGAMILRSYRSAPGVESVPE
SEQ ID NO: 64
Gossypium raimondii
XP_012454458
MATPSWFSYLTPWLATLALILFSLRLCRRRKLNLPPGPKPWPIIGNLNLIGSLPHQSIH
ALSRKYGPIMQLKFGSFPVVVASSVEMAKAVLKTNDVIFTDRPKTAAGKYTTYNYS
DITWSPYGPYWRQARKICLTELFNAKRLESYQYIRREEMNLFLKRLYESSGTQIVLKD
HLSSLSLNVISRMVFGKKYTEGSGENEIVTPNEFKEMLDELFLLNGVLDIGDSIPWLSF
LDLQGYIKRMKALSKRFDRFLEHVLDEHNARREGAEDYVAKDMVDVLLQLSEDPN
LEVKLERHGVKAFTQDMIAGGTESSAVTVEWAISELLKKPEILAKATEELDMVIGRE
RWVEEKDVVSLPYIDSIAKETMRLHPVAPMLVPRVARQDCEIAGYDIPKGTRAFVNV
WTIGRDPSLWDNPNEFWPDRFMGKSIDVKGHDFELLPFGAGRRMCPGYPLGIKVIQA
SLANVLHGFTWKLPNNTTKEDLNMEEIFGLSTPKKYPLEAIAEPRLPLHMYS
SEQ ID NO: 65
Gossypium raimondii
XP_012490769
MESPSWVSYLTAWLATLALILLSLRFRPRRKLNFPPGPKPWPVIGNLDLICSLPHRSIH
ALSQKYGPLMQLKFGSFPVVVASSVEMAKAFLKTHDVIFAGRPKIAAGEYTTYNYS
DITWSPYGPYWRQARKMCMTELFSAKRLESYEYIRREEMKLLLKGLYESSGVPIVLK
DRLSDLSLNVISRMVFGKKYTEGTGENEIVTPKEFKEMLDELFLLNGVLDIGDSIPWL
RFLDLQGNIKRMKALSKKFDKFLEHVLDEHNARRRDVKDYVAKDMVDVLLQLADD
PNLDVKLERHGVKAFSQDMIAGGTESSAVTVEWAISEMLKKPEIFAKATEELDRVIG
RERWVEERDIENLPYIDSIAKETMRLHPVAPMLVPRMTREDCQVDGYDILKGTRALV
NVWTIGRDPTVWDNPNEFCPERFIDKTIDVKGHDFQLLPFGAGRRMCPGYPLGIKVI
QASLANLLHGFTWKLPGNMTKEDLDMEEIFGLSTPKKCPLQAVAVPKLPLHLYSH
SEQ ID NO: 66
Gossypium hirsutum (90% of the world's cotton production)
NP_001314443
MASFVLYSILSAVFLYFVFITSRKRRRLPLPPGPKPWPIIGNLPHMSPVPHQGLAAMA
KVYGPLMHLRLGFVDVVVAASASMAAQFLKVHDSNFSSRPPNAGAKYVAYNYQDL
VFAPYGPRWRLLRKMSSLHLFSGKALDDFRQIREEEIRVLVRALASAKTKVNLGQLL
NVCTVNALGQVMMGKRVFGDGSGGSDPEADEFKSMVVELMQLAGVFNIGDFIPAL
EWLDLQGVQAKMKKLHNKFDRFLSAILEEHKTKARQSNGQVKHKDFLSTLISLENV
DGAEGGKLSDTEIKALLLNMFTAGTDTSSSTVEWAMAELIRHPNIMAQVRKELDSV
VGRDRLVSDLDLPNLTYFQAVIKETFRLHPSTPLSLPRMASDSCDINGYHIPKGATLL
VNVWAISRDPNEWNNPLEFRPERFLPGGERPNADVRGNDFEVIPFGAGRRICAGMSL
GLRMVQLLTATLAHAFEWELADGLMPEKLDMEEAYGLTLQRAAPLMVHPRPRLSK
HAY
SEQ ID NO: 67
Gossypium hirsutum
XP_016741685
MASFVLYSILSTVFLYFVFIISRKRRRLPLPPGPKPWPIIGNLPHMSPVPHQGLAAMAK
VYGPLMHLRLGFVDVVVAASASMAAQFLKVHDSNFSSRPPNAGAKYVAYNYQDLV
FAPYGPRWRLLRKISSVHLFSGKALDDFRHIREEEIRVLVRALASAKTKVNLGQLLNV
CTVNALGQVMMGKRVFGDGSGGADPEADEFKSMVVELMQLAGVFNIGDFIPALEW
LDLQGVQAKMKKLHNRFDRFLSGILEEHKTKARQSNGQVKHKDLLSTLISLENADG
AEGGKLSDTEIKALLLNMFTAGTDTSSSTVEWAMAELIRHPNIMAQVRKELDSVVGR
DRLVSDLDLPNLTYFQAVIKETFRLHPSTPLSLPRMASDSCDINGYHIPKDATLLVNV
WAISRDPNEWNNPLEFRPERFLPGGERPNADVRGNDFEVIPFGAGRRICAGMSLGLH
MVQLLTATLAHAFDWELADGLMPEKLDMEEAYGLTLQRAAPLMVHPRPRLSKHAY
SEQ ID NO: 68
Gossypium hirsutum
ACY06905
MAPFVLYSILSAVFLYFVFITSRKRRRLPLPPGPKPWPSIGNLPHMSPVPHQGLAAMA
KVYGPLMHLRLGFVDVVVAASASMAAQFLKVHDSNFSSRPPNAGAKYVAYNYQDL
VFAPYGPRWRLLRKMSSLHLFSGKALDDFRQIREEEIRVLVRALASAKTKVNLGQLL
NVCTVNALGQVMMGKRVFGDGSGGSDPEADEFKSMVVELMQLAGVFNIGDFIPAL
EWLDLQGVQAKTKKLHNKFDRFLSAILEEHKTKARQSNGQVKHKDFLSTLISLENV
DGAEGGKLSDTEIKALLLNMFTAGTDTSSSTVEWAMAELIRHPNIMAQVRKELDSV
VGRDRLVSDLDLPNLTYFQAVIKETFRLHPSTPLSLPRMASDSCDINGYHIPKGATLL
VNVWAISRDPNEWNNPLEFRPERFLPGGERPNADVRGNDFEVIPFGAGRRICAGMSL
GLRMVQLLTATLAHAFEWELADGLMPEKLDMEEAYGLTLQRAAPLMVHPRPRLSK
HAY
SEQ ID NO: 69
Gossypium hirsutum
NP_001314550
MPSFDTILLRDLVAAACLFFITRYFIRRLLSNPKRTLPPGPKGWPIVGALPLLGSMPHV
ELAKLAKKYGPVMYLKMGTCN
SEQ ID NO: 70
Gossypium hirsutum
NP_001314530
MPSFDTILLRDLVAAACLFFITRYFIRRLLSNPKRTLPPGPKGWPVVGALPLLGSMPH
VELAKLAKKYGPVMYLKMGTCNMVVASTPDTARAFLKTLDLNFSNRPSNAGATHI
AYNSQDMVFAEYGPRWKLLRKLSNLHMLGGKALEDWSQVRAVELGHMLRAMWE
SSRKGEPVVVPEMLTYAMANMIGQVILSRRVFVTKGSESNEFKDMVVELMTSAGLF
NIGDFIPSIAWMDLQGIEGEMKKLHNRWDVLLTKMMKEHEETAYERKGKPDFLDII
MDNRENSAGERLSLTNVKALLLNLFTAGTDTSSSIIEWALAEILKNPKILNKAHEEMD
KVIGRNRRLEESDIPKLPYLQAICKETFRKHPSTPLNLPRVSTQPCEINGYYIPKNTRLS
VNIWAIGRDPDVWGNPLDFTPERFLSGRFAKIDPRGNDFELIPFGAGRRICAGTRMGI
VLVEYILGTLLHSFDWMLPPGTGELNMDESFGLALQKTVPLSAMVRPRLAPTAYVS
SEQ ID NO: 71
Gossypium hirsutum
ACY06904
MPSFDTILLRDLVAAACLFFITRYFIRRLLSNPKRTLPPGPKGWPVVGALPLLGSMPH
VELAKLAKKYGPVMYLKMGTCNMVVASTPDTARAFLKTLDLNFSNRPSNAGATHI
AYNSQDMVFAEYGPRWKLLRKLSNLHMLGGEALEDWSQVRAVELGHMLRAMWE
SSRKGEPVVVPEMLTYAMANMIGQVILSRRVFVTKGSESNEFKDMVVELMTSAGLF
NIGDFIPSIAWMDLQGIEGEMKKLHNRWDVLLTKMMKGHEETAYERKGKPDFLDII
MDNRENSAGERLSLTNVKALLLNLFTAGTDTSSSIIEWALAEILKNPKILNKAHEEMD
RVIGRNRRLEESDIPKLPYLQAICKETFRKHPSTPLNLPRVSTQACEINGYYIPKNTRLS
VNIWAIGRDPDVWGNPLDFTPERFLSGRFAKIDPRGNDFELIPFGAGRRICAGTRMGI
VLVEYILGTLLHSFDWMLPPGTGELNMDEAFGLALQKAVPLSAMVRPRLAPTAYVS
SEQ ID NO: 72
Gossypium hirsutum
XP_016710494
MESPSWVSYLIAWLATLALILLSLRFRPRRKLNLPPGPKPWPVIGNLDLIGSLPHRSIH
SLSQKYGPLMQLKFGSFPVVVASSVEMAKAFLKTHDVIFAGRPKIAAGEYTTYNYSD
ITWSPYGPYWRQARKMCMTELFSAKRLESYEYIRREEMKLLLKGFYESSGVPIVLKD
HLSDLSLNVISRMVFGKKYTEGTGENEIVTPKEFKEMLDELFLLNGVLDIGDSIPWLR
FLDLQGNIKRMKALSKKFDKFLEHVLDEHNARRRDVKDYVAKDMVDVLLQLADDP
NLDVKLERHGVKAFSQDLIAGGTESSAVTVEWAISEMLKKPEIFAKATEELDRVIGRE
RWVEERDIVNLPYIDSIAKETMRLHPVAPMLVPRMTREDCQVDGYDILKGTRALVN
VWTIGRDPTVWDNPNEFFPERFIDKTIDVKGHDFQLLPFGAGRRMCPGYPLGIKVIQA
SLANLLHGFNWKLPGNMTKDDLDMEEIFGLSTPKKCPLQAVAVPKLPLHMYSH
SEQ ID NO: 73
Gossypium hirsutum
KAG4120389
MESPSWVSYLTAWLATLALILLSLRFRPRRKLNFPPGPKPWPVIGNLDLIGSLPHRSIH
ALSQKYGPLMQLKFGSFPVVVASSVEMAKAFLKTHDVIFAGRPKIAAGEYTTYNYS
DITWSPYGPYWRQARKMCMTELFSAKRLESYEYIRREEMKLLLKGLYESSGVPIVLK
DRLSDLSLNVISRMVFGKKYTEGTGENEIVTPKEFKEMLDELFLLNGVLDIGDSIPWL
RFLDLQGNIKRMKALSKKFDKFLEHVLDEHNARRRDVKDYAAKDMVDVLLQLADD
PNLDVKLERHGVKAFSQDLIAGGTESSAVTVEWAISEMLKKPEIFAKATEELDRVIGR
ERWVEERDTVNLPYIDSIAKETMRLHPVAPMLVPRMTREDCQVDGYDILKGTRALV
NVWTIGRDPTVWDNPNEFCPERFIDKTIDVKGHDFQLLPFGAGRRMCPGYPLGIKVI
QASLANLLHGFTWKLPGNMTKENLDMEEIFGLSTPKKCPLQAVAVPKLPLHLYSH
SEQ ID NO: 74
Gossypium hirsutum
NP_001314163.1
MTQQAILLSLRFRPRRKLNFPPGPKPWPVIGNLDLIGSLPHRSIHALSQKYGPLMQLKF
GSFPVVVASSVEMAKAFLKTHDVIFAGRPKIAAGEYTTYNYSDITWSPYGPYWRQA
RKMCMTELFSAKRLESYEYIRREEMKLLLKGLYESSGVPIVLKDRLSDLSLNVISRMV
FGKKYTEGTGENEIVTPKEFKEMLDELFLLNGVLDIGDSIPWLRFLDLQGNIKRMKAL
SKKFDKFLEHVLDEHNARRRDVKDYAAKDMVDVLLQLADDPNLDVKLERHGVKA
FSQDLIAGGTESSAVTVEWAISEMLKKPEIFAKATGELDRVIGRERWVEERDTVNLPY
IDSIAKETMRLHPVAPMLVPRMTREDCQVDGYDILKGTRALVNVWTIGRDPTVWDN
PNEFCPERFIDKTIDVKGHDFQLLPFGAGRRMCPGYPLGIKVIQASLANLLHGFTWKL
PGNMTKENLDMEEIFGLSTPKKCPLQAVAVPKLPLHLYSH
SEQ ID NO: 75
Gossypium barbadense (5% of the world's cotton production)
KAB2053485
MTSFVLYSILSTVFLYFVFIISRKRRRLPLPPGPKPWPIIGNLPHMSPVPHQGLAAMAK
VYGPLMHLRLGFVDVVVAASASMAAQFLKVHDSNFSSRPPNAGAKYVAYNYQDLV
FAPYGPRWRLLRKISSVHLFSGKALDDFRHIREEEIRVLVRALASAKTKVNLGQLLNV
CTVNALGQVMMGKRVFGDGSGGADPEADEFKSMVVELMQLAGVFNIGDFIPALEW
LDLQGVQAKMKKLHNRFDRFLSGILEEHKTKARQSNGQVKHKDLLSTLISLENADG
AEGGKLSNTEIKALLLNMFTAGTDTSSSTVEWAMAELIRHPNIMAQVRKELDSVVGR
DRLVSDLDLPNLTYFQAVIKETFRLHPSTPLSLPRMASDSCDINGYHIPKGATLLVNV
WAISRDPDEWNNPLEFRPERFLPGGERPNADVRGNDFEVIPFGAGRRICAGMSLGLR
MVQLLTATLAHAFDWELADGLMPEKLDMEEAYGLTLQRAAPLMVHPRPRLSKHAY
SEQ ID NO: 76
Gossypium barbadense
KAB1669149
MASFVLYSILSAVFLYFVFITSRKRRRLPLPPGPKPWPIIGNLPHMSPVPHQGLAAMA
KVYGPLMHLRLGFVDVVVAASASMAAQFLKVHDSNFSSRPPNAGAKYVAYNYQDL
VFAPYGPRWRLLRKMSSLHLFSGKALDDFRQIREEEIRVLVRALASAKTKVNLGQLL
NVCTVNALGQVMMGKRVFGDGSGGSDPEADEFKSMVVELMQLAGVFNIGDFIPAL
EWLDLQGVQAKMKKLHNKFDRFLSAILEEHKTKARQSNGQVKHKDFLSTLISLENV
DGAEGGKLSDTEIKALLLNMFTAGTDTSSSTVEWAMAELIRHPNIMAQVRKELDSV
VGRDRLVSDLDLPNLTYFQAVIKETFRLHPSTPLSLPRMASDSCDINGYHIPKGATLL
VNVWAISRDPNEWNNPLEFRPERFLPGGERPNADVRGNDFEVIPFGAGRRICAGMSL
GLRMVQLLTATLAHAFEWELADGLMPDKLDMEEAYGLTLQRAAPLMVHPRPRLSK
HAY
SEQ ID NO: 77
Gossypium barbadense
PPD88185
MASFVLYSILSAVFLYFVFITSRKRRRLPLPPGPKPWPIIGNLPHMSPVPHQGLAAMA
KVYGPLMHLRLGFVDVVVAASASMAAQFLKVHDSNFSSRPPNAGAKYVAYNYQDL
VFAPYGPRWRLLRKMSSLHLFSGKALDDFRQIREEEIRVLVRALASAKTKVNLGQLL
NVCTVNALGQVMMGKRVFGDGSGGSDPEADEFKSMVVELMQLAGVFNIGDFIPAL
EWLDLQGVQAKMKKLHNRFDRFLSGILEEHKTKARQSNGQVKHKDLLSTLISLENA
DGAEGGKLSNTEIKALLLNMFTAGTDTSSSTVEWAMAELIRHPNIMAQVRKELDSV
VGRDRLVSDLDLPNLTYFQAVIKETFRLHPSTPLSLPRMASDSCDINGYHIPKGATLL
VNVWAISRDPNEWNNPLEFRPERFLPGGERPNADVRGNDFEVIPFGAGRRICAGMSL
GLRMVQLLTATLAHAFDWELADGLMPEKLDMEEAYGLTLQRAAPLMVHPRPRLSK
HAY
SEQ ID NO: 78
Gossypium barbadense
PPR81792
MLVPHQGLAAMAKVYGPLMHLRLGFVDVVVAASASMAAQFLKVHDSNFSSRPPNA
GARYVAYNYQDLEEIRVLVRALASAKTKVNLGQLLNVCTVNALGQVMMGKRVFG
DGSGGADPEADEFKSMVVELMQLAGVFNIGDFIPALEWLDLQGVQAKMKKLHNRF
DRFLSGILEEHKTKARQSNGQVKHKDLLSTLISLENADGAEGGKLSNTEIKALLLNMF
TAGTDTSSSTVEWAMAELIRHPNIMAQVRKELDSVVGRDRLVSDLDLPNLTYFQAVI
KETFRLHPSTPLSLPRMASDSCDINGYHIPKGATLLVNVWAISRDPDEWNNPLEFRPE
RFLPGGERPNADVRGNDFEVIPFGAGRRICAGMSLGLRMVQLLTATLAHAFDWELA
DGLMPEKLDMEEAYGLTLQRAAPLMVHPRPRLSKHAY
SEQ ID NO: 79
Gossypium barbadense
KAB2021362
MPSFDTILLRDLVAAACLFFITRYFIRRLLSNPKRTLPPGPKGWPIVGALPLLGSMPHV
ELAKLAKKYGPVMYLKMGTCNMVVASTPDAARAFLKTLDLNFSNRPSNAGATHIA
YNSQDMVFAEYGPRWKLLRKLSNLHMLGGKALEDWSQVRAVELGHMLRAMCESS
RKGEPVVVPEMLTYAMANMIGQVILSRRVFVTKGSESNEFKDMVVELMTSAGLFNV
GDFIPSIAWMDLQGIEGEMKKLHNRWDVLLTKMMKEHEETAYERKGKPDFLDIIMD
NRENSAGERLSLTNVKALLLNLFTAGTDTSSSIIEWALAEILKNPKILNKAHEEMDKV
IGRNRRLEESDIPKLPYLQAICKETFRKHPSTPLNLPRVSTQACEINGYYIPKNTRLSVN
IWAIGRDPDVWGNPLDFTPERFLSGRFAKIDPRGNDFELIPFGAGRRICAGTRMGIVL
VEYILGTLLHSFDWMLPPGTGELNMDEAFGLALQKAVPLSAMVRPRLAPTAYVS
SEQ ID NO: 80
Gossypium barbadense
KAB2074130
MPSFDTILLRDLVAAACLFFITRYFIRRLLSNPKRTLPPGPKGWPVVGALPLLGSMPH
VELAKLAKKYGPVMYLKMGTCNMVVASTPDTARAFLKTLDLNFSNRPSNAGATHI
AYNSQDMVFAEYGPRWKLLRKLSNLHMLGGKALEDWSQVRAVELGHMLRAMWE
SSRKGEPVVVPEMLTYAMANMIGQVILSRRVFVTKGSESNEFKDMVVELMTSAGLF
NIGDFIPSIAWMDLQGIEGEMKKLHKRWDVLLTKMMKEHEETAYERKGKPDFLDII
MENRENSAGERLSLTNVKALLLNLFTAGTDTSSSIIEWALAEILKNPKILNKAHEEMD
KVIGRNRRLEESDIPKLPYLQAICKETFRKHPSTPLNLPRVSTQPCEINGYYIPKNTRLS
VNIWAIGRDPDVWGNPLDFTPERFLSGRFAKIDPRGNDFELIPFGAGRRICAGTRMGI
VLVEYILGTLLHSFDWMLPPGTGELNMDESFGLALQKTVPLSAMVRPRLAPTAYVS
SEQ ID NO: 81
Gossypium barbadense
KAB2074128
MPSFDTILLRDLVAAAFLFFITRYFIRRILSNPKRILPPGPNGWPVVGALPLLGSMPHV
ELAKLAKKYGPVMYLKMGTCNMVVASTPDAARAFLKTLDLNFSNRPSNAGATHIA
YDSQDMVFAEYGPRWKLLRKLSNLHMLGGRALEDWSQVRAVELGHMLRAMCESS
RKGEPVVVPEMLTYAMANMIGQVILSRRVFVTKGSESNEFKDMVVELMTSAGLFNI
GDFIPSIAWMDLQGIEGEMKKLHKRWDVLLTKMMKEHEETAYERKGKPDFLDIIMD
NRENSAGERLSLTNVKALLLNLFTAGTDTSSSIIEWALAEILKNPKILNKAHEEMDKV
IGRNRRLEESDVLKLPYLQAICKETFRKHPSTPLNLPRVSTQPCEINGYYIPKNTRLSV
NIWAIGRDPDVWGNPLDFTPERFLSGRFAKIDPRGNDFELIPFGAGRRICAGTRMGIV
LVEYILGTLLHSFDWMLPPGTGELNMDESFGLALQKTVPLSAMVRPRLAPTAYVS
SEQ ID NO: 82
Gossypium barbadense
KAB2057053
MESPSWVSYLIAWLATLALILLSLRFRPRRKLNLPPGPKPWPVIGNLDLIGSLPHRSIH
SLSQKYGPLMQLKFGSFPVVVASSVEMAKAFLKTHDVIFAGRPKIAAGEYTTYNYSD
ITWSPYGPYWRQARKMCMTELFSAKRLESYEYIRREEMKLLLKGFYESSGVPIVLKD
HLSDLSLNVISRMVFGKKYTEGTGENEIVTPKEFKEMLDELFLLNGVLDIGDSIPWLR
FLDLQGNIKRMKALSKKFDKFLEHVLDEHNARRRDVKDYVAKDMVDVLLQLADDP
NLDVKLERHGVKAFSQDLIAGGTESSAVTVEWAI
SEQ ID NO: 83
Gossypium barbadense
KAB2007859
MATPSWFSYLTPWLATLALILFSLRLCRRRKLNLPPGPKPWPIIGNLNLVGSLPHQSIH
ALSRKYGPIMQLKFGSFPVVVASSVEMAKAVLKTNDVIFTDRPKTAAGKYTTYNYS
DITWSPYGPYWRQARKICLTELFNAKRLESYQYIRREEMNLFLKRLYESSGTQIVLKD
HLSSLSLNVISRMVFGKKYTEGSGENEIVTPNEFKEMLDELFLLNGVLDIGDSIPWLSF
LDLQGYIKRMKALSKRLDRFLEHVLDEHNARREGAEDYVAKDMVDVLLQLSEDPN
LEVKLERHGVKAFTQDMIAGGTESSAVTVEWAISELLKKPEILAKATEELDMVIGRE
RWVEEKDVVSLPYIDSIAKETMRLHPVAPMLVPRVARQDCEIAGYDIPKGTRAFVNV
WTIGRDPSLWDNPNEFWPDRFMGKSIDVKGHDFELLPFGAGRRMCPGYPLGIKVIQA
SLANVLHGFTWKLPNNTTKDDLNMEEIFGLSTPKKYPLEAIAEPRLPLHMYS
SEQ ID NO: 84
Brassica napus cultivar Darmor_v5
BnaC09g47980D
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQEEVGTLMRELARANTKPVNLGQLVNMCVLNALGREMIGRRLF
GADADHKAEEFRSMVTEMMALAGVFNIGDFVPALDCLDLQGVAGKMKRLHKRFD
AFLSSILEEHETMKNGQDQKHTDMLSTLISLKGTDFDGEGGTLTDTEIKALLLNMFTA
GTDTSASTVDWAIAELIRHPEIMRKAQEELDSVVGRGRPINESDLSQLPYLQAVIKEN
FRLHPPTPLSLPHIASESCEINGYHIPKGSTLLTNIWAIARDPDQWTDPLSFRPERFLPG
GEKAGVDVKGNDFELIPFGAGRRICAGLSLGLRTIQLLTATLVHGFEWELAGGVTPE
KLNMEETYGITLQRAVPLVVHPKPRLDMSAYGLGSA
SEQ ID NO: 85
Brassica napus cultivar Darmor_v5
BnaA10g23330D
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWSDPLTFRPERFLPGGEKAGVDVKGNDFELIPFGAGRRICAGLSLGLRTIQLLT
ATLVHGFEWELAGGVTPEKLNMEETYGITLQRAVPLVVHPKLRLDMSAYGLGSA
SEQ ID NO: 86
Brassica napus cultivar ZS11
BnaA10G0256900ZS
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWSDPLTFRPERFLPGGEKAGVDVKGNDFELIPFGAGRRICAGLSLGLRTIQLLT
ATLVHGFEWELAGGVTPEKLNMEETYGITLQRAVPLVVHPKLRLDMSAYGLGSA
SEQ ID NO: 87
Brassica napus cultivar ZS11
BnaC09G0570900ZS
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKPRLDMSAYGLGSA
SEQ ID NO: 88
Brassica napus cultivar Gangan
BnaA10G0251000GG
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWSDPLTFRPERFLPGGEKAGVDVKGNDFELIPFGAGRRICAGLSLGLRTIQLLT
ATLVHGFEWELAGGVTPEKLNMEETYGITLQRAVPLVVHPKPRLDRSAYGLGSA
SEQ ID NO: 89
Brassica napus cultivar Gangan
BnaC09G0516100GG
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKPRLDMSAYGLGSA
SEQ ID NO: 90
Brassica napus cultivar Quinta
BnaA10G0248800QU
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKLRLDMSAYGLGSA
SEQ ID NO: 91
Brassica napus cultivar Quinta
BnaC09G0534300QU
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKPRLDMSAYGLGSA
SEQ ID NO: 92
Brassica napus cultivar Shengli
BnaA10G0220400SL
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWSDPLTFRPERFLPGGEKAGVDVKGNDFELIPFGAGRRICAGLSLGLRTIQLLT
ATLVHGFEWELAGGVTPEKLNMEETYGITLQRAVPLVVHPKLRLDMSAYGLGSA
SEQ ID NO: 93
Brassica napus cultivar Shengli
BnaC09G0396500SL
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKPRLDMSAYGLGSA
SEQ ID NO: 94
Brassica napus cultivar Tapidor
BnaA10G0249900TA
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQVTRTGNSDCFG
SEQ ID NO: 95
Brassica napus cultivar Tapidor
BnaC09G0550200TA
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQVTRTENSDCFG
SEQ ID NO: 96
Brassica napus cultivar Westar
BnaA10G0251800WE
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKLRLDMSAYGLGSA
SEQ ID NO: 97
Brassica napus cultivar Westar
BnaC09G0543700WE
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQAVIKENFRLHPPTPLSLPHIASESCEINGYHIPKGSTLLTNIWAIAR
DPDQWTDPLSFRPERFLPGGEKAGVDVKGNDFELIPFGAGRRICAGLSLGLRTIQLLT
ATLVHGFEWELAGGVTPEKLNMEETYGITLQRAVPLVVHPKPRLDMSAYGLGSA
SEQ ID NO: 98
Brassica napus cultivar Zheyou7
BnaA10G0234400ZY
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHEAMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQVTRTGNSDCFG
SEQ ID NO: 99
Brassica napus cultivar Zheyou7
BnaC09G0517700ZY
MTNLYLTILLPTFIFLIVLVLSRRRNNRLPPGPNPWPIIGNLPHMGPKPHQTLAAMVTT
YGPILHLRLGFADVVVAASKSVAEQFLKVHDANFASRPPNSGAKHMAYNYQDLVFA
PYGQRWRMLRKISSVHLFSAKALEDFKHVRQEEVGTLMRELARANTKPVNLGQLV
NMCVLNALGREMIGRRLFGADADHKAEEFRSMVTEMMALAGVFNIGDFVPALDCL
DLQGVAGKMKRLHKRFDAFLSSILEEHETMKNGQDQKHTDMLSTLISLKGTDFDGE
GGTLTDTEIKALLLNMFTAGTDTSASTVDWAIAELIRHPEIMRKAQEELDSVVGRGR
PINESDLSQLPYLQVTRTENSDCFG
SEQ ID NO: 100
Saccharum hybrid cultivar R570
AGT17103
MELPTWASFLGVVLATVMLLKAILGRRRRVYNLPPGPKPWPIIGNLNLMGALPHRSI
HELSRKYGPLMQLRFGSFPVVVGSSVDMAKFFLKTHDVVFTDRPKTAAGKYTTYNY
RDITWSPYGAYWRQARKMCLTELFSAKRLESYEYIRAAEVRALLRDLHSASGSGRA
VMLKDHLSTVSLNVITRMVLGKKYLDKDEVASAGSVTMTTPEEFKWMLDELFLLN
GVLNIGDSIPWLDWMDLQGYIKRMKKLSKMFDRFLEHVVEEHNQRRLREGKDFVA
KDMVDVLLQIADDPTLEVELNRESVKAFTQDLIAGGTESSAVTVEWAISELLKKPEVI
VKATEELDRVIGRGRWVTEKDIPSLPYVDAIVKETMRLHPVAPMLVPRLSREDTTVA
GYDIPAGTRVLVSVWSIGRDPALWDAPEEFMPERFLSSRLDVKGQDYELLPFGSGRR
MCPGYSLGLKVIQVSLANLLHGFSWSLPYGVTKEELSMEEIFGLSTPRKFPLEAVVEP
KLPAHLYAEP
SEQ ID NO: 101
Saccharum hybrid cultivar R570
AGT17101
MELSAWASVFAVVFTTVVYLGAVHARRRRACNSLPGPKPWPIIGNFNLLGALPHRSL
DALSKRHGPLMRVQFGSFPVVIASSVDMAKFFLKTHDSVFIDRPKMAAGKYTTYNY
SNIAWSPYGAYWRQARKICADELFSARRLESFEHVRQEEVHALLRTLHGTAGQVVP
LKECLSTMSLNIITRMVLGRKCVDKEVVASGGGSVTTWKEFRWMLDELFLLNGVLN
IGDWIPWLSWLDLQGYVRRMKRVGRMFNQFMENVVEEHNERRLREGDAFVPQDM
VDRLLQLADDPSLDVKLTRDSVKAFTQSAAVIVEWAISELLKNPDVFAKATEELDGV
IGRDRWVTEKDIPHLPYMDAIVKETMRLHMVVPLLSPRLSREDTSVGGYDIPAGTRV
LINAWTISRDPALWDAPEEFRPERFVGSKIDVKGQDFELLPFGSGRRMCPGYSLGLKV
IQVTLVNLLHGFAWRLPDGMTEEELSMEEVFGLSTPRKFPLQAVVEPKLPARMYTA
SEQ ID NO: 102
Saccharum hybrid cultivar R570
AGT16621
MDATQDSPLFLFPAAATLLSPLLAVLLVVLSLLWLYPGGPAWALIISRSRATPPPGTP
GVVTALAGPAAHRTLASLSQSLPGGGSALLAFSVGLTRLVVASQPDTARELLASAAF
ADRPVKDAARGLLFHRAMGFAPSGDYWRALRRISSAYLFSPRSVSATAPRRVAIGER
MLRDLSAAATGGGGGGEVVMRRVLHAASLDHVMATVFGARYDADSAEGAELEEM
VKEGYDLLGLFNWGDHLPLLRWLDLQGVRRRCRSLVSRVNVFVARIIEEHRQKKKD
DAANGESAAGDFVDVLLGLEGEEKLSDSDMIAVLWEMIFRGTDTVAILLEWVMAR
MVLHPGIQSKAQAELDAVVGRGRAVSDADVARLPYLQRVVKETLRVHPPGPLLSW
ARLAVHDAVVGGHLVPAGTTAMVNMWAIAHDPVVWAEPSAFRPERFEEEDVSVLG
GDLRLAPFGAGRRVCPGKTLALATVHLWLAQLLHRFQWAPADGGVDLAERLGMSL
EMEKPLVCKPTPRW
SEQ ID NO: 103
Saccharum hybrid cultivar R570
AGT16132
MDATQDSPLFLFPAAATLLSPLLAVLLVVLSLLWLYPGGPAWALIISRSRATPPPGTP
GVVTALAGPAAHRTLASLSQSLPGGGSALLAFSVGLTRLVVASQPDTARELLASAAF
ADRPVKDAARGLLFHRAMGFAPSGDYWRALRRISSAYLFSPRSVSATAPRRVAIGER
MLRDLSAAATGGGGGGEVVMRRVLHAASLDHVMATVFGARYDADSAEGAELEEM
VKEGYDLLGLFNWGDHLPLLRWLDLQGVRRRCRSLVSRVNVFVARIIEEHRQKKKD
DAANGESAAGDFVDVLLGLEGEEKLSDSDMIAVLWEMIFRGTDTVAILLEWVMAR
MVLHPGIQSKAQAELDAVVGRGRAVSDADVARLPYLQRVVKETLRVHPPGPLLSW
ARLAVHDAVVGGHLVPAGTTAMVNMWAIAHDPVVWAEPSAFRPERFEEEDVSVLG
GDLRLAPFGAGRRVCPGKTLALATVHLWLAQLLHRFQWAPADGGVDLAERLGMSL
EMEKPLVCKPTPRW
SEQ ID NO: 104
Saccharum hybrid cultivar R570
AGT17102
MELPTWASFLGVVLATVMLLKAILGRRRRVYNLPPGPKPWPIIGNLNLMGALPHRSI
HELSRKYGPLMQLRFGSFPVVVGSSVDMAKFFLKTHDVVFTDRPKTAAGKYTTYNY
RDITWSPYGAYWRQARKMCLTELFSAKRLESYEYIRAAEVRALLRDLHSASGSGRA
VMLKDHLSTVSLNVITRMVLGKKYLDKDEVASAGSVTMTTPEEFKWMLDELFLLN
GVLNIGDSIPWLDWMDLQGYIKRMKKLSKMFDRFLEHVVEEHNQRRLREGKDFVA
KDMVDVLMQIADDPTLEVELDRESVKAFTQDLIAGGTESSAVTVEWAISELLKKPEV
IAKAT
SEQ ID NO: 105
Saccharum hybrid cultivar R570
AGT16178
MSAGYFKNKHSLGARSVPVHAGSCYASSQGPLWFLVVPLMLELLPFICRRLHHRPN
AGDDDRKRSKPLLPSPPGRLPVIGHLHLIGDLPHVSLRDLATKHDHGGGLMLLQLGT
VPILVVSSPHAAQAVLRTHDHVFASRPAPKVLHNFLYGSSTIAFGPYGEHWRKVRKL
VTTRLFTVKKVRQVMAKLKKAMATGMAVEMSETMNTFANEIMCRVLSGKFFKEDS
RNKTFRELIEMNVALYAGFSLENYFPGLVNSLGIFTRMVSRKADETHERWDDVLENII
SDHERRAEQEESADFVDLMLSVQQEYDLFDAGTGTSYLTLELAMAELMRHPHIMTK
LQAEVRNKIPNGQEMVREEDLASMAYLRAVVKETLRLHPPAPLFLPYQSMVDCEID
GYTIPSGTRVIINSWAVCRHVESWEKAEEFMPERFMDGGSAAAVDFKGNDFQFIPFG
AGRRMCPGINFGLAIVEIMLANLIVLF
SEQ ID NO: 106
Saccharum hybrid cultivar R570
AGT16989
MDEFLYQSLLLSVVALVKLAFIKRRPRLPPGPWKLPVIGSMHHLINVLPHRALRDLA
AVHGPLMMLQLGQTALVVASSKETARAVLKTHDTNFATRPKLLAGQIVGYEWVDIL
FAPSGDYWRKLRQLCAAEILSPKRVLSFRHIREDEVMLRVEEIRAAGPSTPVNLSVMF
HSITNSVVSRAATRAATKAVVGLASGFNIPDLFPGWTTVLAKLTGMTCSLQDIHKTV
HTILEEIIQERKAIRDEKISSGAEDIDENLVDVLLGLQEKGGFGFQLNNSIIKAIILDMFA
GGTGTSGSAMEWGMSELMRNPEVMKKLQPAGADQGGSIEECELDGYTIPAKSRVII
NAWAIGRDPRYWEAADEFKPERFEDGARDFTGSSYEFLPFGSGRRMCPGFNYGLAS
MELAFVGLLYHFDWSLPDGVEEVDMGEAPGLGVRRRTPLLLCATPFVPVDA
SEQ ID NO: 107
Saccharum hybrid cultivar R570
AGT16177
MLLQLGTVSNLVVSSPRAARAVLRTHDHVFASRPTTKVLHNFLYGSSTIAFGPYGEH
WRKVRKLVTTHLFTVKKVNSFCHARQEEVRLVMAKLKKAMATGMEVDMSETMNT
FANDIMCCVVSGKLFREDGRNKTFRELIEMNSALYAGFSLENYFPRLVNSLGIFTRFV
SRKADKTHERWDEVLENIISDHERQSFNYRHGDRAEQEEGTDFVDVMLSVQQEYGIS
RDHIKAVLMDMFDAGTVTSSLVLELAMAELMRHPHLMSKLQAEVRNKTPNGEEMV
KQENLASMSYLRAVVKETLRHLESWEKAEKFMPERFMDGGSAATIDLKGNDFQFIP
FGAGRRMCPGINFGLVTVEIMLANLMYCFDWGLPAGMDKKDIDMTEVFGLTVHRK
EKLMLVPKLPGTASYA
SEQ ID NO: 108
Saccharum hybrid cultivar R570
AGT16905
MSMHQPTSAAATQLHHAAMEASLMSLSFLQLAFTAVAAIAALAVAVAVTRYNRRY
MGLRLPPGPPVWPVVGNLFQVAFSGKLFIHYIRDLRKEYGPILTLRMGERTLVIISSAE
LAHEALVEKGREFASRPRENTTRNIFSSNKFTVNSAVYGAEWRSLRRNMVSGMLSTS
RLREFAHARRRAMDRFVSRMRAEALASPDGASVWVLRNARFAVFCILLDMTFGLLD
LHEEHIVHIDAVMKRVLLAVGVRMDDYLPFLRPFFWRHQRRALAVRREQVDTLLPL
ISRRRAILRDMKSSSPPDPNVAAPFSYLDSVLDLHIEGRDGTPTDDELVTLCAELINGG
TDTTATAIEWGMARIVDNPSIQARLHEEIMQQVGDARPVDDKDTDAMPYLQAFVKE
LLRKHPPTYFSLTHAAVQPGSKLAGYDVPVDANLDIFLPTISEDPKLWDRPTEFDPDR
FVSGGEMGDMTGSGGIRMIPFGAGRRICPGLAMGTTHIALMVARMVQAFEWRAHPS
QPPLDFKDKVEFTVVMDRPLLAAVKPRNLSF
SEQ ID NO: 109
Saccharum hybrid cultivar R570
AGT16500
MSMHQPTSAAATQLHHAAMEASLMSLSFLQLAFTAVAAIAALAVAVAVTRYNRRY
MGLRLPPGPPVWPVVGNLFQVAFSGKLFIHYIRDLRKEYGPILTLRMGERTLVIISSAE
LAHEALVEKGQEFASRPRENTTRNIFSSNKFTVNSAVYGAEWRSLRRNMVSGMLSTS
RLREFAHARRRAMDRFVSRMRAEAAASPDGASVWVLRNARFAVFCILLDMTFGLL
DLHEEHIVHIDAVMKRVLLAVGVRMDDYLPFLRPFFWRHQRRALAVRREQVDTLLP
LISRRRAILRDMKSSSPPDPNVAAPFSYLDSVLDLHIEGRDGAPTDDELVTLCAELING
GTDTTATAIEWGLARIVDNPSIQARLHEEIMHQVGDARPVDDKDTDAMPYLQAFVK
ELLRKHPPTYFSLTHAAVQPGSKLAGYDVPVDANLDIFLPTISEDPKLWDRPTEFDPD
RFVSGGEMGDMTGSGGIRMIPFGAGRRICPGLAMGTTHIALMVARMVQAFEWRAHP
SQPPLDFKDKVEFTVVMDRPLLAAVKPRNLS
SEQ ID NO: 110
Saccharum hybrid cultivar R570
AGT16853
MAKLKKAMATGMEVDMSETMNTFANDIMCCVVSGKLFREDGRNKTFRELIEMNSA
LYAGFSLENYFPRLVNSLGIFTRFVSRKADKTHERWDEVLENIISDHERQSFNYRHGD
RAEQEEGTDFVDVMLSVQQEYGISRDHIKAVLMDMFDAGTVTSSLVLELAMAELM
RHPHLMSKLQAEVRNKTPNGEEMVKQENLASMSYLRAVVKETLRFMDGGSAATID
LKGNDFQFIPFGAGRRMCPGINFGLVTVEIMLANLMYCFDWGLPAGMDKKDIDMTE
VFGLTVHRKEKLMLVPKLPGTASYA
SEQ ID NO: 111
Saccharum hybrid cultivar R570
AGT17443
MGKITPQTQNSQTWISLIFDEMSDMSMMTASDRVAPYIHASLSLHWYGPIFKTNLVG
QPMVVSADPEVNRFIFQQEGKLFRSWYPETANIIIGEKTIDEFNGPTQKFVRNIISRLFG
LEYLKQDLIPELEKDIRDTFAEWTTKPSIDVHDSTPDVIFVLVAKKMLGLHPSESRELR
KNYSSFLQGLISFPIYFPGTTFYQCMQGKNNMLNLMSNLLRKRLSMPEKHGDILDLM
VEELQSENPTIDDKFATDTLSAILFTSFVTLSPNLTLAFKFLSDNPAVLDALKEEHDTIL
RNRKDSSSGFTWEEYKSLTFTTMVINELMRMSNPTPGIFRKTLTDVQVNGYTIPAGW
MVMMSPMAVHLNPAFFEDPLDFNPWRWLDESKRNAQKNFVPFGLGTRACPAAEFS
KLFIALFLHVLVTKYRLLLAHDKSIYTFVMLAAL
SEQ ID NO: 112
Saccharum officinarum
AWA44852
METLHAHDELFSCVVLVLVTTITILYLKQLLLAAFERRAGSPSLPCPRGLPLIGNLHQL
GTAPHDSLAALAAKHAAPLMLLRLGSVPTLVVSTADALRAVFQPNDRAMSGRPALY
AATRITYGLQDIVFSPPDGAFWRAARRASLSELLSAPRVRSFRDVREGEAAALVAAIT
DMSGSGSPVNLSEEVMATSNKILRRVAFGDGGGEESIEAGKVLDETQKLLGGFFVAD
YMPWLGWLDALRGLRRRLERNFHELDAFYEKVIDDHLSKRGAGADASKGEDLVDV
LLRLHGDPAYQSTFNSRDQIKGILTDMFIAGTDTAAATVEWTMTELVRHPDILAKAQ
KEVRAAVVGKDIVLESDLPRLKYLKQVIRESMRVHPPVPLLVPRETIEPCTVYGCEIP
ARTRVFVNAKAIGQDPDAWGPDAARFVPERHEEIADLSDHKPWHDSFSLVPFGVGR
RSCPGVHFATSVVELLLANLLFCFDWRAPHGEVDLEQETGLTVHRKNPLVLVAERR
GVL
SEQ ID NO: 113
Saccharum officinarum
AWA44857
MARALVAMALRFLRDYVRASDLAVAAAVLFVCSAVRSRLSSRPGEPMLWPVVGIIP
TLFAHLAIGDVYDWGAVVLSRCRGTFPYRGTWGGGSSGVITSVPANVEHVLKDNFD
NYPKGPYYRERFAELLGDGIFNADGDSWRAQRKAASAEMHSARFLRFSAATIERLV
CGRLVPLLETLSERGHSVDLQDVLLRFAFDNICAAAFGVEAGCLADGLPDVPFARAF
ERATELSLTRFYTPPFIWKPKRLLCVGSERALVEAARAVREFAERTVADRRAELCKIG
DLAGRCDLLSRLMSSSPPPADAGAGLAAGYSDEFLRDFCISFILAGRDTSSVALTWFF
WLLASHPDVEARVLDDIARVGGGDVGAMDYLHAALTESMRLYPPVPVDFKEALED
DVLPDGTLVRARQRVIYFTYAMGRDKATWGPDCLEFRPERWLNKSGAFAGGAESP
YKYVVFNAGPRLCVGKRFAYTQMKTVAAAVLARFRVEVVPGQEVKPKLNTTLYM
KSGLMVRFVAREQRHELGHPVPAAADDAGGCSLH
SEQ ID NO: 114
Saccharum officinarum
AWA44838
MARALVAMALRFLRDYVRASDLAVAAAVLFVCSAVRSRLSSRPGEPMLWPVVGIIP
TLFEHLAIGDVYDWGAAVLSRCRGTFPYRGTWGGGSSGVITSVPANVEHVLKDNFD
NYPKGPYYRERFAELLGDGIFNADGDSWRAQRKAASAEMHSARFLRFSAATIERLV
RGRLVPLLETLSERGHSVDLQDVLLRFAFDNICAAAFGVEAGCLADGLPDVPFARAF
ERATELSLTRFYTPPFIWKPKRLLCVGSERALVEAARAVREFAERTVADRRAELCKIG
DLAGRCDLLSRLMSSSPPPADAGAGLAAGYSDEFLRDFCISFILAGRDTSSVALTWFF
WLLASHPDVEARVLDDIARVGGGDVGAMDCLHAALTESMRLYPPVPVDFKEALED
DVLPDGTLVRARQRVIYFTYAMGRDKATWGPDCLEFRPERWLNKSGAFAGGAESP
YKYVVFNAGPRLCVGKRFAYTQMKTVAAAVLARFRVEVVPGQEVKPKLNTTLYM
KSGLMVRFVAREQRHELGHPVPAAADDAGGCSLH
SEQ ID NO: 115
Saccharum officinarum
AWA44954
MARALVAMALRFLRDYVRASDLAVAAAVLFVCSAARSRLSSRPGEPMLWPVVGIIP
TLFAHLAIGDVYDWGAAVLSRCRGTFPYRGTWGGGSSGVITSVPANVEHVLKANFD
NYPKGPYYRERFAELLGDGIFNADGDSWRVQRKAASSEMHSARFLQFSAATIERLVR
GRLVPLLETLSERGADDAVVDLQDVLLRFAFDNICAAAFGVEAGCLADGLPDVPFA
HAFERATELSLTRFYTPPFIWKPKRLLCVGSERALVEAARAVREFAERTVADRRAEL
RKVGDLAGRCDLLSRLMSSSPPPADAGAGLAAGYSDEFLRDFCISFILAGRDTSSVAL
TWFFWLLAFHPDVEARVLDDIALAGGDVGATDYLHAALTESMRLYPPVPVDFKEAL
EDDVLPDGTLVRARQRVIYFTYAMGRDKATWGPDCLEFCPERWLNKSGAFAGGAE
SPYKYVVFNAGPRLCVGKRFAYTQMKTVAAAVLARFRVEVVPGQEVKPKLNTTLY
MKSGLMVRFVAREQRHELGHPVPAAADDAGGCSLH
SEQ ID NO: 116
Glycine max
Glyma.06G202300
MSPLIVALATIAAAILIYRIIKFITRPSLPLPPGPKPWPIVGNLPHMGPVPHHSLAALARI
HGPLMHLRLGFVDVVVAASASVAEQFLKIHDSNFSSRPPNAGAKYIAYNYQDLVFAP
YGPRWRLLRKLTSVHLFSGKAMNEFRHLRQEEVARLTCNLASSDTKAVNLGQLLNV
CTTNALARAMIGRRVFNDGNGGCDPRADEFKAMVMEVMVLAGVFNIGDFIPSLEW
LDLQGVQAKMKKLHKRFDAFLTSIIEEHNNSSSKNENHKNFLSILLSLKDVRDDHGN
HLTDTEIKALLLNMFTAGTDTSSSTTEWAIAELIKNPQILAKLQQELDTVVGRDRSVK
EEDLAHLPYLQAVIKETFRLHPSTPLSVPRAAAESCEIFGYHIPKGATLLVNIWAIARD
PKEWNDPLEFRPERFLLGGEKADVDVRGNDFEVIPFGAGRRICAGLSLGLQMVQLLT
AALAHSFDWELEDCMNPEKLNMDEAYGLTLQRAVPLSVHPRPRLAPHVYSMSS*
SEQ ID NO: 117
Glycine max
Glyma.05G021800
MSTWVIGFATIIAAVLIYRVLKPISRPSSSLPLPPGPRPWPIVGNLPHMGPAPHQGLAN
LAQTHGPLMHLRLGFVDVVVAASASVAEQFLKIHDANFCSRPLNFRTTYLAYNKQD
LVFAPYGPKWRFLRKLTTVHMFSAKAMDDFSQLRQEEVARLTCKLARSSSKAVNLR
QLLNVCTTNALTRIMIGRRIFNDDSSGCDPKADEFKSMVGELMTLFGVFNIGDFIPAL
DWLDLQGVKAKTKKLHKKVDAFLTTILEEHKSFENDKHQGLLSALLSLTKDPQEGH
TIVEPEIKAILANMLVAGTDTSSSTIEWAIAELIKNSRIMVQVQQELNVVVGQDRLVT
ELDLPHLPYLQAVVKETLRLHPPTPLSLPRFAENSCEIFNYHIPKGATLLVNVWAIGR
DPKEWIDPLEFKPERFLPGNEKVDVDVKGNNFELIPFGAGRRICVGMSLGLKIVQLLI
ATLAHSFDWELENGTDPKRLNMDETYGITLQKAMPLSVHPHPRLSQHVYSSSSL*
SEQ ID NO: 118
Glycine max
Glyma.05G021900
MSAWVIAFATVVAATLIYRLFKLITVPSLPLPPGPRPWPIVGNLPHMGPAPHQGLAAL
AQTHGPLMHLRLGFVDVVVASSASVAEQFLKIHDANFCSRPCNSRTTYLTYNQQDL
VFAPYGPRWRFLRKLSTVHMFSAKAMDDFRELRQEEVERLTCNLARSSSKVVNLRQ
LLNVCTTNILARIMIGRRIFSDNSSNCDPRADEFKSMVVDLMVLAGVFNIGDFIPCLD
WLDLQGVKPKTKKLYERFDKFLTSILEEHKISKNEKHQDLLSVFLSLKETPQGEHQLI
ESEIKAVLGDMFTAGTDTSSSTVEWAITELIKNPRIMIQVQQELNVVVGQDRLVTELD
LPHLPYLQAVVKETLRLHPPTPLSLPRFAENSCEIFNYHIPKGATLLVNVWAIGRDPK
EWIDPLEFKPERFFPGGEKDDVDVKGNNFELIPFGAGRRICVGMSLGLKVVQLLIATL
SEQ ID NO: 119
Glycine max
Glyma.05G022100
MSPWVIAVATIVAAILIYRVLKHIAGPSLPLPPGPRPWPIVGNLPHMGPAPHQGLAAL
AKTHGPLMHLRLGFVHVVVAASAAVAEQFLKVHDANFCNRPYNFRTTYMTYNKK
DIAFYPYGPRWRFLRKICTVHMFSGKAMDNFSQLRQEEVERLACNLTRSNSKAVNL
DWLDLQGLKTKTKKLHKRFDILLSSILEEHKISKNAKHQDLLSVLLSLKETPQEGHEL
VEEEIKSILGDMFTAGTDTSLSTIEWAIAELIKNPKIMIKVQQELTTIVGQNRLVTELDL
PHLPYLNAVVKETLRLHPPTPLSLPRVAEESCEIFNYHIPKGATLLVNVWAIGRDPKE
WLDPLEFKPERFLPGGEKADVDIRGNNFEVIPFGAGRRICVGMSLGIKVVQLLIASLA
HAFDWELENGYDPKKLNMDEAYGLTLQRAVPLSIHTHPRLSQHVYSSLSL*
SEQ ID NO: 120
Glycine max
Glyma.17G077700
MYLRLGFVDVVVAASASVAEQFLKVHDANFSSRPLNSMTTYMTYNQKDLAFAPYG
PRWRFLRKISSVHMFSVKALDDFRQLRQEEVERLTSNLASSGSTAVNLGQLVNVCTT
NTLARVMIGRRLFNDSRSSWDAKADEFKSMVVELMVLNRVFNIGDFIPILDRLDLQG
VKSKTKKLHKRFDTFLTSILEEHKIFKNEKHQDLYLTTLLSLKEAPQEGYKLDESEIK
AILLDMFTAGTDTSSSTIEWAIAELIRNPRVMVRVQQEMDIVVGRDRRVTELDLPQLP
YLQAVVKETFRLHPPTPLSLPRVATESCEIFDYHIPKGTTLLVNIWAIGRDPNEWIDPL
EFKPERFLLGGEKAGVDVMGTNFEVIPFGAGRRICVGMGLGLKVVQLLTATLAHTF
CYP93G1 orthologs
SEQ ID NO: 121
Oryza sativa ssp. japonica
LOC_Os04g01140
MASLMEVQVPLLGMGTTMGALALALVVVVVVHVAVNAFGRRRLPPSPASLPVIGH
LHLLRPPVHRTFHELAARLGPLMHVRLGSTHCVVASSAEVAAELIRSHEAKISERPLT
AVARQFAYESAGFAFAPYSPHWRFMKRLCMSELLGPRTVEQLRPVRRAGLVSLLRH
VLSQPEAEAVDLTRELIRMSNTSIIRMAASTVPSSVTEEAQELVKVVAELVGAFNADD
YIALCRGWDLQGLGRRAADVHKRFDALLEEMIRPERFLAGGGGEGVEPRGQHFQFM
PFGSGRRGCPGMGLALQSVPAVVAALLQCFDWQCMDNKLIDMEEADGLVCARKHR
LLLHAHPRLHPFPPLL*
SEQ ID NO: 122
Oryza sativa ssp. indica
OsR498G0407413200
MASLMEVQVPLLGMGTTMGALALALVVVVVVHVAVNAFGRRRLPPSPASLPVIGH
LHLLRPPVHRTFHELAARLGPLMHVRLGSTHCVVASSAEVAAELIRSHEAKISERPLT
AVARQFAYESAGFAFAPYSPHWRFMKRLCMSELLGPRTVEQLRPVRRAGLVSLLRH
VLSQPEAEAVDLTRELIRMSNTSIIRMAASTVPGSVTEEAQELVKVVAELVGAFNAD
DYIALCRGWDLQGLGRRAADVHKRFDALLEEMIRHKEEARMRKKTDTDVGSKDLL
DILLDKAEDGAAEVKLTRDNIKAFIIDVVTAGSDTSAAMVEWMVAELMNHPEALRK
VREEIEAVVGRDRIAGEGDLPRLPYLQAAYKETLRLRPAAPIAHRQSTEEIQIRGFRVP
AQTAVFINVWAIGRDPAYWEEPLEFRPERFLAGGGGEGVEPRGQHFQFMPFGSGRR
GCPGMGLALQSVPAVVAALLQCFDWQCMDNKLIDMEEADGLVCARKHRLLLHAH
PRLHPFPPLL*
SEQ ID NO: 123
Brachypodium distachyon
Bradi5g02460
MAMAASSMEQLLQVDPAMATYSILAIALVTAVLVLINRIGGNGAGKQRRHGLPPSPR
RLPVIGHLHLLRPPVHRTFQELASGLGAPLMHIRLGSTHCLVASSAAAATELIRSHEG
KISERPLTAVARQFAYGSDSGFAFAPYGPHWRAMKRLCMSELLGPRTVELLRPVRRA
GLVSLLHTVIRKSPEPVDLTAELIRMSNASIIRMMASTVPGSVTEEAQALVKAVAELV
GAFNVEDYIAVCRGWDLQGLGKRAADVHRRFDALLEDMIAHKEEARAAKKAIRGE
DDQEPETKKTMAESKDLIDILLDKMEDENAAEETKLTREKIKAFTIDVVTAGSDTSA
AMVEWMLAELMNHPECLRKVRDEIDAVVGSNRITGEADIANLPYLQAAYKETLRLR
PAAPIAHRQSTEDMELATGGCFTVPVGTAVFINLWAIGRDPEHWGQTALEFRPERFM
LGGESEKLEPRGQHFQYLPFGSGRRGCPGMGLALQSVPAVVAALVQCFHWTVVPKA
GEEKAVIDMEESDGLVRARKHPLLLRASPRLNPFPAVV*
SEQ ID NO: 124
Triticum aestivum
TraesCS2D02G043500
MCSPEVAGGTLAAMATASSMQQPALLLLRQLTQDPVTASLLAVALATAVLMIAALS
RGGGRKPRLPPSPRGFPVIGHLHLVRPPVHRTFHDLAARLGPLMHIRLGSTHCVVASS
AGVAAELIRTHEGKISERPLTAVARQFAYGDDGFAFAPYGPHWRSMKRLCMSELLG
PRTVEQLRPVRRAGLVSLLQSVLHQASGAEAVDLTAALIRLSNTSIIRMMASTVPGSV
TGEAQALVKAVAELVGAFNVEDYIAVCRGWDLQGLGRRAADVHRRFDALLEQMIR
HKEEAREARKMRGGAEGETPEKKTATGTTTESSKDLLDILLDKLEDDAAAEVKLTR
KKIKAFVIDVVTAGSDTSAAMVEWMLAELMNHPECLRKVREEIDAVVGRDRIAGEG
DVASLPYLQAAYKETLRLRPAAPIAHRQSTEEMVVTAAGGVGGFTVPAGTAVFMNL
WSIARDPANWDAPLEFRPERFMAGGRNEALDPRGQHFQYLPFGSGRRGCPGMGLAL
QSVPAVVAALVQCFDWAVDGDAKKIDMEEADGLVCARKHPLLLRPSPRLSPFPAVV
*
SEQ ID NO: 125
Triticum aestivum
TraesCS2A02G044900
MATASSMQQPALLLLRQLMQDPVIASLLAVALATVIMLIGAVSRGGGRKPRLPPSPR
GFPVIGHLHLVRPPVHRTFHDLAARLGPLMHIRLGSTHCVVASSAGVAAELIRTHEG
KISERPLTAVARQFAYGDDGFAFAPYGPHWRSMKRLCMSELLGPRTVEQLRPVRR
AGLVSLLQSVLHQASGAEAVDLTAALIRLSNTSIIRMMASTVPGSVTEEAQALVKAV
AELVGAFNVEDYIAVCRGWDLQGLRRRAADVHRRFDALLEEMIRHKEEAREARKM
RGGGEGETPEKKTATGTTTESSKDLLDILLDKLEDDAAAEVKLTRKKIKAFVIDVVTA
GSDTSAAMVEWMLAELMNHPECLRKVRAEIDAVVGRDRIAGEGDVASLPYLQAAY
KETLRLRPAAPIAHRQSTEEMVISAAGGGVGGFTVPAGTAVFMNLWSIARDPANW
DAPLEFRPERFMAGGRNEALDPRGQHFQYLPFGSGRRGCPGMGLALQSVPAVVA
ALVQCFDWAVDGDGKKIDMEEADGLVCARKHPLLLRPSPRLSPFPAVV*
SEQ ID NO: 126
Triticum aestivum
TraesCS2B02G057100
MAMASSMQQPALLLLRQLTQDPVTASLLAAALATAVLMIAAVRRGGGRKPRLPPSP
RGFPVIGHLHLVRPPVHRTFHDLAARLGPLMHIRLGSTHCVVASSAGVAAELIRTHE
GKISERPLTAVARQFAYGDDGFAFAPYGPHWRSMKRLCMSELLGPRTVEQLRPVRR
AGLVSLLQSVLHQASGAEAVDLTAALIRLSNTSIIRMMASTVPGSVTEEAQELVKAV
AELVGAFNVEDYIAVCRGWDLQGLGRRAADVHRRFDALLEEMIRHKEEAREARRM
RGGGEGETPEKKTATGTTTESSKDLLDILLDKLEDDAAAEVKLTRKKIKAFVIDVVT
AGSDTSAAMVEWMLAELMNHPECLRKVRSEIDAVVGRDRIAGEGDVASLPYLQAA
YKETLRLRPAAPIAHRQSTEEMVVTAAGGFTVPAGTAVFINLWSIARDPANWDAPLE
FRPERFLAGGRNEALDPRGQHFQYLPFGSGRRGCPGMGLALQSVPAVVAALVQCFD
WAVPADIDGKKIDMEEADGLVCARKHPLLLRPSPRLSPFPAVV*
SEQ ID NO: 127
Triticum turgidum
TRITD2Av1G010200
MCDPEVAGATLAAMATASSMQQPALLLRQLTQDPVTASLLAAALATAVLMIAAVS
RGGGRKPRLPPSPRGFPVIGHLHLVRPPVHRTFHDLAARLGPLMHIRLGSTHCVVASS
AGVAAELIRAHEGKISERPLTAVARQFAYGDDGFAFAPYGPHWRSMKRLCMSELLG
PRTVEQLRPVRRAGLVSLLQSVLHRASGAEAVDLTAALIRLSNTSIIRMMASTVPGSV
TEEAQALVKAVAELVGAFNVEDYIAVCRGWDLQGLRRRAADVHRRFDALLEEMIR
HKEEAREARKMRGGAEGETPEKKTATGTTTESSKDLLDILLDKLEDDAAAEVKLTR
KKIKAFVIDVVTAGSDTSAAMVEWMLAELMNHPECLRKVRAEIDAVVGRDRIAGEG
DVASLPYLQAAYKETLRLRPAAPIAHRQSAEEMVISAAGGFTVPAGTAVFINLWSIAR
DPANWDAPLEFRPERFMAGGRNEALDPRGQHFQYLPFGSGRRGCPGMGLALQSVPA
VVAALVQCFDWAVDGDAEKIDMEEADGLVCARKHPLLLRPSPRLSPFPAVV*
SEQ ID NO: 128
Triticum turgidum
TRITD2Bv1G013440
MAMASSMQQPALLLLRQLTQDPVTASLLAAALATAVLMIAAVRRGGGRKPRLPPSP
RGFPVIGHLHLVRPPVHRTFHDLAARLGPLMHIRLGSTHCVVASSAGVAAELIRTHE
GKISERPLTAVARQFAYGDDGFAFAPYGPHWRSMKRLCMSELLGPRTVEQLRPVRR
AGLVSLLQSVLHQASGAEAVDLTAALIRLSNTSIIRMMASTVPGSVTEEAQELVKAV
AELVGAFNVEDYIAVCRGWDLQGLGRRAADVHRRFDALLEEMIRHKEEAREARRM
RGGGEGETPEKKTATGTTTESSKDLLDILLDKLEDDAAAEVKLTRKKIKAFVIDVVT
AGSDTSAAMVEWMLAELMNHPECLRKVRSEIDAVVGRDRIAGEGDVASLPYLQAA
YKETLRLRPAAPIAHRQSTEEMVVTAAGGFTVPAGTAVFINLWSIARDPANWDAPLE
FRPERFLAGGRNEALDPRGQHFQYLPFGSGRRGCPGMGLALQSVPAVVAALVQCFD
WAVPADIDGKKIDMEEADGLVCARKHPLLLRPSPRLSPFPAVV*
SEQ ID NO: 129
Setaria italica
Seita.1G019400
MAMETEQPLPILLSADSVAVLAVGTLLALALNHLVSSWRSARRLPPSPPGLPVIGHLH
LLRPPAHRTFHELAGKLGPLMHIRLGSTHCVVAGSADVARELIHRHDAAISGRPVTA
LARLFSYSSAGFAFTPYSPRWRFLRRLCVSEVLSPRTVEQLRPVRRAALAPLLRAVLA
ASERGEAADVTGELVRFANASIIRMVASDAPGSVADEAQGLVKAVTELIGAFNVEDY
VPLCRGWDLQGLRSTAAGVHRRFDALLEQMIRHKEEARERGRSCGAIYELEHEQED
EKGSAPATRKRNKDLLDILLEKAEDEAAEVKLTRENIKAFITDVVTAGSDSSAATVE
WMLAELVNHPEVMRKVREEIDAVVTGDCRIVGEADLPRLPYLQAAFKETLRLHPGA
PIAHRVSTAEISVRGFMVPPRTAVFINVWAIGRDPAFWEDPTAFRPERFMPGGAAAG
LEPQPRGHHFQFMPFGGGRRGCPGVGLAQQSVPAVLAALVQCFDWAVADGETGLV
DMEESDVGLVCARKHPLLLRPTPRLNPFPSVV*
SEQ ID NO: 130
Cenchrus americanus
Pgl_GLEAN_10038007
MEMEQPLPMLLSADTVAIMAVVTFLALAVNHLVSSWLSSPRRRLPPSPPGLPVIGHL
HLLRLPAHRTFHELAGKLGPLMHLRLGSTHCVVASSADVARELILRHDAAISGRPVT
ALARLFSYGSVGFAFTPYSPRWRFLRRLCVSELVRFASASIIRMVASDAPGNVSDEAQ
GLVKSVTELIGAFNVEDYVPLCRGWDLQGLRRTADGVHRRFDALLEQMIRHKEEAR
ERARSDMAEHEQHDKKDASASAAPTTRKRNKDLLDILLEKAEDDEAEVKLTRENIK
AFITDVVTAGSDSSAATVEWMLAELVNHPEAMRKVREEIDAAVGEDSRIVSEADLPR
LPYLQAAFKETLRLHPGAPIAHRVSSAEEMAVGGFTVPPRTAXXXXXXXXXXXXXX
XXXXXXXXXXXXXXXXXXXXXXXXXXXXXXXXXXXXXXXXXXXXXXXXXXXX
XXXXXXXXXXXXXXXXXXXPGVGLAQQSVPAVLAALVQCFDWAAVVDGEMSPT
GSLVNMEESDVGLVCARKHSLLLRPTARLNPFPAVV*
SEQ ID NO: 131
Cenchrus americanus
Pgl_GLEAN_10012559
MVASTVPGRVADEAQELVKDVAELVGAFNADDYIALCRGWDLQGLRRRAADVHR
RFDALLEEILRHKEDAREARKLLMLDGGDGARKKKEAATATTAHKDLLDILMDKAE
DKTAEANLTRDNIKAFIIDVVTAGSDTSAAMVEWMLAELMNHPEALRKVVAEIDGV
VGGERIAGEADLPQLPYLMAAYKETLRLHPAAPIAHRQSSEEMVLRGFTVPPQTAVFI
NIWAIGRDPAFWEDPLAFRPERFMPGGAAESLEPRGQHFHFMPFGSGRRGCPGMGL
ALQSVPAVLAALVQCFDWATAAGEPIDMDESDGLVCARKHPLLLRPTPRLNPFPAV
V*
SEQ ID NO: 132
Sorghum bicolor
Sobic.004G108200
MAMDQPAMPMLMSTDSAAAVMVLLSVATLLLALNHHLLSSWRRRSSRRLPPSPPRL
PVIGHLHLLRPPVHRTFHELATRLGAPLMHIRLGSTHCVVAGTADVARELIRDHDAAI
SGRPVSVLSRLFSYGSAGFAFTPNSRHWRFLRRLCVSEVLGTRTVEQLRHVRRGSLA
ELLRAVRASSARGDAVDVTRELIRFSNTAIIRMVASDAAVTDEAQELVKAVTELLGA
FNLEDYVPLCRGWDLQGLRRKATVVHRRFDAVLEQMIRHKEAARDMERRRRGGSG
TLEDKRVEGPPATTCKQRNKDLLDILLDKAEDETAEVKLTRENMKAFIIDVVTAGSD
SSAVTVEWMLAELMNHPEALGKVRDEIDAVVGGGDGRIVGEADLARLPYLQATFK
ETLRLHPGAPIAHRQSTTEMVVRGFTVPPETAVYINLWAIGRDPSFWEDPLAFRPERF
MPGGAAEGLEPRGGGGGGQQFQFMPFGSGRRGCPGMGLAQQSVPAVLAALVQCFD
WAAADDGETAAIGMDESDVGLVCARKHPLVLRPTARLNPFPAVV*
SEQ ID NO: 133
Sorghum bicolor
Sobic.006G001000
MAAMEEQPLSSSSTSMAIVLSLLKNNPADAVLLALVAVVALRHYLISSWRQQEQAR
RLPPGPRRLPVIGHLHLLRPPVHRTFQELASRMGPLMHIQLGSTHCVVASSPEVASELI
RGHEGSISERPLTAVARQFAYDSAGFAFAPYNTHWRFMKRLCMSELLGPRTVEQLRP
IRRAGTVSLLGDLLLAAASSETETETVVDLTRHLIRLSNTSIIRMVASTVPGSVTDEAQ
ELVKAVAELVGAFNADDYIAVIRGWDLQGLRRRAADVHRRFDALLEDILKHKEEAR
AARRRLDDDDGHRVSKKQATAPHSKDLLDILMDKAEDPAAEVKLTRENIKAFIIDVV
TAGSDTSAAMVEWMLAELLNHPETLRKVVEEIDAVVGGDRIASEADLPQLPYLMAA
YKETLRLHPAAPIAHRQSTDEMVVRGFTVPPQTAVFINVWAIGRDPAYWEEPLAFRP
ERFMPGGAADSLEPRGQHFQYMPFGSGRRGCPGMGLALQSVPAVLAALVQCFHWA
TVDGDGDGDSKIDMSESDGLVCARKKPLLLRPTPRLSPFPAVV*
SEQ ID NO: 134
Zea mays B104
Zm00007a00042926
MAMDLLAMPVLLSADSAAAVLVLLSVATVVALKHLLSSWRRSPRRRLPPSPTPLPVI
GHLHLLRPPVHRTFHELATRLGAPLMHIRLGSTHCVVVGSADVARELIHDHDATISG
RPVSVLSRLFSYGSAGFAFTPYSPHWRFLRRLCVSEVLGPRTVEQLRHVRRGSLVSLL
RSVLASSARGDNKVDLTRELIRFSTTSIIRMVASDVGVTDEAQELVKGVAELLGAFNL
EDYVPLCRGWDLQGLRRKANGVHRRFDAVLEQMIRHKEEARDRERGRGGAAQED
KKGWPATCKQRNKDLLDILLDMAENETAEVKLTRENMKAFIVDVVTAGSDSSAAT
VEWMLAELMNHPEALRKVRAEIDAVVGADRIVGEEDLPRLPYLQATFKETLRLHPG
APIAHRESTGEMVVRGFTVPPRTAVFFNLWAIGRDPSCWEEPLAFRPERFMPGGASE
GLPPRGQQFQFIPFGSGRRACPGMGLAQQSVPTVLAALVQCFDWAAVDGETAAMG
MDESDGGLVCARKHPLVLRPTARLNPFPAVV*
SEQ ID NO: 135
Zea mays B104
Zm00007a00044196
MEEQQPRPRPSIMFVLSSLAKNNPESVLALIAVLTVVALRHLISSWRQQAPLPPSPTSL
PVIGHLHLLRPPVHRTFQELASRIGPLMHIRLGSTHCVVASSPEVASELIRGHEGSISER
PLTAVARQFAYDSAGFAFAPYNTHWRFMKRLCMSELLGPRTVEQLRPIRRAGTVSLL
ADLLASSARGETVDLTRHLIRLSNTSIIRMVASTVPGSVTDEAQEVVKDVAELVGAF
NVDDYISLVRGWDLQGLRRRAAGVHRRFDALLEDILRHKEEARAARRLDQDDDGR
GSSKQDKKQATHSKDLLDILMDKADDPAAEIKLTRENIKAFIIDVVTAGSDTSAAMV
EWMLAELMNHPETLRKVAEEIDAVVGGDRIASEADLPQLPYLMAAYKETLRLHPAA
PIAHRQSSEEMVVRGFTVPPQTAVFINVWAIGRDPAYWEEPLAFRPERFMPGGAAES
LEPRGQHFQYMPFGSGRRGCPGMGLALQSVPAVLAALVQCFHWATVDGDGGVNKI
DMSESDGLVCARKKPLLLRPTPRLTPFPAVV*
SEQ ID NO: 136
Zea mays B104
Zm00007a00044088
MKEQQPRPRPSIMFVLSSLAKNNPEAVLALIAVVTVVALRHLISSWRQQAPLPPSPTS
LPVIGHLHLLRPPVHRTFQXWTRRRTRRRRSSSPRENIKAFIIDVVTAGSDTSAAMVE
WMLAELMNHPETLRKVVEEIDAVVGGDRIASEADLPRLPYLMAAYKETLRLHPAAP
IAHRQSSAEMVVRGFTVPPQTAVFINVWAIGRDPAYWEEPLAFRPERFMPGGAAENL
EPRGQHFQYMPFGSGRRGCPGMGLALQSVPAVLAALVQCFHWATVDGDGGVNKID
MSESDGLVCARKKPLLLRATPRLTPFPAVV*
SEQ ID NO: 137
Zea mays B104
Zm00007a00049351
MEEQQLRARPNMMVLSSLAKNNPEAVLALIAFVTVVALRQLISSWRQHGRLPPGPTS
LPVIGHLHLLRPPVHRTLQELASRIGPLMHIRLGSTNCVVASSPEVVSELIRGHEGSISA
RPFTAVARQFSYDSAGFVFEPYNTHWRFMKRLCMSELLGPRTVEQLRPVRRAVTVS
LVSDLLASSARGETVDITRHLIRLTNTSIIRMVASTVSGSVTDEAHELAKAVIEVVGAF
NVDDYIAVVRGWDLQGLGRKAADVHRRFDALLEDILRHKEEARAARRLDDGHGKQ
ATHSKDLLDILMDKAEDPAAEVKLTRENIKAFVIDVVTSGSDTSAAMAEWMLAELM
NHPETLRKVVEEIDAVVGGGRIASEADLPQLPYLMAVYKETLRLHPAGPIAHRQSTE
EMVVHGFTVPPQSTVLIHVWAIGRDPAYWEEPLLFRPERFMPGGAAESLEPRGKHFQ
YIPFGSGRRGCPGMGLAMQSVPAVVAALVQCFYWATVDGGVNKIDMSESDGLVCA
RKKPLLLRPTSRLTPFPPVV*
SEQ ID NO: 138
Zea mays PH207
Zm00008a021549
MAMDLLAMPVLLSADSAAAVLVLLSVATVVALKHLLSSWRRSPRRRLPPSPTPLPVI
GHLHLLRPPVHRTFHELATRLGAPLMHIRLGSTHCVVVGSADVARELIHDHDATISG
RPVSVLSRLFSYGSAGFAFTPYSPHWRFLRRLCVSEVLGPRTVEQLRHVRRGSLVSLL
RSVLASSARGDNKVDLTRELIRFSTTSIIRMVASDVGVTDEAQELVKGVAELLGAFNL
EDYVPLCRGWDLQGLRRKANGVHRRFDAVLEQMIRHKEEARDRERGRGGAAQED
KKGWPATCKQRNKDLLDILLDMAENETAEVKLTRENMKAFIVETLRLHPGAPIAHR
ESTGEMVVRGFTVPPRTAVFFNLWAIGRDPSCWEEPLAFRPERFMPGGASEGLPPRG
QQFQFIPFGSGRRACPGMGLAQQSVPTVLAALVQCFDWAAVDGETAAMGMDESDG
GLVCARKHPLVLRPTARLNPFPAVV*
SEQ ID NO: 139
Zea mays PH207
Zm00008a037571
MFVLSSLAKNNPESVLALIAVLTVVALRHLISSWRQQARLPPSPTSLPVIGHLHLLRPP
VHRTFQELASRIGPLMHIRLGSTHCVVASTPEVASELIRGHEGSISERPLTAVARQFAY
ETVDLTRHLIRLSNTSIIRMVASTVPGSVTDEAQEVVKDVAELVGAFNVDDYISLVRG
WDLQGLRRRAAGVHRRFDALLEDILRHKEEARAARRLDQDDDGRGSSKQDKKQAT
HSKDLLDILMDKADDPAAEIKLTRENIKAFIIDVVTAGSDTSAAMVEWMLAELMNHP
ETLRKVAEEIDAVVGGDRIASEADLPQLPYLMAAYKETLRLHPAAPIAHRQSSEEMV
VRGFTVPPQTAVFINVWAIGRDPAYWEEPLAFRPERFMPGGAAESLEPRGQHFQYMP
FGSGRRGCPGMGLALQSVPAVLAALVQCFHWATVDGDGGVNKIDMSESDGLVCAR
KKPLLLRPTPRLTPFPAVV*
SEQ ID NO: 140
Zea mays PH207
Zm00001d004555
MKEQQPRPRPSIMFVLSSLAKNNPEAVLALIAVVTVVALRHLISSWRQQAPLPPSPTS
LPVIGHLHLLRPPVHRTFQELASRIGPLMHIRLGSTHCVVASSPEVASELIRGHEGSISE
RPLTAVARQFAYDSAGFAFAPYNTHWRFMKRLCMSELLGPRTVEQLRPIRRAGTVS
LLGDLLASSARGETVDLTRHLIRLSNTSIIRMVASTVPGSVTDEAQKVVKDVAELVG
AFNVDDYIAVVRGWDLQGLRRRAADVHRRFDALLEDILRHKEEARAARRLDQDDG
QGISSKQDKKQATHSKDLLDILMDKAEDQAAEVKLTRENIKAFIIDVVTAGSDTSAA
MVEWMLAELMNHQETLRKVVEEIDAVVGGDRIASEADLPRLPYLMAAYKETLRLH
PAAPIAHRQSSEEMVVRGFTVPPQTAVFINVWAIGRDPAYWEEPLAFRPERFMPGGA
AESLEPRGQHFQYMPFGSGRRGCPGMGLALQSVPAVLAALVQCFHWATVDGDGGV
NKIDMSESDGLVCARKKPLLLRPTPRLTPFPAVV*
SEQ ID NO: 141
Zea mays PH207
Zm00008a008017
MKEQQPRPRPSIMFVLSSLAKNNPEAVLALIAVVTVVALRHLISSWRQQAPLPPSPTS
LPVIGHLHLLRPPVHRTFQELASRIGPLMHIRLGSTHCVVASSPEKVVKDVAELVGAF
NVDDYIAVVRGWDLQGLRRRAADVHRRFDALLEDILRHKEEARAARRLDQDDGQG
ISSKQDKKQATHSKDLLDILMDKAEDQAAEVKLTRENIKAFIIDVVTAGSDTSAAMV
EWMLAELMNHPETLRKVVEEIDAVVGGDRIASEADLPRLPYLMAAYKETLRLHPAA
PIAHRQSSAEMVVRGFTVPPQTAVFINVWAIGRDPAYWEEPLAFRPERFMPGGAAEN
LEPRGQHFQYMPFGSGRRGCPGMGLALQSVPAVLAALVQCFHWATVDGDGGVNKI
DMSESDGLVCARKKPLLLRATPRLTPFPAVV*
SEQ ID NO: 142
Zea mays PH207
Zm00008a037570
MMVLSSLAKNNPEAVLALIAFVTVVALRHLISSWRQHGRLPPGPTSLPVIGHLHLLRP
PVHRTLQELASRIGPLMHIRLGSTNCVVASSPEVASELIRGHEGSISARPFTAVARKFS
YDSAGFVFEPYNTHWRFMKRLCMSELLGPRTVEQLRPVRRAVTVSLVSDLLASSAR
GETVDITRHLIRLTNTSIIRMVASTVSGSVTDEAHELAKAVIEVVGAFNVDDYIAVVR
GWDFQGLGRKAADVHRRFDALLEDILRHKEEARAARRLDDGHGKQATHSKDLLDI
LMDKAEDPAAEVKLTRENIKAFVIDVVTSGSDTSAAMAEWMLAELMNHPETLRKV
VEEIDAVVGGGRIASEADLPQLPYLMAVYKETLRLHPAGPIAHRQSTEEMVVHGFTV
PPQSTVLIHVWAIGRDPAYWEEPLLFRPERFMPGGAAESLEPRGKHFQYIPFGSGRRG
CPGMGLAMQSVPAVVAALVQCFHWSTVDGGMDKIDMSESDGLVCARKKPLLLRPT
SRLTPFPPVV*
SEQ ID NO: 143
Zea mays B73
Zm00001d016151
MAMDLLAMPVLLSADSAAAVLVLLSVATVVALKHLLSSWRRSPRRRLPPSPTPLPVI
GHLHLLRPPVHRTFHELATRLGAPLMHIRLGSTHCVVVGSADVARELIHDHDATISG
RPVSVLSRLFSYGSAGFAFTPYSPHWRFLRRLCVSEVLGPRTVEQLRHVRRGSLVSLL
RSVLASSARGDNKVDLTRELIRFSTTSIIRMVASDVGVTDEAQELVKGVAELLGAFNL
EDYVPLCRGWDLQGLRRKANGVHRRFDAVLEQMIRHKEEARDRERSRGGAAQEDK
KGWPATCKQRNKDLLDILLDMAENETAEVKLTRENMKAFIVATFKETLRLHPGAPIA
HRESTGEMVVRGFTVPPRTAVFFNLWAIGRDPSCWEEPLAFRPERFMPGGASEGLPP
RGQQFQFIPFGSGRRACPGMGLAQQSVPTVLAALVQCFDWAAVDGETAAMGMDES
DGGLVCARKHPLVLRPTARLNPFPAVV*
SEQ ID NO: 144
Zea mays B73
Zm00001d024946
MEEQQPRPRPSIMFVLSSLAKNNPESVLALIAVLTVVALRHLISSWRQQARLPPSPTSL
PVIGHLHLLRPPVHRTFQELASRIGPLMHIRLGSTHCVVASTPEVASELIRGHEGSISER
PLTAVARQFAYDSAGFAFAPYNTHWRFMKRLCMSELLGPRTVEQLRPVRRAGTVSL
LADLLASSARGETVDLTRHLIRLSNTSIIRMVASTVPGSVTDEAQEVVKDVAELVGAF
NVDDYISLVRGWDLQGLRRRAAGVHRRFDALLEDILRHKEEARAARRLDQDDDGR
GSSKQDKKQATHSKDLLDILMDKADDPAAEIKLTRENIKAFIIDVVTAGSDTSAAMV
EWMLAELMNHPETLRKVAEEIDAVVGGDRIASEADLPQLPYLMAAYKETLRLHPAA
PIAHRQSSEEMVVRGFTVPPQTAVFINVWAIGRDPAYWEEPLAFRPERFMPGGAAES
LEPRGQHFQYMPFGSGRRGCPGMGLALQSVPAVLAALVQCFHWATVDGDGGVNKI
DMSESDGLVCARKKPLLLRPTPRLTPFPAVV*
SEQ ID NO: 145
Zea mays B73
Zm00001d024943
MEEQQLRARPNMMVLSSLAKNNPEAVLALIAFVTVVALRHLISSWRQHGRLPPGPTS
LPVIGHLHLLRPPVHRTLQELASRIGPLMHIRLGSTNCVVASSPEVASELIRGHEGSISA
RPFTAVARKFSYDSAGFVFEPYNTHWRFMKRLCMSELLGPRTVEQLRPVRRAVTVS
LVSDLLASSARGETVDITRHLIRLTNTSIIRMVASTVSGSVTDEAHELAKAVIEVVGAF
NVDDYIAVVRGWDFQGLGRKAADVHRRFDALLEDILRHKEEARAARRLDDGHGKQ
ATHSKDLLDILMDKAEDPAAEVKLTRENIKAFVIDVVTSGSDTSAAMAEWMLAELM
NHPETLRKVVEEIDAVVGGGRIASEADLPQLPYLMAVYKETLRLHPAGPIAHRQSTE
EMVVHGFTVPPQSTVLIHVWAIGRDPAYWEEPLLFRPERFMPGGAAESLEPRGKHFQ
YIPFGSGRRGCPGMGLAMQSVPAVVAALVQCFYWATVDGGVDKIDMSESDGLVCA
RKKPLLLRPTSRLTPFPPVV*

1. A method of increasing the ability of a crop plant to assimilate atmospheric nitrogen, the method comprising modifying the expression of a gene involved in flavone biosynthesis or degradation in one or more cells of the plant such that the plant produces an increased amount of one or more flavones, wherein the one or more flavones are exuded from the plant's roots.

2. The method of claim 1, wherein the one or more flavones induces biofilm formation in N₂-fixing bacteria present in the soil in proximity to the plant's roots.

3. The method of claim 1, wherein the biofilm formation leads to an increase in the ability of the bacteria to fix atmospheric nitrogen, and wherein the fixed atmospheric nitrogen is assimilated by the plant.

4. The method of claim 1, wherein at least one of the one or more flavones is glycosylated.

5. The method of claim 1, wherein the one or more flavones comprise apigenin, apigenin-7-glucoside, or luteolin.

6. The method of claim 1, wherein the expression of the gene in the one or more cells of the plant is modified by editing an endogenous copy of the gene.

7. The method of claim 6, wherein the endogenous copy of the gene is modified by introducing into one or more cells of the plant a guide RNA targeting the gene and an RNA-guided nuclease.

8. The method of claim 7, further comprising introducing into the one or more cells a donor template comprising sequences homologous to the genomic region surrounding the target site of the guide RNA, wherein the RNA-guided nuclease cleaves the DNA at the target site and the DNA is repaired using the donor template.

9. The method of claim 7, wherein the RNA-guided nuclease is Cas9 or Cpf1.

10. The method of claim 6, wherein the endogenous copy of the gene is modified so as to reduce or eliminate its expression.

11. The method of claim 10, wherein the endogenous copy of the gene is deleted.

12. The method of claim 10, wherein the gene is CYP75B3 or CYP75B4, or a homolog or ortholog thereof.

13. The method of claim 12, wherein the gene comprises a nucleotide sequence that is substantially identical (sharing at least 50%, 55%, 60%, 65%, 70%, 75%, 80%, 85%, 90%, 95%, 96%, 97%, 98%, 99% or more identity) to any one of SEQ ID NOS: 2, 4, 6 or 8, or encodes a polypeptide comprising an amino acid sequence that is substantially identical (sharing at least 50%, 55%, 60%, 65%, 70%, 75%, 80%, 85%, 90%, 95%, 96%, 97%, 98%, 99% or more identity) to any one of SEQ ID NOS: 1, 3, 5, 7, or 14-120.

14. The method of claim 7, wherein the guide RNA comprises a target sequence that is substantially identical (e.g., comprising 0, 1, 2, or 3 mismatches) to any one of SEQ ID NOS:11-13.

15. The method of claim 7, wherein the guide RNA comprises a target sequence that is substantially identical (e.g., comprising 0, 1, 2, or 3 mismatches) to a sequence within SEQ ID NO: 9 or SEQ ID NO:10.

16. The method of claim 6, wherein the endogenous copy of the gene is modified so as to increase its expression.

17. The method of claim 16, wherein the endogenous copy of the gene is modified by replacing the endogenous promoter with a heterologous promoter.

18. The method of claim 17, wherein the heterologous promoter is an inducible promoter.

19. The method of claim 17, wherein the heterologous promoter is a constitutive promoter.

20. The method of claim 17, wherein the heterologous promoter is a tissue- or organ-specific promoter.

21. The method of claim 20, wherein the organ is the root and/or the tissue is a root tissue.

22. The method of claim 16, wherein the gene is CYP 93G1, or a homolog or ortholog thereof.

23. The method of claim 22, wherein the gene encodes a polypeptide comprising an amino acid sequence that is substantially identical (sharing at least 50%, 55%, 60%, 65%, 70%, 75%, 80%, 85%, 90%, 95%, 96%, 97%, 98%, 99% or more identity) to any one of SEQ ID NOS: 121-145.

24. The method of claim 1, further comprising generating a stable plant line from the one or more cells of the plant.

25. The method of claim 1, wherein the crop plant is a grain crop.

26. The method of claim 25, wherein the grain crop is rice.

27. The method of claim 1, wherein the plant is selected from the group consisting of corn, wheat, rice, soy, cotton, canola, and sugarcane.

28. A genetically modified crop plant produced using the method of claim 1.

29. A genetically modified crop plant comprising:
i) a mutation or deletion in a CYP75B3 or CYP75B4 gene, or homolog or ortholog thereof, that causes a reduced amount of CYP75B3 or CYP75B4 enzyme and/or enzymatic activity compared to a wild-type plant without the mutation or deletion in the CYP75B3 or CYP75B4 gene; or
ii) an expression cassette comprising a polynucleotide encoding a CYP 93G1 gene, or a homolog or ortholog thereof, operably linked to a promoter, such that the plant comprises an increased amount of CYP93G1 enzyme and/or enzymatic activity compared to a wild-type plant without the expression cassette; wherein
the genetically modified crop plant produces an increased amount of one or more flavones as compared to a wild-type plant that is not genetically modified, wherein the one or more flavones are exuded from the genetically modified crop plant's roots.

30. The genetically modified crop plant of claim 28, wherein the crop plant is selected from the group consisting of corn, wheat, rice, soy, cotton, canola, and sugarcane.

31. A method of increasing the assimilation of atmospheric nitrogen in a grain crop plant grown under reduced inorganic nitrogen conditions, the method comprising:
providing a genetically modified crop plant in which the expression of a gene involved in flavone biosynthesis or degradation has been modified in one or more cells such that the roots of the plant exude greater amounts of one or more flavones than a wild-type plant; and
growing the plant in soil comprising an amount of inorganic nitrogen that is lower than a standard or recommended amount for the crop plant.

32. The method of claim 31, wherein the amount of inorganic nitrogen is less than 50% of the standard or recommended amount for the crop plant.

33. The method of claim 31, wherein the crop plant is rice, and wherein the amount of inorganic nitrogen in the soil is less than 50 ppm.

34. The method of claim 32, wherein the amount of inorganic nitrogen in the soil is about 25 ppm.

35. The method of claim 31, wherein the genetically modified plant is the plant comprises:
i) a mutation or deletion in a CYP75B3 or CYP75B4 gene, or homolog or ortholog thereof, that causes a reduced amount of CYP75B3 or CYP75B4 enzyme and/or enzymatic activity compared to a wild-type plant without the mutation or deletion in the CYP75B3 or CYP75B4 gene; or
ii) an expression cassette comprising a polynucleotide encoding a CYP 93G1 gene, or a homolog or ortholog thereof, operably linked to a promoter, such that the plant comprises an increased amount of CYP93G1 enzyme and/or enzymatic activity compared to a wild-type plant without the expression cassette; wherein
the genetically modified crop plant produces an increased amount of one or more flavones as compared to a wild-type plant that is not genetically modified, wherein the one or more flavones are exuded from the genetically modified crop plant's roots.

36. The method of claim 31, wherein the crop plant is selected from the group consisting of corn, wheat, rice, soy, cotton, canola, and sugarcane.

37. The method of claim 31, wherein N₂-fixing bacteria in the soil in which the genetically modified plant is grown show greater biofilm formation than control N₂-fixing bacteria in soil in which a wild-type plant is grown.

38. The method of claim 37, wherein N₂-fixing bacteria in the soil in which the genetically modified plant is grown show greater adherence to the root surface and/or inside the root tissue of the plant than control N₂-fixing bacteria in soil in which a wild-type plant is grown.

39. The method of claim 31, wherein the crop plant is a grain crop, and wherein the number of tillers, tassels, or spikes in the genetically modified plant grown in the soil comprising the reduced amount of inorganic nitrogen is at least 30% greater than in a wild-type plant grown in equivalent soil.

40. The method of claim 31, wherein the number of grain or seed-bearing organs and/or the seed yield in the genetically modified plant grown in the soil comprising the reduced amount of inorganic nitrogen is at least 30% greater than in a wild-type plant grown in equivalent soil.

41. The method of claim 31, wherein the genetically modified plant grown in the soil comprising the reduced amount of inorganic nitrogen assimilates at least twice the amount of atmospheric nitrogen than the amount assimilated by a wild-type plant grown in equivalent soil.
